(19) United States
(12) Patent Application Publication
(10) Pub. No.: US 2016/0297854 A1
(43) Pub. Date: Oct. 13, 2016

(54) COMPOSITIONS AND METHODS FOR LONG ACTING MOLECULES

(71) Applicants: Joy GHOSH, Boston, MA (US); Michael ROGUSKA, Ashland, MA (US); Andrew Anh NGUYEN, Brookline, MA (US); Thomas PIETZONKA, Basel (CH); Matthias MACHACEK, Allschwil (CH); Andrei GOLOSOV, Cambridge, MA (US)

(72) Inventors: Joy GHOSH, Boston, MA (US); Michael ROGUSKA, Ashland, MA (US); Andrew Anh NGUYEN, Brookline, MA (US); Thomas PIETZONKA, Basel (CH); Matthias MACHACEK, Allschwil (CH); Andrei GOLOSOV, Cambridge, MA (US)

(73) Assignee: Novartis AG, Basel (CH)

(21) Appl. No.: 15/186,025

(22) Filed: Jun. 17, 2016

Related U.S. Application Data
(63) Continuation of application No. 14/109,426, filed on Dec. 17, 2013, now abandoned.
(60) Provisional application No. 61/738,488, filed on Dec. 18, 2012.

(51) Int. Cl. C07K 14/00; A61K 31/713; A61K 47/48; C07K 16/22; C12N 15/115

(52) U.S. Cl. CPC C07K 14/00; C07K 16/22; C12N 15/115; A61K 47/48246; A61K 31/713; C12N 2310/3513; C07K 2317/55; C07K 2317/565; C07K 2317/56; C07K 2317/51; C07K 2317/515; A61K 38/00

(57) ABSTRACT

The invention relates, in part, to compositions and methods that utilize a peptide tag that binds to hemagglutanin (HA). The HA tag can be linked to a molecule such as a protein or nucleic acid which, when administered to the eye, results in an increase in ocular half-life and/or mean residence time, and or a decrease in ocular clearance of the protein or nucleic acid. The invention also encompasses methods for treating ocular disease, including retinal vascular disease, by administering a protein or nucleic acid linked to an HA peptide tag.

### SEQUENCE LISTING

The instant application contains a Sequence Listing which has been submitted electronically in ASCII format and is hereby incorporated by reference in its entirety. Said ASCII copy, created on Feb. 17, 2014, is named PAT055248-US-NP_SL.txt and is 285,061 bytes in size.

### BACKGROUND OF THE INVENTION

Retinal diseases including neovascular (wet) AMD, diabetic retinopathy, and retinal vein occlusions have an angiogenic component that leads to loss of vision. Clinical trials have demonstrated that these diseases can be treated effectively with monthly intravitreal injections of an anti-VEGF therapy such as ranibizumab or bevacizumab or bi-monthly treatment with aflibercept. Despite the efficacy of these therapies, monthly or bi-monthly treatment is a significant health-care burden for patients and physicians (Oishi et al. (2011) Eur J Ophthalmol. November-December; 21(6):777-82.). Thus there is a need for an ocular therapy that can be delivered less frequently, yet still provide the same treatment benefit seen with monthy or bi-monthly treatment with these agents.

The eye is a complex tissue that has several distinct compartments including the cornea, aqueous humor, lens, vitreous humor, retina, the retinal pigment epithelium, and choroid. The composition of these compartments varies, but they are generally described in literature to consist of cells, and include extracellular macromolecules such as hyaluronic acid. The present invention describes peptide tags that binds hyaluronic acid in the vitreous enabling the molecules to which they are linked to have longer ocular half-life, longer ocular retention and a longer duration of action in ocular diseases.

The present invention provides peptide tags that can be linked to a therapeutic molecule in order to decrease the clearance of the therapeutic molecule from the eye, thereby increasing its ocular half-life. For example, peptide tagged molecules are described herein with increased duration of efficacy in the eye relative to an untagged molecule, which clinically will lead to less frequent intraocular injections and improved patient treatment.

### SUMMARY OF THE INVENTION

The present invention relates to peptide tags, as described herein, that bind hyaluronan (HA) in an eye. In certain aspects the invention relates to a peptide tag, as described herein, that bind hyaluronan (HA) in an eye with a K_{D} of less than or equal to 9.0 uM. For example, the peptide tag can bind HA with a K_{D} of less than or equal to 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In one aspect the peptide tag binds HA with a KD of less than or equal to 9.0 uM. In one aspect the peptide tag binds HA with a KD of less than or equal to 8.0 uM. In one aspect the peptide tag binds HA with a KD of less than or equal to 7.2 uM. In one aspect the peptide tag binds HA with a KD of less than or equal to 5.5 uM. The invention also relates to an isolated peptide tag that binds, or is capable of binding, HA comprising the sequence of SEQ ID NO: 32, SEQ ID NO: 33, SEQ ID NO: 34, SEQ ID NO: 35 or SEQ ID NO: 36.

The present invention also relates to a peptide tagged molecule comprising one or more peptide tags linked to a protein or nucleic acid, where the peptide tag comprises the sequence of SEQ ID NO: 32, SEQ ID NO: 33, SEQ ID NO: 34, SEQ ID NO: 35 or SEQ ID NO: 36. Where a peptide tag is linked to a protein, the tag can be linked to an amino acid of such protein. Where the peptide tag is linked to a nucleic acid, the tag can be linked to a nucleotide of such nucleic acid. In certain aspects is it contemplated that the peptide tag is linked to the N-terminus and/or C-terminus of a protein molecule or at the 5′ and/or 3′ end of a nucleic acid. In addition the peptide tag may be linked directly to the protein or nucleic acid, or the peptide tag may be linked indirectly to the protein or nucleic acid via a linker. It is contemplated that the peptide tagged molecules described herein may be useful as a medicament.

In certain aspects of the invention the peptide tagged molecule comprises a peptide tag linked to protein, for example, an antibody, or antigen binding fragment, a therapeutic protein, a protein receptor, or a designed-ankyrin repeat protein (DARPin). In certain aspects of the invention the peptide tagged molecule comprises a peptide tag linked to an aptamer. It is contemplated that the peptide tagged molecule binds VEGF, C5, Factor P, Factor D, EPO, EPOR, IL-1β, IL-17A, TNFα, FGFR2 and/or PDGF-BB.

The present invention also relates to a peptide tagged molecule comprising an isolated antibody or antigen binding fragment that binds VEGF and comprises heavy chain CDR1, 2, and 3 sequences of SEQ ID NOs: 1, 2 and 3, respectively and light chain CDR1, 2, and 3 sequences of SEQ ID NOs: 11, 12 and 13, respectively. The present invention also relates to a peptide tagged molecule comprising an isolated antibody or antigen binding fragment that binds C5 and comprises heavy chain CDR1, 2, and 3 sequences of SEQ ID NOs: 37, 38, and 39 respectively and light chain CDR1, 2, and 3 sequences of SEQ ID NOs: 46, 47, and 48, respectively. The present invention also relates to a peptide tagged molecule comprising an isolated antibody or antigen binding fragment that binds Factor P and comprises heavy chain CDR1, 2, and 3 sequences of SEQ ID NOs: 53, 54, and 55 respectively and light chain CDR1, 2, and 3 sequences of SEQ ID NOs: 65, 66, and 67, respectively. The present invention also relates to a peptide tagged molecule comprising an isolated antibody or antigen binding fragment that binds EPO and comprises heavy chain CDR1, 2, and 3 sequences of SEQ ID NOs: 75, 76, and 77 respectively and light chain CDR1, 2, and 3 sequences of SEQ ID NOs: 86, 87, and 88, respectively. The present invention also relates to a peptide tagged molecule comprising an isolated antibody or antigen binding fragment that binds TNFα and comprises heavy chain CDR1, 2, and 3 sequences of SEQ ID NOs: 108, 109, and 110 respectively and light chain CDR1, 2, and 3 sequences of SEQ ID NOs: 117, 118, and 119, respectively. The present invention also relates to a peptide tagged molecule comprising an isolated antibody or antigen binding fragment that binds IL-1β and comprises heavy chain CDR1, 2, and 3 sequences of SEQ ID NOs: 189, 190, and 191 respectively and light chain CDR1, 2, and 3 sequences of SEQ ID NOs: 198, 199, and 200, respectively.

The present invention also relates to a peptide tagged molecule comprising an isolated antibody or antigen binding fragment further comprising a variable heavy chain domain and a variable light chain domain having the sequences of SEQ ID NO: 7 and SEQ ID NO: 17, respectively; SEQ ID NO: 40 and SEQ ID NO: 49, respectively; SEQ ID NO: 59 and SEQ ID NO: 71, respectively; SEQ ID NO: 81 and SEQ ID NO: 92, respectively; SEQ ID NO: 111 and SEQ ID NO: 120, respectively; or SEQ ID NO: 193 and SEQ ID NO: 201, respectively. In certain aspects, the invention relates to a peptide tagged molecule comprising an isolated antibody or antigen binding fragment having a heavy chain and a light chain sequence of SEQ ID NO: 9 and SEQ ID NO: 19, respectively; SEQ ID NO: 42 and SEQ ID NO: 51, respectively; SEQ ID NO: 61 and SEQ ID NO: 73, respectively; SEQ ID NO: 83 and SEQ ID NO: 85, respectively; SEQ ID NO: 113 and SEQ ID NO: 122, respectively; SEQ ID NO: 194 and SEQ ID NO: 202, respectively. More specifically, the peptide tagged molecule comprises, respectively, the tagged heavy chain sequence and light chain sequence of SEQ ID NOs: 21 and 19; SEQ ID NOs: 23 and 19; SEQ ID NOs: 25 and 19; SEQ ID NOs: 27 and 19; SEQ ID NOs: 29 and 19; SEQ ID NOs: 44 and 51; SEQ ID NOs: 63 and 73; SEQ ID NOs: 85 and 95; SEQ ID NOs: 115 and 122; or SEQ ID NOs: 196 and 202.

The present invention also relates to a peptide tag or peptide tagged molecule as described in Tables 1, 2, 8, 8b, 9 or 9b. More specifically, in certain aspects the peptide tagged molecule is NVS1, NVS2, NVS3, NVS36, NVS37, NVS70T, NVS71T, NVS72T, NVS73T, NVS74T, NVS75T, NVS76T, NVS77T, NVS78T, NVS80T, NVS81T, NVS82T, NVS83T, NVS84T, NVS1b, NVS1c, NVS1d, NVS1e, NVS1f, NVS1g, NVS1h or NVS1j.

The invention also relates to compositions comprising the peptide tag, for example a peptide tag having the sequence of SEQ ID NO: 32, SEQ ID NO: 33, SEQ ID NO: 34, SEQ ID NO: 35 or SEQ ID NO: 36. The invention further relates to peptide tagged molecules as described herein, specifically peptide tagged molecules comprising a peptide tag having the sequence of SEQ ID NO: 32, SEQ ID NO: 33, SEQ ID NO: 34, SEQ ID NO: 35 or SEQ ID NO: 36. In certain aspects the compositions described herein further comprise a pharmaceutically acceptable excipient, diluent or carrier. It is also contemplated that the compositions may be formulated for ocular delivery (e.g., intraocular). In certain aspects the compositions for ocular delivery may comprise a peptide tag that binds HA with a KD of less than or equal to 9.0 uM. For example, the peptide tag can bind HA with a KD of less than or equal to, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In one aspect the peptide tag binds HA with a KD of less than or equal to 9.0 uM. In one aspect the peptide tag binds HA with a KD of less than or equal to 8.0 uM. In one aspect the peptide tag binds HA with a KD of less than or equal to 7.2 uM. In one aspect the peptide tag binds HA with a KD of less than or equal to 5.5 uM. In certain aspects the composition includes 12 mg or less of the peptide tagged molecule. In a further aspect, the composition is formulated to deliver 12 mg/eye or less of a peptide tagged molecule per dose. In certain aspects the compositions described herein comprise 6 mg/50 ul or less of a peptide tagged molecule. In certain aspects of the invention it is contemplated that the composition includes 12 mg or less of the peptide tag.

Another aspect of the invention provides for a nucleic acid molecule encoding a peptide tag comprising a sequence of SEQ ID NO: 33, SEQ ID NO: 34, SEQ ID NO: 35 or SEQ ID NO: 36. More specifically, the nucleic acid molecule may encode the peptide tag HA10.1, HA10.2, HA11 or HA11.1. Further aspects of the invention provide for a nucleic acid molecule encoding peptide tagged molecule as described Tables 1, 2, 8, 8b, 9, or 9b. In certain aspects the nucleic acid molecule may encode NVS1, NVS2, NVS3, NVS36, NVS37, NVS70T, NVS71T, NVS72T, NVS73T, NVS74T, NVS75T, NVS76T, NVS77T, NVS78T, NVS80T, NVS81T, NVS82T, NVS83T, NVS84T, NVS1 b, NVS1c, NVS1 d, NVS1e, NVS1f, NVS1g, NVS1h or NVS1j. In certain specific aspects the nucleic acid comprises the sequence SEQ ID NO: 10, 20, 22, 24, 26, 28, and/or 30.

The present invention relates to expression vectors comprising the nucleic acids described herein. More specifically, for example, the expression vectors may comprise nucleic acids as described in Tables 1 and 2. In certain aspects the invention further provide a host cell comprising one or more expression vectors as described herein, wherein the host cell may be used for the production of a peptide tag having a sequence of SEQ ID NO: 33, SEQ ID NO: 34, SEQ ID NO: 35 or SEQ ID NO: 36. Alternatively, a host cell comprising one or more expression vectors as described herein may be used for the production of a peptide tagged molecule as described in Tables 1, 2, 8, 8b, 9 or 9b. In certain aspects it is contemplated that the host cell is a mammalian cell.

It is contemplated that the host cells described herein are useful for producing the peptide tags and peptide tagged molecules of the invention. Thus, the invention further relates to a process for producing a peptide tag and/or a peptide tagged molecule as described herein, for example a peptide tag or peptide tagged molecule as described in Tables 1, 2, 8, 8b 9, or 9b. It is contemplated that the process further includes a step of culturing the host cell under appropriate conditions for the production of a peptide tag or peptide tagged molecule, and further isolating the peptide tag or peptide tagged molecule.

The invention still further relates to compositions comprising the peptide tag or peptide tagged molecules described herein. It is also contemplated that the peptide tag, peptide tagged molecules and/or compositions may be useful for therapy, more specifically for ocular therapy. In addition, the peptide tag, peptide tagged molecules and/or compositions may be useful for treating a condition or disorder associated with retinal vascular disease in a subject. In certain aspects, the retinal vascular disease may be neovascular age-related macular degeneration (wet AMD), diabetic retinopathy, diabetic macular edema, proliferative diabetic retinopathy, non-proliferative diabetic retinopathy, macular edema, retinal vein occlusion, multifocal choroiditis, myopic choroidal neovascularization or retinopathy of prematurity. Alternatively, the peptide tag, peptide tagged molecules and/or compositions may be useful for treating a condition or disorder associated with macular edema in a subject. In certain aspects, the condition or disorder associated with macular edema is diabetic retinopathy, diabetic macular edema, proliferative diabetic retinopathy, non-proliferative diabetic retinopathy, neovascular age-related macular degeneration, retinal vein occlusion, multifocal choroiditis, myopic choroidal neovascularization, or retinopathy of prematurity.

In certain specific aspects of the invention compositions comprising a peptide tagged molecules comprising an anti-VEGF antibody or antigen binding fragment thereof may be useful for treating a VEGF-mediated disorder in a subject. In certain aspects, the VEGF-mediated disorder may be age-related macular degeneration, neovascular glaucoma, diabetic retinopathy, macular edema, diabetic macular edema, pathologic myopia, retinal vein occlusions, retinopathy of prematurity, retrolental fibroplasia, abnormal vascular proliferation associated with phakomatoses, edema (such as that associated with brain tumors), Meigs' syndrome, rheumatoid arthritis, psoriasis and atherosclerosis. In certain specific aspects, the composition useful for treating VEGF mediated disorders comprises an anti-VEGF antibody or antigen binding fragment comprising heavy chain CDR1, 2, and 3 sequences of SEQ ID NOs: 1, 2 and 3, respectively and light chain CDR1, 2, and 3 sequences of SEQ ID NOs: 11, 12 and 13, respectively.

The invention also relates to a method of treating a condition or disorder associated with retinal vascular disease in a subject, wherein the method comprises administering to the subject a composition comprising the peptide tag and/or peptide tagged molecule described herein. In certain specific aspects the method comprises administering a composition comprising a peptide tag or peptide tagged molecule, wherein the peptide tag binds HA with a KD of less than or equal to 9.0 uM. For example, the peptide tag can bind HA with a KD of less than or equal to, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In certain specific aspects the peptide tag binds HA with a KD of less than or equal to 8.0 uM. In certain specific aspects the peptide tag binds HA with a KD of less than or equal to 7.2 uM. In certain specific aspects the peptide tag binds HA with a KD of less than or equal to 5.5 uM.

In certain aspects, the condition or disorder associated with retinal vascular disease is neovascular age-related macular degeneration (wet AMD), diabetic retinopathy, diabetic macular edema, proliferative diabetic retinopathy, non-proliferative diabetic retinopathy, macular edema, retinal vein occlusion, multifocal choroiditis, myopic choroidal neovascularization or retinopathy of prematurity.

The invention further relates to a method of treating a condition or disorder associated with macular edema in a subject, wherein the method comprises administering to the subject a composition comprising a peptide tag and/or peptide tagged molecule as described herein. In certain specific aspects the method comprises administering a composition comprising a peptide tag or peptide tagged molecule, wherein the peptide tag binds HA with a KD of less than or equal to 9.0 uM. For example, the peptide tag can bind HA with a KD of less than or equal to, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In one aspect the peptide tag binds HA with a KD of less than or equal to 8.0 uM. In one aspect the peptide tag binds HA with a KD of less than or equal to 7.2 uM. In one aspect the peptide tag binds HA with a KD of less than or equal to 5.5 uM. In certain aspects, the condition or disorder associated with macular edema is diabetic retinopathy, diabetic macular edema, proliferative diabetic retinopathy, non-proliferative diabetic retinopathy, neovascular age-related macular degeneration, retinal vein occlusion, multifocal choroiditis, myopic choroidal neovascularization, or retinopathy of prematurity.

The invention further relates to a method of treating a VEGF-mediated disorder in a subject, wherein the method comprises the step of administering to the subject a composition comprising a peptide tag that binds HA with a KD of less than or equal to 9.0 uM linked to an anti-VEGF antibody or antigen binding fragment thereof. For example, the peptide tag can bind HA with a KD of less than or equal to, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In one aspect the peptide tag binds HA with a KD of less than or equal to 8.0 uM. In one aspect the peptide tag binds HA with a KD of less than or equal to 7.2 uM. In one aspect the peptide tag binds HA with a KD of less than or equal to 5.5 uM. In certain aspects the method relates to treating a VEGF-mediated disorder in the eye of a subject. The invention still further relates to a method of treating a VEGF-mediated disorder in a subject, wherein the method comprises the step of administering to the subject a composition comprising a peptide tag comprising a sequence of SEQ ID NO: 33, SEQ ID NO: 34, SEQ ID NO: 35 or SEQ ID NO: 36 linked to an anti-VEGF antibody or antigen binding fragment thereof. It is contemplated that the anti-VEGF antibody or antigen binding fragment thereof comprises heavy chain CDR1, 2, and 3 sequences of SEQ ID NOs: 1, 2 and 3, respectively and light chain CDR1, 2, and 3 sequences of SEQ ID NOs: 12, 13 and 14, respectively. In certain specific aspects, the VEGF-mediated disorder is age-related macular degeneration, neovascular glaucoma, diabetic retinopathy, macular edema, diabetic macular edema, pathologic myopia, retinal vein occlusions, retinopathy of prematurity, retrolental fibroplasia, abnormal vascular proliferation associated with phakomatoses, edema (such as that associated with brain tumors), Meigs' syndrome, rheumatoid arthritis, psoriasis and atherosclerosis.

The invention also relates to a method of increasing half-life, mean residence time, or terminal concentration of molecule in the eye or decreasing clearance of a molecule from the eye comprising the step of administering a composition comprising a peptide tagged molecule to the eye of the subject, wherein the peptide tag binds HA with a KD of less than or equal to 9.0 uM. For example, the peptide tag can bind HA with a Kd of less than or equal to 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In one aspect the peptide tag binds HA with a KD of less than or equal to 9.0 uM. In one aspect the peptide tag binds HA with a KD of less than or equal to 8.0 uM. In one aspect the peptide tag binds HA with a KD of less than or equal to 7.2 uM. In one aspect the peptide tag binds HA with a KD of less than or equal to 5.5 uM.

The invention also relates to methods of increasing the ocular half-life of a molecule comprising the step of linking the molecule to a peptide tag that binds HA with a KD of less than or equal to 9.0 uM. In certain aspects the invention relates to methods of increasing the ocular mean residence time of a molecule comprising the step of linking the molecule to a peptide tag that binds HA with a KD of less than or equal to 9.0 uM. In certain aspects the invention relates to methods of increasing the ocular terminal concentration of a molecule comprising the step of linking the molecule to a peptide tag that binds HA with a KD of less than or equal to 9.0 uM. In certain aspects the invention relates to methods of decreasing the ocular clearance of a molecule comprising the step of linking the molecule to a peptide tag that binds HA with a KD of less than or equal to 9.0 uM. In each of the foregoing methods, the peptide tag binds HA with a KD of less than or equal to 9.0 uM, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In one aspect, the peptide tag binds HA with a KD of less than or equal to 9.0 uM. In one aspect, the peptide tag binds HA with a KD of less than or equal to 8.0 uM. In one aspect, the peptide tag binds HA with a KD of less than or equal to 7.2 uM. In one aspect, the peptide tag binds HA with a KD of less than or equal to 5.5 uM. In one aspect, the peptide tag comprises the sequence of SEQ ID NO: 32, SEQ ID NO: 33, SEQ ID NO: 34, SEQ ID NO: 35 or SEQ ID NO: 36.

The invention further relates to a method of producing a composition for ocular delivery comprising the step of linking a peptide tag that binds HA with a KD of less than or equal to 9.0 uM to a molecule that binds a target in the eye. For example, the epeptide tag can bind HA with a KD of less than or equal to 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. The invention still further relates to a method of making a peptide tagged molecule comprising a sequence of SEQ ID NO: 32, SEQ ID NO: 33, SEQ ID NO: 34, SEQ ID NO: 35 or SEQ ID NO: 36 is linked to a molecule, for example, a protein or nucleic acid. In certain aspects it is contemplated that linking the peptide tag to a molecule creates a peptide tagged molecule, that when administered to the eye, has a decreased ocular clearance, increased ocular mean residence time, and/or increased ocular terminal concentration compared to the molecule without the tag.

### DEFINITIONS

Unless defined otherwise, all technical and scientific terms used herein have the same meaning as commonly understood by those of ordinary skill in the art to which this invention pertains.

The term “antibody” as used herein means a whole antibody. A whole antibody is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen binding fragment” of an antibody, as used herein, refers to one or more fragments of an antibody that retain the ability to specifically bind to a given antigen (e.g., vascular endothelial cell growth factor: VEGF). Antigen binding functions of an antibody can be performed by fragments of an intact antibody. Examples of binding fragments encompassed within the term antigen binding fragment of an antibody include, but are not limited to, a Fab fragment, a monovalent fragment consisting of the VL, VH, CL and CH1 domains; a F(ab)₂ fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody (scFv); a single domain antibody (dAb) fragment (Ward et al., 1989 Nature 341:544-546), which consists of a VH domain or a VL domain; and an isolated complementarity determining region (CDR).

Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies may include one or more antigen binding fragments of an antibody. These antigen binding fragments are obtained using conventional techniques known to those of skill in the art, and the fragments are screened for utility in the same manner as are intact antibodies.

Antigen binding fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136). Antigen binding portions of antibodies can be grafted into scaffolds based on polypeptides such as Fibronectin type III (Fn3) (see U.S. Pat. No. 6,703,199, which describes fibronectin polypeptide monobodies).

Antigen binding fragments can be incorporated into single chain molecules comprising a pair of tandem Fv segments (VH-CH1-VH-CH1) which, together with complementary light chain polypeptides, form a pair of antigen binding regions (Zapata et al., 1995 Protein Eng. 8(10):1057-1062; and U.S. Pat. No. 5,641,870).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “complement C5 protein” or “C5” are used interchangeably, and refers to the complement component 5 protein in different species. For example, human C5 has the sequence as set in SEQ ID NO: 99 (see Table 2b). Human C5 is known in the art and can be obtained from Quidel (Cat. Number A403).

The term “conditions or disorders associated with retinal disease” refers to any number of conditions or diseases in which the retina degenerates or becomes dysfunctional. This includes diabetic retinopathy (DR), macular edema, diabetic macular edema (DME), proliferative diabetic retinopathy (PDR), non-proliferative diabetic retinopathy (NPDR), neovascular age-related macular degeneration (wet AMD, neovascular AMD), retinal vein occlusion (RVO), multifocal choroiditis, myopic choroidal neovascularization, or retinopathy of prematurity. Anatomic characteristics of retinal vascular disease that may be treated by VEGF inhibition include macular edema, venous dilation, vessel tortuosity, vascular leakage as measured by fluorescein angiography, retinal hemorrhage, and microvascular anomalies (e.g. microaneurysm, cotton-wool spots, IRMA), capillary dropout, leukocyte adhesion, retinal ischemia, neovascularization of the optic disk, neovascularization of the posterior pole, iris neovascularization, intraretinal hemorrhage, vitreous hemorrhage, macular scar, subretinal fibrosis, and retinal fibrosis.

The term “condition or disorder associated with retinal vascular disease” refers to a condition in which there is abberent vascularization (e.g., increased or decreased) of the retina. A condition or disorder associated with retinal vascular disease includes neovascular age-related macular degeneration (wet AMD), diabetic retinopathy, diabetic macular edema, proliferative diabetic retinopathy, non-proliferative diabetic retinopathy, macular edema, retinal vein occlusion, multifocal choroiditis, myopic choroidal neovascularization and retinopathy of prematurity.

The term “conditions or disorders associated with diabetic retinopathy” refers to any of a number of conditions in which the retina degenerates or becomes dysfunctional, as a consequence of effects of diabetes mellitus (Type 1 or Type 2) on retinal vasculature, retinal metabolism, retinal pigment epithelium, the blood-retinal barrier, or ocular levels of advanced glycation end products (AGEs), aldose reductase activity, glycosylated hemoglobin, and protein kinase C. Visual loss in patients with diabetic retinopathy can be a result of retinal ischemia, macular edema, vascular leakage, vitreous hemorrhage, or direct effects of elevated glucose levels on retinal neurons. Anatomic characteristics of diabetic retinopathy that may be treated by VEGF inhibition include microaneurysm, cotton wool spots, venous dilation, macular edema, intra-retinal microvascular abnormalities (IRMA), intra-retinal hemorrhage, vascular proliferation, neovascularization of the disk, rubeosis, and retinal ischemia. “Diabetic macular edema” occurs in a subject with diabetic retinopathy and can occur at any stage of the disease.

The term “conditions or disorders associated with macular edma”, refers to any number of conditions or disorders in which swelling or thickening of the macula occurs as a result of retinal blood vessels leaking fluid, “macular edema”. Macular edema occurs in, and is often a complication of, retinal vascular disease. Specific conditions or disorders associated with macular edema include, diabetic retinopathy, diabetic macular edema, proliferative diabetic retinopathy, non-proliferative diabetic retinopathy, age-related macular degeneration, retinal vein occlusion, multifocal choroiditis, myopic choroidal neovascularization, or retinopathy of prematurity. Treatment of macular edema by the inhibition of VEGF can be determined by funduscopic examination, optical coherence tomography, and improved visual acuity.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some embodiments, the term “conservative sequence modifications” or “conservative modifications” are used to refer to amino acid modifications that do not significantly affect or alter the binding characteristics of the antibody containing the amino acid sequence.

As used herein, the term “DARPin” (an acronym for designed ankyrin repeat proteins) refers to an antibody mimetic protein typically exhibiting highly specific and high-affinity target protein binding. They are typically genetically engineered and derived from natural ankyrin proteins and consist of at least three, usually four or five repeat motifs of these proteins. Their molecular mass is about 14 or 18 kDa (kilodaltons) for four- or five-repeat DARPins, respectively. Examples of DARPins can be found, for example in U.S. Pat. No. 7,417,130.

The term “dose” refers to the quantity of peptide tag, peptide tagged molecule, protein or nucleic acid administered to a subject all at one time (unit dose), or in two or more administrations over a defined time interval. For example, dose can refer to the quantity of protein (e.g., a peptide tagged molecule, for example, a peptide tagged protein comprising an anti-VEGF antigen binding fragment and a peptide tag the binds HA) administered to a subject over the course of three weeks or one, two, three or more months (e.g., by a single administration, or by two or more administrations). The interval between doses can be any desired amount of time and is referred to as the “dosing interval”. The term “pharmaceutically effective” when referring to a dose means sufficient amount of the protein (e.g.: antibody or antigen binding fragment), peptide tag or other pharmaceutically active agent to provide the desired effect. The amount that is “effective” will vary from subject to subject, depending on the age and general condition of the individual, the particular drug or pharmaceutically active agent and the like. Thus, it is not always possible to specify an exact “effective” amount applicable for all patients. However, an appropriate “effective” dose in any individual case may be determined by one of ordinary skill in the art using routine experimentation.

The terms “Epo protein” or “Epo antigen” or “EPO” or “Epo” are used interchangeably, and refer to the erythropoietin protein in different species. For example, human EPO has the sequence as set out in Table 2b: SEQ ID NO: 98. The protein sequences for human, cynomolgus, mouse, rat, and rabbit Epo are publicly available. Human EPO can also be hyperglycosylated.

The terms “Epo Receptor” or “EPOR” are used interchangeably, and refer to the erythropoietin receptor protein, and refer to the erythropoietin receptor protein in different species. EPOR has been described by Winkelmann J. C., Penny L. A., Deaven L. L., Forget B. G., Jenkins R. B. Blood 76:24-30(1990).

The term “Factor D protein” or “Factor D antigen” or “Factor D” are used interchangeably, and refers to the Factor D protein in different species. The sequence of Human Factor D has been described by Johnson et al. (FEBS Lett. 1984 Jan. 30; 166(2):347-51). Antibodies to Factor D are known in the art and described in U.S. Pat. No. 8,273,352.

The term “Factor P protein” or “Factor P antigen” or “Factor P” are used interchangeably, and refers to the Factor P protein in different species. For example, human Factor P has the sequence as set out in Table 2b: SEQ ID NO: 100. Human Factor P can be obtained from Complement Tech, Tyler, Tex. Cynomolgus Factor P can be purified from cynomolgus serum (protocol adapted from Nakano et al., (1986) J Immunol Methods 90:77-83). Factor P is also know in the art as “Properdin”.

The term “FGFR2” refers to fibroblast growth factor receptor 2 in different species. FGFR2 has been described by Dionne C. A., Crumley G. R., Bellot F., Kaplow J. M., Searfoss G., Ruta M., Burgess W. H., Jaye M., Schlessinger J. EMBO J. 9:2685-2692(1990).

The term “hyaluronan” or “hyaluronic acid” or “HA” refers a large polymeric glycosamine containing repeating disaccharide units of N-acetyl glucosamine and glucuronic acid that occurs in extracellular matrix and on cell surfaces. Hyaluronan, is further described in J. Necas, L. Bartosikova, P. Brauner, J. Kolar, Veterinarni Medicina, 53, 2008 (8): 397-411.

The term “hyaladherin” or “hyaluronan binding proteins” or “HA binding proteins” refers to a protein or a family of proteins that bind Hyaluronan. Examples of HA binding proteins are known in the art (Day, et al. 2002 J Bio. Chem 277:7, 4585 and Yang, et al. 1994, EMBO J 13:2, 286-296) (e.g.: Link, CD44, RHAMM, Aggrecan, Versican, bacterial HA synthase, collagen VI, and TSG-6). Many HA binding proteins, and peptide fragments, contain a common structural domain of ˜100 amino acids in length involved in HA binding; the structural domain is referred to as a “LINK Domain” (Yang, et al. 1994, EMBO J 13:2, 286-296 and Mahoney, et al. 2001, J Bio. Chem 276:25, 22764-22771). For example, the LINK Domain of TSG-6, an HA binding protein, includes amino acid residues 36-128 of the human TSG-6 sequence (SEQ ID NO: 30).

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo).

The term “human monoclonal antibody” refers to antibodies displaying a single binding specificity which have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

A “humanized” antibody is an antibody that retains the reactivity of a non-human antibody while being less immunogenic in humans. This can be achieved, for instance, by retaining the non-human CDR regions and replacing the remaining parts of the antibody with their human counterparts (i.e., the constant region as well as the framework portions of the variable region). See, e.g., Morrison et al., Proc. Natl. Acad. Sci. USA, 81:6851-6855, 1984; Morrison and Oi, Adv. Immunol., 44:65-92, 1988; Verhoeyen et al., Science, 239:1534-1536, 1988; Padlan, Molec. Immun., 28:489-498, 1991; and Padlan, Molec. Immun., 31:169-217, 1994. Other examples of human engineering technology include, but are not limited to Xoma technology disclosed in U.S. Pat. No. 5,766,886.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch, J. Mol. Biol. 48:443, 1970, by the search for similarity method of Pearson and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., Current Protocols in Molecular Biology, John Wiley & Sons, Inc. (Ringbou ed., 2003)).

Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol, Biol. 48:444-453, 1970) algorithm which has been incorporated into the GAP program in the GCG software package (available on the world wide web at gcg.com), using either a Blossom 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the two nucleic acid sequences.

The term “isolated antibody” refers to an antibody that is substantially free of other antibodies or other proteins having different antigenic specificities (e.g., an isolated antibody that specifically binds VEGF is substantially free of antibodies that specifically bind antigens other than VEGF). An isolated antibody that specifically binds VEGF may, however, have cross-reactivity to other antigens. Moreover, an isolated antibody may be substantially free of other cellular material and/or chemicals, for example, an antibody isolated from a cell supernatant.

The term “IL-113” refers to refers to the Interleukin-1 beta protein a cytokine that is encoded in humans by the IL1B gene. For example, human IL-1β has the sequence as set out in Table 2b: SEQ ID NO: 102.

The terms “IL-10” or “IL10” are used interchangeably, and refer to the interleukin-10 protein, and refer to the interleukin-10 protein in different species. IL10 has been described by Vieira P., de Waal-Malefyt R., Dang M.-N., Johnson K. E., Kastelein R., Fiorentino D. F., Devries J. E., Roncarolo M.-G., Mosmann T. R., Moore K. W. Proc. Natl. Acad. Sci. U.S.A. 88:1172-1176(1991).

The term “IL-17A” refers to Interleukin 17A, is a 155-amino acid protein that is a disulfide-linked, homodimeric, secreted glycoprotein with a molecular mass of 35 kDa (Kolls J K, Linden A 2004, Immunity 21:467-76).

The term “isotype” refers to the antibody class (e.g., IgM, IgE, IgG such as IgG1 or IgG4) that is provided by the heavy chain constant region genes. Isotype also includes modified versions of one of these classes, where modifications have been made to alter the Fc function, for example, to enhance or reduce effector functions or binding to Fc receptors.

The term “linked” or “linking” refers to the attachment of a peptide tag, such as, for example, the peptide tags that bind HA listed in Table 1 and 2, to a molecule, for example a protein or a nucleic acid. Attachment of the peptide tag to a protein or nucleic acid molecule, can occur, for example, at the amino or carboxy terminus of the molecule. The peptide tag can also be attached to both the amino and carboxy termini of the molecule. The peptide tag can also be attached to one or more amino acids or nucleic acids within the protein or nucleic acid molecule, respectively. In addition, “linked” can also refer to the association of two or more peptide tags to each other and/or the association of two or more peptide tags to distinct sites on a molecule. Linking of the peptide tag to a molecule may be accomplished by several methods know in the art, including, but not limited to, expression of the peptide tag(s) and molecule as a fusion protein, linkage of two or more peptide tags via a “peptide linker” between tags and/or molecule, or by chemically joining peptide tags to a molecule after translation, either directly to each other, or through a linker by disulfide bonds, etc.

The term “peptide linker” refers to an amino acid sequence that functions to covalently join the peptide tag to a molecule. The peptide linker may be covalently attached to one or both of the amino or carboxy termini of a peptide tag and/or a protein or nucleic acid molecule. The peptide linker may also be conjugated to an amino acid or nucleic acid within the sequence of a protein or nucleic acid molecule, respectively. It is contemplated that peptide linkers may be, for example, about 2 to 25 residues in length.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985; and Rossolini et al., Mol. Cell. Probes 8:91-98, 1994).

The term “clearance” refers to is the volume of a substance (e.g.: matrix, tissue, plasma, or other substance such as a drug or such as a peptide tagged molecule) cleared per unit time (Shargel, L and Yu, ABC: Applied Biopharmaceutics & Pharmacokinetics, 4^{th} Edition (1999)). “Ocular clearance” refers to clearance of a substance such as a peptide tagged molecule from the eye.

The term “operably linked” refers to a functional relationship between two or more polynucleotide (e.g., DNA) segments. Typically, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, the term, “optimized” means that a nucleotide sequence has been altered to encode an amino acid sequence using codons that are preferred in the production cell or organism, generally a eukaryotic cell, for example, a cell of Pichia, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence. The optimized sequences herein have been engineered to have codons that are preferred in mammalian cells. However, optimized expression of these sequences in other eukaryotic cells or prokaryotic cells is also envisioned herein. The amino acid sequences encoded by optimized nucleotide sequences are also referred to as optimized.

The term “PDGF-BB” refers to platelet-derived growth factor subunit B, this protein has been as described by Josephs S. F., Ratner L., Clarke M. F., Westin E. H., Reitz M. S., Wong-Staal F. Science 225:636-639(1984).

The term “peptide tag” or “protein tag”, are used interchangeably to refer to a short protein sequence, peptide fragment, or peptidomimetic, that binds molecules found in various ocular compartments including: vitreous, retina, RPE, choroid, aqueous humor, trabecular meshwork, cornea, or cilliary body. For example, the ocular molecules bound by the peptide tag may include structural vitreal, retinal, and RPE proteins including: collagen and laminin: extracellular proteins including elastin, fibronectin and vitronectin; soluble proteins including albumin; transmembrane proteins including integrins; and carbohydrate containing molecules including hyaluronic acid, glycosamineglycans and other extracellular proteoglycans. Specific examples of peptide tags include, for example, peptide tags that bind HA (i.e.: HA-binding peptide tags). Peptide tags of the invention, including peptide tags that bind HA may increase ocular half-life (T_{1/2} or t_{1/2}), and/or increase mean ocular mean residence time, and/or decrease ocular clearance rate, and/or increase the dosing interval of a peptide tagged molecule (e.g.: protein or nucleic acid) as compared to the same molecule not linked to a peptide tag, (i.e.: an untagged molecule).

Peptide tags can be linked to form a multimer by several methods known in the art, including, but not limited to, expression of the protein tags as a fusion protein, linkage of two or more protein tags via a peptide linker between tags, or by chemically joining peptide tags after translation, either directly to each other, or through a linker by disulfide bonds, etc. The term “peptide tagged molecule” refers to a molecule that is linked to one or more peptide tags of the invention. The molecule may be, but is not limited to, a protein or nucleic acid. The term “tagged antibody” or “peptide tagged antibody” refers to an antibody, or antigen binding fragment thereof, that is linked to one or more protein tags of the invention. The term “peptide tagged antigen binding fragment” refers to an antigen binding fragment that is linked to one or more protein tags of the invention.

The term “half-life”, as used herein, refers to the time required for the concentration of a drug to fall by one-half (Rowland M and Towzer T N: Clinical Pharmacokinetics. Concepts and Applications. Third edition (1995) and Bonate P L and Howard D R (Eds): Pharmacokinetics in Drug Development, Volume 1 (2004)).

As used herein, the term “mean residence time” or “MRT” is the average time that the drug (e.g.: a peptide tagged molecule) resides in the body, including in a specific organ or tissue (e.g., the eye).

As used herein, the term “Ctrough” refers to the lowest concentration of drug measured in a matrix or tissue throughout the dosing interval, most often occurring immediately prior to repeat dose administration.

As used herein, the term “protein” refers to any organic compounds made of amino acids arranged in one or more linear chains and folded into a globular form. The amino acids in a polymer chain are joined together by the peptide bonds between the carboxyl and amino groups of adjacent amino acid residues. The term “protein” further includes, without limitation, peptides, single chain polypeptide or any complex molecules consisting primarily of two or more chains of amino acids. It further includes, without limitation, glycoproteins or other known post-translational modifications. It further includes known natural or artificial chemical modifications of natural proteins, such as without limitation, glycoengineering, pegylation, hesylation and the like, incorporation of non-natural amino acids, and amino acid modification for chemical conjugation with another molecule.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “subject” includes human and non-human animals. Non-human animals include all vertebrates (e.g.: mammals and non-mammals) such as, non-human primates (e.g.: cynomolgus monkey), sheep, dog, cow, chickens, amphibians, and reptiles. Except when noted, the terms “patient” or “subject” are used herein interchangeably. As used herein, the terms “cyno” or “cynomolgus” refer to the cynomolgus monkey (Macaca fascicularis).

The term “terminal concentration” refers to the concentration of the peptide tag, peptide tagged molecule, etc. that is measured at the end of the experiment or study. An “increase in terminal drug concentration” refers to an at least 25% increase in terminal concentration of the peptide tagged molecule.

As used herein, the term “treating” or “treatment” of any conditions or disorders associated with retinal vascular disease, conditions or disorders associated with diabetic retinopathy, and/or conditions or disorders associated with macular edema refers in one aspect, to ameliorating the disease or disorder (i.e., slowing or arresting or reducing the development of the disease or at least one of the clinical symptoms thereof). In another aspect “treating” or “treatment” refers to alleviating or ameliorating at least one physical parameter including those which may not be discernible by the patient. In yet another aspect, “treating” or “treatment” refers to modulating the disease or disorder, either physically, (e.g., stabilization of a discernible symptom), physiologically, (e.g., stabilization of a physical parameter), or both. In yet another aspect, “treating” or “treatment” refers to preventing or delaying the onset or development or progression of the disease or disorder. “Prevention” as it relates to indications described herein, including, conditions or disorders associated with retinal vascular disease, conditions or disorders associated with diabetic retinopathy, and/or conditions or disorders associated with macular edema, means any action that prevents or slows a worsening in visual function, retinal anatomy, retinal vascular disease parameter, diabetic retinopathy disease parameter, and/or macular edema disease parameter, as described below, in a patient at risk for said worsening. More specifically, “treatment” of conditions or disorders associated with retinal vascular disease, conditions or disorders associated with diabetic retinopathy, and/or conditions or disorders associated with macular edema means any action that results in, or is contemplated to result in, the improvement or preservation of visual function and/or retinal anatomy. Methods for assessing treatment and/or prevention of disease are known in the art and described herein below.

The term “TNFα” refers to tumor necrosis factor alpha (also known as, cachectin), a naturally occurring mammalian cytokine produced by numerous cell types, including monocytes and macrophages in response to endotoxin or other stimuli. TNFα is a major mediator of inflammatory, immunological, and pathophysiological reactions (Grell, M., et al. (1995) Cell, 83: 793-802). Soluble TNFα is formed by the cleavage of a precursor transmembrane protein (Kriegler, et al. (1988) Cell 53: 45-53), and the secreted 17 kDa polypeptides assemble to soluble homotrimer complexes (Smith, et al. (1987), J. Biol. Chem. 262: 6951-6954; for reviews of TNFα, see Butler, et al. (1986), Nature 320:584; Old (1986), Science 230: 630). The sequence for human TNFα is described in Table 2b and has the sequence of SEQ ID NO: 101.

The term “TSG-6” refers to Tumor Necrosis Factor-Inducible Gene 6. TSG-6 is a member of an HA binding protein family and contains a LINK Domain. (Lee et al. J Cell Bio (1992) 116:2, 545-57). The LINK Domain from TSG-6 is also referred to herein as the “TSG-6 LINK Domain”.

The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, such as an adeno-associated viral vector (AAV, or AAV2), wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “VEGF” refers to the 165-amino acid vascular endothelial cell growth factor, and related 121-, 189-, and 206-amino acid vascular endothelial cell growth factors, as described by Leung et al., Science 246:1306 (1989), and Houck et al., Mol. Endocrin. 5:1806 (1991) together with the naturally occurring allelic and processed forms of those growth factors. The sequence for human VEGF is described in Table 2b and has a sequence of SEQ ID NO: 97.

The term “VEGF-mediated disorder” refers to any disorder, the onset, progression or the persistence of the symptoms or disease states of which requires the participation of VEGF. Exemplary VEGF-mediated disorders include, but are not limited to, age-related macular degeneration, neovascular glaucoma, diabetic retinopathy, macular edema, diabetic macular edema, pathologic myopia, retinal vein occlusions, retinopathy of prematurity, abnormal vascular proliferation associated with phakomatoses, edema (such as that associated with brain tumors), Meigs' syndrome, rheumatoid arthritis, psoriasis and atherosclerosis.

As used herein, the term “therapeutic protein” refers to a protein that is useful to treat, prevent or ameliorate a disease, condition or disorder.

As used herein, the term “protein receptor” refers to a protein that is a cellular receptor and binds a ligand.

### BRIEF DESCRIPTION OF THE DRAWINGS

FIG. 1. Shows 4-point PK curves for ranibizumab and NVS4 in rabbit vitreous.

FIG. 2. Shows dose response of hVEGF in the rabbit leakage model.

FIG. 3. Shows a time-course of the inhibition of fluorescein leakage with untagged antibodies.

FIG. 4. Shows correlation between efficacy and terminal vitreal concentrations of tagged antibodies in the rabbit leakage model.

FIG. 5. Shows duration of efficacy in the rabbit leakage model for collagen-binding peptide tags

FIG. 6. Shows duration of efficacy in the rabbit leakage model of NVS1, NVS2, NVS3, NVS36, and NVS37.

FIG. 7. Shows 2-point PK plots for ranibizumab, NVS1, NVS2, and NVS3.

FIG. 8. Shows extended duration of efficacy of tagged antibody in the rabbit leakage model.

FIG. 9. Shows extended duration of efficacy of tagged antibodies in the rabbit leakage model.

FIG. 10. Shows 2 and 6-point PK plots for NVS1.

FIG. 11. Shows a pilot study in cynomolgus monkeys.

FIG. 12. Shows 2-point ocular PK plots derived from the terminal drug levels measured in a 28-day cynomolgus tolerability study.

FIG. 13. Shows 3-point ocular PK curves derived from the terminal drug levels measured in a 59-day cynomolgus efficacy study.

FIGS. 14. A, B, and C show a model prediction of peptide tagged antibody concentrations in the vitreous relative to ranibizumab in humans. FIGS. 14A and 14B: dose range predictions. FIG. 14C duration of efficacy. FIG. 14D shows a model which illustrates the effect of increasing the half-life of a molecule with an HA-binding peptide tag on the percent of the molecule remaining in the eye over time after the initial dose. FIG. 14E shows the duration of efficacy in the eye for a peptide tagged molecule (e.g.: NVS2) compared and IVT doses of: ranibizumab (0.5 mg), aflibercept (2 mg), and bevacizumab (1.25 mg). FIG. 14F shows the predicted serum total drug Cₐᵥₑ (nM) after one year dosing with a dosing interval as shown in FIG. 14E.

FIG. 15. Shows rabbit duration of efficacy studies with non-NVS4 anti-VEGF proteins

FIG. 16. Shows rabbit efficacy of high and low affinity variants challenged with VEGF

FIG. 17. Shows bio-distribution of a peptide tagged molecule and untagged molecule by PET imaging

### DETAILED DESCRIPTION

The present invention is based, in part, on the discovery of peptide tags that increase the half-life and/or mean residence time of proteins or nucleic acids in the eye. In certain aspects the invention peptide tags increase the half-life and/or mean residence time of antibodies and antigen binding fragments, therapeutic proteins, protein receptors, DARPins and/or aptamers in the eye. The invention also relates to the discovery of long acting antibody molecules that specifically bind ocular proteins (e.g.: HA and/or VEGF) and exhibit an increased half-life and/or mean residence time in the eye. The invention relates to both full IgG format antibodies as well as antigen binding fragments, such as Fab fragments, linked to a protein tag.

### Peptide Tags

Many factors may affect a protein's half-life in vivo. For example, kidney filtration, metabolism in the liver, degradation by proteolytic enzymes (proteases), and immunogenic responses (e.g., protein neutralization by antibodies and uptake by macrophages and dendritic cells). A variety of strategies can be used to extend the serum half-life of antibodies, antigen binding fragments, or antibody mimetics. For example, by attaching polysialic acid (PSA), hydroxyethyl starch (HES), albumin-binding ligands, and carbohydrate shields; by genetic fusion to proteins binding to serum proteins, such as albumin, IgG, FcRn, and transferrin; by coupling (genetically or chemically) to other binding moieties that bind to serum proteins, such as nanobodies, Fabs, DARPins, avimers, affibodies, and anticalins; by genetic fusion to albumin or a domain of albumin, albumin-binding proteins, an antibody Fc region; or by incorporation into nanocarriers, slow release formulations, or medical devices.

The present invention provides peptide tags that specifically bind hyaluronan in the eye. hyaluronan is present in the body in various sizes in many organs in tissues. For example, the human eye and synovial fluid contain the highest concentrations of hyaluronan concentrations with 0.14-0.338 mg/ml and 1.42-3.6 mg/ml respectively, while other tissues/fluids contain much lower concentrations of hyaluronan such as serum in which hyaluronan concentrations are 0.00001-0.0001 mg/ml (Laurent and Fraser, 1986 Ciba Found Symp. 1986; 124:9-29.). Non-ocular hyaluronan mainly consists of low molecular weight polymers that are rapidly degraded and turned over. In humans, hyaluronan has a half-life of 2.5-5 minutes in blood (Fraser J R, Laurent T C, Pertoft H, Baxter E. Biochem J. 1981 Nov. 15; 200(2):415-24.). In contrast, ocular hyaluronan mainly consists of high molecular weight polymers (>0.5×10̂5 daltons) and has a slower turnover rate of days or weeks (Laurent and Fraser, Exp. Eye Res. 1983; 36, 493-504). Due to these differences in the size and turnover of hyaluronan in the eye, the hyaluronan in the eye is hypothesized to serve as a sustained release scaffold for intravitreal proteins and nucleic acids linked to an HA-binding peptide tag.

Putative hyaluronan binding proteins have been described in the art (J. Necas, L. Bartosikova, P. Brauner, J. Kolar. Veterinarni Medicina, 53, 2008 (8): 397-411), for example, Tumor necrosis factor-inducible gene 6 protein (TSG6), hyaluronana mediated motility receptor (RHAMM), CD44 antigen, hyaluronan and proteoglycan link protein 4, Neurocan core protein, Stabilin-2, and human glial hyaluronate-binding protein. However, most putative hyluronan binding proteins tested did not bind HA, nor increase the ocular half-life of proteins or nucleic acids linked to the putative HA-binding proteins. The present invention is based on the surprising discovery of peptide tags that bind HA in the eye and are suitable for extending the half-life of a protein or nucleic acid in the eye, increasing the terminal concentration of a protein or nucleic acid in the eye, decreasing the ocular clearance of a protein or nucleic acid in the eye, and/or increasing mean residence time of a protein or nucleic acid in the eye. In certain aspects of the invention the peptide tag binds HA in the eye with a KD of less than or equal to 9.0 uM, less than or equal to 8.5 uM, less than or equal to 8.0 uM, less than or equal to 7.5 uM, less than or equal to 7.0 uM, less than or equal to 6.5 uM, less than or equal to 6.0 uM, less than or equal to 5.5 uM, less than or equal to 5.0 uM, less than or equal to 4.5 uM, less than or equal to 4.0 uM, less than or equal to 3.5 uM, less than or equal to 3.0 uM, less than or equal to 2.5 uM, less than or equal to 2.0 uM, less than or equal to 1.5 uM, less than or equal to 1.0 uM, less than or equal to 0.5 uM, or less than or equal to 100 nM. In more specific aspects, for example, the peptide tag binds HA in the eye with a KD of less than or equal to 8.0 uM, less than or equal to 7.2 uM, less than or equal to 6.0 uM, or less than or equal to 5.5 uM. In some aspects of the invention the peptide tag that binds HA has a LINK domain. In certain other aspects of the invention the LINK domain is a TSG-6 LINK domain. Still other aspects of the invention are based on the discovery of modified versions of the peptide tag that also resist proteolytic cleavage and/or glycosylation. More specifically the invention may include a peptide tag that binds, or is capable of binding, HA comprising a sequence of SEQ ID NO: 32, 33, 34, 35 or 36. It is contemplated that the peptide tag comprising a sequence of SEQ ID NO: 32, 33, 34, 35 or 36, binds, or is capable of binding, HA in the eye of a subject. It is contemplated that the peptide tag may be any one of the peptide tags listed in Table 1. More specifically, the peptide tag may be HA10, HA10.1, HA10.2, HA11 or HA11.1.

In certain aspects, the peptide tag can have a sequence comprising 30, 35, 40, 45, 50, 55, 60, 65, 70, 75, 80, 85, 90, 91, 92, 93, 94, 95, 96, 97 or 98 consecutive amino acids of SEQ ID NOs: 32, 33, 34, 35 or 36. In certain other aspects, it is contemplated that a peptide tag is a truncated variant of a peptide tag comprising a sequence of SEQ ID NO: 32, 33, 34, 35 or 36. Amino acids may be cleaved from the N-terminus, C-terminus or both of the peptide tag comprising a sequence of SEQ ID NO: 32, 33, 34, 35 or 36 to produce a truncated variant of the peptide tags HA10, HA10.1, HA10.2, HA11 or HA11.1. It is further contemplated that the sequence may cleaved from the N-terminus of SEQ ID NO: 32, 33, 34, 35 or 36 up to and (but not including) the first N-terminal cysteine. It is further contemplated that the sequence may cleaved from the C-terminus of SEQ ID NO: 32, 33, 34, 35 or 36 up to and (but not including) the first C-terminal cysteine. It is further contemplated that the sequence may cleaved from both the N-terminus and the C-terminus of SEQ ID NO: 32, 33, 34, 35 or 36 up to (but not including) the first N-terminal cysteine and (but not including) the first C-terminal cysteine. For example, with respect to SEQ ID NO: 32, one of skill in the art could remove up to 22 amino acids from the N-terminal end (bold) and/or up to six amino acids from the C-terminal end (underline):

(SEQ ID NO: 32)
GVYHREARSGKYKLTYAEAKAVCEFEGGHLATYKQLEAARKIGFHVCAAG
WMAKGRVGYPIVKPGPNCGFGKTGIIDYGIRLNRSERWDAYCYNPHAK

The peptide tag of the invention can be linked to a molecule to extend the ocular half-life of the molecule, for example the molecule may be a protein or nucleic acid. Specific examples of proteins and nucleic acids that can be modified by the protein tags described herein include, but are not limited to, antibodies, antigen binding fragments, therapeutic proteins, protein receptors, DARPins, and/or aptamers, as well as multivalent combinations proteins and nucleic acids. In certain aspects, these proteins and nucleic acids bind a target protein in the eye, for example, VEGF, C5, Factor P, Factor D, EPO, EPOR, II-1β, IL-17A, TNFα, IL-10 or FGFR2. Without being bound to any particular theory, the peptide tags of the invention, when linked to a protein or nucleic acid that binds a target protein in the eye, decrease ocular clearance, increase the mean residence time, increase half-life (T_{1/2}), and/or increase terminal drug concentration of the tagged molecule (e.g.: protein or nucleic acid) in the eye relative to the untagged molecule.

The invention also relates to the surprising finding that linking a peptide tag that binds, or is capable of binding. HA in the eye to a molecule (e.g.: a protein or nucleic acid) significantly improves the biophysical properties of the peptide tagged molecule compared to the molecule without the tag. It is contemplated the biophysical properties of the peptide tagged molecule improve a statistically significant amount (i.e.: p<0.05) compared to the molecule without a peptide tag, including, but not limited to improved solubility, improved isoelectric point (pI) and/or improved binding affinity of the peptide tagged molecule to its target relative to an untagged version of the molecule. In specific aspects the invention relates to a method of increasing the solubility of a molecule comprising the step of linking the molecule to a peptide tag that binds HA in the eye. In specific aspects the invention relates to a method of increasing the pI of a molecule comprising the step of linking the molecule to a peptide tag that binds HA in the eye. In certain aspects the linking a peptide tag to a molecule increases the pI up to 3 fold compared to the untagged molecule. In other aspects the pI of a peptide tagged molecule increases up to 2.8, 2.5, 2.0, 1.75, 1.5, 1.0, or 0.5 fold as compared to the untagged molecule.

In specific aspects the invention relates to a method of increasing the binding affinity of a molecule to its target comprising the step of linking the molecule to a peptide tag that binds HA in the eye. In certain specific aspects the linking a peptide tag to a molecule improves the binding affinity of the molecule for the primary target by 135 fold, 130 fold, 120 fold, 110 fold, 100 fold, 90 fold, 80 fold, 75 fold, 50 fold, 40 fold, 30 fold, 20 fold, 15 fold 10 fold, 7.5 fold, 5 fold, 4 fold, 2 fold, 1.75 fold. It is contemplated that the peptide tagged molecule binds HA in the eye with a KD of less than or equal to 9.0 uM, 8.0 uM, 6.0 uM, or 5.5 uM. It is further contemplated that the peptide tag comprising a sequence of SEQ ID NO: 32, 33, 34, 35 or 36 improves the biophysical properties of a molecule to which it is linked by a statistically significant amount when compared to the molecule without the tag. It is still further contemplates that multiple peptide tags may be used in any of the methods described herein to improve the binding affinity for HA in the eye, more specifically for example a peptide tagged molecule comprising more than one peptide tag binds HA with a KD of less than or equal to 1.0 uM, 0.9 uM, 0.8 uM, 0.7 uM, 0.6 uM, 0.5 uM, 0.4 uM, 0.3 uM, 0.2 uM, or 0.1 uM.

In certain aspects of the invention it is contemplated that a single peptide tag is linked to a molecule, for example a protein or nucleic acid molecule. In other aspects of the invention it is contemplated that two, three, four or more peptide tags maybe linked to the protein or nucleic acid. It is contemplated that the peptide tag is linked either to the carboxy-terminus or the amino-terminus of the protein. It is also contemplated that the peptide tag may be linked to the heavy chain or light chain of an antibody, or antigen binding fragment thereof, or alternatively linked to both chains. It is contemplated that peptide tag may be linked to the 5′ and/or 3′ of the nucleic acid molecule. Multiple tags may be concatenated and/or linked to multiple protein chains (e.g.: linked to heavy and light chains). It is also contemplated that the protein tags and/or proteins and/or nucleic acids may be chemically joined after translation, either directly to each other, or through disulfide bond linkage, peptide linkers, etc. Peptide linkers and methods of linking protein tags to proteins (e.g.: antibodies and antigen binding fragments) or nucleic acids are known in the art and described herein.

### Peptide Tagged Molecules

Another aspect of the invention includes peptide tagged molecules. In certain aspects of the invention, the peptide tagged molecules may comprise a peptide tag that binds, or is capable of binding, HA. In certain aspects the peptide tagged molecule comprises a peptide tag that binds HA in the eye with a KD of less than or equal to 9.0 uM. For example, the peptide tag can bind HA with a KD of less than or equal to, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In one aspect the peptide tag binds HA with a KD of less than or equal to 8.0 uM. In one aspect the peptide tag binds HA with a KD of less than or equal to 7.2 uM. In one aspect the peptide tag binds HA with a KD of less than or equal to 5.5 uM. In certain specific aspects, the peptide tag may comprise a sequence of SEQ ID NO: 32, 33, 34, 35 or 36. It is also contemplated that the peptide tag is linked to a molecule that is protein or a molecule that is a nucleic acid. Examples of molecules that can be linked to protein tags are described herein.

### Protein Molecules

The present invention provides proteins that can be linked to peptide tags of the invention. In certain aspects of the invention the protein may be an isolated antibody, or antigen binding fragment thereof (e.g.: Fab, scFv, Fc Trap, etc.), a protein that is a therapeutic protein (e.g. EPO, Insulin, cytokines, etc.), a protein receptor (e.g.: EPO receptor, FGFR2, etc), or DARPins. In certain aspects of the invention the protein binds, or is capable of binding, VEGF, C5, Factor P, Factor D, EPO, EPOR, IL-1β, IL-17A, TNFα, IL-10 or FGFR2. It is further contemplated that the protein binding occurs in the eye.

One aspect of the invention includes proteins that bind VEGF. Numerous VEGF binding proteins are known in the art and described herein, see for example Tables 1, 9 and 9b. In certain aspects, the anti-VEGF binding proteins may have the sequences of NVS4, NVS80, NVS81, NVS82, NVS83, NVS84 or NVS85. In certain specific aspects, for example, the invention also provides antibodies and antigen binding fragments that specifically bind VEGF. VEGF antibodies and antigen binding fragments of the invention include, but are not limited to the antibodies and fragments, isolated and described in US patent application US20120014958 or WO1998045331, as well as antibodies and antigen binding fragments as described herein, for example in Table 1 and the examples. Other anti-VEGF antibodies, VEGF antagonists, and VEGF receptor antagonists that can be linked to the protein tags described herein and used in the methods described herein include, for example: ranibizumab (Ferrara N, Damico L, Shams N, Lowman H, Kim R. Retina. 2006 October; 26(8):859-70), bevacizumab (Ferrara N, Hillan K J, Gerber H P, Novotny W. Nat Rev Drug Discov. 2004 May; 3(5):391-400.), aflibercept (Stewart M W, Grippon S, Kirkpatrick P. Nat Rev Drug Discov. 2012 Mar. 30; 11(4):269-70.), KH902 (Zhang M, Zhang J, Yan M, Li H, Yang C, Yu D. Mol Vis. 2008 Jan. 10; 14:37-49.), MP0112 (Campochiaro P A, Channa R, Berger B B, Heier J S, Brown D M, Fiedler U, Hepp J, Stumpp M T. Am J Ophthalmol. 2013 April; 155(4):697-704), pegaptanib Gragoudas E S, Adamis A P, Cunningham E T Jr, Feinsod M, Guyer D R. N Engl J Med. 2004 Dec. 30; 351(27):2805-16.), CT-322 (Dineen S P, Sullivan L A, Beck A W, Miller A F, Carbon J G, Mamluk R, Wong H, Brekken R A. BMC Cancer. 2008 Nov. 27; 8:352. doi: 10.1186/1471-2407-8-352.) and anti-VEGF antibodies and fragments as described in US20120014958.

A particular aspect of the invention provides antibodies that specifically bind a VEGF protein, wherein the antibodies comprise a VH domain comprising an amino acid sequence of SEQ ID NO: 7. The present invention also provides antibodies that specifically bind a VEGF protein wherein the antibodies, antigen binding fragments comprise a heavy chain having an amino acid sequence of SEQ ID NO: 9. The present invention also provides antibodies that specifically bind a VEGF protein wherein the antibodies, antigen binding fragments having a peptide tagged heavy chain comprising an amino acid sequence of SEQ ID NO: 21, 23, 25, 27 or 29. The present invention also provides antibodies that specifically bind to a VEGF protein (e.g., human, cynomolgus, rat and/or mouse VEGF), wherein the antibodies comprise a VH CDR having an amino acid sequence of any one of the VH CDRs listed in Table 1, infra. In particular, the invention provides antibodies that specifically bind to a VEGF protein, wherein the antibodies comprise (or alternatively, consist of) one, two, three, or more VH CDRs having an amino acid sequence of any of the VH CDRs listed in Table 1, infra.

The present invention provides antibodies that specifically bind to a VEGF protein, said antibodies comprising a VL domain having an amino acid sequence of SEQ ID NO:17. The present invention also provides antibodies that specifically bind a VEGF protein wherein the antibodies, antigen binding fragments comprise a light chain having an amino acid sequence of SEQ ID NO: 19. The present invention also provides antibodies that specifically bind to a VEGF protein, said antibodies comprising a VL CDR having an amino acid sequence of any one of the VL CDRs listed in Table 1, infra. In particular, the invention provides antibodies that specifically bind to a VEGF protein, said antibodies comprising (or alternatively, consisting of) one, two, three or more VL CDRs having an amino acid sequence of any of the VL CDRs listed in Table 1, infra.

Alternate aspects of the invention provide additional proteins that can be linked to the peptide tags described herein. In certain aspects, the protein is an antibody or antigen binding fragment that binds Factor P, Factor D, Epo, C5, TNFα, II-1β, II-17a, and/or FGFR2. In certain aspects the protein may be a therapeutic protein such as erythropoietin, Insulin, human growth factor, interleukin-10, complement factor H, CD35, CD46, CD55, CD59, complement factor I, complement receptor 1-related (CRRY), nerve growth factor, angiostatin, pigment epithelium-derived factor, endostatin, ciliary neurotrophic factor, complement factor 1 inhibitor, complement factor like-1, complement factor I or the like. In other aspects, the protein may be a receptor such as EPOR. Additional examples of proteins that can be linked to peptide tags are provided in Table 2, 8 and 8b. More specifically, the proteins may be NVS70, NVS71, NVS72, NVS73, NVS74, NVS75, NVS76, NVS77, NVS78 or NVS90.

Other proteins of the invention include amino acids that have been mutated, yet have at least 60, 70, 80, 85, 90, 95, 96, 97, 98 or 99 percent identity to the sequences described in Table 1, 2, 8b or 9b. In some embodiments, it includes mutant amino acid sequences wherein no more than 1, 2, 3, 4 or 5 amino acids have been mutated in the sequence described in Table 1, 2, 8b or 9b.

The present invention also provides nucleic acid sequences that encode the protein molecules described herein. Such nucleic acid sequences can be optimized for expression in mammalian cells.

### Nucleic Acid Molecules

The present invention provides nucleic acids that can be linked to peptide tags of the invention. In certain aspects the nucleic acid that is linked to a peptide tag may be an mRNA or an RNAi agent, a ribozyme or an antisense oligonucleotide. More specifically, RNAi agents linked to the peptide tag may be an siRNA, shRNA, microRNA (i.e.: miRNA), anti-microRNA oligonucleotide, aptamer, or the like. In certain specific aspects, the nucleic acid molecule may be an aptamer. In particular, the aptamer may bind PDGF-BB. More specifically, the nucleic acid may be NVS79.

TABLE 1
Examples of peptide tagged anti-VEGF molecules and component
sequences: including, the untagged anti-VEGF molecule (NVS4), linkers and peptide tags.

| | | SEQUENCE (OR SEQ ID NO: #) |
|---|---|---|
| NVS4 | | |
| SEQ ID NO: 1 (Kabat) | HCDR1 | DYYYMT |
| SEQ ID NO: 2 (Kabat) | HCDR2 | FIDPDDDPYYATWAKG |
| SEQ ID NO: 3 (Kabat) | HCDR3 | GDHNSGWGLDI |
| SEQ ID NO: 4 (Chothia) | HCDR1 | GFSLTDYY |
| SEQ ID NO: 5 (Chothia) | HCDR2 | DPDDD |
| SEQ ID NO: 6 (Chothia) | HCDR3 | GDHNSGWGLDI |
| SEQ ID NO: 7 | VH | EVQLVESGGGLVQPGGSLRLSCTASGFSLTDYYYMTWVRQA |
| | | PGKGLEWVGFIDPDDDPYYATWAKGRFTISRDNSKNTLYLQ |
| | | MNSLRAEDTAVYYCAGGDHNSGWGLDIWGQGTLVTVSS |
| SEQ ID NO: 8 | DNA of VH | GAGGTGCAGCTGGTGGAATCAGGCGGCGGACTGGTGCAG |
| | SEQ ID NO: 7 | CCTGGCGGTAGCCTGAGACTGAGCTGCACCGCTAGTGGCT |
| | | TTAGCCTGACCGACTACTACTATATGACCTGGGTCAGACAG |
| | | GCCCCTGGTAAAGGCCTGGAGTGGGTCGGCTTTATCGACC |
| | | CCGACGACGACCCCTACTACGCTACCTGGGCTAAGGGCCG |
| | | GTTCACTATCTCTAGGGATAACTCTAAGAACACCCTGTACCT |
| | | GCAGATGAATAGCCTGAGAGCCGAGGACACCGCCGTCTAC |
| | | TACTGCGCCGGCGGCGATCACAATAGCGGCTGGGGCCTGG |
| | | ATATCTGGGGTCAGGGCACCCTGGTCACCGTGTCTAGC |
| SEQ ID NO: 9 | Heavy Chain | EVQLVESGGGLVQPGGSLRLSCTASGFSLTDYYYMTWVRQA |
| | | PGKGLEWVGFIDPDDDPYYATWAKGRFTISRDNSKNTLYLQ |
| | | MNSLRAEDTAVYYCAGGDHNSGWGLDIWGQGTLVTVSSAS |
| | | TKGPSVFPLAPSSKSTSGGTAALGCLVKDYFPEPVTVSWNSGA |
| | | LTSGVHTFPAVLQSSGLYSLSSVVTVPSSSLGTQTYICNVNHKP |
| | | SNTKVDKRVEPKSC |
| SEQ ID NO: 10 | DNA of Heavy | GAGGTGCAATTGGTTGAATCTGGGGGCGGACTGGTGCAGC |
| | Chain SEQ ID | CCGGTGGATCTTTGCGCCTGTCCTGTACAGCTTCTGGCTTCT |
| | NO: 9 | CCTTGACCGACTACTATTACATGACTTGGGTTCGCCAAGCC |
| | | CCAGGCAAAGGGCTTGAATGGGTGGGGTTCATTGACCCCG |
| | | ACGATGATCCTTACTACGCCACATGGGCAAAGGGCCGGTTT |
| | | ACTATCAGCCGGGATAATTCCAAAAACACATTGTATTTGCA |
| | | AATGAACTCACTGAGAGCAGAAGATACGGCTGTGTACTAT |
| | | TGCGCAGGCGGCGATCATAACTCCGGCTGGGGCCTGGACA |
| | | TCTGGGGGCAGGGGACCCTGGTGACAGTCAGCTCAGCCTC |
| | | AACGAAGGGGCCCAGCGTGTTTCCTTTGGCCCCAAGCAGC |
| | | AAGTCCACGTCCGGTGGGACTGCAGCTCTTGGTTGTCTGGT |
| | | CAAGGATTATTTCCCAGAACCCGTGACCGTGTCTTGGAACA |
| | | GTGGTGCATTGACATCAGGAGTGCATACATTCCCAGCTGTG |
| | | CTGCAGAGCTCTGGCCTGTATAGCCTTTCCTCTGTTGTCACG |
| | | GTGCCCAGCTCCAGCCTGGGGACGCAGACCTATATTTGTAA |
| | | CGTGAACCATAAACCCTCCAACACCAAGGTTGATAAAAGA |
| | | GTGGAGCCCAAGTCTTGT |
| SEQ ID NO: 11 (Kabat) | LCDR1 | QASEIIHSWLA |
| SEQ ID NO: 12 (Kabat) | LCDR2 | LASTLAS |
| SEQ ID NO: 13 (Kabat) | LCDR3 | QNVYLASTNGAN |
| SEQ ID NO: 14 (Chothia) | LCDR1 | SEIIHSW |
| SEQ ID NO: 15 (Chothia) | LCDR2 | LAS |
| SEQ ID NO: 16 (Chothia) | LCDR3 | VYLASTNGA |
| SEQ ID NO: 17 | VL | EIVMTQSPSTLSASVGDRVIITCQASEIIHSWLAWYQQKPGKA |
| | | PKLLIYLASTLASGVPSRFSGSGSGAEFTLTISSLQPDDFATYYC |
| | | QNVYLASTNGANFGQGTKLTVLK |
| SEQ ID NO: 18 | DNA of VL | GAGATCGTGATGACTCAGTCACCTAGCACCCTGAGCGCTA |
| | SEQ ID NO: 17 | GTGTGGGCGATAGAGTGATTATCACCTGTCAGGCTAGTGA |
| | | AATTATTCACTCCTGGCTGGCCTGGTATCAGCAGAAGCCCG |
| | | GTAAAGCCCCTAAGCTGCTGATCTACCTGGCCTCTACCCTG |
| | | GCTAGTGGCGTGCCCTCTAGGTTTAGCGGTAGCGGTAGTG |
| | | GCGCCGAGTTCACCCTGACTATCTCTAGCCTGCAGCCCGAC |
| | | GACTTCGCTACCTACTACTGTCAGAACGTCTACCTGGCTAG |
| | | TACTAACGGCGCTAACTTCGGTCAGGGCACTAAGCTGACC |
| | | GTGCTGAAG |
| SEQ ID NO: 19 | Light Chain | EIVMTQSPSTLSASVGDRVIITCQASEIIHSWLAWYQQKPGKA |
| | | PKLLIYLASTLASGVPSRFSGSGSGAEFTLTISSLQPDDFATYYC |
| | | QNVYLASTNGANFGQGTKLTVLKRTVAAPSVFIFPPSDEQLKS |
| | | GTASVVCLLNNFYPREAKVQWKVDNALQSGNSQESVTEQDS |
| | | KDSTYSLSSTLTLSKADYEKHKVYACEVTHQGLSSPVTKSFNRG |
| | | EC |
| SEQ ID NO: 20 | DNA of Light | GAGATCGTGATGACTCAGTCACCTAGCACCCTGAGCGCTA |
| | Chain | GTGTGGGCGATAGAGTGATTATCACCTGTCAGGCTAGTGA |
| | SEQ ID NO: 19 | AATTATTCACTCCTGGCTGGCCTGGTATCAGCAGAAGCCCG |
| | | GTAAAGCCCCTAAGCTGCTGATCTACCTGGCCTCTACCCTG |
| | | GCTAGTGGCGTGCCCTCTAGGTTTAGCGGTAGCGGTAGTG |
| | | GCGCCGAGTTCACCCTGACTATCTCTAGCCTGCAGCCCGAC |
| | | GACTTCGCTACCTACTACTGTCAGAACGTCTACCTGGCTAG |
| | | TACTAACGGCGCTAACTTCGGTCAGGGCACTAAGCTGACC |
| | | GTGCTGAAGCGGACCGTGGCCGCTCCTAGTGTGTTTATCTT |
| | | CCCACCTAGCGACGAGCAGCTGAAGTCAGGCACCGCTAGT |
| | | GTCGTGTGCCTGCTGAACAACTTCTACCCTAGAGAAGCTAA |
| | | GGTGCAGTGGAAAGTGGATAACGCCCTGCAGTCAGGTAAT |
| | | AGTCAGGAATCAGTCACCGAGCAGGACTCTAAGGATAGCA |
| | | CCTATAGCCTGTCTAGCACACTGACCCTGTCTAAGGCCGAC |
| | | TACGAGAAGCACAAGGTCTACGCCTGCGAAGTGACTCACC |
| | | AGGGACTGTCTAGCCCCGTGACTAAGTCCTTTAATAGAGGC |
| | | GAGTGC |
| NVS1 | | |
| SEQ ID NO: 1 (Kabat) | HCDR1 | 1 |
| SEQ ID NO: 2 (Kabat) | HCDR2 | 2 |
| SEQ ID NO: 3 (Kabat) | HCDR3 | 3 |
| SEQ ID NO: 4 (Chothia) | HCDR1 | 4 |
| SEQ ID NO: 5 (Chothia) | HCDR2 | 5 |
| SEQ ID NO: 6 (Chothia) | HCDR3 | 6 |
| SEQ ID NO: 7 | VH | 7 |
| SEQ ID NO: 8 | DNA of VH | 8 |
| | SEQ ID NO: 7 | |
| SEQ ID NO: 9 | Heavy Chain | 9 |
| SEQ ID NO: 21 | Heavy Chain + | EVQLVESGGGLVQPGGSLRLSCTASGFSLTDYYYMTWVRQA |
| | Linker + | PGKGLEWVGFIDPDDDPYYATWAKGRFTISRDNSKNTLYLQ |
| | protein tag | MNSLRAEDTAVYYCAGGDHNSGWGLDIWGQGTLVTVSSAS |
| | (SEQ ID NO: 9 + | TKGPSVFPLAPSSKSTSGGTAALGCLVKDYFPEPVTVSWNSGA |
| | SEQ ID NO: | LTSGVHTFPAVLQSSGLYSLSSVVTVPSSSLGTQTYICNVNHKP |
| | 31 + SEQ ID | SNTKVDKRVEPKSCGSGGGGVYHREARSGKYKLTYAEAKAVC |
| | NO: 32) | EFEGGHLATYKQLEAARKIGFHVCAAGWMAKGRVGYPIVKP |
| | | GPNCGFGKTGIIDYGIRLNRSERWDAYCYNPHA |
| SEQ ID NO: 22 | DNA of Heavy | GAGGTGCAGCTGGTGGAATCAGGCGGCGGACTGGTGCAG |
| | Chain + Linker + | CCTGGCGGTAGCCTGAGACTGAGCTGCACCGCTAGTGGCT |
| | protein tag | TTAGCCTGACCGACTACTACTATATGACCTGGGTCAGACAG |
| | SEQ ID NO: 21 | GCCCCTGGTAAAGGCCTGGAGTGGGTCGGCTTTATCGACC |
| | | CCGACGACGACCCCTACTACGCTACCTGGGCTAAGGGCCG |
| | | GTTCACTATCTCTAGGGATAACTCTAAGAACACCCTGTACCT |
| | | GCAGATGAATAGCCTGAGAGCCGAGGACACCGCCGTCTAC |
| | | TACTGCGCCGGCGGCGATCACAATAGCGGCTGGGGCCTGG |
| | | ATATCTGGGGTCAGGGCACCCTGGTCACCGTGTCTAGCGCC |
| | | TCTACTAAGGGACCTAGCGTGTTCCCCCTGGCCCCTAGCTC |
| | | TAAGTCTACTAGCGGCGGCACCGCCGCTCTGGGCTGCCTG |
| | | GTCAAGGACTACTTCCCCGAGCCCGTGACCGTCAGCTGGA |
| | | ATAGCGGCGCTCTGACTAGCGGAGTGCACACCTTCCCCGCC |
| | | GTGCTGCAGTCTAGCGGCCTGTATAGCCTGTCTAGCGTCGT |
| | | GACCGTGCCTAGCTCTAGCCTGGGCACTCAGACCTATATCT |
| | | GTAACGTGAACCACAAGCCCTCTAACACTAAGGTGGACAA |
| | | GCGGGTGGAACCTAAGTCCTGCGGTAGCGGCGGAGGCGG |
| | | AGTCTATCACAGAGAGGCTAGATCAGGCAAGTATAAGCTG |
| | | ACCTACGCCGAGGCTAAGGCCGTGTGCGAGTTCGAGGGCG |
| | | GTCACCTGGCTACCTATAAGCAGCTGGAAGCCGCTAGAAA |
| | | GATCGGCTTTCACGTGTGCGCCGCTGGCTGGATGGCTAAG |
| | | GGTAGAGTGGGCTACCCTATCGTGAAGCCTGGCCCTAACT |
| | | GCGGCTTCGGTAAAACCGGAATTATCGACTACGGGATTAG |
| | | GCTGAATAGATCAGAGCGCTGGGACGCCTACTGCTATAAC |
| | | CCTCACGCT |
| SEQ ID NO: 11 (Kabat) | LCDR1 | 11 |
| SEQ ID NO: 12 (Kabat) | LCDR2 | 12 |
| SEQ ID NO: 13 (Kabat) | LCDR3 | 13 |
| SEQ ID NO: 14 (Chothia) | LCDR1 | 14 |
| SEQ ID NO: 15 (Chothia) | LCDR2 | 15 |
| SEQ ID NO: 16 (Chothia) | LCDR3 | 16 |
| SEQ ID NO: 17 | VL | 17 |
| SEQ ID NO: 18 | DNA of VL | 18 |
| | SEQ ID NO: 17 | |
| SEQ ID NO: 19 | Light Chain | 19 |
| SEQ ID NO: 20 | DNA of Light | 20 |
| | Chain SEQ ID | |
| | NO: 19 | |
| NVS2 | | |
| SEQ ID NO: 1 (Kabat) | HCDR1 | 1 |
| SEQ ID NO: 2 (Kabat) | HCDR2 | 2 |
| SEQ ID NO: 3 (Kabat) | HCDR3 | 3 |
| SEQ ID NO: 4 (Chothia) | HCDR1 | 4 |
| SEQ ID NO: 5 (Chothia) | HCDR2 | 5 |
| SEQ ID NO: 6 (Chothia) | HCDR3 | 6 |
| SEQ ID NO: 7 | VH | 7 |
| SEQ ID NO: 8 | DNA of VH | 8 |
| | SEQ ID NO: 7 | |
| SEQ ID NO: 9 | Heavy Chain | 9 |
| SEQ ID NO: 23 | Heavy Chain + | EVQLVESGGGLVQPGGSLRLSCTASGFSLTDYYYMTWVRQA |
| | Linker + | PGKGLEWVGFIDPDDDPYYATWAKGRFTISRDNSKNTLYLQ |
| | protein tag | MNSLRAEDTAVYYCAGGDHNSGWGLDIWGQGTLVTVSSAS |
| | (SEQ ID NO: 9 + | TKGPSVFPLAPSSKSTSGGTAALGCLVKDYFPEPVTVSWNSGA |
| | SEQ ID NO: | LTSGVHTFPAVLQSSGLYSLSSVVTVPSSSLGTQTYICNVNHKP |
| | 31 + SEQ ID | SNTKVDKRVEPKSCGSGGGGVYHREAQSGKYKLTYAEAKAVC |
| | NO: 33) | EFEGGHLATYKQLEAARKIGFHVCAAGWMAKGRVGYPIVKP |
| | | GPNCGFGKTGIIDYGIRLNRSERWDAYCYNPHA |
| SEQ ID NO: 24 | DNA of Heavy | GAGGTGCAGCTGGTGGAATCAGGCGGCGGACTGGTGCAG |
| | Chain + Linker | CCTGGCGGTAGCCTGAGACTGAGCTGCACCGCTAGTGGCT |
| | + protein tag | TTAGCCTGACCGACTACTACTATATGACCTGGGTCAGACAG |
| | SEQ ID NO: 23 | GCCCCTGGTAAAGGCCTGGAGTGGGTCGGCTTTATCGACC |
| | | CCGACGACGACCCCTACTACGCTACCTGGGCTAAGGGCCG |
| | | GTTCACTATCTCTAGGGATAACTCTAAGAACACCCTGTACCT |
| | | GCAGATGAATAGCCTGAGAGCCGAGGACACCGCCGTCTAC |
| | | TACTGCGCCGGCGGTGATCACAATAGCGGCTGGGGCCTGG |
| | | ATATCTGGGGTCAAGGCACCCTGGTCACCGTGTCTAGCGCC |
| | | TCTACTAAGGGCCCCTCAGTGTTCCCCCTGGCCCCTAGCTCT |
| | | AAGTCTACTAGCGGCGGCACCGCCGCTCTGGGCTGCCTGG |
| | | TCAAGGACTACTTCCCCGAGCCCGTGACCGTCAGCTGGAAT |
| | | AGCGGCGCTCTGACTAGCGGAGTGCACACCTTCCCCGCCGT |
| | | GCTGCAGTCTAGCGGCCTGTATAGCCTGTCTAGCGTCGTGA |
| | | CCGTGCCTAGCTCTAGCCTGGGCACTCAGACCTATATCTGT |
| | | AACGTGAACCACAAGCCCTCTAACACTAAGGTGGACAAGC |
| | | GGGTGGAACCTAAGTCCTGCGGTAGCGGCGGAGGCGGAG |
| | | TCTATCACAGAGAGGCTCAGTCAGGCAAGTATAAGCTGAC |
| | | CTACGCCGAGGCTAAGGCCGTGTGCGAGTTCGAGGGCGGT |
| | | CACCTGGCTACCTATAAGCAGCTGGAAGCCGCTAGAAAGA |
| | | TCGGCTTTCACGTGTGCGCCGCTGGCTGGATGGCTAAGGG |
| | | TAGAGTGGGCTACCCTATCGTGAAGCCTGGCCCTAACTGCG |
| | | GCTTCGGTAAAACCGGAATTATCGACTACGGGATTAGGCT |
| | | GAATAGATCAGAGCGCTGGGACGCCTACTGCTATAACCCTC |
| | | ACGCC |
| SEQ ID NO: 11 (Kabat) | LCDR1 | 11 |
| SEQ ID NO: 12 (Kabat) | LCDR2 | 12 |
| SEQ ID NO: 13 (Kabat) | LCDR3 | 13 |
| SEQ ID NO: 14 (Chothia) | LCDR1 | 14 |
| SEQ ID NO: 15 (Chothia) | LCDR2 | 15 |
| SEQ ID NO: 16 (Chothia) | LCDR3 | 16 |
| SEQ ID NO: 17 | VL | 17 |
| SEQ ID NO: 18 | DNA of VL | 18 |
| | SEQ ID NO: 18 | |
| SEQ ID NO: 19 | Light Chain | 19 |
| SEQ ID NO: 20 | DNA of Light | 10 |
| | Chain SEQ ID | |
| | NO: 20 | |
| NVS3 | | |
| SEQ ID NO: 1 (Kabat) | HCDR1 | 1 |
| SEQ ID NO: 2 (Kabat) | HCDR2 | 2 |
| SEQ ID NO: 3 (Kabat) | HCDR3 | 3 |
| SEQ ID NO: 4 (Chothia) | HCDR1 | 4 |
| SEQ ID NO: 5 (Chothia) | HCDR2 | 5 |
| SEQ ID NO: 6 (Chothia) | HCDR3 | 6 |
| SEQ ID NO: 7 | VH | 7 |
| SEQ ID NO: 8 | DNA of VH | 8 |
| | SEQ ID NO: 7 | |
| SEQ ID NO: 9 | Heavy Chain | 9 |
| SEQ ID NO: 25 | Heavy Chain + | EVQLVESGGGLVQPGGSLRLSCTASGFSLTDYYYMTWVRQA |
| | Linker + | PGKGLEWVGFIDPDDDPYYATWAKGRFTISRDNSKNTLYLQ |
| | protein tag | MNSLRAEDTAVYYCAGGDHNSGWGLDIWGQGTLVTVSSAS |
| | (SEQ ID NO: 9 + | TKGPSVFPLAPSSKSTSGGTAALGCLVKDYFPEPVTVSWNSGA |
| | SEQ ID NO: | LTSGVHTFPAVLQSSGLYSLSSVVTVPSSSLGTQTYICNVNHKP |
| | 31 + SEQ ID | SNTKVDKRVEPKSCGSGGGGVYHREAASGKYKLTYAEAKAVC |
| | NO: 34) | EFEGGHLATYKQLEAARKIGFHVCAAGWMAKGRVGYPIVKP |
| | | GPNCGFGKTGIIDYGIRLNRSERWDAYCYNPHA |
| SEQ ID NO: 26 | DNA of Heavy | GAGGTGCAGCTGGTGGAATCAGGCGGCGGACTGGTGCAG |
| | Chain + Linker + | CCTGGCGGTAGCCTGAGACTGAGCTGCACCGCTAGTGGCT |
| | protein tag | TTAGCCTGACCGACTACTACTATATGACCTGGGTCAGACAG |
| | SEQ ID NO: 25 | GCCCCTGGTAAAGGCCTGGAGTGGGTCGGCTTTATCGACC |
| | | CCGACGACGACCCCTACTACGCTACCTGGGCTAAGGGCCG |
| | | GTTCACTATCTCTAGGGATAACTCTAAGAACACCCTGTACCT |
| | | GCAGATGAATAGCCTGAGAGCCGAGGACACCGCCGTCTAC |
| | | TACTGCGCCGGCGGTGATCACAATAGCGGCTGGGGCCTGG |
| | | ATATCTGGGGTCAAGGCACCCTGGTCACCGTGTCTAGCGCC |
| | | TCTACTAAGGGCCCCTCAGTGTTCCCCCTGGCCCCTAGCTCT |
| | | AAGTCTACTAGCGGCGGCACCGCCGCTCTGGGCTGCCTGG |
| | | TCAAGGACTACTTCCCCGAGCCCGTGACCGTCAGCTGGAAT |
| | | AGCGGCGCTCTGACTAGCGGAGTGCACACCTTCCCCGCCGT |
| | | GCTGCAGTCTAGCGGCCTGTATAGCCTGTCTAGCGTCGTGA |
| | | CCGTGCCTAGCTCTAGCCTGGGCACTCAGACCTATATCTGT |
| | | AACGTGAACCACAAGCCCTCTAACACTAAGGTGGACAAGC |
| | | GGGTGGAACCTAAGTCCTGCGGTAGCGGCGGAGGCGGAG |
| | | TCTATCACAGAGAGGCTGCTAGCGGTAAATACAAGCTGAC |
| | | CTACGCCGAGGCTAAGGCCGTGTGCGAGTTCGAGGGCGGT |
| | | CACCTGGCTACCTATAAGCAGCTGGAAGCCGCTAGAAAGA |
| | | TCGGCTTTCACGTGTGCGCCGCTGGCTGGATGGCTAAGGG |
| | | TAGAGTGGGCTACCCTATCGTGAAGCCTGGCCCTAACTGCG |
| | | GCTTCGGTAAAACCGGAATTATCGACTACGGGATTAGGCT |
| | | GAATAGATCAGAGCGCTGGGACGCCTACTGCTATAACCCTC |
| | | ACGCC |
| SEQ ID NO: 11 (Kabat) | LCDR1 | 11 |
| SEQ ID NO: 12 (Kabat) | LCDR2 | 12 |
| SEQ ID NO: 13 (Kabat) | LCDR3 | 13 |
| SEQ ID NO: 14 (Chothia) | LCDR1 | 14 |
| SEQ ID NO: 15 (Chothia) | LCDR2 | 15 |
| SEQ ID NO: 16 (Chothia) | LCDR3 | 16 |
| SEQ ID NO: 17 | VL | 17 |
| SEQ ID NO: 18 | DNA of VL | 18 |
| | SEQ ID NO: 18 | |
| SEQ ID NO: 19 | Light Chain | 19 |
| SEQ ID NO: 20 | DNA of Light | 20 |
| | Chain SEQ ID | |
| | NO: 19 | |
| NVS36 | | |
| SEQ ID NO: 1 (Kabat) | HCDR1 | 1 |
| SEQ ID NO: 2 (Kabat) | HCDR2 | 2 |
| SEQ ID NO: 3 (Kabat) | HCDR3 | 3 |
| SEQ ID NO: 4 (Chothia) | HCDR1 | 4 |
| SEQ ID NO: 5 (Chothia) | HCDR2 | 5 |
| SEQ ID NO: 6 (Chothia) | HCDR3 | 6 |
| SEQ ID NO: 7 | VH | 7 |
| SEQ ID NO: 8 | DNA of VH | 8 |
| | SEQ ID NO: 7 | |
| SEQ ID NO: 9 | Heavy Chain | 9 |
| SEQ ID NO: 27 | Heavy Chain + | EVQLVESGGGLVQPGGSLRLSCTASGFSLTDYYYMTWVRQA |
| | Linker + | PGKGLEWVGFIDPDDDPYYATWAKGRFTISRDNSKNTLYLQ |
| | protein tag | MNSLRAEDTAVYYCAGGDHNSGWGLDIWGQGTLVTVSSAS |
| | (SEQ ID NO: 9 + | TKGPSVFPLAPSSKSTSGGTAALGCLVKDYFPEPVTVSWNSGA |
| | SEQ ID NO: | LTSGVHTFPAVLQSSGLYSLSSVVTVPSSSLGTQTYICNVNHKP |
| | 31 + SEQ ID | SNTKVDKRVEPKSCGSGGGACGVYHREAQSGKYKLTYAEAKA |
| | NO: 35) | VCEFEGGHLATYKQLECARKIGFHVCAAGWMAKGRVGYPIV |
| | | KPGPNCGFGKTGIIDYGIRLNRSERWDAYCYNPHA |
| SEQ ID NO : 28 | DNA of Heavy | GAAGTGCAGCTGGTGGAAAGCGGCGGAGGCCTGGTGCAG |
| | Chain + Linker + | CCTGGCGGATCTCTGAGACTGAGCTGTACCGCCAGCGGCTT |
| | protein tag | CAGCCTGACCGACTACTACTACATGACCTGGGTCCGACAGG |
| | SEQ ID NO: 27 | CCCCTGGCAAGGGACTGGAATGGGTCGGATTCATCGACCC |
| | | CGACGACGACCCCTACTACGCCACATGGGCCAAGGGCCGG |
| | | TTCACCATCAGCCGGGACAACAGCAAGAACACCCTGTACCT |
| | | GCAGATGAACAGCCTGCGGGCCGAGGACACCGCCGTGTAC |
| | | TATTGTGCCGGCGGAGATCACAACAGCGGCTGGGGCCTGG |
| | | ATATCTGGGGACAGGGAACACTGGTCACCGTGTCTAGCGC |
| | | CAGCACCAAGGGCCCTAGCGTGTTCCCTCTGGCCCCTAGCA |
| | | GCAAGAGCACATCTGGCGGAACAGCCGCCCTGGGCTGCCT |
| | | GGTCAAGGACTACTTTCCCGAGCCCGTGACCGTGTCCTGGA |
| | | ACTCTGGCGCTCTGACAAGCGGCGTGCACACCTTTCCAGCC |
| | | GTGCTGCAGAGCAGCGGCCTGTACTCTCTGAGCAGCGTGG |
| | | TCACAGTGCCCAGCTCTAGCCTGGGAACCCAGACCTACATC |
| | | TGCAACGTGAACCACAAGCCCAGCAACACCAAGGTGGACA |
| | | AGCGGGTGGAACCCAAGAGCTGCGGATCCGGCGGAGGCG |
| | | CCTGTGGCGTGTATCACAGGGAGGCCCAGAGCGGCAAGTA |
| | | CAAGCTCACCTACGCCGAGGCCAAGGCCGTGTGCGAATTC |
| | | GAGGGCGGCCACCTGGCCACCTACAAGCAGCTGGAGTGCG |
| | | CCAGGAAGATCGGCTTCCACGTGTGTGCCGCCGGCTGGAT |
| | | GGCCAAAGGCAGAGTGGGCTACCCCATCGTGAAACCCGGC |
| | | CCCAACTGCGGCTTCGGCAAGACAGGCATCATCGACTACG |
| | | GCATCAGGCTGAACAGGAGCGAGAGGTGGGACGCCTACT |
| | | GCTACAACCCCCACGCC |
| SEQ ID NO: 11 (Kabat) | LCDR1 | 11 |
| SEQ ID NO: 12 (Kabat) | LCDR2 | 12 |
| SEQ ID NO: 13 (Kabat) | LCDR3 | 13 |
| SEQ ID NO: 14 (Chothia) | LCDR1 | 14 |
| SEQ ID NO: 15 (Chothia) | LCDR2 | 15 |
| SEQ ID NO: 16 (Chothia) | LCDR3 | 16 |
| SEQ ID NO: 17 | VL | 17 |
| SEQ ID NO: 18 | DNA of VL | 18 |
| | SEQ ID NO: 18 | |
| SEQ ID NO: 19 | Light Chain | 19 |
| SEQ ID NO: 20 | DNA of Light | 20 |
| | Chain SEQ ID | |
| | NO: 19 | |
| NVS37 | | |
| SEQ ID NO: 1 (Kabat) | HCDR1 | 1 |
| SEQ ID NO: 2 (Kabat) | HCDR2 | 2 |
| SEQ ID NO: 3 (Kabat) | HCDR3 | 3 |
| SEQ ID NO: 4 (Chothia) | HCDR1 | 4 |
| SEQ ID NO: 5 (Chothia) | HCDR2 | 5 |
| SEQ ID NO: 6 (Chothia) | HCDR3 | 6 |
| SEQ ID NO: 7 | VH | 6 |
| SEQ ID NO: 8 | DNA of VH | 8 |
| | SEQ ID NO: 7 | |
| SEQ ID NO: 9 | Heavy Chain | 9 |
| SEQ ID NO: 29 | Heavy Chain + | EVQLVESGGGLVQPGGSLRLSCTASGFSLTDYYYMTWVRQA |
| | Linker + | PGKGLEWVGFIDPDDDPYYATWAKGRFTISRDNSKNTLYLQ |
| | protein tag | MNSLRAEDTAVYYCAGGDHNSGWGLDIWGQGTLVTVSSAS |
| | (SEQ ID NO: 9 + | TKGPSVFPLAPSSKSTSGGTAALGCLVKDYFPEPVTVSWNSGA |
| | SEQ ID NO: | LTSGVHTFPAVLQSSGLYSLSSVVTVPSSSLGTQTYICNVNHKP |
| | 31 + SEQ ID | SNTKVDKRVEPKSCGSGGGGVYHREAQSGKYKLTYAEAKAVC |
| | NO: 36) | EFEGGHLCTYKQLEAARKIGFHVCAAGWMAKGRVGYPIVKP |
| | | GPNCGFGKTGIIDYGIRLNRSERWDAYCCNPHA |
| SEQ ID NO: 30 | DNA of Heavy | GAAGTGCAGCTGGTGGAAAGCGGCGGAGGCCTGGTGCAG |
| | Chain + Linker + | CCTGGCGGATCTCTGAGACTGAGCTGTACCGCCAGCGGCTT |
| | protein tag | CAGCCTGACCGACTACTACTACATGACCTGGGTCCGACAGG |
| | SEQ ID NO: 29 | CCCCTGGCAAGGGACTGGAATGGGTCGGATTCATCGACCC |
| | | CGACGACGACCCCTACTACGCCACATGGGCCAAGGGCCGG |
| | | TTCACCATCAGCCGGGACAACAGCAAGAACACCCTGTACCT |
| | | GCAGATGAACAGCCTGCGGGCCGAGGACACCGCCGTGTAC |
| | | TATTGTGCCGGCGGAGATCACAACAGCGGCTGGGGCCTGG |
| | | ATATCTGGGGACAGGGAACACTGGTCACCGTGTCTAGCGC |
| | | CAGCACCAAGGGCCCTAGCGTGTTCCCTCTGGCCCCTAGCA |
| | | GCAAGAGCACATCTGGCGGAACAGCCGCCCTGGGCTGCCT |
| | | GGTCAAGGACTACTTTCCCGAGCCCGTGACCGTGTCCTGGA |
| | | ACTCTGGCGCTCTGACAAGCGGCGTGCACACCTTTCCAGCC |
| | | GTGCTGCAGAGCAGCGGCCTGTACTCTCTGAGCAGCGTGG |
| | | TCACAGTGCCCAGCTCTAGCCTGGGAACCCAGACCTACATC |
| | | TGCAACGTGAACCACAAGCCCAGCAACACCAAGGTGGACA |
| | | AGCGGGTGGAACCCAAGAGCTGCGGATCCGGCGGCGGCG |
| | | GAGTGTATCACAGAGAGGCCCAGAGCGGCAAGTACAAGCT |
| | | GACCTACGCCGAGGCCAAGGCCGTGTGTGAGTTCGAGGGC |
| | | GGCCACCTGTGCACCTACAAGCAGCTGGAGGCCGCCAGGA |
| | | AGATCGGCTTCCACGTGTGTGCCGCCGGCTGGATGGCTAA |
| | | AGGCAGGGTGGGCTACCCCATTGTGAAGCCCGGCCCCAAT |
| | | TGCGGCTTCGGCAAGACCGGCATCATCGACTACGGCATCA |
| | | GGCTGAACAGGAGCGAGAGGTGGGACGCCTACTGCTGCA |
| | | ACCCCCACGCC |
| SEQ ID NO: 11 (Kabat) | LCDR1 | 11 |
| SEQ ID NO: 12 (Kabat) | LCDR2 | 12 |
| SEQ ID NO: 13 (Kabat) | LCDR3 | 13 |
| SEQ ID NO: 14 (Chothia) | LCDR1 | 14 |
| SEQ ID NO: 15 (Chothia) | LCDR2 | 15 |
| SEQ ID NO: 16 (Chothia) | LCDR3 | 16 |
| SEQ ID NO: 17 | VL | 17 |
| SEQ ID NO: 18 | DNA of VL | 18 |
| | SEQ ID NO: 18 | |
| SEQ ID NO: 19 | Light Chain | 19 |
| SEQ ID NO: 20 | DNA of Light | 20 |
| | Chain SEQ ID | |
| | NO: 19 | |
| Tag and Linker Sequences | | |
| SEQ ID NO: 31 | Linker | GSGGG |
| SEQ ID NO: 32 | Protein tag 1 | GVYHREARSGKYKLTYAEAKAVCEFEGGHLATYKQLEAARKIG |
| | (HA10) | FHVCAAGWMAKGRVGYPIVKPGPNCGFGKTGIIDYGIRLNRS |
| | | ERWDAYCYNPHAK |
| SEQ ID NO: 33 | Protein tag 2 | GVYHREAQSGKYKLTYAEAKAVCEFEGGHLATYKQLEAARKIG |
| | (HA10.1) | FHVCAAGWMAKGRVGYPIVKPGPNCGFGKTGIIDYGIRLNRS |
| | | ERWDAYCYNPHA |
| SEQ ID NO: 34 | Protein tag 3 | GVYHREAASGKYKLTYAEAKAVCEFEGGHLATYKQLEAARKIG |
| | (HA 10.2) | FHVCAAGWMAKGRVGYPIVKPGPNCGFGKTGIIDYGIRLNRS |
| | | ERWDAYCYNPHA |
| SEQ ID NO: 35 | Protein tag 4 | ACGVYHREAQSGKYKLTYAEAKAVCEFEGGHLATYKQLECAR |
| | (HA 11) | KIGFHVCAAGWMAKGRVGYPIVKPGPNCGFGKTGIIDYGIRL |
| | | NRSERWDAYCYNPHA |
| SEQ ID NO: 36 | Protein tag 5 | GVYHREAQSGKYKLTYAEAKAVCEFEGGHLCTYKQLEAARKIG |
| | (HA 11.1) | FHVCAAGWMAKGRVGYPIVKPGPNCGFGKTGIIDYGIRLNRS |
| | | ERWDAYCCNPHA |
| SEQ ID NO: 103 | DNA of | GGAGTCTATCACAGAGAGGCTAGATCAGGCAAGTATAAGC |
| | SEQ ID NO: 32 | TGACCTACGCCGAGGCTAAGGCCGTGTGCGAGTTCGAGGG |
| | (HA10) | CGGTCACCTGGCTACCTATAAGCAGCTGGAAGCCGCTAGA |
| | | AAGATCGGCTTTCACGTGTGCGCCGCTGGCTGGATGGCTA |
| | | AGGGTAGAGTGGGCTACCCTATCGTGAAGCCTGGCCCTAA |
| | | CTGCGGCTTCGGTAAAACCGGAATTATCGACTACGGGATTA |
| | | GGCTGAATAGATCAGAGCGCTGGGACGCCTACTGCTATAA |
| | | CCCTCACGCTAAG |
| SEQ ID NO: 104 | DNA of | GGAGTCTATCACAGAGAGGCTCAGTCAGGCAAGTATAAGC |
| | SEQ ID NO: 33 | TGACCTACGCCGAGGCTAAGGCCGTGTGCGAGTTCGAGGG |
| | (HA10.1) | CGGTCACCTGGCTACCTATAAGCAGCTGGAAGCCGCTAGA |
| | | AAGATCGGCTTTCACGTGTGCGCCGCTGGCTGGATGGCTA |
| | | AGGGTAGAGTGGGCTACCCTATCGTGAAGCCTGGCCCTAA |
| | | CTGCGGCTTCGGTAAAACCGGAATTATCGACTACGGGATTA |
| | | GGCTGAATAGATCAGAGCGCTGGGACGCCTACTGCTATAA |
| | | CCCTCACGCC |
| SEQ ID NO: 105 | DNA of | GGAGTCTATCACAGAGAGGCTGCTAGCGGTAAATACAAGC |
| | SEQ ID NO: 34 | TGACCTACGCCGAGGCTAAGGCCGTGTGCGAGTTCGAGGG |
| | (HA10.2) | CGGTCACCTGGCTACCTATAAGCAGCTGGAAGCCGCTAGA |
| | | AAGATCGGCTTTCACGTGTGCGCCGCTGGCTGGATGGCTA |
| | | AGGGTAGAGTGGGCTACCCTATCGTGAAGCCTGGCCCTAA |
| | | CTGCGGCTTCGGTAAAACCGGAATTATCGACTACGGGATTA |
| | | GGCTGAATAGATCAGAGCGCTGGGACGCCTACTGCTATAA |
| | | CCCTCACGCC |
| SEQ ID NO: 106 | DNA of | GGCGCCTGTGGCGTGTATCACAGGGAGGCCCAGAGCGGC |
| | SEQ ID NO: 35 | AAGTACAAGCTCACCTACGCCGAGGCCAAGGCCGTGTGCG |
| | (HA11) | AATTCGAGGGCGGCCACCTGGCCACCTACAAGCAGCTGGA |
| | | GTGCGCCAGGAAGATCGGCTTCCACGTGTGTGCCGCCGGC |
| | | TGGATGGCCAAAGGCAGAGTGGGCTACCCCATCGTGAAAC |
| | | CCGGCCCCAACTGCGGCTTCGGCAAGACAGGCATCATCGA |
| | | CTACGGCATCAGGCTGAACAGGAGCGAGAGGTGGGACGC |
| | | CTACTGCTACAACCCCCACGCC |
| SEQ ID NO: 107 | DNA of | GGAGTGTATCACAGAGAGGCCCAGAGCGGCAAGTACAAG |
| | SEQ ID NO: 36 | CTGACCTACGCCGAGGCCAAGGCCGTGTGTGAGTTCGAGG |
| | (HA11.1) | GCGGCCACCTGTGCACCTACAAGCAGCTGGAGGCCGCCAG |
| | | GAAGATCGGCTTCCACGTGTGTGCCGCCGGCTGGATGGCT |
| | | AAAGGCAGGGTGGGCTACCCCATTGTGAAGCCCGGCCCCA |
| | | ATTGCGGCTTCGGCAAGACCGGCATCATCGACTACGGCATC |
| | | AGGCTGAACAGGAGCGAGAGGTGGGACGCCTACTGCTGC |
| | | AACCCCCACGCC |

TABLE 2
Examples of additional peptide tagged molecules (e.g.: NVS70T, NVS71T,
NVS72T and NVS75T), untagged molecules (e.g.: NVS70, NVS71, NVS72 and NVS75)
and component sequences.

NVS70 and NVS70T

| | | |
|---|---|---|
| SEQ ID NO: 37 | HCDR1 | SYAIS |
| SEQ ID NO: 38 | HCDR2 | GIGPFFGTANYAQKFQG |
| SEQ ID NO: 39 | HCDR3 | DTPYFDY |
| SEQ ID NO: 40 | VH | EVQLVQSGAEVKKPGSSVKVSCKASGGTFSSYAISWVRQAPG |
| | | QGLEWMGGIGPFFGTANYAQKFQGRVTITADESTSTAYMEL |
| | | SSLRSEDTAVYYCARDTPYFDYWGQGTLVTVSS |
| SEQ ID NO: 41 | DNA of VH | GAGGTGCAATTGGTTCAGTCTGGCGCGGAAGTGAAAAAAC |
| | SEQ ID NO: 40 | CGGGCAGCAGCGTGAAAGTGAGCTGCAAAGCCTCCGGAG |
| | | GCACTTTTTCTTCTTATGCCATTTCTTGGGTGCGCCAAGCCC |
| | | CTGGGCAGGGTCTCGAGTGGATGGGCGGTATCGGTCCGTT |
| | | TTTTGGCACTGCGAATTACGCGCAGAAGTTTCAGGGCCGG |
| | | GTGACCATTACCGCGGATGAAAGCACCAGCACCGCGTATA |
| | | TGGAACTGAGCAGCCTGCGTAGCGAAGATACGGCCGTGTA |
| | | TTATTGCGCGCGTGATACTCCTTATTTTGATTATTGGGGCCA |
| | | AGGCACCCTGGTGACGGTTAGCTCA |
| SEQ ID NO: 42 | Heavy Chain | EVQLVQSGAEVKKPGSSVKVSCKASGGTFSSYAISWVRQAPG |
| | | QGLEWMGGIGPFFGTANYAQKFQGRVTITADESTSTAYMEL |
| | | SSLRSEDTAVYYCARDTPYFDYWGQGTLVTVSSASTKGPSVFP |
| | | LAPSSKSTSGGTAALGCLVKDYFPEPVTVSWNSGALTSGVHTF |
| | | PAVLQSSGLYSLSSVVTVPSSSLGTQTYICNVNHKPSNTKVDKR |
| | | VEPKSC |
| SEQ ID NO: 43 | DNA of Heavy | GAGGTGCAATTGGTCCAAAGCGGCGCTGAGGTCAAGAAG |
| | Chain SEQ ID | CCTGGCAGCAGCGTGAAGGTCTCCTGCAAGGCCAGCGGCG |
| | NO: 42 | GCACATTCTCCAGCTATGCTATCAGCTGGGTCAGACAAGCC |
| | | CCCGGCCAAGGACTGGAATGGATGGGAGGAATCGGCCCTT |
| | | TCTTCGGAACCGCCAACTACGCCCAGAAGTTTCAGGGAAG |
| | | GGTGACCATCACCGCCGATGAGAGCACATCCACAGCCTAT |
| | | ATGGAGCTCTCCAGCCTGAGATCCGAAGACACCGCCGTCTA |
| | | CTACTGCGCTAGGGACACCCCCTACTTCGACTATTGGGGCC |
| | | AGGGCACACTCGTGACCGTGAGCTCAGCCAGCACCAAAGG |
| | | CCCTAGCGTCTTCCCCCTGGCTCCTTCCAGCAAGAGCACAA |
| | | GCGGAGGAACAGCTGCTCTCGGCTGCCTGGTCAAGGACTA |
| | | CTTCCCCGAGCCTGTCACAGTGTCCTGGAATAGCGGAGCCC |
| | | TGACCAGCGGCGTGCATACATTCCCCGCTGTGCTCCAGAGC |
| | | TCCGGCCTCTACAGCCTCAGCTCCGTGGTCACCGTCCCTAG |
| | | CTCCTCCCTGGGCACACAGACCTACATCTGCAACGTCAACC |
| | | ACAAGCCCTCCAACACCAAGGTGGACAAGAGGGTGGAGCC |
| | | CAAAAGCTGT |
| SEQ ID NO: 44 | Heavy Chain + | EVQLVQSGAEVKKPGSSVKVSCKASGGTFSSYAISWVRQAPG |
| | Linker + | QGLEWMGGIGPFFGTANYAQKFQGRVTITADESTSTAYMEL |
| | protein tag | SSLRSEDTAVYYCARDTPYFDYWGQGTLVTVSSASTKGPSVFP |
| | (SEQ ID NO: | LAPSSKSTSGGTAALGCLVKDYFPEPVTVSWNSGALTSGVHTF |
| | 42 + SEQ ID | PAVLQSSGLYSLSSVVTVPSSSLGTQTYICNVNHKPSNTKVDKR |
| | NO: 31 + SEQ | VEPKSCGSGGGGVYHREAQSGKYKLTYAEAKAVCEFEGGHLA |
| | ID NO: 34) | TYKQLEAARKIGFHVCAAGWMAKGRVGYPIVKPGPNCGFGK |
| | | TGIIDYGIRLNRSERWDAYCYNPH |
| | | A |
| SEQ ID NO: 45 | DNA of Heavy | GAGGTGCAATTGGTCCAAAGCGGCGCTGAGGTCAAGAAG |
| | Chain + Linker + | CCTGGCAGCAGCGTGAAGGTCTCCTGCAAGGCCAGCGGCG |
| | protein tag | GCACATTCTCCAGCTATGCTATCAGCTGGGTCAGACAAGCC |
| | SEQ ID NO: 44 | CCCGGCCAAGGACTGGAATGGATGGGAGGAATCGGCCCTT |
| | | TCTTCGGAACCGCCAACTACGCCCAGAAGTTTCAGGGAAG |
| | | GGTGACCATCACCGCCGATGAGAGCACATCCACAGCCTAT |
| | | ATGGAGCTCTCCAGCCTGAGATCCGAAGACACCGCCGTCTA |
| | | CTACTGCGCTAGGGACACCCCCTACTTCGACTATTGGGGCC |
| | | AGGGCACACTCGTGACCGTGAGCTCAGCCAGCACCAAAGG |
| | | CCCTAGCGTCTTCCCCCTGGCTCCTTCCAGCAAGAGCACAA |
| | | GCGGAGGAACAGCTGCTCTCGGCTGCCTGGTCAAGGACTA |
| | | CTTCCCCGAGCCTGTCACAGTGTCCTGGAATAGCGGAGCCC |
| | | TGACCAGCGGCGTGCATACATTCCCCGCTGTGCTCCAGAGC |
| | | TCCGGCCTCTACAGCCTCAGCTCCGTGGTCACCGTCCCTAG |
| | | CTCCTCCCTGGGCACACAGACCTACATCTGCAACGTCAACC |
| | | ACAAGCCCTCCAACACCAAGGTGGACAAGAGGGTGGAGCC |
| | | CAAAAGCTGTGGATCCGGAGGAGGCGGCGTGTATCATAGA |
| | | GAGGCCCAGTCCGGCAAGTACAAGCTGACCTACGCCGAAG |
| | | CCAAGGCCGTGTGTGAGTTCGAGGGCGGACACCTGGCTAC |
| | | CTACAAACAGCTCGAAGCCGCTAGGAAGATCGGATTCCAC |
| | | GTGTGCGCCGCCGGATGGATGGCCAAAGGCAGAGTGGGC |
| | | TACCCCATTGTCAAGCCCGGACCCAACTGCGGATTCGGCAA |
| | | GACCGGCATCATCGACTACGGCATCAGGCTCAACAGGTCC |
| | | GAGAGATGGGACGCTTACTGCTACAATCCCCACGCC |
| SEQ ID NO: 46 | LCDR1 | SGDSIPNYYVY |
| SEQ ID NO: 47 | LCDR2 | DDSNRPS |
| SEQ ID NO: 48 | LCDR3 | QSFDSSLNAEV |
| SEQ ID NO: 49 | VL | SYELTQPLSVSVALGQTARITCSGDSIPNYYVYWYQQKPGQAP |
| | | VLVIYDDSNRPSGIPERFSGSNSGNTATLTISRAQAGDEADYYC |
| | | QSFDSSLNAEVFGGGTKLTVL |
| SEQ ID NO: 50 | DNA of VL SEQ | TCCTATGAACTCACACAGCCCCTGAGCGTGAGCGTGGCCCT |
| | ID NO: 49 | GGGCCAGACCGCCCGGATCACCTGCTCCGGCGACAGCATC |
| | | CCCAACTACTACGTGTACTGGTACCAGCAGAAGCCCGGCCA |
| | | GGCCCCCGTGCTGGTGATCTACGACGACAGCAACCGGCCC |
| | | AGCGGCATCCCCGAGCGGTTCAGCGGCAGCAACAGCGGCA |
| | | ACACCGCCACCCTGACCATTTCCAGAGCACAGGCAGGCGA |
| | | CGAGGCCGACTACTACTGCCAGAGCTTCGACAGCAGCCTG |
| | | AACGCCGAGGTGTTCGGCGGAGGGACCAAGTTAACCGTCC |
| | | TA |
| SEQ ID NO: 51 | Light Chain | SYELTQPLSVSVALGQTARITCSGDSIPNYYVYWYQQKPGQAP |
| | | VLVIYDDSNRPSGIPERFSGSNSGNTATLTISRAQAGDEADYYC |
| | | QSFDSSLNAEVFGGGTKLTVLGQPKAAPSVTLFPPSSEELQAN |
| | | KATLVCLISDFYPGAVTVAWKADSSPVKAGVETTTPSKQSNN |
| | | KYAASSYLSLTPEQWKSHRSYSCQVTHEGSTVEKTVAPTECS |
| SEQ ID NO: 52 | DNA of Light | AGCTACGAGCTGACCCAGCCCCTGAGCGTGAGCGTGGCCC |
| | Chain SEQ ID | TGGGCCAGACCGCCAGGATCACCTGCAGCGGCGACAGCAT |
| | NO: 51 | CCCCAACTACTACGTGTACTGGTATCAGCAGAAGCCCGGCC |
| | | AGGCCCCCGTGCTGGTGATCTACGACGACAGCAACAGGCC |
| | | CAGCGGCATCCCCGAGAGGTTCAGCGGCAGCAACAGCGGC |
| | | AACACCGCCACCCTGACCATCAGCAGAGCCCAGGCCGGCG |
| | | ACGAGGCCGACTACTACTGCCAGAGCTTCGACAGCTCACTG |
| | | AACGCCGAGGTGTTCGGCGGAGGGACCAAGCTGACCGTG |
| | | CTGGGCCAGCCTAAGGCTGCCCCCAGCGTGACCCTGTTCCC |
| | | CCCCAGCAGCGAGGAGCTGCAGGCCAACAAGGCCACCCTG |
| | | GTGTGCCTGATCAGCGACTTCTACCCAGGCGCCGTGACCGT |
| | | GGCCTGGAAGGCCGACAGCAGCCCCGTGAAGGCCGGCGT |
| | | GGAGACCACCACCCCCAGCAAGCAGAGCAACAACAAGTAC |
| | | GCCGCCAGCAGCTACCTGAGCCTGACCCCCGAGCAGTGGA |
| | | AGAGCCACAGGTCCTACAGCTGCCAGGTGACCCACGAGGG |
| | | CAGCACCGTGGAAAAGACCGTGGCCCCAACCGAGTGCAGC |
| NVS71 and NVS71T | | |
| SEQ ID NO: 53 (Kabat) | HCDR1 | SYAIS |
| SEQ ID NO: 54 (Kabat) | HCDR2 | RIIPIFGTANYAQKFQG |
| SEQ ID NO: 55 (Kabat) | HCDR3 | HGGYSFDS |
| SEQ ID NO: 56 (Chothia) | HCDR1 | GGTFNSY |
| SEQ ID NO: 57 (Chothia) | HCDR2 | IPIFGT |
| SEQ ID NO: 58 (Chothia) | HCDR3 | HGGYSFDS |
| SEQ ID NO: 59 | VH | EVQLVQSGAEVKKPGSSVKVSCKASGGTFNSYAISWVRQAPG |
| | | QGLEWMGRIIPIFGTANYAQKFQGRVTITADESTSTAYMELSS |
| | | LRSEDTAVYYCARHGGYSFDSWGQGTLVTVSS |
| SEQ ID NO: 60 | DNA of VH | GAGGTGCAGCTGGTGCAGAGCGGAGCCGAAGTGAAGAAA |
| | SEQ ID NO: 59 | CCCGGCAGCAGCGTGAAGGTGTCCTGCAAGGCCAGCGGC |
| | | GGCACCTTCAACAGCTACGCCATCAGCTGGGTGCGCCAGG |
| | | CTCCTGGACAGGGCCTGGAATGGATGGGCCGGATCATCCC |
| | | CATCTTCGGCACCGCCAACTACGCCCAGAAATTCCAGGGCA |
| | | GAGTGACCATCACCGCCGACGAGAGCACCAGCACCGCCTA |
| | | CATGGAACTGAGCAGCCTGAGAAGCGAGGACACCGCCGT |
| | | GTACTACTGTGCCCGGCACGGCGGCTACAGCTTCGATAGCT |
| | | GGGGCCAGGGCACCCTGGTGACCGTGAGCTCA |
| SEQ ID NO: 61 | Heavy Chain | EVQLVQSGAEVKKPGSSVKVSCKASGGTFSSYAISWVRQAPG |
| | | QGLEWMGRIIPIFGTANYAQKFQGRVTITADESTSTAYMELSS |
| | | LRSEDTAVYYCARHGGYSFDSWGQGTLVTVSSASTKGPSVFP |
| | | LAPSSKSTSGGTAALGCLVKDYFPEPVTVSWNSGALTSGVHTF |
| | | PAVLQSSGLYSLSSVVTVPSSSLGTQTYICNVNHKPSNTKVDKR |
| | | VEPKSC |
| SEQ ID NO: 62 | DNA of Heavy | GAGGTGCAGCTGGTGCAGAGCGGAGCCGAAGTGAAGAAA |
| | Chain SEQ ID | CCCGGCAGCAGCGTGAAGGTGTCCTGCAAGGCCAGCGGC |
| | NO: 61 | GGCACCTTCAACAGCTACGCCATCAGCTGGGTGCGCCAGG |
| | | CTCCTGGACAGGGCCTGGAATGGATGGGCCGGATCATCCC |
| | | CATCTTCGGCACCGCCAACTACGCCCAGAAATTCCAGGGCA |
| | | GAGTGACCATCACCGCCGACGAGAGCACCAGCACCGCCTA |
| | | CATGGAACTGAGCAGCCTGAGAAGCGAGGACACCGCCGT |
| | | GTACTACTGTGCCCGGCACGGCGGCTACAGCTTCGATAGCT |
| | | GGGGCCAGGGCACCCTGGTGACCGTGAGCTCAGCCTCCAC |
| | | CAAGGGTCCATCGGTCTTCCCCCTGGCACCCTCCTCCAAGA |
| | | GCACCTCTGGGGGCACAGCGGCCCTGGGCTGCCTGGTCAA |
| | | GGACTACTTCCCCGAACCGGTGACGGTGTCGTGGAACTCA |
| | | GGCGCCCTGACCAGCGGCGTGCACACCTTCCCGGCTGTCCT |
| | | ACAGTCCTCAGGACTCTACTCCCTCAGCAGCGTGGTGACCG |
| | | TGCCCTCCAGCAGC-1TGGGCACCCAGACCTACATCTGCAAC |
| | | GTGAATCACAAGCCCAGCAACACCAAGGTGGACAAGAGAG |
| | | TTGAGCCCAAATCTTGT |
| SEQ ID NO: 63 | Heavy Chain + | EVQLVQSGAEVKKPGSSVKVSCKASGGTFSSYAISWVRQAPG |
| | Linker + | QGLEWMGRIIPIFGTANYAQKFQGRVTITADESTSTAYMELSS |
| | protein tag | LRSEDTAVYYCARHGGYSFDSWGQGTLVTVSSASTKGPSVFP |
| | (SEQ ID NO: | LAPSSKSTSGGTAALGCLVKDYFPEPVTVSWNSGALTSGVHTF |
| | 61 + SEQ ID | PAVLQSSGLYSLSSVVTVPSSSLGTQTYICNVNHKPSNTKVDKR |
| | NO: 31 + SEQ | VEPKSCGSGGGGVYHREAQSGKYKLTYAEAKAVCEFEGGHLA |
| | ID NO: 33) | TYKQLEAARKIGFHVCAAGWMAKGRVGYPIVKPGPNCGFGK |
| | | TGIIDYGIRLNRSERWDAYCYNPHA |
| SEQ ID NO: 64 | DNA of Heavy | GAGGTGCAATTGGTGCAGAGCGGAGCTGAGGTGAAGAAG |
| | Chain + Linker + | CCCGGCAGCTCCGTCAAGGTGAGCTGCAAAGCCTCCGGAG |
| | protein tag | GCACCTTTTCCTCCTACGCTATCTCCTGGGTGAGGCAAGCC |
| | SEQ ID NO: 63 | CCCGGACAAGGACTGGAGTGGATGGGCAGGATCATCCCCA |
| | | TCTTCGGAACCGCCAACTACGCCCAGAAATTCCAGGGCAG |
| | | GGTGACCATCACCGCCGACGAAAGCACCAGCACCGCCTAC |
| | | ATGGAGCTCTCCAGCCTGAGGAGCGAGGACACCGCTGTGT |
| | | ACTACTGCGCCAGACACGGCGGCTACTATTTCGACAGCTGG |
| | | GGCCAGGGCACACTGGTGACCGTGAGCTCAGCAAGCACCA |
| | | AAGGACCCTCCGTCTTTCCTCTGGCCCCCAGCAGCAAGTCC |
| | | ACAAGCGGAGGAACCGCTGCCCTGGGATGTCTCGTGAAGG |
| | | ACTACTTCCCTGAGCCCGTGACAGTGTCCTGGAATAGCGGC |
| | | GCCCTGACAAGCGGCGTGCACACATTTCCCGCCGTCCTGCA |
| | | AAGCTCCGGCCTCTATAGCCTGAGCTCCGTCGTGACAGTCC |
| | | CCTCCAGCTCCCTGGGAACCCAGACCTACATCTGCAACGTC |
| | | AACCACAAGCCCAGCAACACAAAGGTGGACAAGAGGGTC |
| | | GAGCCTAAGAGCTGTGGATCCGGCGGCGGAGGAGTGTAC |
| | | CATAGGGAGGCCCAGAGCGGAAAGTACAAGCTGACCTATG |
| | | CCGAGGCTAAGGCCGTCTGCGAATTCGAGGGCGGCCATCT |
| | | GGCCACCTACAAGCAACTGGAGGCCGCTAGGAAGATCGGC |
| | | TTCCACGTCTGCGCCGCTGGATGGATGGCCAAGGGCAGAG |
| | | TGGGCTATCCCATCGTGAAGCCCGGCCCCAACTGCGGCTTC |
| | | GGAAAGACAGGCATCATCGACTACGGCATCAGGCTCAACA |
| | | GGAGCGAGAGGTGGGACGCTTACTGCTACAACCCCCATGC |
| | | C |
| SEQ ID NO: 65 (Kabat) | LCDR1 | SGDNLGSKYVD |
| SEQ ID NO: 66 (Kabat) | LCDR2 | SDNNRPS |
| SEQ ID NO: 67 (Kabat) | LCDR3 | QTYTSGNNYL |
| SEQ ID NO: 68 (Chothia) | LCDR1 | DNLGSKY |
| SEQ ID NO: 69 (Chothia) | LCDR2 | SDN |
| SEQ ID NO: 70 (Chothia) | LCDR3 | YTSGNNYL |
| SEQ ID NO: 71 | VL | SYELTQPPSVSVAPGQTARISCSGDNLGSKYVDWYQQKPGQ |
| | | APVLVIYSDNNRPSGIPERFSGSNSGNTATLTISGTQAEDEADY |
| | | YCQTYTSGNNYLVFGGGTKLTVL |
| SEQ ID NO: 72 | DNA of VL | AGCTACGAGCTGACTCAGCCCCCTTCTGTGTCTGTGGCCCC |
| | SEQ ID NO: 71 | TGGCCAGACCGCCAGAATCAGCTGCAGCGGCGACAACCTG |
| | | GGCAGCAAATACGTGGACTGGTATCAGCAGAAGCCCGGCC |
| | | AGGCTCCCGTGCTGGTGATCTACAGCGACAACAACCGGCC |
| | | CAGCGGCATCCCTGAGCGGTTCAGCGGCAGCAACAGCGGC |
| | | AATACCGCCACCCTGACCATCAGCGGCACCCAGGCCGAGG |
| | | ACGAGGCCGACTACTACTGCCAGACCTACACCAGCGGCAA |
| | | CAACTACCTGGTGTTCGGAGGCGGAACAAAGTTAACCGTC |
| | | CTA |
| SEQ ID NO: 73 | Light Chain | SYELTQPPSVSVAPGQTARISCSGDNLGSKYVDWYQQKPGQ |
| | | APVLVIYSDNNRPSGIPERFSGSNSGNTATLTISGTQAEDEADY |
| | | YCQTYTSGNNYLVFGGGTKLTVLGQPKAAPSVTLFPPSSEELQ |
| | | ANKATLVCLISDFYPGAVTVAWKADSSPVKAGVETTTPSKQS |
| | | NNKYAASSYLSLTPEQWKSHRSYSCQVTHEGSTVEKTVAPTEC |
| | | S |
| SEQ ID NO: 74 | DNA of Light | AGCTACGAGCTGACTCAGCCCCCTTCTGTGTCTGTGGCCCC |
| | Chain | TGGCCAGACCGCCAGAATCAGCTGCAGCGGCGACAACCTG |
| | SEQ ID NO: 73 | GGCAGCAAATACGTGGACTGGTATCAGCAGAAGCCCGGCC |
| | | AGGCTCCCGTGCTGGTGATCTACAGCGACAACAACCGGCC |
| | | CAGCGGCATCCCTGAGCGGTTCAGCGGCAGCAACAGCGGC |
| | | AATACCGCCACCCTGACCATCAGCGGCACCCAGGCCGAGG |
| | | ACGAGGCCGACTACTACTGCCAGACCTACACCAGCGGCAA |
| | | CAACTACCTGGTGTTCGGAGGCGGAACAAAGTTAACCGTC |
| | | CTAGGTCAGCCCAAGGCTGCCCCCTCGGTCACTCTGTTCCC |
| | | GCCCTCCTCTGAGGAGCTTCAAGCCAACAAGGCCACACTG |
| | | GTGTGTCTCATAAGTGACTTCTACCCGGGAGCCGTGACAGT |
| | | GGCCTGGAAGGCAGATAGCAGCCCCGTCAAGGCGGGAGT |
| | | GGAGACCACCACACCCTCCAAACAAAGCAACAACAAGTAC |
| | | GCGGCCAGCAGCTATCTGAGCCTGACGCCTGAGCAGTGGA |
| | | AGTCCCACAGAAGCTACAGCTGCCAGGTCACGCATGAAGG |
| | | GAGCACCGTGGAGAAGACAGTGGCCCCTACAGAATGTTCA |
| NVS72 and NVS72T | | |
| SEQ ID NO: 75 (Kabat) | HCDR1 | SYWIG |
| SEQ ID NO: 76 (Kabat) | HCDR2 | WIDPYRSEIRYSPSFQG |
| SEQ ID NO: 77 (Kabat) | HCDR3 | VSSEPFDS |
| SEQ ID NO: 78 (Chothia) | HCDR1 | GYSFTSY |
| SEQ ID NO: 79 (Chothia) | HCDR2 | DPYRSE |
| SEQ ID NO: 80 (Chothia) | HCDR3 | VSSEPFDS |
| SEQ ID NO: 81 | VH | EVQLVQSGAEVKKPGESLKISCKGSGYSFTSYWIGWVRQMPG |
| | | KGLEWMGWIDPYRSEIRYSPSFQGQVTISADKSISTAYLQWSS |
| | | LKASDTAMYYCARVSSEPFDSWGQGTLVTVSS |
| SEQ ID NO: 82 | DNA of VH | GAGGTCCAATTGGTCCAATCCGGAGCCGAAGTCAAGAAAC |
| | SEQ ID NO: 81 | CCGGCGAGTCCCTCAAAATCAGCTGCAAGGGCTCCGGCTA |
| | | CTCCTTCACCAGCTACTGGATCGGATGGGTGAGGCAGATG |
| | | CCCGGCAAAGGCCTCGAGTGGATGGGCTGGATCGACCCCT |
| | | ATAGGTCCGAGATTAGGTACAGCCCCTCCTTCCAGGGCCAG |
| | | GTCACCATCTCCGCCGACAAGAGCATCAGCACCGCCTACCT |
| | | CCAATGGTCCTCCCTCAAGGCCTCCGATACCGCCATGTATT |
| | | ACTGCGCCAGGGTCAGCAGCGAGCCCTTTGACAGCTGGGG |
| | | CCAGGGAACCCTCGTGACCGTCAGCTCA |
| SEQ ID NO: 83 | Heavy Chain | EVQLVQSGAEVKKPGESLKISCKGSGYSFTSYWIGWVRQMPG |
| | | KGLEWMGWIDPYRSEIRYSPSFQGQVTISADKSISTAYLQWSS |
| | | LKASDTAMYYCARVSSEPFDSWGQGTLVTVSSASTKGPSVFP |
| | | LAPSSKSTSGGTAALGCLVKDYFPEPVTVSWNSGALTSGVHTF |
| | | PAVLQSSGLYSLSSVVTVPSSSLGTQTYICNVNHKPSNTKVDKR |
| | | VEPKSC |
| SEQ ID NO: 84 | DNA of Heavy | GAGGTCCAATTGGTCCAATCCGGAGCCGAAGTCAAGAAAC |
| | Chain SEQ ID | CCGGCGAGTCCCTCAAAATCAGCTGCAAGGGCTCCGGCTA |
| | NO: 83 | CTCCTTCACCAGCTACTGGATCGGATGGGTGAGGCAGATG |
| | | CCCGGCAAAGGCCTCGAGTGGATGGGCTGGATCGACCCCT |
| | | ATAGGTCCGAGATTAGGTACAGCCCCTCCTTCCAGGGCCAG |
| | | GTCACCATCTCCGCCGACAAGAGCATCAGCACCGCCTACCT |
| | | CCAATGGTCCTCCCTCAAGGCCTCCGATACCGCCATGTATT |
| | | ACTGCGCCAGGGTCAGCAGCGAGCCCTTTGACAGCTGGGG |
| | | CCAGGGAACCCTCGTGACCGTCAGCTCAGCCAGCACCAAA |
| | | GGACCTAGCGTGTTCCCCCTCGCTCCCTCCTCCAAGAGCAC |
| | | ATCCGGCGGAACCGCTGCTCTGGGATGTCTCGTCAAGGAC |
| | | TACTTCCCCGAGCCCGTGACCGTGAGCTGGAATAGCGGCG |
| | | CCCTGACCTCCGGAGTCCACACATTCCCCGCTGTCCTGCAG |
| | | AGCAGCGGCCTGTATAGCCTGTCCTCCGTCGTGACCGTCCC |
| | | TAGCAGCTCCCTGGGAACCCAGACCTACATCTGCAACGTCA |
| | | ACCACAAGCCTAGCAACACCAAGGTGGACAAGAGGGTGG |
| | | AGCCCAAATCCTGC |
| SEQ ID NO: 85 | Heavy Chain + | EVQLVQSGAEVKKPGESLKISCKGSGYSFTSYWIGWVRQMPG |
| | Linker + | KGLEWMGWIDPYRSEIRYSPSFQGQVTISADKSISTAYLQWSS |
| | protein tag | LKASDTAMYYCARVSSEPFDSWGQGTLVTVSSASTKGPSVFP |
| | (SEQ ID NO: | LAPSSKSTSGGTAALGCLVKDYFPEPVTVSWNSGALTSGVHTF |
| | 83 + SEQ ID | PAVLQSSGLYSLSSVVTVPSSSLGTQTYICNVNHKPSNTKVDKR |
| | NO: 31 + SEQ | VEPKSCGSGGGGVYHREAQSGKYKLTYAEAKAVCEFEGGHLA |
| | ID NO: 33) | TYKQLEAARKIGFHVCAAGWMAKGRVGYPIVKPGPNCGFGK |
| | | TGIIDYGIRLNRSERWDAYCYNPHA |
| SEQ ID NO: 86 | DNA of Heavy | GAGGTCCAATTGGTCCAATCCGGAGCCGAAGTCAAGAAAC |
| | Chain + Linker + | CCGGCGAGTCCCTCAAAATCAGCTGCAAGGGCTCCGGCTA |
| | protein tag | CTCCTTCACCAGCTACTGGATCGGATGGGTGAGGCAGATG |
| | SEQ ID NO: 85 | CCCGGCAAAGGCCTCGAGTGGATGGGCTGGATCGACCCCT |
| | | ATAGGTCCGAGATTAGGTACAGCCCCTCCTTCCAGGGCCAG |
| | | GTCACCATCTCCGCCGACAAGAGCATCAGCACCGCCTACCT |
| | | CCAATGGTCCTCCCTCAAGGCCTCCGATACCGCCATGTATT |
| | | ACTGCGCCAGGGTCAGCAGCGAGCCCTTTGACAGCTGGGG |
| | | CCAGGGAACCCTCGTGACCGTCAGCTCAGCCAGCACCAAA |
| | | GGACCTAGCGTGTTCCCCCTCGCTCCCTCCTCCAAGAGCAC |
| | | ATCCGGCGGAACCGCTGCTCTGGGATGTCTCGTCAAGGAC |
| | | TACTTCCCCGAGCCCGTGACCGTGAGCTGGAATAGCGGCG |
| | | CCCTGACCTCCGGAGTCCACACATTCCCCGCTGTCCTGCAG |
| | | AGCAGCGGCCTGTATAGCCTGTCCTCCGTCGTGACCGTCCC |
| | | TAGCAGCTCCCTGGGAACCCAGACCTACATCTGCAACGTCA |
| | | ACCACAAGCCTAGCAACACCAAGGTGGACAAGAGGGTGG |
| | | AGCCCAAATCCTGCGGATCCGGAGGAGGCGGCGTGTATCA |
| | | CAGAGAGGCCCAGAGCGGCAAGTACAAGCTCACATACGCT |
| | | GAGGCCAAAGCCGTGTGCGAATTCGAGGGCGGACATCTG |
| | | GCCACATATAAGCAGCTGGAGGCCGCCAGGAAGATCGGCT |
| | | TCCACGTGTGCGCTGCCGGCTGGATGGCCAAAGGCAGAGT |
| | | GGGCTACCCTATCGTCAAGCCCGGCCCCAACTGCGGCTTTG |
| | | GCAAGACCGGCATCATCGACTACGGCATCAGGCTCAACAG |
| | | GTCCGAAAGGTGGGATGCCTACTGCTACAATCCCCACGCC |
| SEQ ID NO: 87 (Kabat) | LCDR1 | SGDKLGDHYAY |
| SEQ ID NO: 88 (Kabat) | LCDR2 | DDSKRPS |
| SEQ ID NO: 89 (Kabat) | LCDR3 | ATWTFEGDYV |
| SEQ ID NO: 90 (Chothia) | LCDR1 | DKLGDHY |
| SEQ ID NO: 91 (Chothia) | LCDR2 | DDS |
| SEQ ID NO: 92 (Chothia) | LCDR3 | WTFEGDY |
| SEQ ID NO: 93 | VL | SYVLTQPPSVSVAPGKTARITCSGDKLGDHYAYWYQQKPGQ |
| | | APVLVIYDDSKRPSGIPERFSGSNSGNTATLTISRVEAGDEADY |
| | | YCATWTFEGDYVFGGGTKLTVL |
| SEQ ID NO: 94 | DNA of VL | TCCTACGTCCTGACACAACCTCCCAGCGTGAGCGTCGCTCC |
| | SEQ ID NO: 93 | TGGCAAGACAGCCAGAATCACCTGCAGCGGCGACAAGCTG |
| | | GGCGACCACTACGCCTACTGGTATCAGCAGAAACCCGGCC |
| | | AAGCTCCCGTGCTGGTGATCTATGACGACAGCAAGAGACC |
| | | CTCCGGCATCCCTGAGAGATTCAGCGGAAGCAACTCCGGC |
| | | AACACCGCCACCCTGACCATCAGCAGGGTCGAAGCCGGCG |
| | | ATGAGGCCGACTACTACTGCGCCACCTGGACCTTTGAGGG |
| | | CGACTACGTGTTCGGAGGCGGCACCAAGTTAACCGTCCTA |
| SEQ ID NO: 95 | Light Chain | SYVLTQPPSVSVAPGKTARITCSGDKLGDHYAYWYQQKPGQ |
| | | APVLVIYDDSKRPSGIPERFSGSNSGNTATLTISRVEAGDEADY |
| | | YCATWTFEGDYVFGGGTKLTVLGQPKAAPSVTLFPPSSEELQA |
| | | NKATLVCLISDFYPGAVTVAWKADSSPVKAGVETTTPSKQSN |
| | | NKYAASSYLSLTPEQWKSHRSYSCQVTHEGSTVEKTVAPTECS |
| SEQ ID NO: 96 | DNA of Light | TCCTACGTCCTGACACAACCTCCCAGCGTGAGCGTCGCTCC |
| | Chain SEQ ID | TGGCAAGACAGCCAGAATCACCTGCAGCGGCGACAAGCTG |
| | NO: 95 | GGCGACCACTACGCCTACTGGTATCAGCAGAAACCCGGCC |
| | | AAGCTCCCGTGCTGGTGATCTATGACGACAGCAAGAGACC |
| | | CTCCGGCATCCCTGAGAGATTCAGCGGAAGCAACTCCGGC |
| | | AACACCGCCACCCTGACCATCAGCAGGGTCGAAGCCGGCG |
| | | ATGAGGCCGACTACTACTGCGCCACCTGGACCTTTGAGGG |
| | | CGACTACGTGTTCGGAGGCGGCACCAAGTTAACCGTCCTA |
| | | GGACAGCCTAAGGCCGCTCCCTCCGTGACACTGTTTCCCCC |
| | | TAGCAGCGAGGAGCTGCAGGCCAACAAGGCCACCCTCGTG |
| | | TGCCTCATCTCCGACTTCTACCCTGGCGCCGTCACAGTCGCC |
| | | TGGAAAGCCGACAGCTCCCCCGTCAAAGCTGGCGTGGAGA |
| | | CCACCACCCCCAGCAAGCAGAGCAACAACAAGTACGCCGC |
| | | CTCCTCCTATCTGAGCCTGACCCCCGAGCAGTGGAAGAGCC |
| | | ACAGGAGCTACTCCTGCCAGGTGACACACGAGGGCAGCAC |
| | | CGTCGAGAAGACCGTCGCTCCCACCGAGTGCAGC |
| NVS73 and NVS73T | | |
| SEQ ID NO: 108 | HCDR1 | GFTISRSYWIC |
| SEQ ID NO: 109 | HCDR2 | CIYGDNDITPLYANWAKG |
| SEQ ID NO: 110 | HCDR3 | LGYADYAYDL |
| SEQ ID NO: 111 | VH | EVQLVESGGGSVQPGGSLRLSCTASGFTISRSYWICWVRQAP |
| | | GKGLEWVGCIYGDNDITPLYANWAKGRFTISRDTSKNTVYLQ |
| | | MNSLRAEDTATYYCARLGYADYAYDLWGQGTTVTVSS |
| SEQ ID NO: 112 | DNA of VH | GAGGTCCAGCTGGTGGAGAGCGGAGGAGGAAGCGTCCAG |
| | SEQ ID NO: 111 | CCTGGAGGCAGCCTGAGACTGAGCTGCACCGCCAGCGGCT |
| | | TCACCATCAGCAGGAGCTACTGGATCTGCTGGGTGAGGCA |
| | | GGCTCCTGGCAAGGGACTCGAGTGGGTGGGCTGCATCTAC |
| | | GGCGACAACGACATCACCCCCCTCTACGCCAACTGGGCTAA |
| | | GGGCAGGTTCACCATTAGCAGGGACACCAGCAAGAACACC |
| | | GTGTACCTCCAGATGAACAGCCTGAGGGCCGAGGATACCG |
| | | CCACCTACTATTGCGCCAGGCTGGGCTACGCCGATTACGCC |
| | | TATGACCTCTGGGGCCAGGGCACCACAGTGACCGTCAGCT |
| | | CA |
| SEQ ID NO: 113 | Heavy Chain | EVQLVESGGGSVQPGGSLRLSCTASGFTISRSYWICWVRQAP |
| | | GKGLEWVGCIYGDNDITPLYANWAKGRFTISRDTSKNTVYLQ |
| | | MNSLRAEDTATYYCARLGYADYAYDLWGQGTIVTVSSASTK |
| | | GPSVFPLAPSSKSTSGGTAALGCLVKDYFPEPVTVSWNSGALT |
| | | SGVHTFPAVLQSSGLYSLSSVVTVPSSSLGTQTYICNVNHKPSN |
| | | TKVDKRVEPKSC |
| SEQ ID NO: 114 | DNA of Heavy | GAGGTCCAGCTGGTGGAGAGCGGAGGAGGAAGCGTCCAG |
| | Chain SEQ ID | CCTGGAGGCAGCCTGAGACTGAGCTGCACCGCCAGCGGCT |
| | NO: 113 | TCACCATCAGCAGGAGCTACTGGATCTGCTGGGTGAGGCA |
| | | GGCTCCTGGCAAGGGACTCGAGTGGGTGGGCTGCATCTAC |
| | | GGCGACAACGACATCACCCCCCTCTACGCCAACTGGGCTAA |
| | | GGGCAGGTTCACCATTAGCAGGGACACCAGCAAGAACACC |
| | | GTGTACCTCCAGATGAACAGCCTGAGGGCCGAGGATACCG |
| | | CCACCTACTATTGCGCCAGGCTGGGCTACGCCGATTACGCC |
| | | TATGACCTCTGGGGCCAGGGCACCACAGTGACCGTCAGCT |
| | | CAGCCTCCACCAAGGGACCTTCCGTGTTCCCCCTGGCCCCT |
| | | AGCTCCAAGTCCACCAGCGGAGGAACAGCCGCTCTGGGCT |
| | | GTCTGGTGAAGGACTACTTCCCCGAGCCTGTGACCGTGTCC |
| | | TGGAATTCCGGCGCCCTCACAAGCGGAGTGCATACCTTCCC |
| | | CGCCGTGCTGCAAAGCTCCGGACTGTACTCCCTCTCCAGCG |
| | | TGGTGACAGTGCCTTCCAGCAGCCTCGGCACCCAGACCTAC |
| | | ATCTGCAACGTGAACCACAAGCCCTCCAATACCAAGGTGG |
| | | ACAAGAGGGTCGAGCCTAAAAGCTGT |
| SEQ ID NO: 115 | Heavy Chain + | EVQLVESGGGSVQPGGSLRLSCTASGFTISRSYWICWVRQAP |
| | Linker + | GKGLEWVGCIYGDNDITPLYANWAKGRFTISRDTSKNTVYLQ |
| | protein tag | MNSLRAEDTATYYCARLGYADYAYDLWGQGTIVTVSSASTK |
| | (SEQ ID NO: | GPSVFPLAPSSKSTSGGTAALGCLVKDYFPEPVTVSWNSGALT |
| | 113 + SEQ ID | SGVHTFPAVLQSSGLYSLSSVVTVPSSSLGTQTYICNVNHKPSN |
| | NO: 31 + SEQ | TKVDKRVEPKSCGSGGGGVYHREAQSGKYKLTYAEAKAVCEF |
| | ID NO: 33) | EGGHLATYKQLEAARKIGFHVCAAGWMAKGRVGYPIVKPGP |
| | | NCGFGKTGIIDYGIRLNRSERWDAYCYNPHA |
| SEQ ID NO: 116 | DNA of Heavy | GAGGTCCAGCTGGTGGAGAGCGGAGGAGGAAGCGTCCAG |
| | Chain + Linker + | CCTGGAGGCAGCCTGAGACTGAGCTGCACCGCCAGCGGCT |
| | protein tag | TCACCATCAGCAGGAGCTACTGGATCTGCTGGGTGAGGCA |
| | SEQ ID NO: | GGCTCCTGGCAAGGGACTCGAGTGGGTGGGCTGCATCTAC |
| | 115 | GGCGACAACGACATCACCCCCCTCTACGCCAACTGGGCTAA |
| | | GGGCAGGTTCACCATTAGCAGGGACACCAGCAAGAACACC |
| | | GTGTACCTCCAGATGAACAGCCTGAGGGCCGAGGATACCG |
| | | CCACCTACTATTGCGCCAGGCTGGGCTACGCCGATTACGCC |
| | | TATGACCTCTGGGGCCAGGGCACCACAGTGACCGTCAGCT |
| | | CAGCCTCCACCAAGGGACCTTCCGTGTTCCCCCTGGCCCCT |
| | | AGCTCCAAGTCCACCAGCGGAGGAACAGCCGCTCTGGGCT |
| | | GTCTGGTGAAGGACTACTTCCCCGAGCCTGTGACCGTGTCC |
| | | TGGAATTCCGGCGCCCTCACAAGCGGAGTGCATACCTTCCC |
| | | CGCCGTGCTGCAAAGCTCCGGACTGTACTCCCTCTCCAGCG |
| | | TGGTGACAGTGCCTTCCAGCAGCCTCGGCACCCAGACCTAC |
| | | ATCTGCAACGTGAACCACAAGCCCTCCAATACCAAGGTGG |
| | | ACAAGAGGGTCGAGCCTAAAAGCTGTGGATCCGGAGGAG |
| | | GCGGCGTGTATCATAGAGAGGCCCAGTCCGGCAAGTACAA |
| | | GCTGACCTACGCCGAAGCCAAGGCCGTGTGTGAGTTCGAG |
| | | GGCGGACACCTGGCTACCTACAAACAGCTCGAAGCCGCTA |
| | | GGAAGATCGGATTCCACGTGTGCGCCGCCGGATGGATGGC |
| | | CAAAGGCAGAGTGGGCTACCCCATTGTCAAGCCCGGACCC |
| | | AACTGCGGATTCGGCAAGACCGGCATCATCGACTACGGCA |
| | | TCAGGCTCAACAGGTCCGAGAGATGGGACGCTTACTGCTA |
| | | CAATCCCCACGCC |
| SEQ ID NO: 117 | LCDR1 | QSSQSVYGNIWMA |
| SEQ ID NO: 118 | LCDR2 | QASKLAS |
| SEQ ID NO: 119 | LCDR3 | QGNFNTGDRYA |
| SEQ ID NO: 120 | VL | EIVMTQSPSTLSASVGDRVIITCQSSQSVYGNIWMAWYQQK |
| | | PGRAPKLLIYQASKLASGVPSRFSGSGSGAEFTLTISSLQPDDFA |
| | | TYYCQGNENTGDRYAFGQGTKLTVLKR |
| SEQ ID NO: 121 | DNA of VL SEQ | GAGATCGTCATGACCCAGAGCCCCAGCACACTCAGCGCCTC |
| | ID NO: 120 | CGTGGGAGACAGGGTGATCATCACCTGCCAGTCCTCCCAG |
| | | TCCGTGTACGGCAACATCTGGATGGCCTGGTACCAGCAGA |
| | | AGCCCGGCAGAGCCCCCAAGCTGCTGATCTACCAGGCCAG |
| | | CAAGCTCGCCTCCGGAGTGCCCAGCAGATTTTCCGGCTCCG |
| | | GATCCGGAGCCGAGTTCACACTGACCATCAGCAGCCTGCA |
| | | GCCCGATGACTTCGCCACCTACTATTGCCAGGGCAACTTCA |
| | | ACACCGGCGACAGGTACGCCTTTGGCCAGGGCACCAAGCT |
| | | GACCGTCCTCAAGCGT |
| SEQ ID NO: 122 | Light Chain | EIVMTQSPSTLSASVGDRVIITCQSSQSVYGNIWMAWYQQK |
| | | PGRAPKLLIYQASKLASGVPSRFSGSGSGAEFTLTISSLQPDDFA |
| | | TYYCQGNFNTGDRYAFGQGTKLTVLKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVTKSF |
| | | NRGEC |
| SEQ ID NO: 123 | DNA of Light | GAGATCGTCATGACCCAGAGCCCCAGCACACTCAGCGCCTC |
| | Chain SEQ ID | CGTGGGAGACAGGGTGATCATCACCTGCCAGTCCTCCCAG |
| | NO: 122 | TCCGTGTACGGCAACATCTGGATGGCCTGGTACCAGCAGA |
| | | AGCCCGGCAGAGCCCCCAAGCTGCTGATCTACCAGGCCAG |
| | | CAAGCTCGCCTCCGGAGTGCCCAGCAGATTTTCCGGCTCCG |
| | | GATCCGGAGCCGAGTTCACACTGACCATCAGCAGCCTGCA |
| | | GCCCGATGACTTCGCCACCTACTATTGCCAGGGCAACTTCA |
| | | ACACCGGCGACAGGTACGCCTTTGGCCAGGGCACCAAGCT |
| | | GACCGTCCTCAAGCGTACGGTGGCTGCTCCCAGCGTCTTCA |
| | | TCTTCCCCCCCAGCGATGAGCAGCTCAAGAGCGGCACAGC |
| | | CTCCGTGGTGTGCCTCCTGAACAACTTCTACCCTAGGGAGG |
| | | CCAAGGTGCAATGGAAGGTGGACAACGCCCTGCAGAGCG |
| | | GCAACAGCCAGGAGTCCGTGACCGAGCAGGACTCCAAGG |
| | | ACAGCACCTACAGCCTGAGCAGCACACTCACCCTGAGCAAA |
| | | GCCGACTACGAGAAGCACAAGGTCTACGCCTGCGAGGTGA |
| | | CCCATCAGGGCCTGTCCAGCCCCGTGACCAAGAGCTTCAAC |
| | | AGAGGCGAGTGC |
| NVS75 and NVS75T | | |
| SEQ ID NO: 189 | HCDR1 | GFTFSVYGMN |
| SEQ ID NO: 190 | HCDR2 | IIWYDGDNQYYADSVKG |
| SEQ ID NO: 191 | HCDR3 | DLRTGPFDY |
| SEQ ID NO: 192 | VH | QVQLVESGGGVVQPGRSLRLSCAASGFTFSVYGMNWVRQA |
| | | PGKGLEWVAIIWYDGDNQYYADSVKGRFTISRDNSKNTLYLQ |
| | | MNGLRAEDTAVYYCARDLRTGPFDYWGQGTLVTVSS |
| SEQ ID NO: 193 | DNA of VH | CAGGTGCAGCTGGTGGAATCTGGCGGCGGAGTGGTGCAG |
| | SEQ ID NO: | CCTGGCAGAAGCCTGAGACTGAGCTGTGCCGCCAGCGGCT |
| | 192 | TCACCTTCAGCGTGTACGGCATGAACTGGGTGCGCCAGGC |
| | | CCCTGGCAAAGGCCTGGAATGGGTGGCCATCATTTGGTAC |
| | | GACGGCGACAACCAGTACTACGCCGACAGCGTGAAGGGCC |
| | | GGTTCACCATCAGCCGGGACAACAGCAAGAACACCCTGTA |
| | | CCTGCAGATGAACGGCCTGCGGGCCGAGGATACCGCCGTG |
| | | TACTACTGCGCCAGGGACCTGAGAACAGGCCCCTTCGATTA |
| | | TTGGGGCCAGGGCACCCTCGTGACCGTGTCTAGC |
| SEQ ID NO: 194 | Heavy Chain | QVQLVESGGGVVQPGRSLRLSCAASGFTFSVYGMNWVRQA |
| | | PGKGLEWVAIIWYDGDNQYYADSVKGRFTISRDNSKNTLYLQ |
| | | MNGLRAEDTAVYYCARDLRTGPFDYWGQGTLVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSNT |
| | | KVDKRVEPKSC |
| SEQ ID NO: 195 | DNA of Heavy | CAGGTGCAGCTGGTGGAATCTGGCGGCGGAGTGGTGCAG |
| | Chain ID NO: | CCTGGCAGAAGCCTGAGACTGAGCTGTGCCGCCAGCGGCT |
| | 194 | TCACCTTCAGCGTGTACGGCATGAACTGGGTGCGCCAGGC |
| | | CCCTGGCAAAGGCCTGGAATGGGTGGCCATCATTTGGTAC |
| | | GACGGCGACAACCAGTACTACGCCGACAGCGTGAAGGGCC |
| | | GGTTCACCATCAGCCGGGACAACAGCAAGAACACCCTGTA |
| | | CCTGCAGATGAACGGCCTGCGGGCCGAGGATACCGCCGTG |
| | | TACTACTGCGCCAGGGACCTGAGAACAGGCCCCTTCGATTA |
| | | TTGGGGCCAGGGCACCCTCGTGACCGTGTCTAGCGCCTCTA |
| | | CAAAGGGCCCCAGCGTGTTCCCTCTGGCCCCTAGCAGCAA |
| | | GTCTACCAGCGGAGGAACAGCCGCCCTGGGCTGCCTCGTG |
| | | AAGGACTACTTTCCCGAGCCCGTGACAGTGTCCTGGAACTC |
| | | TGGCGCCCTGACAAGCGGCGTGCACACCTTTCCAGCCGTGC |
| | | TGCAGAGCAGCGGCCTGTACTCTCTGAGCAGCGTCGTGAC |
| | | TGTGCCCAGCAGCTCTCTGGGCACCCAGACCTACATCTGCA |
| | | ACGTGAACCACAAGCCCAGCAACACCAAGGTGGACAAGCG |
| | | GGTGGAACCCAAGAGCTGT |
| SEQ ID NO: 196 | Heavy Chain + | QVQLVESGGGVVQPGRSLRLSCAASGFTFSVYGMNWVRQA |
| | Linker + | PGKGLEWVAIIWYDGDNQYYADSVKGRFTISRDNSKNTLYLQ |
| | protein tag | MNGLRAEDTAVYYCARDLRTGPFDYWGQGTLVTVSSASTKG |
| | (SEQ ID NO: | PSVFPLAPSSKSTSGGTAALGCLVKDYFPEPVTVSWNSGALTS |
| | 194 + SEQ ID | GVHTFPAVLQSSGLYSLSSVVTVPSSSLGTQTYICNVNHKPSNT |
| | NO: 31 + SEQ | KVDKRVEPKSCGSGGGGVYHREAQSGKYKLTYAEAKAVCEFE |
| | ID NO: 33) | GGHLATYKQLEAARKIGFHVCAAGWMAKGRVGYPIVKPGPN |
| | | CGFGKTGIIDYGIRLNRSERWDAYCYNPHA |
| SEQ ID NO: 197 | DNA of Heavy | CAGGTGCAGCTGGTGGAATCTGGCGGCGGAGTGGTGCAG |
| | Chain SEQ ID | CCTGGCAGAAGCCTGAGACTGAGCTGTGCCGCCAGCGGCT |
| | NO: 196 | TCACCTTCAGCGTGTACGGCATGAACTGGGTGCGCCAGGC |
| | | CCCTGGCAAAGGCCTGGAATGGGTGGCCATCATTTGGTAC |
| | | GACGGCGACAACCAGTACTACGCCGACAGCGTGAAGGGCC |
| | | GGTTCACCATCAGCCGGGACAACAGCAAGAACACCCTGTA |
| | | CCTGCAGATGAACGGCCTGCGGGCCGAGGATACCGCCGTG |
| | | TACTACTGCGCCAGGGACCTGAGAACAGGCCCCTTCGATTA |
| | | TTGGGGCCAGGGCACCCTCGTGACCGTGTCTAGCGCCTCTA |
| | | CAAAGGGCCCCAGCGTGTTCCCTCTGGCCCCTAGCAGCAA |
| | | GTCTACCAGCGGAGGAACAGCCGCCCTGGGCTGCCTCGTG |
| | | AAGGACTACTTTCCCGAGCCCGTGACAGTGTCCTGGAACTC |
| | | TGGCGCCCTGACAAGCGGCGTGCACACCTTTCCAGCCGTGC |
| | | TGCAGAGCAGCGGCCTGTACTCTCTGAGCAGCGTCGTGAC |
| | | TGTGCCCAGCAGCTCTCTGGGCACCCAGACCTACATCTGCA |
| | | ACGTGAACCACAAGCCCAGCAACACCAAGGTGGACAAGCG |
| | | GGTGGAACCCAAGAGCTGT |
| SEQ ID NO: 198 | LCDR1 | RASQSIGSSLH |
| SEQ ID NO: 199 | LCDR2 | YASQSFS |
| SEQ ID NO: 200 | LCDR3 | HQSSSLPFT |
| SEQ ID NO: 201 | VL | EIVLTQSPDFQSVTPKEKVTITCRASQSIGSSLHWYQQKPDQS |
| | | PKLLIKYASQSFSGVPSRFSGSGSGTDFTLTINSLEAEDAAAYYC |
| | | HQSSSLPFTFGPGTKVDIKR |
| SEQ ID NO: [[20]]214 | DNA of VL SEQ | GAGATCGTGCTGACCCAGAGCCCCGACTTTCAGAGCGTGA |
| | ID NO: 201 | CCCCCAAAGAAAAAGTGACCATCACCTGTCGGGCCAGCCA |
| | | GAGCATCGGCTCTAGCCTGCACTGGTATCAGCAGAAGCCC |
| | | GACCAGTCCCCCAAGCTGCTGATTAAGTACGCCAGCCAGTC |
| | | CTTCAGCGGCGTGCCCAGCAGATTTTCTGGCAGCGGCTCCG |
| | | GCACCGACTTCACCCTGACCATCAACAGCCTGGAAGCCGA |
| | | GGACGCCGCTGCCTACTACTGTCACCAGAGCAGCAGCCTG |
| | | CCCTTCACCTTTGGCCCTGGCACCAAGGTGGACATCAAGCG |
| | | G |
| SEQ ID NO: 202 | Light Chain | EIVLTQSPDFQSVTPKEKVTITCRASQSIGSSLHWYQQKPDQS |
| | | PKLLIKYASQSFSGVPSRFSGSGSGTDFTLTINSLEAEDAAAYYC |
| | | HQSSSLPFTFGPGTKVDIKRTVAAPSVFIFPPSDEQLKSGTASV |
| | | VCLLNNFYPREAKVQWKVDNALQSGNSQESVTEQDSKDSTY |
| | | SLSSTLTLSKADYEKHKVYACEVTHQGLSSPVTKSFNRGEC |
| SEQ ID NO: 203 | DNA of Light | GAGATCGTGCTGACCCAGAGCCCCGACTTTCAGAGCGTGA |
| | Chain SEQ ID | CCCCCAAAGAAAAAGTGACCATCACCTGTCGGGCCAGCCA |
| | NO: 202 | GAGCATCGGCTCTAGCCTGCACTGGTATCAGCAGAAGCCC |
| | | GACCAGTCCCCCAAGCTGCTGATTAAGTACGCCAGCCAGTC |
| | | CTTCAGCGGCGTGCCCAGCAGATTTTCTGGCAGCGGCTCCG |
| | | GCACCGACTTCACCCTGACCATCAACAGCCTGGAAGCCGA |
| | | GGACGCCGCTGCCTACTACTGTCACCAGAGCAGCAGCCTG |
| | | CCCTTCACCTTTGGCCCTGGCACCAAGGTGGACATCAAGCG |
| | | GACAGTGGCCGCTCCCTCCGTGTTCATCTTCCCACCTAGCG |
| | | ACGAGCAGCTGAAGTCTGGCACAGCCAGCGTCGTGTGCCT |
| | | GCTGAACAACTTCTACCCCCGCGAGGCCAAGGTGCAGTGG |
| | | AAAGTGGACAACGCCCTGCAGAGCGGCAACAGCCAGGAA |
| | | AGCGTGACCGAGCAGGACAGCAAGGACTCCACCTACAGCC |
| | | TGAGCAGCACCCTGACACTGAGCAAGGCCGACTACGAGAA |
| | | GCACAAGGTGTACGCCTGCGAAGTGACCCACCAGGGCCTG |
| | | TCTAGCCCCGTGACCAAGAGCTTCAACCGGGGCGAGTGC |

TABLE 2b
Sequence of Ocular Proteins

| | |
|---|---|
| Human VEGF | SEQ ID NO: 97 |
| | APMAEGGGQNHHEVVKFMDVYQRSYCHPIETLV |
| | DIFQEYPDEIEYIFKPSCVPLMRCGGCCNDEGL |
| | ECVPTEESNITMQIMRIKPHQGQHIGEMSFLQH |
| | NKCECRPKKDRARQEKKSVRGKGKGQKRKRKKS |
| | RYKSWSVYVGARCCLMPWSLPGPHPCGPCSERR |
| | KHLFVQDPQTCKCSCKNTDSRCKARQLELNERT |
| | CRCDKPRR |
| Human EPO | SEQ ID NO: 98 |
| | APPRLICDSRVLERYLLEAKEAENITTGCAEHC |
| | SLNENITVPDTKVNFYAWKRMEVGQQAVEVWQG |
| | LALLSEAVLRGQALLVNSSQPWEPLQLHVDKAV |
| | SGLRSLTTLLRALGAQKEAISPPDAASAAPLRT |
| | ITADTFRKLFRVYSNFLRGKLKLYTGEACRTGD |
| | R |
| Human C5 | SEQ ID NO: 99 |
| | QEQTYVISAPKIFRVGASENIVIQVYGYTEAFD |
| | ATISIKSYPDKKFSYSSGHVHLSSENKFQNSAI |
| | LTIQPKQLPGGQNPVSYVYLEVVSKHFSKSKRM |
| | PITYDNGFLFIHTDKPVYTPDQSVKVRVYSLND |
| | DLKPAKRETVLTFIDPEGSEVDMVEEIDHIGII |
| | SFPDFKIPSNPRYGMWTIKAKYKEDFSTIGTAY |
| | FEVKEYVLPHFSVSIEPEYNFIGYKNFKNFEIT |
| | IKARYFYNKVVTEADVYITFGIREDLKDDQKEM |
| | MQTAMQNTMLINGIAQVTFDSETAVKELSYYSL |
| | EDLNNKYLYIAVTVIESTGGFSEEAEIPGIKYV |
| | LSPYKLNLVATPLFLKPGIPYPIKVQVKDSLDQ |
| | LVGGVPVTLNAQTIDVNQETSDLDPSKSVTRVD |
| | DGVASFVLNLPSGVTVLEFNVKTDAPDLPEENQ |
| | AREGYRAIAYSSLSQSYLYIDWTDNHKALLVGE |
| | HLNIIVTPKSPYIDKITHYNYLILSKGKIIHFG |
| | TREKFSDASYQSINIPVTQNMVPSSRLLVYYIV |
| | TGEQTAELVSDSVWLNIEEKCGNQLQVHLSPDA |
| | DAYSPGQTVSLNMATGMDSWVALAAVDSAVYGV |
| | QRGAKKPLERVFQFLEKSDLGCGAGGGLNNANV |
| | FHLAGLTFLTNANADDSQENDEPCKEILRPRRT |
| | LQKKIEEIAAKYKHSVVKKCCYDGACVNNDETC |
| | EQRAARISLGPRCIKAFTECCVVASQLRANISH |
| | KDMQLGRLHMKTLLPVSKPEIRSYFPESWLWEV |
| | HLVPRRKQLQFALPDSLTTWEIQGVGISNTGIC |
| | VADTVKAKVFKDVFLEMNIPYSVVRGEQIQLKG |
| | TVYNYRTSGMQFCVKMSAVEGICTSESPVIDHQ |
| | GTKSSKCVRQKVEGSSSHLVTFTVLPLEIGLHN |
| | INFSLETWFGKEILVKTLRVVPEGVKRESYSGV |
| | TLDPRGIYGTISRRKEFPYRIPLDLVPKTEIKR |
| | ILSVKGLLVGEILSAVLSQEGINILTHLPKGSA |
| | EAELMSVVPVFYVFHYLETGNHWNIFHSDPLIE |
| | KQKLKKKLKEGMLSIMSYRNADYSYSVWKGGSA |
| | STWLTAFALRVLGQVNKYVEQNQNSICNSLLWL |
| | VENYQLDNGSFKENSQYQPIKLQGTLPVEAREN |
| | SLYLTAFTVIGIRKAFDICPLVKIDTALIKADN |
| | FLLENTLPAQSTFTLAISAYALSLGDKTHPQFR |
| | SIVSALKREALVKGNPPIYRFWKDNLQHKDSSV |
| | PNTGTARMVETTAYALLTSLNLKDINYVNPVIK |
| | WLSEEQRYGGGFYSTQDTINAIEGLTEYSLLVK |
| | QLRLSMDIDVSYKHKGALHNYKMTDKNFLGRPV |
| | EVLLNDDLIVSTGFGSGLATVHVTTVVHKTSTS |
| | EEVCSFYLKIDTQDIEASHYRGYGNSDYKRIVA |
| | CASYKPSREESSSGSSHAVMDISLPTGISANEE |
| | DLKALVEGVDQLFTDYQIKDGHVILQLNSIPSS |
| | DFLCVRFRIFELFEVGFLSPATFTVYEYHRPDK |
| | QCTMFYSTSNIKIQKVCEGAACKCVEADCGQMQ |
| | EELDLTISAETRKQTACKPEIAYAYKVSITSIT |
| | VENVFVKYKATLLDIYKTGEAVAEKDSEITFIK |
| | KVTCTNAELVKGRQYLIMGKEALQIKYNFSFRY |
| | IYPLDSLTWIEYWPRDTTCSSCQAFLANLDEFA |
| | EDIFLNGC |
| Human | SEQ ID NO: 100 |
| Factor P | DPVLCFTQYEESSGKCKGLLGGGVSVEDCCLNT |
| | AFAYQKRSGGLCQPCRSPRWSLWSTWAPCSVIC |
| | SEGSQLRYRRCVGWNGQCSGKVAPGTLEWQLQA |
| | CEDQQCCPEMGGWSGWGPWEPCSVTCSKGTRTR |
| | RRACNHPAPKCGGHCPGQAQESEACDTQQVCPT |
| | HGAWATWGPWTPCSASCHGGPHEPKETRSRKCS |
| | APEPSQKPPGKPCPGLAYEQRRCTGLPPCPVAG |
| | GWGPWGPVSPCPVTCGLGQTMEQRTCNHPVPQH |
| | GGPFCAGDATRTHICNTAVPCPVDGEWDSWGEW |
| | SPCIRRNMKSISCQEIPGQQSRGRTCRGRKFDG |
| | HRCAGQQQDIRHCYSIQHCPLKGSWSEWSTWGL |
| | CMPPCGPNPTRARQRLCTPLLPKYPPTVSMVEG |
| | QGEKNVTFWGRPLPRCEELQGQKLVVEEKRPCL |
| | HVPACKDPEEEEL |
| Human TNFα | SEQ ID NO: 101 |
| | VRSSSRTPSDKPVAHVVANPQAEGQLQWLNRRA |
| | NALLANGVELRDNQLVVPSEGLYLIYSQVLFKG |
| | QGCPSTHVLLTHTISRIAVSYQTKVNLLSAIKS |
| | PCQRETPEGAEAKPWYEPIYLGGVFQLEKGDRL |
| | SAEINRPDYLDFAESGQVYFGIIAL |
| Human IL-1β | SEQ ID NO: 102 |
| | MAEVPELASEMMAYYSGNEDDLFFEADGPKQMK |
| | CSFQDLDLCPLDGGIQLRISDHHYSKGFRQAAS |
| | VVVAMDKLRKMLVPCPQTFQENDLSTFFPFIFE |
| | EEPIFFDTWDNEAYVHDAPVRSLNCTLRDSQQK |
| | SLVMSGPYELKALHLQGQDMEQQVVFSMSFVQG |
| | EESNDKIPVALGLKEKNLYLSCVLKDDKPTLQL |
| | ESVDPKNYPKKKMEKRFVFNKIEINNKLEFESA |
| | QFPNWYISTSQAENMPVFLGGTKGGQDITDFTM |
| | QFVSS |

### Peptide Linkers

In certain aspects of the invention the protein tags maybe linked to a molecule by a linker. More specifically, the protein tags maybe linked to a protein or a nucleic acid, by a peptide linker (e.g., a (Glyₙ-Serₙ)ₙ or (Serₙ-Glyₙ)ₙ linker) with an optimized length and/or amino acid composition. It is known that peptide linker length can greatly affect how the connected proteins fold and interact. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

The peptide linker sequence may be at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues in length. The peptide linker sequence may be comprised of a naturally, or non-naturally, occurring amino acids. In some aspects, the linker is a glycine polymer. In some aspects, the amino acids glycine and serine comprise the amino acids within the linker sequence. In certain aspects, the linker region comprises sets of glycine repeats (GlySerGly₃)ₙ (SEQ ID NO: 31), where n is a positive integer equal to or greater than 1. More specifically, the linker sequence may be GlySerGlyGlyGly (SEQ ID NO: 31). Alternatively, the linker sequence may be GlySerGlyGly (SEQ ID NO: 124). In certain other aspects, the linker region orientation comprises sets of glycine repeats (SerGly₃)ₙ (SEQ ID NO: 155), where n is a positive integer equal to or greater than 1.

The peptide linkers may also include, but are not limited to, (Gly₄Ser)₄(SEQ ID NO: 186) or (Gly₄Ser)₃ (SEQ ID NO: 204). The amino acid residues Glu and Lys can be interspersed within the Gly-Ser peptide linkers for better solubility. In certain aspects, the peptide linkers may include multiple repeats of (Gly₃Ser) (SEQ ID NO: 205), (Gly₂Ser) or (GlySer). In certain aspects, the peptide linkers may include multiple repeats of (SerGly₃) (SEQ ID NO: 155), (SerGly₂) or (SerGly). In other aspects, the peptide linkers may include combinations and multiples of (Gly₃Ser)+(Gly₄Ser)+(GlySer) (SEQ ID NOS 205-206, respectively). In still other aspects, Ser can be replaced with Ala e.g., (Gly₄Ala) (SEQ ID NO: 207) or (Gly₃Ala) (SEQ ID NO: 208). In yet other aspects, the linker comprises the motif (GluAlaAlaAlaLys)ₙ (SEQ ID NO: 209), where n is a positive integer equal to or greater than 1. In certain aspects, peptide linkers may also include cleavable linkers.

Peptide linkers can be of varying lengths. In particular, a peptide linker is from about 5 to about 50 amino acids in length; from about 10 to about 40 amino acids in length; from about 15 to about 30 amino acids in length; or from about 15 to about 20 amino acids in length. Variation in peptide linker length may retain or enhance activity, giving rise to superior efficacy in activity studies. Peptide linkers can be introduced into polypeptide and protein sequences using techniques known in the art. For example, PCR mutagenesis can be used. Modifications can be confirmed by DNA sequence analysis. Plasmid DNA can be used to transform host cells for stable production of the polypeptides produced.

Peptide linkers, peptide tags and proteins (e.g.: antibodies or antigen binding fragments) or nucleic acids, or a combination thereof, can be encoded in the same vector and expressed and assembled in the same host cell. Alternatively, each peptide linker, protein tag and protein or nucleic acid can be generated separately and then conjugated to one another. Peptide linkers, peptide tags and proteins or nucleic acids can be prepared by conjugating the constituent components, using methods known in the art. Site-specific conjugation can be achieved using sortase-mediated enzymatic conjugation (Mao H, Hart S A, Schink A, Pollok B A. J Am Chem Soc. 2004 Mar. 10; 126(9):2670-1). A variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohexane-1-carboxylate (sulfo-SMCC) (see e.g., Karpovsky et al., 1984 J. Exp. Med. 160:1686; Liu, M A et al., 1985 Proc. Natl. Acad. Sci. USA 82:8648). Other methods include those described in Paulus, 1985 Behring Ins. Mitt. No. 78, 118-132; Brennan et al., 1985 Science 229:81-83), and Glennie et al., 1987 J. Immunol. 139: 2367-2375). Conjugating agents are SATA and sulfo-SMCC, both available from Pierce Chemical Co. (Rockford, Ill.).

Engineered and Modified Molecules with Extended Half Life

Production of Peptide Tagged Molecules

The present invention provides peptide tags that can be recombinantly fused (i.e.: linked) or chemically conjugated (including both covalent and non-covalent conjugations) to other molecules, for example other proteins or nucleic acids. In certain aspects one, two, three, four or more peptide tags may be recombinantly fused, linked or chemically conjugated to a protein or nucleic acid. In certain aspects the peptide tag binds HA. In other aspects, the peptide tag binds HA and comprises a LINK Domain. In other aspects, the peptide tag binds HA and comprises a TSG-6 LINK Domain. More specifically, it is contemplated that the peptide tag may be HA10 (SEQ ID NO: 32), HA10.1 (SEQ ID NO: 33), HA10.2 (SEQ ID NO: 34), HA11 (SEQ ID NO: 35) or HA11.1 (SEQ ID NO: 36). In addition, the protein may be any of the proteins, antibodies or antigen binding fragments described herein, including, but not limited to, proteins, antibodies and antigen binding fragments as described above and in Tables 1, 2, 2b, 8b and 9b, as well as US20120014958, WO2012015608, WO2012149246, U.S. Pat. No. 8,273,352, WO1998045331, US2012100153, and WO2002016436.

In certain specific aspects, the invention provides peptide tagged molecules comprising antibodies, or antigen binding fragments, and a peptide tag. In particular, the invention provides peptide tagged molecules comprising an antigen-binding fragment of an antibody described herein (e.g., a Fab fragment, Fd fragment, Fv fragment, (Fab′)2 fragment, a VH domain, a VH CDR, a VL domain or a VL CDR) and a peptide tag. Methods for linking, fusing or conjugating proteins, polypeptides, or peptides to an antibody or an antigen binding fragment are known in the art and may be performed using standard molecular biology techniques known to those of skill in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341; Hermanson (2008) Bioconjugate Techniques (2^{nd} edition). Elsevier, Inc.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates) and/or to alter the activity of a peptide tag or protein (e.g., peptide tags and/or proteins with higher affinities and lower dissociation rate). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313, (Pluckthun, 2012), (Wttrup, 2001), (Levin and Weiss, 2006). Antibodies or fragments thereof, or the encoded antibodies or fragments thereof, may be altered by being subjected to random mutagenesis by error-prone PCR, random nucleotide insertion or other methods prior to recombination. A polynucleotide encoding an antibody or fragment thereof that specifically binds to a therapeutic target in the eye, (e.g: the protein VEGF) may be recombined with one or more components, motifs, sections, parts, domains, fragments, etc. of one or more heterologous molecules and/or peptide tags that bind HA.

Moreover, the antibodies, or antigen binding fragments, and/or peptide tags can be fused to marker sequences, such as a peptide to facilitate purification. For example, the marker amino acid sequence is a hexa-histidine peptide (SEQ ID NO: 188), such as the marker provided in a pQE vector (QIAGEN®, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine (SEQ ID NO: 188) provides for convenient purification of the fusion protein. Other tags useful for purification include, but are not limited to, the hemagglutinin tag, which corresponds to an epitope derived from the influenza hemagglutinin protein (Wilson et al., 1984, Cell 37:767), and the “flag” tag.

In other embodiments, antibodies, or antigen binding fragments, and/or peptide tags may be conjugated to a diagnostic or detectable agent. Such antibodies and/or peptide tags can be useful for monitoring or prognosing the onset, development, progression and/or severity of a disease or disorder as part of a clinical testing procedure, such as determining the efficacy of a particular therapy. Such diagnosis and detection can accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidinlbiotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (131I, 125I, 123I, and 121I,), carbon (14C), sulfur (35S), tritium (3H), indium (115In, 113In, 112In, and 111In,), technetium (99Tc), thallium (201Ti), gallium (68Ga, 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142 Pr, 105Rh, 97Ru, 68Ge, 57Co, 65Zn, 85Sr, 32P, 153Gd, 169Yb, 51Cr, 54Mn, 75Se, 113Sn, and 117Tin; and positron emitting metals using various positron emission tomographies, and non-radioactive paramagnetic metal ions.

Antibodies, or antigen binding fragments, and peptide tags may also be attached to solid supports, which are particularly useful for immunoassays or purification of the target antigen. Such solid supports include, but are not limited to, gass, cellulose, polyacrylamide, nylon, polystyrene, polyvinyl chloride or polypropylene.

Binding of the peptide tags or peptide tagged molecules to their specific targets can be confirmed by, for example, enzyme-linked immunosorbent assay (ELISA), radioimmunoassay (REA), FACS analysis, bioassay (e.g., growth inhibition), or Western Blot assay. Each of these assays generally detects the presence of protein-ligand complexes of particular interest by employing a labeled reagent (e.g., an antibody) specific for the complex of interest.

Anti-VEGF Antibodies and Antigen Binding Fragments Linked to Peptide Tags

The invention also provides for the peptide tags to be linked to anti-VEGF antibodies, or antigen binding fragments, thereby extending the ocular half-life of the anti-VEGF antibodies, or antigen binding fragments.

In certain aspects the peptide tag is a peptide tag that binds HA, which is linked to a anti-VEGF antibody. In one aspect, the peptide tagged molecule comprises a peptide tag that binds HA in the eye with a KD of less than or equal to 9.0 uM. For example, the peptide tag can bind HA with a KD of less than or equal to, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In one aspect the peptide tag binds HA with a KD of less than or equal to 8.0 uM. In one aspect the peptide tag binds HA with a KD of less than or equal to 7.2 uM. In one aspect the peptide tag binds HA with a KD of less than or equal to 5.5 uM. The peptide tag that binds HA can be a LINK Domain, a TSG-6 LINK Domain, or a specific peptide tag with a sequence of SEQ ID NO: 32, 33, 34, 35 or 36. In certain aspects, the peptide tag is linked to a VEGF binding antibody, or antigen binding fragment (e.g.: such as a Fab) comprising the heavy chain CDRs having the sequence of SEQ ID NOs: 1, 2 and 3, respectively. In other aspects, a peptide tag is linked to a VEGF binding antibody, or antigen binding fragment comprising the light chain CDRs having the sequence of SEQ ID NOs: 11, 12 and 13, respectively. More specifically, a peptide tag is linked to a VEGF binding antibody, or antigen binding fragment comprising the heavy chain CDRs having the sequence of SEQ ID NOs: 1, 2 and 3, respectively and the light chain CDRs having the sequence of SEQ ID NOs: 11, 12 and 13, respectively. In still other aspects, a peptide tag is linked to a VEGF binding antibody, or antigen binding fragment comprising the variable heavy chain having the sequence of SEQ ID NOs: 7. In still other aspects, a peptide tag is linked to a VEGF binding antibody, or antigen binding fragment thereof comprising the variable light chain having the sequence of SEQ ID NOs: 17. In further aspects, a peptide tag is linked to a VEGF binding antibody, or antigen binding fragment comprising the variable heavy chain and variable light chain having the sequence of SEQ ID NOs: 7 and 17, respectively. In still other aspects, a peptide tag is linked to a VEGF binding antibody, or antigen binding fragment comprising the heavy chain having the sequence of SEQ ID NOs: 9. In still other aspects, a peptide tag is linked to a VEGF binding antibody, or antigen binding fragment comprising the light chain having the sequence of SEQ ID NOs: 19. In further aspects, a peptide tag is linked to a VEGF binding antibody, or antigen binding fragment comprising the heavy chain and light chain having the sequence of SEQ ID NOs: 9 and 29, respectively. In further aspects, a peptide tag is linked to a VEGF binding antibody, or antigen binding fragment comprising the heavy chain and light chain having the sequence of SEQ ID NOs: 9 and 29, respectively. More specifically, the heavy chain linked to a peptide tag may have the sequence of SEQ ID NO: 21, 23, 25, 27 or 29. In other specific aspects, the VEGF binding antibody, or antigen binding fragment, linked to a peptide tag, has a peptide tagged heavy chain and light chain with a sequence of SEQ ID NO: 21 & 19, respectively; SEQ ID NO: 23 & 19, respectively; SEQ ID NO: 25 & 19, respectively; SEQ ID NO: 27 & 19, respectively; SEQ ID NO: 29 & 19, respectively; SEQ ID NO: 163 & 164, respectively. In still other aspects, the VEGF binding antigen binding fragment, linked to a peptide tag, is a scFV with a sequence of SEQ ID NO: 166.

In certain aspects a VEGF binding antibody, or antigen binding fragment comprising the heavy chain CDRs having the sequence of SEQ ID NOs: 1, 2 and 3, respectively and the light chain CDRs having the sequence of SEQ ID NOs: 11, 12 and 13, respectively, may have a peptide tag linked to the light chain, the heavy chain and/or have multiple tags on one chain or both chains. More specifically, the peptide tagged VEGF binding antibody, or antigen binding fragment may have heavy chain and light chain with a sequence of SEQ ID NO: 173 & 174, respectively; 175 & 176, respectively; 177 & 178, respectively; 179 & 180, respectively; 181 & 182, respectively.

It is contemplated that a peptide tag with a sequence of SEQ ID NO: 32, 33, 34, 35 or 36, may be linked to ranibizumab (Ferrara et al., 2006), bevacizumab (Ferrara et al., 2004), MP0112 (Campochiaro et al, 2013), KH902 (Zhang et al., 2008), or aflibercept (Stewart et al., 2012).

Other Antibodies or Antigen Binding Fragments Linked to Peptide Tags

The invention also provides for the peptide tags comprising a sequence of SEQ ID NO: 32, 33, 34, 35 or 36 to be linked to antibodies or antigen binding fragments that bind C5, Factor P, EPO, Factor D, TNFα, or II-1β, thereby extending the ocular half-life of the antibodies, or antigen binding fragments. In certain aspects, a peptide tag having a sequence of SEQ ID NO: 32, 33, 34, 35 or 36 is linked to a C5 binding antibody, or antigen binding fragment (e.g.: such as a Fab) comprising the heavy chain CDRs having the sequence of SEQ ID NOs: 37, 38 and 39, respectively. In other aspects, the peptide tag is linked to a C5 binding antibody, or antigen binding fragment comprising the light chain CDRs having the sequence of SEQ ID NOs: 46, 47 and 48, respectively. More specifically, the peptide tag is linked to a C5 binding antibody, or antigen binding fragment comprising the heavy chain CDRs having the sequence of SEQ ID NOs: 37, 38 and 39 respectively and the light chain CDRs having the sequence of SEQ ID NOs: 46, 47 and 48 respectively. In still other aspects, the peptide tag linked to a C5 binding antibody, or antigen binding fragment comprising the variable heavy chain having the sequence of SEQ ID NOs: 40. In still other aspects, the peptide tag linked to a C5 binding antibody, or antigen binding fragment comprising the variable light chain having the sequence of SEQ ID NOs: 49. In further aspects, the peptide tag is linked to a C5 binding antibody, or antigen binding fragment comprising the variable heavy chain and variable light chain having the sequence of SEQ ID NOs: 40 and 49, respectively. In certain aspects, the heavy chain linked to a peptide tag may have the sequence of SEQ ID NO: 44. More specifically, the C5 binding antibody, or antigen binding fragment, linked to a peptide tag has a peptide tagged heavy chain and light chain with a sequence of SEQ ID NO: 44 & 51, respectively.

In certain aspects, a peptide tag having a sequence of SEQ ID NO: 32, 33, 34, 35 or 36 is linked to an Epo binding antibody, or antigen binding fragment (e.g.: such as a Fab) comprising the heavy chain CDRs having the sequence of SEQ ID NOs: 75, 76 and 77, respectively. In other aspects, the peptide tag is linked to a Epo binding antibody, or antigen binding fragment comprising the light chain CDRs having the sequence of SEQ ID NOs: 86, 87 and 88, respectively. More specifically, the peptide tag is linked to a Epo binding antibody, or antigen binding fragment comprising the heavy chain CDRs having the sequence of SEQ ID NOs: 75, 76 and 77, respectively and the light chain CDRs having the sequence of SEQ ID NOs: 86, 87 and 88, respectively. In still other aspects, the peptide tag linked to a Epo binding antibody, or antigen binding fragment comprising the variable heavy chain having the sequence of SEQ ID NOs: 81. In still other aspects, the peptide tag linked to a Epo binding antibody, or antigen binding fragment comprising the variable light chain having the sequence of SEQ ID NOs: 92. In further aspects, the peptide tag is linked to a Epo binding antibody, or antigen binding fragment comprising the variable heavy chain and variable light chain having the sequence of SEQ ID NOs: 81 and 92, respectively. In certain aspects, the heavy chain linked to a peptide tag may have the sequence of SEQ ID NO: 85. More specifically, the Epo binding antibody, or antigen binding fragment, linked to a peptide tag has a peptide tagged heavy chain and light chain with a sequence of SEQ ID NO: 85 & 95, respectively.

In certain aspects, a peptide tag having a sequence of SEQ ID NO: 32, 33, 34, 35 or 36 is linked to a Factor P binding antibody, or antigen binding fragment (e.g.: such as a Fab) comprising the heavy chain CDRs having the sequence of SEQ ID NOs: 53, 54 and 55, respectively. In other aspects, the peptide tag is linked to a Factor P binding antibody, or antigen binding fragment comprising the light chain CDRs having the sequence of SEQ ID NOs: 65, 66 and 67, respectively. More specifically, the peptide tag is linked to a Factor P binding antibody, or antigen binding fragment comprising the heavy chain CDRs having the sequence of SEQ ID NOs: 53, 54 and 55, respectively and the light chain CDRs having the sequence of SEQ ID NOs: 65, 66 and 67, respectively. In still other aspects, the peptide tag linked to a Factor P binding antibody, or antigen binding fragment comprising the variable heavy chain having the sequence of SEQ ID NOs: 59. In still other aspects, the peptide tag linked to a Factor P binding antibody, or antigen binding fragment comprising the variable light chain having the sequence of SEQ ID NOs: 71. In further aspects, the peptide tag is linked to a Factor P binding antibody, or antigen binding fragment comprising the variable heavy chain and variable light chain having the sequence of SEQ ID NOs: 59 and 71, respectively. In certain aspects, the heavy chain linked to a peptide tag may have the sequence of SEQ ID NO: 63. More specifically, the Factor P binding antibody, or antigen binding fragment, linked to a peptide tag has a peptide tagged heavy chain and light chain with a sequence of SEQ ID NO: 63 & 73, respectively.

In certain aspects, a peptide tag having a sequence of SEQ ID NO: 32, 33, 34, 35 or 36 is linked to a TNFα binding antibody, or antigen binding fragment (e.g.: such as a Fab) comprising the heavy chain CDRs having the sequence of SEQ ID NOs: 108, 109 and 110, respectively. In other aspects, the peptide tag is linked to a TNFα binding antibody, or antigen binding fragment comprising the light chain CDRs having the sequence of SEQ ID NOs: 117, 118 and 119, respectively. More specifically, the peptide tag is linked to a TNFα binding antibody, or antigen binding fragment comprising the heavy chain CDRs having the sequence of SEQ ID NOs: 108, 109 and 110, respectively and the light chain CDRs having the sequence of SEQ ID NOs: 117, 118 and 119, respectively. In still other aspects, the peptide tag linked to a TNFα binding antibody, or antigen binding fragment comprising the variable heavy chain having the sequence of SEQ ID NOs: 111. In still other aspects, the peptide tag linked to a TNFα binding antibody, or antigen binding fragment comprising the variable light chain having the sequence of SEQ ID NOs: 120. In further aspects, the peptide tag is linked to a TNFα binding antibody, or antigen binding fragment comprising the variable heavy chain and variable light chain having the sequence of SEQ ID NOs: 111 and 120, respectively. In certain aspects, the heavy chain linked to a peptide tag may have the sequence of SEQ ID NO: 113. More specifically, the TNFα binding antibody, or antigen binding fragment, linked to a peptide tag has a peptide tagged heavy chain and light chain with a sequence of SEQ ID NO: 115 & 122, respectively.

In certain aspects, a peptide tag having a sequence of SEQ ID NO: 32, 33, 34, 35 or 36 is linked to a IL-1β binding antibody, or antigen binding fragment (e.g.: such as a Fab) comprising the heavy chain CDRs having the sequence of SEQ ID NOs: 189, 190 and 191, respectively. In other aspects, the peptide tag is linked to a IL-1β binding antibody, or antigen binding fragment comprising the light chain CDRs having the sequence of SEQ ID NOs: 198, 199 and 200, respectively. More specifically, the peptide tag is linked to a IL-1β binding antibody, or antigen binding fragment comprising the heavy chain CDRs having the sequence of SEQ ID NOs: 189, 190 and 191, respectively and the light chain CDRs having the sequence of SEQ ID NOs: 198, 199 and 200, respectively. In still other aspects, the peptide tag linked to a IL-1β binding antibody, or antigen binding fragment comprising the variable heavy chain having the sequence of SEQ ID NOs: 193. In still other aspects, the peptide tag linked to a IL-1β binding antibody, or antigen binding fragment comprising the variable light chain having the sequence of SEQ ID NOs: 201. In further aspects, the peptide tag is linked to a IL-1β binding antibody, or antigen binding fragment comprising the variable heavy chain and variable light chain having the sequence of SEQ ID NOs: 193 and 201, respectively. In certain aspects, the heavy chain linked to a peptide tag may have the sequence of SEQ ID NO: 194. More specifically, the TNFα binding antibody, or antigen binding fragment, linked to a peptide tag has a peptide tagged heavy chain and light chain with a sequence of SEQ ID NO: 196 & 202, respectively.

In certain aspects, a peptide tag having a sequence of SEQ ID NO: 32, 33, 34, 35 or 36 is linked to an antibody or antigen binding fragment that binds C5, Epo or Factor P as described in WO2010/015608, or WO2012/149246 and herein incorporated by reference.

### Homologous Proteins

The invention also provides proteins and peptide tags that are homologous to the sequences described herein. More specifically, the present invention provides for a protein comprising amino acid sequences that are homologous to the sequences described in Table 1, 2, 8, 8b, 9 and 9b and the protein or peptide tag binds to the respective ocular target, and retains the desired functional properties of those proteins and peptide tags described in Table 1, 2, 8, 8b, 9, 9b and the examples.

For example, the invention provides for anti-VEGF antibodies or antigen binding fragments and peptide tags that are homologous to the sequences described herein. More specifically, the invention provides an antibody, or an antigen binding fragment thereof, comprising a heavy chain variable domain and a light chain variable domain, wherein the heavy chain variable domain comprises an amino acid sequence that is at least 80%, 90%, 95%, 96%, 97%, 98% or 99% identical to the amino acid sequence of SEQ ID NOs: 7; the light chain variable domain comprises an amino acid sequence that is at least 80%, 90%, 95%, 96%, 97%, 98% or 99% identical to the amino acid sequence of SEQ ID NOs: 17; and the antibody specifically binds to VEGF. In certain aspects of the invention the heavy and light chain sequences further comprise HCDR1, HCDR2, HCDR3, LCDR1, LCDR2, and LCDR3 sequences as defined by Kabat, for example SEQ ID NOs: 1, 2, 3, 11, 12, and 13, respectively. In certain other aspects of the invention the heavy and light chain sequences further comprise HCDR1, HCDR2, HCDR3, LCDR1, LCDR2, and LCDR3 sequences as defined by chothia, for example SEQ ID NOs: 4, 5, 6, 14, 15, and 16, respectively.

In other embodiments, the VH and/or VL amino acid sequences may be greater than or equal to 80%, 90%, 95%, 96%, 97%, 98% or 99% identical to the sequences set forth in Tables 1 and 2. In other embodiments, the VH and/or VL amino acid sequences may be identical except for an amino acid substitution in no more than 1, 2, 3, 4 or 5 amino acid positions. An antibody having VH and VL regions having <100% sequence identity to the VH and VL regions of those described in Tables 1 and 2 can be obtained by mutagenesis (e.g., site-directed or PCR-mediated mutagenesis) of nucleic acid molecules described in Tables 1 and 2 (e.g.: for example, nucleic acid molecules encoding SEQ ID NOs: 7 and SEQ ID NOs: 17, respectively) followed by testing of the encoded altered antibody for retained function using the functional assays described herein and in US20120014958.

In other embodiments, the full length heavy chain and/or full length light chain amino acid sequences may be greater than or equal to 80%, 90%, 95%, 96%, 97%, 98% or 99% identical to the sequences set forth in Tables 1 and 2. An antibody having a heavy chain and light chain having high (i.e., 80% or greater) identity to the heavy chains and light chains described in Tables 1 and 2 (e.g.: the heavy chains of any of SEQ ID NOs: 9, 21, 23, 25, 17 or 29 and light chain of SEQ ID NOs: 19) can be obtained by mutagenesis (e.g., site-directed or PCR-mediated mutagenesis) of nucleic acid molecules encoding such polypeptides, followed by testing of the encoded altered antibody for retained function using the functional assays described herein.

In other embodiments, the full length heavy chain and/or full length light chain nucleotide sequences may be greater than or equal to 80%, 90%, 95%, 96%, 97%, 98% or 99% identical to the sequences set forth in Table 1 and Table 2.

In other embodiments, the variable regions of heavy chain and/or the variable regions of light chain nucleotide sequences may be greater than or equal to 80%, 90%, 95%, 96%, 97%, 98% or 99% identical to the sequences set forth in Table 1 and Table 2. It is contemplated that the variability may be in the CDR or framework regions.

In addition, the present invention also provides for a peptide tag comprising amino acid sequences that are homologous to the sequences described in Table 1, and the peptide tag binds to HA and retains the desired functional properties of those peptide tags described herein. More specifically, the amino acid sequences of the peptide tags may be greater than or equal to 80%, 90%, 95%, 96%, 97%, 98% or 99% identical to the sequences set forth in Table 1 and retain the desired functional properties of those the peptide tags described herein.

As used herein, the percent identity between the two sequences is a function of the number of identical positions shared by the sequences (i.e., % identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

Additionally or alternatively, the protein sequences of the present invention can further be used as a “query sequence” to perform a search against public databases to, for example, identify related sequences. For example, such searches can be performed using the BLAST program (version 2.0) of Altschul, et al., 1990 J. Mol. Biol. 215:403-10.

Proteins with Conservative Modifications

Further included within the scope of the invention are isolated peptide tags and peptide tagged molecules, with conservative modifications. More specifically, the invention is related to peptide tags and peptide tagged molecules with conservative modification to the peptide tags and peptide tagged molecules of Table 1. Also included within the scope of the invention are isolated antibodies, or antigen binding fragments, with conservative modifications. In certain aspects, the peptide tagged antibody of the invention has a heavy chain variable region comprising CDR1, CDR2, and CDR3 sequences and a light chain variable region comprising CDR1, CDR2, and CDR3 sequences, wherein one or more of these CDR sequences have specified amino acid sequences based on the antibodies described herein or conservative modifications thereof, and wherein the antibody retains the desired functional properties of the antibodies of the invention. For example, the invention provides a peptide tag linked to a VEGF-binding isolated antibody, or an antigen binding fragment thereof, consisting of a heavy chain variable region comprising CDR1, CDR2, and CDR3 sequences and a light chain variable region comprising CDR1, CDR2, and CDR3 sequences, wherein: the heavy chain variable region CDR1 amino acid sequence is SEQ ID NO: 1, and conservative modifications thereof; the heavy chain variable region CDR2 amino acid sequence is SEQ ID NO: 2, and conservative modifications thereof; the heavy chain variable region CDR3 amino acid sequence is SEQ ID NO: 3, and conservative modifications thereof; the light chain variable regions CDR1 amino acid sequence is SEQ ID NO: 11, and conservative modifications thereof; the light chain variable regions CDR2 amino acid sequence is SEQ ID NO: 12, and conservative modifications thereof; the light chain variable regions of CDR3 amino acid sequence is SEQ ID NO: 13, and conservative modifications thereof; and the antibody or antigen binding fragment thereof specifically binds to VEGF.

In other embodiments, the antibody of the invention is optimized for expression in a mammalian cell and has a full length heavy chain sequence and a full length light chain sequence, wherein one or more of these sequences have specified amino acid sequences based on the antibodies described herein or conservative modifications thereof, and wherein the antibodies retain the desired functional properties of the VEGF binding antibodies of the invention. Accordingly, the invention provides an isolated antibody optimized for expression in a mammalian cell comprising, for example, a variable heavy chain and a variable light chain wherein the variable heavy chain comprises the amino acid sequence of SEQ ID NOs: 7, and conservative modifications thereof; and the variable light chain comprises and amino acid sequence of SEQ ID NOs: 17, and conservative modifications thereof; and the antibody specifically binds to VEGF. The invention further provides an isolated antibody linked to a peptide tag and optimized for expression in a mammalian cell comprising, for example, a variable heavy chain and a variable light chain and a peptide tag wherein the variable heavy chain comprises the amino acid sequence of SEQ ID NOs: 7, and conservative modifications thereof; and the variable light chain comprises an amino acid sequence of SEQ ID NOs: 17, and conservative modifications thereof; and the peptide tag comprises an amino acid sequence selected from SEQ ID NOs: 32, 33, 34, 35 and 36, and the antibody specifically binds to VEGF and the peptide tag specifically binds to HA. The invention provides an isolated antibody optimized for expression in a mammalian cell consisting of a heavy chain and a light chain and a peptide linker and a peptide tag wherein the heavy chain comprising an amino acid sequence of SEQ ID NOs: 9, and conservative modifications thereof; and the light chain comprising an amino acid sequence of SEQ ID NOs: 19, and conservative modifications thereof; and the peptide tag comprising an amino acid sequence selected from SEQ ID NOs: 32, 33, 34, 35 and 36; and the antibody specifically binds to VEGF and the peptide tag specifically binds to HA. More specifically, the invention provides an isolated antibody, or antigen binding fragment thereof, linked to a peptide tag, wherein the linked antibody or fragment is optimized for expression in a mammalian cell consisting of a heavy chain having the amino acid sequence selected from SEQ ID NOs: 21, 23, 25, 27 and 29, and conservative modifications thereof; and a light chain having the amino acid sequence of SEQ ID NOs: 19; and the isolated antibody specifically binds to VEGF and the peptide tag specifically binds to HA.

### Methods of Producing Antibodies & Tags of the Invention

### Nucleic Acids Encoding the Antibodies & Peptide Tags

The invention provides substantially purified nucleic acid molecules which encode the peptide tags, and/or peptide tagged molecules described herein. In certain aspects the invention provides substantially purified nucleic acid molecules which encode peptide tagged proteins, for example, the peptide tagged proteins described Tables 1, 2, 2b, 8b and 9b. More specifically, the invention provides substantially purified nucleic acid molecules which encode NVS1, NVS2, NVS3, NVS4, NVS36, NVS37, NVS70, NVS70T, NVS71, NVS71T, NVS72, NVS72T, NVS72, NVS73T, NVS74, NVS74T, NVS75, NVS75T, NVS76, NVS76T, NVS77, NVS77T, NVS78, NVS78T, NVS79, NVS79T, NVS80, NVS80T, NVS81, NVS81T, NVS82, NVS82T, NVS83, NVS83T, NVS84, NVS84T, NVS1b, NVS1c, NVS1d, NVS1e, NVS1f, NVS1g, NVS1h or NVS1j. Also provided in the invention are nucleic acid molecules which encode at least one peptide tag having a peptide sequence of SEQ ID NO: 32, 33, 34, 35 and/or 36. More specifically, for example, the nucleotide sequence encoding the peptide tag may include the nucleotide sequence of SEQ ID NO: 102, 103, 104, 105 and/or 106.

The invention provides substantially purified nucleic acid molecules which encode the proteins described herein, for example, proteins comprising the anti-VEGF, anti-EPO, anti-05, anti-Factor P, anti-TNFα or anti-IL-1β antibodies or antigen binding fragments, peptide tags, and/or peptide tagged molecules described above. More specifically, some of the nucleic acids of the invention comprise the nucleotide sequence encoding the heavy chain variable region shown in SEQ ID NO: 7, and/or the nucleotide sequence encoding the light chain variable region shown in SEQ ID NO: 17. In certain specific embodiments, the nucleic acid molecules are those identified in Table 1 or Table 2. Some other nucleic acid molecules of the invention comprise nucleotide sequences that are substantially identical (e.g., at least 65, 80%, 95%, or 99%) to the nucleotide sequences of those identified in Table 1 or Table 2. When expressed from appropriate expression vectors, polypeptides encoded by these polynucleotides are capable of exhibiting target antigen binding capacity, such as, for example, anti-VEGF, anti-EPO, anti-05, anti-Factor P, anti-TNFα or anti-IL-1β antigen binding capacity.

Also provided in the invention are polynucleotides which encode at least one CDR region and usually all three CDR regions from the heavy or light chain of the antibody set forth above. Some other polynucleotides encode all or substantially all of the variable region sequence of the heavy chain and/or the light chain of the antibody set forth above. Because of the degeneracy of the code, a variety of nucleic acid sequences may encode each of the immunoglobulin amino acid sequences.

The nucleic acid molecules of the invention can encode both a variable region and a constant region of the antibody. Some of the nucleic acid sequences of the invention comprise nucleotides encoding a modified heavy chain sequence that is substantially identical (e.g., at least 80%, 90%, or 99%) to the original heavy chain sequence (e.g.: substantially identical to the heavy chain of NVS4). Some other nucleic acid sequences comprising nucleotide encoding a modified light chain sequence that is substantially identical (e.g., at least 80%, 90%, or 99%) to the original light chain sequence (e.g.: substantially identical to the light chain of NVS4).

The polynucleotide sequences can be produced by de novo solid-phase DNA synthesis or by PCR mutagenesis of an existing sequence (e.g., sequences as described in the Examples below) encoding a VEGF antibody or its binding fragment. Direct chemical synthesis of nucleic acids can be accomplished by methods known in the art, such as the phosphotriester method of Narang et al., 1979, Meth. Enzymol. 68:90; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991.

Also provided in the invention are expression vectors and host cells for producing the peptide tags, proteins, antibodies or antigen binding fragments, or peptide tagged molecules described above, for example peptide tagged antibodies or antigen binding fragments described herein. More specifically, the invention provides an expression vector comprising a nucleic acid encoding a peptide tag having the sequence of SEQ ID NO: 32, 33, 34, 35 and/or 36, or alternatively, an expression vector comprising a nucleic acid encoding a peptide tagged molecule as described herein. In certain aspects the expression vector comprises a nucleic acid encoding any one of the peptide tagged molecules described in Tables 1, 2, 8 or 9, for example, NVS1, NVS2, NVS3, NVS4, NVS36, NVS37, NVS70, NVS70T, NVS71, NVS71T, NVS72, NVS72T, NVS72, NVS73T, NVS74, NVS74T, NVS75, NVS75T, NVS76, NVS76T, NVS77, NVS77T, NVS78, NVS78T, NVS79, NVS79T, NVS80, NVS80T, NVS81, NVS81T, NVS82, NVS82T, NVS83, NVS83T, NVS84, NVS84T, NVS1b, NVS1c, NVS1d, NVS1e, NVS1f, NVS1g, NVS1h or NVS1j.

Various expression vectors can be employed to express the polynucleotides encoding the peptide tags, the proteins, the antibody chains or antigen binding fragments or peptide tagged antibodies or antigen binding fragments. Both viral-based and non-viral expression vectors can be used to produce the antibodies in a mammalian host cell. Non-viral vectors and systems include plasmids, episomal vectors, typically with an expression cassette for expressing a protein or RNA, and human artificial chromosomes (see, e.g., Harrington et al., Nat Genet 15:345, 1997). For example, non-viral vectors useful for expression of the peptide tags or VEGF polynucleotides and polypeptides in mammalian (e.g., human) cells include pThioHis A, B & C, pcDNA3.1/His, pEBVHis A, B & C, (Invitrogen, San Diego, Calif.), MPSV vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

Methods for generating virus vectors are well known in the art and would allow for the skilled artisan to generate the virus vectors of the invention (See, e.g., U.S. Pat. No. 7,465,583).

The choice of expression vector depends on the intended host cells in which the vector is to be expressed. Typically, the expression vectors contain a promoter and other regulatory sequences (e.g., enhancers) that are operably linked to the polynucleotides encoding a antibody chain or fragment, a peptide tag, or a peptide tagged antibody chain or fragment. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements may also be required or desired for efficient expression of an antibody chain or fragment, a peptide tag, or a peptide tagged antibody chain or fragment. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence positioned to form a fusion protein with polypeptides encoded by inserted peptide tag, antibody, or peptide tagged antibody sequences. More often, such inserted sequences are linked to a signal sequences before inclusion in the vector. Vectors to be used to receive sequences encoding antibody light and heavy chain variable domains, or peptide tagged antibody domains, sometimes also encode constant regions or parts thereof. Such vectors allow expression of the variable regions as fusion proteins with the constant regions thereby leading to production of intact antibodies or antigen binding fragments. Typically, such constant regions are human.

The host cells for harboring and expressing the peptide tags, antibody chains, or peptide tagged molecules (e.g.: peptide tagged antibody or antigen binding fragments), can be either prokaryotic or eukaryotic. E. coli is one prokaryotic host useful for cloning and expressing the polynucleotides of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express antibodies, or peptide tagged molecules (e.g.: peptide tagged antibodies or antigen binding fragments), or peptide tags of the invention. Insect cells in combination with baculovirus vectors can also be used.

In some preferred embodiments, mammalian host cells are used to express and produce the peptide tags, peptide tagged molecules, and/or untagged molecules described herein (e.g. the peptide tagged antibodies or antigen binding fragments) of the present invention. For example, they can be either a hybridoma cell line expressing endogenous immunoglobulin genes (e.g., the 1D6.C9 myeloma hybridoma clone as described in the Examples) or a mammalian cell line harboring an exogenous expression vector (e.g., the SP2/0 myeloma cells exemplified below). These include any normal mortal or normal or abnormal immortal animal or human cell. For example, a number of suitable host cell lines capable of secreting intact immunoglobulins have been developed, are known to those of skill in the art, and include CHO cell lines, various Cos cell lines, HeLa cells, myeloma cell lines, transformed B-cells and hybridomas. The use of mammalian tissue cell culture to express polypeptides is discussed generally in, e.g., Winnacker, FROM GENES TO CLONES, VCH Publishers, N.Y., N.Y., 1987. Expression vectors for mammalian host cells can include expression control sequences, such as an origin of replication, a promoter, and an enhancer (see, e.g., Queen, et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts. (See generally Sambrook, et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express the peptide tags, the antibody chains or antigen binding fragments, or the peptide tagged antibody chains or antigen binding fragments, can be prepared using expression vectors of the invention which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type. The invention further provides for process for producing the peptide tags and/or peptide tagged molecules described herein, wherein a host cell capable of producing a peptide tag or peptide tagged molecule as described herein is cultured under appropriate conditions for the production of one or more peptide tags and/or peptide tagged molecules. The process may further include isolating the peptide tags and/or peptide tagged molecules of the invention.

Expression vectors containing nucleic acid sequences encoding the peptide tags, proteins and/or antibodies or antigen binding fragments peptide tags, of the invention can be used for delivering a gene to the eye. In certain aspects of the invention, the expression vector encodes an antibody is linked to one or more peptide tags of the invention and is suitable for delivery to the eye. In other aspects of the invention, the antibody, or antigen binding fragment, and peptide tags are encoded in one or more expression vectors suitable for delivery to the eye. Methods for delivering a gene product to the eye are known in the art (See, e.g., US05/0220768).

### Generation of Monoclonal Antibodies

Monoclonal antibodies (mAbs) can be produced by a variety of techniques, including conventional monoclonal antibody methodology e.g., the standard somatic cell hybridization technique of Kohler and Milstein, 1975 Nature 256: 495. Many techniques for producing monoclonal antibody can be employed e.g., viral or oncogenic transformation of B lymphocytes. For example, methods of producing anti-VEGF antibodies or antigen binding fragments of the invention are described herein, in the examples, and in WO20120014958.

Animal systems for preparing hybridomas include the murine, rat and rabbit systems. Hybridoma production in the mouse is an established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al.

In a certain embodiment, the antibodies of the invention are human monoclonal antibodies. Such human monoclonal antibodies directed against VEGF can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HuMAb Mouse® (Medarex, Inc.) contains human immunoglobulin gene miniloci that encode un-rearranged human heavy (μ and γ) and κ light chain immunoglobulin sequences, together with targeted mutations that inactivate the endogenous p and K chain loci (see e.g., Lonberg, et al., 1994 Nature 368(6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg, N. et al., 1994 supra; reviewed in Lonberg, N., 1994 Handbook of Experimental Pharmacology 113:49-101; Lonberg, N. and Huszar, D., 1995 Intern. Rev. Immunol. 13: 65-93, and Harding, F. and Lonberg, N., 1995 Ann. N. Y. Acad. Sci. 764:536-546). The preparation and use of HuMAb mice, and the genomic modifications carried by such mice, is further described in Taylor, L. et al., 1992 Nucleic Acids Research 20:6287-6295; Chen, J. et at., 1993 International Immunology 5: 647-656; Tuaillon et al., 1993 Proc. Natl. Acad. Sci. USA 94:3720-3724; Choi et al., 1993 Nature Genetics 4:117-123; Chen, J. et al., 1993 EMBO J. 12: 821-830; Tuaillon et al., 1994 J. Immunol. 152:2912-2920; Taylor, L. et al., 1994 International Immunology 579-591; and Fishwild, D. et al., 1996 Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92103918, WO 93/12227, WO 94/25585, WO 97113852, WO 98/24884 and WO 99/45962, all to Lonberg and Kay; and PCT Publication No. WO 01/14424 to Korman et al.

In another embodiment, human antibodies of the invention can be raised using a mouse that carries human immunoglobulin sequences on transgenes and transchromosomes such as a mouse that carries a human heavy chain transgene and a human light chain transchromosome. Such mice, referred to herein as “KM mice”, are described in detail in PCT Publication WO 02/43478 to Ishida et al.

Still further, alternative transgenic animal systems expressing human immunoglobulin genes are available in the art and can be used to raise antibodies of the invention. For example, an alternative transgenic system referred to as the Xenomouse (Abgenix, Inc.) can be used. Such mice are described in, e.g., U.S. Pat. Nos. 5,939,598; 6,075,181; 6,114,598; 6, 150,584 and 6,162,963 to Kucherlapati et al.

Moreover, alternative transchromosomic animal systems expressing human immunoglobulin genes are available in the art and can be used to raise VEGF antibodies of the invention. For example, mice carrying both a human heavy chain transchromosome and a human light chain tranchromosome, referred to as “TC mice” can be used; such mice are described in Tomizuka et al., 2000 Proc. Natl. Acad. Sci. USA 97:722-727. Furthermore, cows carrying human heavy and light chain transchromosomes have been described in the art (Kuroiwa et al., 2002 Nature Biotechnology 20:889-894) and can be used to raise VEGF antibodies of the invention.

Human monoclonal antibodies of the invention can also be prepared using phage display methods for screening libraries of human immunoglobulin genes. Such phage display methods for isolating human antibodies are established in the art or described in the examples below. See for example: U.S. Pat. Nos. 5,223,409; 5,403,484; and U.S. Pat. No. 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

### Methods of Engineering Altered Proteins & Peptide Tags

As discussed above, the peptide tags, proteins, antibodies and antigen binding fragments shown herein can be used to create new peptide tags, proteins, antibodies and antigen binding fragments by modifying the amino acid sequences described. Thus, in another aspect of the invention, the structural features of a peptide tagged antibody of the invention are used to create structurally related peptide tagged antibodies that retain at least one functional property of the peptide tagged antibodies of the invention, such as, for example, binding to human VEGF and also inhibiting one or more functional properties of VEGF (e.g., inhibit VEGF binding to the VEGF receptor).

For example, one or more CDR regions of the antibodies of the present invention, or mutations thereof, can be combined recombinantly with known framework regions and/or other CDRs to create additional, recombinantly-engineered, antibodies of the invention, as discussed above. Other types of modifications include those described in the previous section. The starting material for the engineering method is one or more of the VH and/or

VL sequences provided herein, or one or more CDR regions thereof. To create the engineered antibody, it is not necessary to actually prepare (i.e., express as a protein) an antibody having one or more of the VH and/or VL sequences provided herein, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Accordingly, in another embodiment, the invention provides a method for preparing a peptide tagged anti-VEGF antibody or antigen binding fragment consisting of a heavy chain variable region antibody sequence having a CDR1 sequence of SEQ ID NO: 1, a CDR2 sequence of SEQ ID NO: 2, and/or a CDR3 sequence of SEQ ID NO: 3; and a light chain variable region antibody sequence having a CDR1 sequence of SEQ ID NO: 11, a CDR2 sequence of SEQ ID NO: 12, and/or a CDR3 sequence of SEQ ID NO: 13; altering at least one amino acid residue within the heavy chain variable region antibody sequence and/or the light chain variable region antibody sequence to create at least one altered antibody sequence; and expressing the altered antibody sequence as a protein.

The altered antibody sequence can also be prepared by screening antibody libraries having fixed CDR3 sequences or minimal essential binding determinants as described in US20050255552 and diversity on CDR1 and CDR2 sequences. The screening can be performed according to any screening technology appropriate for screening antibodies from antibody libraries, such as phage display technology.

Standard molecular biology techniques can be used to prepare and express the altered peptide tag or peptide tagged molecule sequence. The peptide tag or peptide tagged molecule encoded by the altered sequence(s) is one that retains one, some or all of the functional properties of the peptide tag or peptide tagged molecule, for example the proteins or peptide tagged antibodies described herein, such as, for example, NVS1, NVS2, NVS3, NVS4, NVS36, or NVS37.

In certain embodiments of the methods of engineering antibodies or peptide tags of the invention, mutations can be introduced randomly or selectively along all or part of an VEGF antibody coding sequence or peptide tag and the resulting modified VEGF antibodies or peptide tag can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

In certain embodiments of the invention antibodies and peptide tags may be engineered to remove sites of deamidation. Deamidation is known to cause structural and functional changes in a peptide or protein. Deamindation can result in decreased bioactivity, as well as alterations in pharmacokinetics and antigenicity of the protein pharmaceutical. (Anal Chem. 2005 Mar. 1; 77(5):1432-9). In certain other aspects of the invention antibodies and peptide tags can be engineered to add or remove sites of protease cleavage. Examples of peptide tag modifications are described in Table 4 and the examples.

The functional properties of the altered antibodies can be assessed using standard assays available in the art and/or described herein, such as those set forth in the Examples.

### Other Antibody Formats

### Camelid Antibodies

Antibody proteins obtained from members of the camel and dromedary (Camelus bactrianus and Calelus dromaderius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals. See PCT/EP93/02214 (WO 94/04678 published 3 Mar. 1994).

A region of the camelid antibody which is the small single variable domain identified as VHH can be obtained by genetic engineering to yield a small protein having high affinity for a target, resulting in a low molecular weight antibody-derived protein known as a “camelid nanobody”. See U.S. Pat. No. 5,759,808 issued Jun. 2, 1998; see also Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J 17: 3512-3520. Engineered libraries of camelid antibodies and antigen binding fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies being extremely thermostable, stable to extreme pH and to proteolytic digestion, and poorly antigenic. Another consequence is that camelid nanobodies readily move from the circulatory system into tissues. Nanobodies can further facilitate drug transport across the blood brain barrier. See U.S. patent application 20040161738 published Aug. 19, 2004. Further, these molecules can be fully expressed in prokaryotic cells such as E. coli and are expressed as fusion proteins with bacteriophage and are functional.

Accordingly, a feature of the present invention is a camelid antibody or nanobody having, for example, high affinity for VEGF. In certain embodiments herein, the camelid antibody or nanobody is naturally produced in the camelid animal, i.e., is produced by the camelid following immunization with VEGF or a peptide fragment thereof, using techniques described herein for other antibodies. Alternatively, a camelid nanobody is engineered (i.e., produced by selection, for example) from a library of phage displaying appropriately mutagenized camelid nanobody proteins using panning procedures with an appropriate target. Engineered nanobodies can further be customized by genetic engineering. The camelid nanobodiy can be linked to peptide tags as described herein to extend mean residence time, terminal drug concentration and/or increase dose interval, relative to the untagged camelid nanobody. In a specific aspects, the camelid antibody or nanobody is obtained by grafting the CDRs sequences of the heavy or light chain of the human antibodies of the invention into nanobody or single domain antibody framework sequences, as described for example in PCT/EP93/02214.

### Bi-Specific Molecules and Multivalent Antibodies

In another aspect, the present invention features bi-specific or multi-specific molecules comprising a peptide tag of the invention. More specifically, it is contemplated that the present invention features bi-specific or multi-specific molecules comprising a peptide tag, and more than one protein and/or nucleic acid molecule. For example, a multi-specific molecule may comprise a peptide tag, an antibody, or antigen binding fragment thereof, and a nucleic acid molecule of the invention.

An antibody of the invention, or antigen-binding fragment thereof, can be derivatized or linked to another functional molecule, e.g., another peptide or protein (e.g., another antibody or ligand for a receptor) to generate a bi-specific molecule that binds to at least two different binding sites or target molecules. The antibody of the invention may in fact be derivatized or linked to more than one other functional molecule to generate multi-specific molecules that bind to more than two different binding sites and/or target molecules; such multi-specific molecules are also intended to be encompassed by the term “bi-specific molecule” as used herein. To create a bi-specific molecule of the invention, an antibody of the invention can be functionally linked (e.g., by chemical coupling, genetic fusion, non-covalent association or otherwise) to one or more other binding molecules, such as another antibody, antigen binding fragment, peptide, or binding mimetic, such that a bi-specific molecule results.

Accordingly, the present invention includes bi-specific molecules comprising at least one first binding specificity for VEGF and a second binding specificity for a second target epitope. For example, the second target epitope is another epitope of VEGF different from the first target epitope. Alternatively, the second target epitope is an epitope of an alternate ocular molecule. Alternatively, the second target epitope is an epitope of HA.

Additionally, for the invention in which the bi-specific molecule is multi-specific, the molecule can further include a third binding specificity, in addition to the first and second target epitope. Alternatively, the second target epitope is an epitope of an alternate ocular molecule.

In one embodiment, a bi-specific molecule can comprise as a binding specificity at least one antibody, or an antigen binding fragment thereof, including, e.g., a Fab, Fab′, F(ab′)2, Fv, or a single chain Fv. The antibody may also be a light chain or heavy chain dimer, or any minimal fragment thereof such as a Fv or a single chain construct as described in Ladner et al. U.S. Pat. No. 4,946,778.

Diabodies are bivalent, bi-specific molecules in which VH and VL domains are expressed on a single polypeptide chain, connected by a linker that is too short to allow for pairing between the two domains on the same chain. The VH and VL domains pair with complementary domains of another chain, thereby creating two antigen binding sites (see e.g., Holliger et al., 1993 Proc. Natl. Acad. Sci. USA 90:6444-6448; Poljak et al., 1994 Structure 2:1121-1123). Diabodies can be produced by expressing two polypeptide chains with either the structure VHA-VLB and VHB-VLA (VH-VL configuration), or VLA-VHB and VLB-VHA (VL-VH configuration) within the same cell. Most of them can be expressed in soluble form in bacteria. Single chain diabodies (scDb) are produced by connecting the two diabody-forming polypeptide chains with linker of approximately 15 amino acid residues (see Holliger and Winter, 1997 Cancer Immunol. Immunother., 45(3-4):128-30; Wu et al., 1996 Immunotechnology, 2(1):21-36). scDb can be expressed in bacteria in soluble, active monomeric form (see Holliger and Winter, 1997 Cancer Immunol. Immunother., 45(34): 128-30; Wu et al., 1996 Immunotechnology, 2(1):21-36; Pluckthun and Pack, 1997 Immunotechnology, 3(2): 83-105; Ridgway et al., 1996 Protein Eng., 9(7):617-21). A diabody can be fused to Fc to generate a “di-diabody” (see Lu et al., 2004 J. Biol. Chem., 279(4):2856-65).

Other antibodies which can be employed in the bi-specific molecules of the invention are murine, chimeric and humanized monoclonal antibodies.

Bi-specific molecules can be prepared by conjugating the constituent binding specificities, using methods known in the art. For example, each binding specificity of the bi-specific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohexane-1-carboxylate (sulfo-SMCC) (see e.g., Karpovsky et al., 1984 J. Exp. Med. 160:1686; Liu, M A et al., 1985 Proc. Natl. Acad. Sci. USA 82:8648). Other methods include those described in Paulus, 1985 Behring Ins. Mitt. No. 78, 118-132; Brennan et al., 1985 Science 229:81-83), and Glennie et al., 1987 J. Immunol. 139: 2367-2375). Conjugating agents are SATA and sulfo-SMCC, both available from Pierce Chemical Co. (Rockford, Ill.).

When the binding specificities are antibodies, they can be conjugated by sulfhydryl bonding of the C-terminus hinge regions of the two heavy chains. In a particularly embodiment, the hinge region is modified to contain an odd number of sulfhydryl residues, for example one, prior to conjugation.

Alternatively, both binding specificities can be encoded in the same vector and expressed and assembled in the same host cell. This method is particularly useful where the bi-specific molecule is a mAb×mAb, mAb×Fab, Fab×F(ab′)2, ligand x Fab, peptide tag×mAb, peptide tag×Fab fusion protein. A bi-specific molecule of the invention can be a single chain molecule comprising one single chain antibody and a binding determinant, or a single chain bi-specific molecule comprising two binding determinants. Bi-specific molecules may comprise at least two single chain molecules. Methods for preparing bi-specific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

Binding of the bi-specific, or multivalent, molecules to their specific targets can be confirmed by, for example, enzyme-linked immunosorbent assay (ELISA), radioimmunoassay (REA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

In another aspect, the present invention provides multivalent molecules comprising at least two identical or different antigen-binding portions of the antibodies of the invention binding to VEGF. In a further aspect, the present invention provides multivalent compounds comprising at least two identical or different antigen-binding portions of the peptide tags of the invention binding to HA. The antigen-binding portions can be linked together via protein fusion or covalent or non-covalent linkage. Alternatively, methods of linkage have been described for the multi-specific molecules. Tetravalent compounds can be obtained for example by cross-linking antibodies of the antibodies of the invention with an antibody that binds to the constant regions of the antibodies of the invention, for example the Fc or hinge region.

Trimerizing domain are described for example in Borean patent EP 1 012 280B1. Pentamerizing modules are described for example in PCT/EP97/05897.

### Prophylactic and Therapeutic Uses

Many ocular diseases, specifically, for example retinal vascular diseases, are treated with therapies that require intravitreal injection weekly, bi-weekly, or monthly. The method and frequency of treatment poses a significant health-care burden to doctors and patients. In addition there also a significant risk to patients associated with frequent intravitreal injections, due to the risk of endophthalmitis and intraocular pressure due to intravitreal injections. In certain cases, like Glaucoma, the administration of these therapies is challenging and not used routinely in the clinic. Thus, the ability to administer therapies dosed quarterly or less frequently will provide the best improvements in visual outcomes while reducing the treatment burden and risks associated with frequent intravitreal injections.

Retinal diseases including neovascular (wet) AMD, diabetic retinopathy, and retinal vein occlusions have an angiogenic component that leads to loss of vision. Clinical trials have demonstrated that these diseases can be treated effectively with monthly intravitreal injections of ocular biologic thereapies, for example anti-VEGF therapies such as, ranibizumab or bevacizumab or bi-monthly treatment with aflibercept. Despite the efficacy of these therapies, monthly or bi-monthly treatment is a significant health-care burden for patients and physicians (Oishi et al. (2011). Thus there is often a need for an ocular therapy that can be delivered less frequently, yet still provide the same treatment benefit seen with monthy or bi-monthly treatment. Anti-VEGF therapies are generally safe and well-tolerated by most patients, but there is a slight risk of endophthalmitis due to the intravitreal procedure (Day et al., 2011). Recent clinical data indicate that there may be a trend towards increased non-ocular adverse events with bevacizumab, a full-length IgG as compared to ranibizumab, an antigen binding fragment (e.g., Fab). A major difference and potential cause of the systemic adverse events of bevacizumab compared to ranibizumab is the higher systemic exposure of bevacizumab which is accompanied by higher suppression of VEGF in circulation (Comparison of Age-related Macular Degeneration Treatments Trials (CATT) Research Group, Martin D F, Maguire M G, Fine S L, Ying G S, Jaffe G J, Grunwald J E, Toth C, Redford M, Ferris F L 3rd. Ophthalmology. 2012 July; 119(7):1388-98.). Thus an anti-VEGF therapy that could be administered less frequently would have a safety benefit due to the reduced number of intravitreal procedures and lower systemic suppression of VEGF.

In the pivotal MARINA trial (Rosenfeld et al., 2006), monthly injections of ranibizumab resulted in a gain of 10-15 letters in best corrected visual acuity (BCVA), while patients that did not receive treatment lost an average ˜10 letters of vision. Subsequent studies in wet AMD patients assessed different dosing paradigms to see whether visual acuity gains could be maintained with fewer intravitreal treatments (PIER, PRONTO, EXCITE, SUSTAIN, HORIZON, CATT). These trials have demonstrated that monthly dosing resulted in superior visual outcomes compared to less frequent dosing regimens (Patel et al., 2011). There is a need for anti-VEGF therapies that have longer duration of action that will result in patients needing injections less frequently than monthly or bi-monthly while still maintaining the efficacy that is achieved with monthly or bi-monthly dosing regimens.

In addition to VEGF, other proangiogenic, inflammatory, or growth factor mediators are involved in the retinal diseases, such as, for example, neovascular (wet) AMD, diabetic retinopathy, and retinal vein occlusions. Examples of these proangiogenic, inflammatory, or growth factor mediator molecules include but are not limited to PDGF (Boyer, 2013), angiopoietin (Oliner et al., 2012), S1P (Kaiser, 2013), integrins αvβ3, αvβ5, α5β1(Kaiser et al., 2013; Patel, 2009a; Patel, 2009b), betacellulin (Anand-Apte et al., 2010), apelin/APJ (Hara et al., 2013), erythropoietin (Watanabe et al., 2005; Aiello, 2005), complement factor D, TNFα, and proteins linked to AMD risk by genetic association studies such as proteins of the complement pathway including C2, factor B, factor H, CFH R3, C3b, C5, C5a, and C3a, and HtrA1, ARMS2, TIMP3, HLA, IL8, CX3CR1, TLR3, TLR4, CETP, LIPC, COL10A1, and TNFRSF10A (Nussenblatt et al., 2013). As therapies are developed that effectively target these molecules and pathways, there will be a need to provide the improvements in visual outcomes while reducing the treatment burden and risks associated with frequent intravitreal injections. Another retinal disease is Dry AMD, the most common form of AMD that is characterized by the presence of drusen, deposits of debris seen as yellow spots on the retina. Dry AMD may progress to more severe forms such as neovascular (wet) AMD or geographic atrophy. Dry AMD and geographic atrophy are chronic diseases and thus therapies will potentially have to be administered for many years. Thus, there is a need to improve visual outcomes while simultaneously reducing the treatment burden and risks associated with frequent intravitreal injections. Other ocular diseases that include but are not limited to glaucoma, dry eye, or uveitis may also be amenable to treatment with therapies delivered intravitreally.

The present invention provides peptide tags that can be attached to a therapeutic molecule to slow the clearance of the therapeutic molecule from the eye, thereby increasing its ocular half-life. The invention relates to peptide tags and peptide tagged molecules with increased duration of efficacy relative to an untagged molecule, which will lead to less frequent intraocular injections and improved patient treatment in the clinic.

The peptide tagged molecules described herein can be used as a medicament. In particular the peptide tagged molecules of the invention may be used for treating a condition or disorder associated with retinal vascular disease in a subject. For example, peptide tagged antibodies or antigen binding fragments that bind VEGF as described herein, can be used at a therapeutically useful concentration for the treatment of an ocular disease or disorder associated with increased VEGF levels and/or activity by administering to a subject in need thereof an effective amount of the tagged antibodies or antigen binding fragments of the invention.

The present invention provides a method of treating conditions or disorders associated with retinal vascular disease by administering to a subject in need thereof an effective amount of the peptide tagged molecules of the invention. The present invention provides a method of treating conditions or disorders associated with diabetic retinopathy (DR) by administering to a subject in need thereof an effective amount of the peptide tagged molecules of the invention. The present invention provides a method of treating conditions or disorders associated with macular edema administering to a subject in need thereof an effective amount of the peptide tagged molecules of the invention. The invention also provides a method of treating diabetic macular edema (DME) by administering to a subject in need thereof an effective amount of the peptide tagged molecules of the invention. The present invention further provides a method of treating proliferative diabetic retinopathy (PDR) by administering to a subject in need thereof an effective amount of the peptide tagged molecules of the invention. Still further, the present invention provides methods for treating age-related macular edema (AMD), retinal vein occlusion (RVO), angioedema, multifocal choroiditis, myopic choroidal neovascularization, and/or retinopathy of prematurity, by administering to a subject in need thereof an effective amount of the peptide tagged molecules of the invention. Further still, the invention relates to a method of treating a VEGF-mediated disorder by administering to a subject in need thereof an effective amount of the peptide tagged molecules of the invention. It is contemplated that the peptide tagged molecules comprises a peptide tag that binds HA in the eye with a KD of less than or equal to 9.0 uM. For example, the peptide tag can bind HA with a KD of less than or equal to, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. It is contemplated that the peptide tagged molecules is a peptide tagged antibody or antigen binding fragment as described herein. In one aspect, the peptide tagged molecule comprises a peptide tag that binds HA in the eye with a KD of less than or equal to 8.0 uM. In one aspect, the peptide tagged molecule comprises a peptide tag that binds HA in the eye with a KD of less than or equal to 7.2 uM. In one aspect, the peptide tagged molecule comprises a peptide tag that binds HA in the eye with a KD of less than or equal to 6.0 uM. In one aspect, the peptide tagged molecule comprises a peptide tag that binds HA in the eye with a KD of less than or equal to 5.5 uM. In certain specific aspects, the peptide tag may comprise a sequence of SEQ ID NO: 32, 33, 34, 35 or 36. In a further aspect, the foregoing methods further comprise, prior to the step of administering, the step of diagnosing a subject with such condition or disorder.

In one aspect, the invention relates to a method of treating a VEGF-mediated disorder in a subject that is refractory to anti-VEGF therapy by administering to the subject in need thereof an effective amount of the peptide tagged molecules of the invention. It is contemplated that the peptide tagged molecules comprises a peptide tag that binds HA in the eye with a KD of less than or equal to 9.0 uM. For example, the peptide tag can bind HA with a KD of less than or equal to, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In certain specific aspects, the peptide tag may comprise a sequence of SEQ ID NO: 32, 33, 34, 35 or 36. As used here, “refractory to anti-VEGF therapy” refers to the inability to achieve a satisfactory physiological response with known anti-VEGF therapy, such as ranibizumab, bevacizumab, aflibercept, or pegaptanib therapy. Such patients have less than a 20% decrease in abnormal central retina thickness (center 1 mm² area of the macula) after 3 intravitreal injections of ranibizumab, bevacizumab, or aflibercept (or 3 intravitreal injections of a combination of any of the foregoing therapies). In one embodiment, a patient who is refractory to anti-VEGF therapy experiences a continuing worsening of vision despite ranibizumab, bevacizumab, aflibercept, or pegaptanib therapy. In another embodiment, a patient who is refractory to anti-VEGF therapy experiences thickening of the retina despite ranibizumab, bevacizumab, aflibercept, or pegaptanib therapy. In some embodiments, patients refractory to anti-VEGF therapy demonstrate negligible anatomical improvement despite receiving ranibizumab, bevacizumab, aflibercept, or pegaptanib therapy.

The peptide tagged molecules (e.g.: peptide tagged antibodies or antigen binding fragments) of the invention can be used, inter alia, to prevent progression of conditions or disorders associated with retinal vascular disease (for example, DR, DME, NPDR, PDR, age-related macular degeneration (AMD), retinal vein occlusion (RVO), angioedema, multifocal choroiditis, myopic choroidal neovascularization, and/or retinopathy of prematurity), to treat or prevent macular edema associated with retinal vascular disease, to reduce the frequency of intravitreal injections compared to the frequency of injections needed with current anti-VEGF drugs (e.g., ranibizumab, bevacizumab, aflibercept), and to improve vision lost due to retinal vascular disease progression. The peptide tagged molecules (e.g.: the peptide tagged antibodies or antigen binding fragments) of the invention can also be used in combination with, for example, other anti-VEGF therapies, other anti-PDGF therapies, other anti-complement therapies, or other anti-EPO therapies, or other anti-inflammatory therapies for the treatment of patients with retinal vascular disease.

Treatment and/or prevention of retinal vascular disease, macular edema, diabetic retinopathy, diabetic macular edema, proliferative diabetic retinopathy, and VEGF-mediated disorder, and other conditions or disorders associated with retinal vascular disease can be determined by an ophthalmologist or health care professional using clinically relevant measurements of visual function and/or retinal anatomy. Treatment of conditions or disorders associated with retinal vascular disease means any action (e.g., administration of a peptide tagged anti-VEGF antibody described herein) that results in, or is contemplated to result in, the improvement or preservation of visual function and/or retinal anatomy. In addition, prevention as it relates to conditions or disorders associated with retinal vascular disease means any action (e.g., administration of a peptide tagged anti-VEGF antibody described herein) that prevents or slows a worsening in visual function, retinal anatomy, and/or a retinal vascular disease parameter, as defined herein, in a patient at risk for said worsening.

Visual function may include, for example, visual acuity, visual acuity with low illumination, visual field, central visual field, peripheral vision, contrast sensitivity, dark adaptation, photostress recovery, color discrimination, reading speed, dependence on assistive devices (e.g., large typeface, magnifying devices, telescopes), facial recognition, proficiency at operating a motor vehicle, ability to perform one or more activities of daily living, and/or patient-reported satisfaction related to visual function.

Exemplary measures of visual function include Snellen visual acuity, ETDRS visual acuity, low-luminance visual acuity, Amsler grid, Goldmann visual field, Humphrey visual field, microperimetry, Pelli-Robson charts, SKILL card, Ishihara color plates, Farnsworth D15 or D100 color test, standard electroretinography, multifocal electroretinography, validated tests for reading speed, facial recognition, driving simulations, and patient reported satisfaction. Thus, treatment of vascular disease and/or macular edema can be said to be achieved upon a gain of or failure to lose 2 or more lines (or 10 letters) of vision on an ETDRS scale. In addition, treatment of vascular disease and/or macular edema can be said to occur where a subject exhibits at least a 10% an increase or lack of 10% decrease in reading speed (words per minute). In addition, treatment of vascular disease and/or macular edema can be said to occur where a subject exhibits at least a 20% increase or lack of a 20% decrease in the proportion of correctly identified plates on an Ishihara test or correctly sequenced disks on a Farnsworth test. Further, treatment of retinal vascular disease and/or macular edema, can be said to occur if a subject has, for example, at least 10% decrease or lack of a 10% or more increase in time to a pre-specified degree of dark adaptation. In addition, treatment of retinal vascular disease and/or macular edema can be said to occur where a subject exhibits, for example, at least a 10% reduction or lack of a 10% or more increase in total area of visual scotoma expressed as a visual angle determined by a qualified health care professional (i.e., opthalmologist).

Undesirable aspects of retinal anatomy that may be treated or prevented include, for example, microaneurysm, macular edema, cotton-wool spot, intraretinal microvascular abnormality (IRMA), capillary dropout, leukocyte adhesion, retinal ischemia, neovascularization of the optic disk, neovascularization of the posterior pole, iris neovascularization, intraretinal hemorrhage, vitreous hemorrhage, macular scar, subretinal fibrosis, and retinal fibrosis, venous dilation, vascular tortuosity, vascular leakage. Thus, treatment of, for example, macular edema can be determined by a 20% or more reduction in thickness of the central retinal sub-field as measured by optical coherence tomography.

Exemplary means of assessing retinal anatomy include funduscopy, fundus photography, fluorescein angiography, indocyanine green angiography, optical coherence tomography (OCT), spectral domain optical coherence tomography, scanning laser ophthalmoscopy, confocal microscopy, adaptive optics, fundus autofluorescence, biopsy, necropsy, and immunohistochemistry. Thus, vascular disease and/or macular edema can be said to be treated in a subject upon a 10% reduction in leakage area as determined by fluorescein angiography.

Subjects to be treated with therapeutic agents of the present invention can also be administered other therapeutic agents with known methods of treating conditions associated with diabetes mellitus, such as all forms of insulin and anti-hypertensive medications.

Treatment and/or prevention of ocular disease such as age-related macular degeneration (AMD), retinal vein occlusion (RVO), angioedema, multifocal choroiditis, myopic choroidal neovascularization, and/or retinopathy of prematurity can be determined by an ophthalmologist or health care professional using clinically relevant measurements of visual function and/or retinal anatomy by any of the measures described above. Although the measures described herein don't apply to each and every ocular disease herein, one of skill in the art would recognize the clinically relevant measurement of visual function and/or retinal anatomy that could be used to treat the given ocular disease.

When the therapeutic agents of the present invention are administered together with another agent, the two can be administered sequentially in either order or simultaneously. In some aspects, a tagged antibody or antigen binding fragment of the present invention is administered to a subject who is also receiving therapy with a second agent (e.g., Lucentis). In other aspects, the binding molecule is administered in conjunction with surgical treatments.

Suitable agents for combination treatment with a tagged antibody or antigen binding fragment of the invention include agents known in the art that are able to modulate the activities of VEGF, VEGF receptors, other receptor tyrosine kinase inhibitors, or other entities that modulate HIF-1 mediated pathways. Other agents have been reported to inhibit these pathways include ranibizumab, bevicizumab, pegaptanib, aflibercept, pazopanib, sorafinib, sunitinib, and rapamycin. Combination treatments with anti-inflammatory agents such as corticosteroids, NSAIDS, and TNF-α inhibitors could also be beneficial in the treatment of retinal vascular disease and macular edema, for example, diabetic retinopathy and DME.

A combination therapy regimen may be additive, or it may produce synergistic results (e.g., reductions in retinopathy severity more than expected for the combined use of the two agents). In some embodiments, the present invention provides a combination therapy for preventing and/or treating retinal vascular diseases and macular edema, specifically AMD and diabetic retinopathy, including DME and/or PDR as described above, with a tagged antibody or antigen binding fragment of the invention and an anti-angiogenic, such as second anti-VEGF agent. In certain other embodiments, the present invention provides a combination therapy for preventing and/or treating retinal vascular diseases and macular edema, specifically neovascular AMD and diabetic retinopathy, including DME and/or PDR as described above, with a peptide tagged antibody or peptide tagged antigen binding fragment of the invention and an agent that inhibits other ocular targets such as VEGF, PDGF, EPO, components of the complement pathway (e.g.: C5, Factor D, Factor P, C3), SDF1, Apelin, Betacellulin, or an anti-inflammatory agent (e.g: steroid).

In one aspect, the invention relates to a method of extending the duration of efficacy of an intravitreally-administered therapeutic. Extending duration of efficacy (e.g., increasing dosing interval) can be achieved by increasing the ocular half-life, decreasing ocular clearance, or increasing the ocular mean residence time of the therapeutic. Half-life or mean residence time can be increased (and clearance decreased) by linking the therapeutic (e.g., a protein or nucleic acid) to a peptide tag that binds HA. Accordingly, in one aspect, the invention relates to a method of increasing the half-life, mean residence time, and/or decreasing the clearance of a molecule in the eye. In particular the invention relates to a method of increasing the half-life and/or mean residence time, or decreasing the clearance of a protein or nucleic acid in the eye by linking the protein or nucleic acid to a peptide tag described herein.

An increase in dosing interval results from the increased half-life, increased mean residence time, increased terminal concentration, and/or decreased clearance rate of a molecule from the eye. The invention also provides for methods for increasing half-life of molecule in the eye comprising the step of administering, to the eye of the subject, a composition comprising the molecule linked to a peptide tag that binds HA with a KD of less than or equal to 9.0 uM. In certain specific aspects, the method comprises administering a composition comprising the molecule linked to a peptide tag that binds HA with a KD of less than or equal to 8.0 uM. In certain specific aspects, the method comprises administering a composition comprising the molecule linked to a peptide tag that binds HA with a KD of less than or equal to 7.2 uM. In certain specific aspects, the method comprises administering a composition comprising the molecule linked to a peptide tag that binds HA with a KD of less than or equal to 5.5 uM. The invention provides for methods for increasing mean residence time, increasing terminal concentration and/or decreasing clearance of molecule in/from the eye comprising the step of administering, to the eye of the subject, a composition comprising the molecule linked to a peptide tag that binds HA with a KD of less than or equal to 9.0 uM. In certain specific aspects, the method comprises administering a composition comprising the molecule linked to a peptide tag that binds HA with a KD of less than or equal to 8.0 uM. In certain specific aspects, the method comprises administering a composition comprising the molecule linked to a peptide tag that binds HA with a KD of less than or equal to 7.2 uM. In certain specific aspects, the method comprises administering a composition comprising the molecule linked to a peptide tag that binds HA with a KD of less than or equal to 5.5 uM. In certain aspects the peptide tag comprises the sequence of SEQ ID NO: 32, 33, 34, 36, or 37. It is contemplated that the composition comprises a peptide tag that binds HA with a KD of less than or equal to 9.0 uM, 8.0 uM, 7.2 uM, or 5.5 uM linked to a protein or nucleic acid, for example, an antibody or antigen binding fragment, more specifically, for example, an anti-VEGF antibody or antigen binding fragment.

Half-life as described herein, refers to the time required for the concentration of a drug to fall by one-half (Rowland M and Towzer T N: Clinical Pharmacokinetics. Concepts and Applications. Third edition (1995) and Bonate P L and Howard D R (Eds): Pharmacokinetics in Drug Development, Volume 1 (2004)). Details may also be found in Kenneth, A et al: Chemical Stability of Pharmaceuticals: A Handbook for Pharmacists and in Peters et al, Pharmacokinetic analysis: A Practical Approach (1996). Reference is also made to “Pharmacokinetics”, M Gibaldi & D Perron, published by Marcel Dekker, 2 nd Rev. ex edition (1982), which describes pharmacokinetic parameters such as alpha half-life and beta half-life and area under the curve (AUC). Optionally, all pharmacokinetic parameters and values quoted herein are to be read as being values in a human. Optionally, all pharmacokinetic parameters and values quoted herein are to be read as being values in a mouse or rat or Cynomolgus monkey.

In one aspect, at least a 25% increase (e.g. from 5 to 6.25 days) in half-life by binding to HA is contemplated. In another aspect at least a 50% increase (e.g. from 5 to 7.5 days) in half-life is contemplated. In another aspect at least a 75% increase (e.g. from 5 to 8.75 days) in half-life is contemplated. In another aspect, at least a 100% increase (e.g. from 5 to 10 days) in half-life is contemplated. In another aspect, a greater than 100% increase (e.g., 150%, 200%) in half-life is contemplated. In one aspect, linking a peptide tag to a molecule as described herein can increase the ocular half-life by at least 1.5 fold, at least 2 fold, at least 2.5 fold, at least 3 fold, at least 3.5 fold, and at least 4 fold or more relative to the ocular half-life of the molecule without the tag. Relative increases in ocular half-life for an HA-binding peptide tagged molecule compared to an untagged molecule can be determined by administering the molecules by intravitreal injection and measuring the concentrations remaining at various time points using analytical methods known in the art, for example ELISA, mass spectrometry, western blot, radio-immunoassay, or fluorescent labeling. Clearance from the vitreous of an intravitreally administered biologic molecule has been shown to fit a first-order exponential decay function (equation 1) (Krohne et al., 2008; Krohne et al., 2012; Bakri et al., 2007b; Bakri et al., 2007a; Gaudreault et al., 2007; Gaudreault et al., 2005).

Ct=Ct=0*e^{-kt} (1)

The rate constant k is:

$$k = \frac{ln 2}{t 1 \text{/} 2} \qquad ( 2 )$$

Cₜ is the concentration at time t after intravitreal administration.

Cₜ₌₀ is the concentration at time 0 after intravitreal administration.

T_{1/2} is the ocular half-life after intravitreal administration.

The effects of increasing the intravitreal half-life can be modeled using equations (1) and (2).

Methods for pharmacokinetic analysis and determination of mean residence time and/or half-life of a peptide tagged molecule will be familiar to those skilled in the art. In addition, details related to methods for pharmacokinetic analysis and determination of mean residence time of a peptide tagged molecule may be found in Shargel, L and Yu, ABC: Applied Biopharmaceutics & Pharmacokinetics, 4^{th} Edition (1999), Rowland M and Towzer T N: Clinical Pharmacokinetics. Concepts and Applications. Third edition (1995) and Bonate P L and Howard D R (Eds): Pharmacokinetics in Drug Development, Volume 1 (2004), which describes pharmacokinetic parameters such as Mean Residence Time. Mean residence time and AUC can be determined from a curve of matrix or tissue (e.g.: serum) concentration of a drug (e.g.: therapeutic protein, peptide tagged protein, peptide tag, etc.) against time. Phoenix WinNonlin software, eg version 6.1 (available from Pharsight Corp., Cary, N.C., USA) can be used, for example, to analyze and/or model such data. The mean residence time is the average time that the drug resides in the body and encompasses absorption, distribution and elimination processes. MRT represents the time when 63.2% of the dose has been eliminated.

In one aspect, the invention relates to a method of increasing mean residence time of a molecule (such as a protein or nucleic acid) by linking the molecule to a peptide tag as described herein. In one aspect linking a peptide tag to a molecule as described herein can increase the mean residence time of the molecule in the eye by 10% or more. In a further aspect linking a peptide tag to a molecule as described here in can increase the mean residence time of the molecule in the eye by 20%, 30%, 40%, 50%, 60%, 70%, 80%, 90%, or 100% or more.

In a further aspect, the invention relates to a method of decreasing ocular clearance of the molecule (such as a protein or nucleic acid) by linking the molecule to a peptide tag as described herein. In one aspect, linking a peptide tag to a molecule as described herein can decrease ocular clearance of the molecule in the eye by 10% or more. In a further aspect, thinking a peptide tag to a molecule as described herein can decrease ocular clearance of the molecule in the eye by 20%, 30%, 40%, 50%, 60%, 70%, 80%, 90%, or 100% or more.

### Pharmaceutical Compositions

Delivery of Peptide Tags & Peptide Tagged Molecules

The invention provides compositions comprising a peptide tag of the invention, for example a peptide tag that binds HA in the eye with a KD of less than or equal to 9.0 uM, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM, or 0.5 uM. In certain specific aspects the peptide tag may comprise the sequence of SEQ ID NO: 32, 33, 34, 35, or 36, formulated together, or separately, with a pharmaceutically acceptable excipient, diluent or carrier. The invention also provides compositions comprising a peptide tagged molecules (e.g.: a peptide tag linked to a protein or a nucleic acid), formulated together, or separately, with a pharmaceutically acceptable excipient, diluent or carrier. In certain aspects the peptide tagged molecule comprises a peptide tag that binds HA in the eye as described above. The invention also provides compositions comprising peptide tagged antibodies, or peptide tagged antigen binding fragments, and/or a peptide tag, formulated together, or separately, with a pharmaceutically acceptable excipient, diluent or carrier. In certain aspects, the invention provides compositions comprising a VEGF antibody, or antigen binding fragment thereof, linked to a peptide tag, formulated together with a pharmaceutically acceptable excipient, diluent or carrier. In more specific aspects, the invention provides compositions comprising the peptide tagged molecule: NVS1, NVS2, NVS3, NVS36 or NVS37. In still more specific aspects, the invention provides compositions comprising the peptide tagged molecule in any of Tables 1, 2, 8, 8b, 9, or 9b. The compositions described herein may be formulated together with a pharmaceutically acceptable excipient, diluent or carrier. The compositions can additionally contain one or more other therapeutic agents that are suitable for treating or preventing, for example, conditions or disorders associated with retinal vascular disease. Pharmaceutically acceptable carriers enhance or stabilize the composition, or can be used to facilitate preparation of the composition. Pharmaceutically acceptable carriers include solvents, dispersion media, coatings, antibacterial and antifungal agents, isotonic and absorption delaying agents, and the like that are physiologically compatible.

A pharmaceutical composition of the present invention can be administered by a variety of methods known in the art. The route and/or mode of administration vary depending upon the desired results. It is preferred that the composition be suitable for administration to the eye, more specifically, the composition may be suitable for intravitreal administration. The pharmaceutically acceptable excipient, diluent or carrier should be suitable for administration to the eye. (e.g., by injection, subconjunctival or topical administration), more specifically, for intravitreal administration. Depending on the route of administration, the active compound (i.e., antibody, bi-specific and multi-specific molecule), may be coated in a material to protect the compound from the action of acids and other natural conditions that may inactivate the compound. The invention also provides for methods of producing a composition for ocular delivery wherein the method includes the step of linking a peptide tag that binds HA in the eye with a KD of less than or equal to 9.0 uM, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM, or 0.5 uM to a molecule (e.g.: a protein or nucleic acid) that binds or is capable of binding a target in the eye (e.g.: VEGF, Factor P, Factor D, EPO, TNFα, C5, IL-1β, etc).

The composition should be sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions of the invention can be prepared in accordance with methods well known and routinely practiced in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the molecule employed in the pharmaceutical compositions of the invention. The peptide tagged molecules are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors. Dosage level may be selected and/or adjusted to achieve a therapeutic response as determined using one or more of the ocular/visual assessments described herein.

A physician or veterinarian can start doses of the peptide tagged molecules of the invention employed in the pharmaceutical composition at levels lower than that required to achieve the desired therapeutic effect and gradually increase the dosage until the desired effect is achieved. In general, effective doses of the compositions of the present invention, for the treatment of an retinal vascular disease described herein vary depending upon many different factors, including means of administration, target site, physiological state of the patient, whether the patient is human or an animal, other medications administered, and whether treatment is prophylactic or therapeutic. Treatment dosages need to be titrated to optimize safety and efficacy Dosage for intravitreal administration with a peptide tagged molecule may range from 0.1 mg/eye to 6 mg/eye per injection. A single dose per eye may be carried out in 2 injections per eye. For example, a single dose of 12 mg/eye may be delivered in 2 injections of 6 mg each, resulting in a total dose of 12 mg. In certain specific aspects, a dose may be 12 mg/eye, 11 mg/eye, 10 mg/eye, 9 mg/eye, 8 mg/eye, 7 mg/eye, 6 mg/eye, 5 mg/eye, 4.5 mg/eye, 4 mg/eye, 3.5 mg/eye, 3 mg/eye, 2.5 mg/eye, 2 mg/eye, 1.5 mg/eye, 1 mg/eye, 0.9 mg/eye, 0.8 mg/eye, 0.7 mg/eye, 0.6 mg/eye, 0.5 mg/eye, 0.4 mg/eye, 0.3 mg/eye, 0.2 mg/eye, or 0.1 mg/eye or lower. Each dose may be carried out in one or more injections per eye. The volume per injection may be between 10 microliters and 50 micoliters, while the volume per dose may be between 10 microliters and 100 micoliters. For example, doses include 0.1 mg/50 ul, 0.2 mg/50 ul, 0.3 mg/50 ul, 0.4 mg/50 ul, 0.5 mg/50 ul, 0.6 mg/50 ul, 0.7 mg/50 ul, 0.8 mg/50 ul, 0.9 mg/50 ul, 1.0 mg/50 ul, 1.1 mg/50 ul, 1.2 mg/50 ul, 1.3 mg/50 ul, 1.4 mg/50 ul, 1.5 mg/50 ul, 1.6 mg/50 ul, 1.7 mg/50 ul, 1.8 mg/50 ul, 1.9 mg/50 ul, 2.0 mg/50 ul, 2.1 mg/50 ul, 2.2 mg/50 ul, 2.3 mg/50 ul, 2.4 mg/50 ul, 2.5 mg/50 ul, 2.6 mg/50 ul, 2.7 mg/50 ul, 2.8 mg/50 ul, 2.9 mg/50 ul, 3.0 mg/50 ul, 3.1 mg/50 ul, 3.2 mg/50 ul, 3.3 mg/50 ul, 3.4 mg/50 ul, 3.5 mg/50 ul, 3.6 mg/50 ul, 3.7 mg/50 ul, 3.8 mg/50 ul, 3.9 mg/50 ul, 4.0 mg/50 ul, 4.1 mg/50 ul, 4.2 mg/50 ul, 4.3 mg/50 ul, 4.4 mg/50 ul, 4.5 mg/50 ul, 4.6 mg/50 ul, 4.7 mg/50 ul, 4.8 mg/50 ul, 4.9 mg/50 ul, 5.0 mg/50 ul, 5.1 mg/50 ul, 5.2 mg/50 ul, 5.3 mg/50 ul, 5.4 mg/50 ul, 5.5 mg/50 ul, 5.6 mg/50 ul, 5.7 mg/50 ul, 5.8 mg/50 ul, 5.9 mg/50 ul, or 6.0 mg/50 ul per eye per injection. An exemplary treatment regime entails IVT administration once per every two weeks or once a month or once every 2 months or once every 3 to 6 months or as needed (PRN). The peptide tagged molecules allow for an increase in dosing intervals which improve the treatment regime of current therapies and is described in further detail below.

FDA approved doses and regimes suitable for use with Lucentis are considered. Other doses and regimes suitable for use with anti-VEGF antibodies or antigen binding fragments are described in US 20120014958.

A composition of a peptide tag or peptide tagged molecule may be administered on multiple occasions. Intervals between single dosages can be weekly, monthly or yearly. Intervals can also be irregular as indicated by the need for retreatment in the patient, based for example on visual acuity or macular edema. In addition alternative dosing intervals can be determined by a physician and administered monthly or as necessary to be efficacious. Efficacy is based on lesion growth, rate of anti-VEGF rescue, retinal thickness as determined by Optical Coherence Tomography (OCT), and visual acuity. Dosage and frequency may vary depending on the half-life of the peptide tagged molecule in the patient and levels of the therapeutic target (e.g., VEGF, C5, EPO, Factor P, etc.). Extending the duration of efficacy of a therapeutic molecule administered IVT can be achieved by increasing the ocular T_{1/2} and/or increasing its ocular mean residence time and/or decreasing clearance. Extending the duration of efficacy can be achieved, for example by linking an HA-binding peptide tag to a molecule to slow its clearance from the vitreous, retina and/or RPE/choroid resulting in an increased ocular half-life of the peptide tagged molecule. Relative increases in ocular half-life for a peptide tagged molecule that binds HA compared to an untagged molecule can be determined by administering the molecules by intravitreal injection and measuring the concentrations remaining at various time points using analytical methods known in the art, for example ELISA, mass spectrometry, western blot, radio-immunoassay, or fluorescent labeling. Blood concentrations can also be measured and used to calculate the rate of clearance from the eye as described (Xu L et al., Invest Ophthalmol Vis Sci., 54(3):1616-24 (2013))

In general, molecules (for example, antibodies or fragments) linked to peptide tags of the invention show longer ocular half-life than that of untagged molecules. For example, a molecule linked to a peptide tag that binds HA in the eye can have a 25% increase (e.g. from 5 to 6.25 days) in half-life compared to the untagged molecule, a 50% increase (e.g. from 5 to 7.5 days) in half-life compared to the untagged molecule, a 75% increase (e.g. from 5 to 8.75 days) in half-compared to the untagged molecule, or a 100% increase (e.g. from 5 to 10 days) in half-life compared to the untagged molecule. In certain aspects, it is contemplated that half-life of the peptide tagged molecule may increase more than 100% compared to the untagged molecule (e.g.: from 5 to 15, 20 or 30 days; from 1 week to 3 weeks, 4 weeks or more; etc.).

The dosage and frequency of administration can vary depending on whether the treatment is prophylactic or therapeutic and is directly affected by the half-life of the molecule dosed. Administration of the peptide tags or peptide tagged molecules described herein lead to a clinically meaningful improvement of dose and dosing frequency. For example, the peptide tags or peptide tagged molecules can be dosed at lower frequency compared to untagged molecules. Achieving a clinically meaningful improvement in dose and dosing frequency can vary depending on the initial starting dose of a composition. For example, for molecules that are dosed daily, weekly, bi-weekly, monthly or bi-monthly, a clinically meaningful improvement in dosing frequency that could be achieved with the peptide tagged molecule would be, for example, at least a 25%, 30%, 50%, 75%, or 100% increase in the dosing interval. In certain aspects, for example a clinically meaningful improvement of dosing frequency occurs by reducing the dosing frequency from daily to every other day, weekly to every two weeks, or monthly to every six weeks or bimonthly, or longer respectively.

More specifically the peptide tag of the invention may be used to improve the dosing interval of current ocular therapies. In certain aspects a peptide tag may be useful for increasing the dosing interval of a molecule by at least 25%. For example, the dosing interval can be increased by 30%, 40%, 50%, 60%, 70%, 75%, 80%, 90%, 100%, or more. The ocular dosing interval of a molecule may be increased by linking the molecule to a peptide tag that binds HA in the eye with a KD of less than or equal to 7.5 uM, less than or equal to 7.0 uM, less than or equal to 6.5 uM, less than or equal to 6.0 uM, less than or equal to 5.5 uM, less than or equal to 5.0 uM, less than or equal to 4.5 uM, less than or equal to 4.0 uM, less than or equal to 3.5 uM, less than or equal to 3.0 uM, less than or equal to 2.5 uM, less than or equal to 2.0 uM, less than or equal to 1.5 uM, less than or equal to 1.0 uM, less than or equal to 0.5 uM, or less than or equal to 100 nM. For example, the anti-VEGF Fab, ranibizumab, and the anti-VEGF IgG, bevacizumab, are currently dosed every month to achieve maximum visual benefit to Wet AMD and DME patients. Linking an HA-binding peptide tag to ranibizumab or bevacizumab would be expected to reduce the dosing frequency to bi-monthly or quarterly dosing (i.e.: at least a 50% increase in dosing interval). Similarly, the anti-VEGF aptamer, pegaptanib, is currently prescribed for dosing every six weeks in Wet AMD patients. Linking pegaptanib to an HA-binding peptide tag is expected to increase the dosing interval to 2 months or longer (i.e.: at least a 30% increase in dosing interval). For other molecules that require dosed frequencies of every two months, or longer, a clinically meaningful improvement would be increasing the dosing interval by an additional month or longer (i.e. at least 50% increase in dosing interval). For example, the anti-VEGF Fc trap, aflibercept, is currently prescribed for dosing bi-monthly in Wet AMD patients, linking aflibercept to an HA-binding peptide tag is expected to enable dosing every 3 months or longer, resulting in at least a 50% increase in the dosing interval.

In certain specific aspects the composition is formulated to deliver 12 mg, 11 mg, 10 mg, 9 mg, 8 mg, 7 mg, 6 mg, 5 mg, 4.5 mg, 4 mg, 3.5 mg, 3 mg, 2.5 mg, 2 mg, 1.5 mg, 1 mg, 0.9 mg, 0.8 mg, 0.7 mg, 0.6 mg, 0.5 mg, 0.4 mg, 0.3 mg, 0.2 mg, or 0.1 mg of the peptide tagged molecule per dose. In certain specific aspects the composition is formulated to deliver 6 mg, 5 mg, 4.5 mg, 4 mg, 3.5 mg, 3 mg, 2.5 mg, 2 mg, 1.5 mg, 1 mg, 0.9 mg, 0.8 mg, 0.7 mg, 0.6 mg, 0.5 mg, 0.4 mg, 0.3 mg, 0.2 mg, 0.1 mg, or 0.05 mg of the peptide tagged molecule per injection. In a particular aspect the composition is formulated to deliver 12 mg of the peptide tagged molecule per dose and/or 6 mg of the peptide tagged molecule per injection. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

### EXAMPLES

The Examples herein describe hyaluronan (HA) binding peptide tags that extend the half-life of molecules in the eye, for example the molecules may be proteins or nucleic acids. Two animal models were used to assess differences in the duration of efficacy between proteins that were linked with HA binding peptide tags and naked unmodified (i.e.: untagged) proteins or nucleic acids: the rabbit VEGF-induced leakage model, a model of retinal edema, and the cynomolgus laser-induced choroidal neovascularization (laser CNV) model, a model of neovascular (wet) AMD.

### Example 1

### Generation of a VEGF Fab (NVS4) and a Peptide Tagged VEGF Fab (NVS1)

Conversion of Anti-VEGF scFv to Anti-VEGF Fab (NVS4)

The starting point for generating the anti-VEGF Fab (NVS4) was the anti-VEGF scFV (1008 scFV). 1008 scFV was previously disclosed in US20120014958 and identified as 578minimaxT84N_V89L or Protein No: 1008.

To convert the 1008 scFv to its Fab version (NVS4), the amino acid sequence of the 1008 scFv was aligned with published human IgG framework sequences and determined to have high homology with the Kappa framework. Consequently, the 1008 scFv was converted to NVS4 by adding 1) human immunoglobulin kappa chain constant region sequence KRTVAAPSVFIFPPSDEQLKSGTASVVCLLNNFYPREAKVQWK VDNALQSGNSQESVTEQDSKDSTYSLSSTLTLSKADYEKHKVYACEVTHQGLSSPVTKSF NRGEC (SEQ ID NO: 125), to the C-terminal end of the 1008 scFv light chain and ii) human immunoglobulin first constant Ig domain of the heavy chain (CH1 domain) sequence ASTKGPSVFPLAPSSKSTSGGTAALGCLVKDYFPEP VTVSWNSGALTSGVHTFPAVLQSSGLYSLSSVVTVPSSSLGTQTYICNVNHKPSNTKVDKR VEPKSC (SEQ ID NO: 126) to the C-terminal end of the 1008 scFv heavy chain. In addition, the allotypes selected correlate with G1m(f)3 of heavy chain and Km3 of kappa light chain as these allotypes are used for our antibody therapeutics.

Tagged and untagged recombinant antibodies and proteins were expressed by transient transfections of mammalian expression vectors in HEK293 cells and purified using standard affinity resins for example, KappaSelect (Cat #17-5458-01, GE Healthcare Biosciences).

### Example 2

### Benchmarking of Unmodified VEGF Antibody (NVS4) to Ranibizumab

### Rabbit Traditional Ocular PK Determination

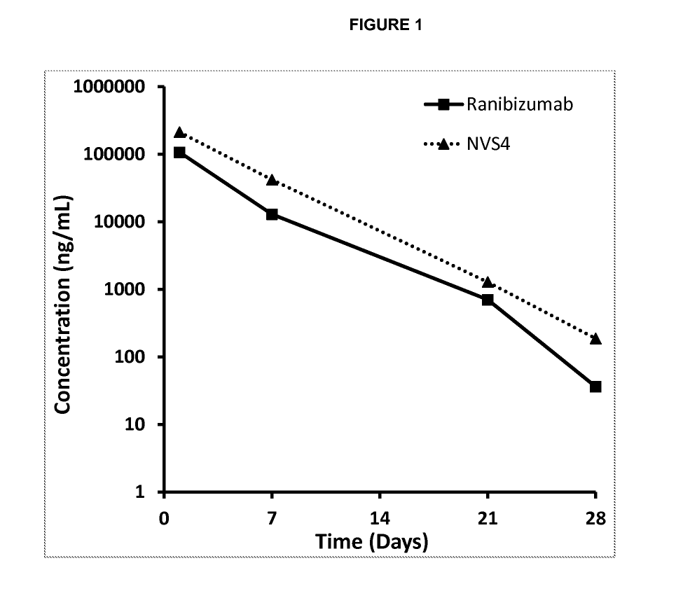

Ocular PK profiles of NVS4 and ranibizumab (CAS#: 347396-82-1) in rabbit vitreous were compared using traditional methods as described below and shown in FIG. 1.

150 μg/eye ranibizumab or NVS4 were injected intravitreally into rabbit eyes (N=6 eyes per antibody). Rabbits were sacrificed at 1 hr, and 7, 14, 21 and 28 days after injection and eyes were enucleated. The enucleated eyes were dissected and the vitreous was separated from other tissues and further homogenized mechanically using a TissueLyzer (QIAGEN®).

Antibody levels in the vitreous were measured by ELISA. The Maxisorp 384 well plates (Nunc 464718) were coated with a Goat Anti-Human IgG (H+L) (Thermo Fisher® 31119) in carbonate buffer (Pierce® 28382) overnight at 4 C. In between incubations, plates were washed 3 times with TBST (THERMO SCIENTIFIC® 28360) using a BioTek® plate washer. The next day, the plates were blocked for 2 hours at room temperature (or overnight at 4 C) with blocking buffer (5% BSA (SIGMA® A4503), 0.1% Tween-20 (SIGMA® P1379), 0.1% Triton X-100 (SIGMA® P234729) in TBS. Samples were diluted in diluent (2% BSA (SIGMA® A4503), 0.1% Tween-20 (SIGMA® P1379), 0.1% Triton X-100 (SIGMA® P234729) in TBS). Samples were incubated on the plate for 1 hour at room temperature with gentle shaking. The detection antibody was a Goat Anti-Human IgG [F(ab′)2]) conjugated to HRP (Thermo Fisher 31414). The detection antibody was added to the plates for 30 minutes at room temperature with gentle shaking. Ultra TMB is added for 15 minutes (Thermo Fisher® 34028). The reaction was quenched with 2N sulfuric acid (Ricca 8310-32). The absorbance of the samples was read on the SpectraMax® (450-570 nm). To back-calculate Fab recovery levels from eye tissues, a purified standard was used. For the standard, the top concentration used was 200 ng/mL with 2-fold dilutions. Different pairs of antibodies can be used for Fab recovery from rabbit tissues.

NVS4 and ranibizumab demonstrated equivalent ocular PK profiles as shown in FIG. 1. The half-life values for ranibizumab and NVS4 were 2.5 and 2.7 days respectively indicating equivalency of PK for both unrelated anti-VEGF Fabs, thus peptide tagged anti-VEGF Fabs may be compared to either ranibizumab or NVS4.

### Rabbit VEGF Challenge Model

In the rabbit VEGF-induced leakage model, human VEGF (hVEGF) was administered to rabbit eyes by intravitreal (IVT) injection. Human VEGF induces dose-dependent vascular changes including increased vessel diameter, tortuosity and permeability. Vascular permeability can be assessed using fluorescein angiography combined with either quantitative image processing or fluorescein leakage scoring (methods described below).

### Intravitreal (IVT) Injections in Rabbits

Rabbit eyes were dilated with topical 1% cyclopentolate and 2.5 or 10% phenylephrine and the cornea anesthetized with topical 0.5% proparacaine. The rabbits were then anesthetized with an intramuscular injection of ketamine/xylazine mix (17.5-35 and 2.5-5 mg/kg). Under direct visualization of a surgical microscope, 50 μL of the treatment was injected into the vitreous. The 30 gauge needle was inserted superotemporally approximately 2 mm from the limbus into the middle of the vitreous. The rabbit eye was examined for complications from the injection (e.g., hemorrhage, retinal detachment or a lens injury) and then the procedure was repeated on the fellow eye. Antibiotic ointment was applied to both eyes for all studies (in a subset of studies the antibiotic ointment additionally contained dexamethasone). 400 ng of recombinant hVEGF was injected into the vitreous of male Dutch belted rabbits with body weight approximately 1.6-2 kg. The human VEGF (Peprotech; cat AF 100-20, Lot 0508AF10) was diluted in sterile 0.9% saline. 48 hours after intravitreal injection of the VEGF challenge, the rabbit retinal vasculature was imaged as described below.

### Image Acquisition

Human VEGF-induced retinal vessel changes were quantified through acquisition of images of retinal vessels after intravenous fluorescent dye administration. Images acquired after fluorescein delivery were utilized to determine vessel permeability in all efficacy studies. Studies generating quantitative fluorescein leakage also required imaging of a fluorescent dye selected to label the vessels (fluorescein isothiocyanate (FITC)-conjugated dextran). Ocular images were acquired 48 hours post-VEGF. Images were an average of up to 40 registered scanning laser ophthalmoscope (SLO) images acquired with a 30 degree lens on the nasal medullary ray adjacent to the optic nerve. The fluorescein channel from a 6-mode Spectralis® (Heidelberg Engineering) was used for all image acquisition. Prior to imaging, rabbits received 1-2 drops of 1% cyclopentolate and 1-2 drops of phenylephrine (2.5 or 10%) topically for dilation. 0.5% proparacaine was also applied as a topical anesthetic. Rabbits were subsequently anesthetized as previously described. Vessels were labeled approximately 5 minutes before image acquisition with an intravenous injection of 1 mL of a solution of FITC-conjugated 2000 kD dextran (SIGMA®) into the marginal ear vein. The concentration of FITC-dextran used (35-70 mg/mL) was chosen empirically for each lot based on the fluorescence signal necessary to generate high quality images. Images of the labeled retinal vasculature were subsequently acquired. Retinal vessel permeability was then assessed through injection of 0.3 mL of a 10% fluorescein solution into the marginal earl vein. Images were then acquired either 3 minutes after fluorescein injection in one eye only, or 3 minutes after injection in one eye followed by an image approximately 4-6 minutes after fluorescein injection in the fellow eye, depending on the study.

### Image Analysis

The effects of VEGF on vessel permeability were assessed using two different techniques applied to the 3-6 minute fluorescein images. Regardless of the approach used, the steps used to generate and acquire the data were the same with the exception of FITC-dextran injection as previously described. Analysis was performed either quantitatively with custom-designed software developed for this purpose using MATLAB® (Mathworks®) or by grading the fluorescein leakage in each image using a qualitative scoring system. Exclusions were made prior to unmasking in cases of insufficient image quality, if there was noted inflammation, or in cases where there were issues with injections. For both approaches the data are reported either for individual studies or as a combination of multiple studies. Both methods are described below.

### Quantitative Image Processing Analysis

Fluorescein leakage was quantified in some studies with image processing techniques using the method described below.

First, post-VEGF FITC-dextran and fluorescein images were aligned to each other using vessel features common to both images, then:
1. Regions outside of the medullary ray were then cropped from the co-registered images along with any localized areas with insufficient image quality for analysis.
2. Several regions of interest in the retinal vessels were delineated in both images and the intensity of one image was boosted until the signal in the region of interest was equal in both images (normalization).
3. The aligned FITC-dextran image was subtracted from the fluorescein leakage image yielding an image comprised of extravasated fluorescein.
4. Fluorescein leakage was reported for each eye as the average intensity of the pixels contained in the cropped region of interest in the extravasated dye image.

Inhibition of fluorescein leakage in each group was calculated versus the saline control group. Statistical analysis was performed with either a two-tailed Student's t-test or a one way analysis of variance with a Dunnett's multiple comparison test.

### Qualitative Fluorescein Image Leakage Scoring

Retinal vessel permeability was assessed in some studies using a three step scoring system developed for application to the fluorescein angiography images. A reader assigned each fluorescein leakage image into one of three categories. A score of 0 indicated no signs of leakage from retinal vessels. A score of 1 indicated a haze suggestive of fluorescein leakage. If the perceived leakage was subtle, an increase in vessel tortuosity could be used to confirm a score of 1. A score of 2 indicated unambiguous fluorescein leakage over most or all of the retinal vessel area. Image assessment was made on masked, randomized data.

Regardless of the method used for assessing vascular permeability, the measured fluorescence signal or perceived increase in extravasated dye is proportional to vascular leakage. Efficacy is defined as a reduction in the measured fluorescence signal intensity or perceived extravagated dye relative to the signal observed in animals that received saline injections. A lower value of the average fluorescence signal or image score corresponds to a greater inhibition of leakage and, therefore, greater efficacy.

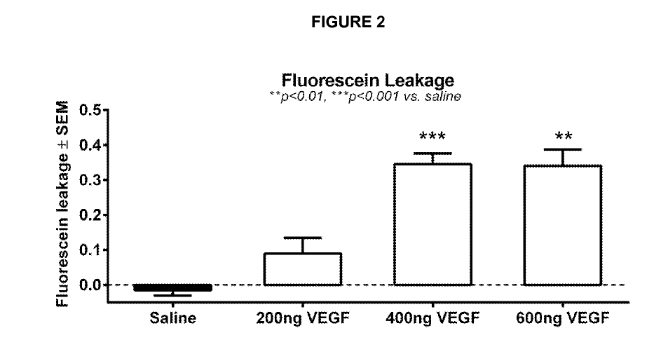

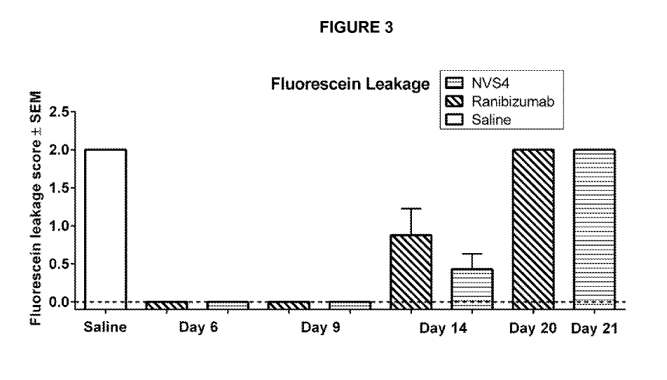

Intravitreal administration of 400 ng/eye of human VEGF resulted in maximal leakage at 48 hrs post treatment (FIG. 2). This vessel leakage can be completely inhibited by prior IVT administration of an anti-VEGF molecule such as ranibizumab, bevacizumab, aflibercept, or NVS4 (FIG. 3). To determine the duration of action of an anti-VEGF molecule, the anti-VEGF molecule was administered at different times prior to hVEGF challenge. The interval between administration of the anti-VEGF molecule and the hVEGF challenge determines the duration of action of the anti-VEGF molecule. Four to 28 days before the hVEGF challenge (6 to 30 days prior to imaging), anti-VEGF antibodies were injected into the vitreous. Each rabbit cohort consisted of 3-5 animals (6-10 eyes) injected with the same antibody at the same time.

To determine the duration of efficacy in the rabbit leakage model, 5 μg per eye of an unmodified anti-VEGF antibody (e.g.: ranibizumab or NVS4) was administered to each eye intravitreally at various times from 4 to 19 days prior to hVEGF challenge (6 to 21 days prior to imaging, FIG. 3). Both ranibizumab and NVS4 had similar duration of efficacy profiles as determined by fluorescein leakage scores. When 5 μg/eye of ranibizumab or NVS4 were administered 4 and 7 days prior to the hVEGF challenge, complete inhibition of fluorescein leakage was observed. When administered 12 days prior to the VEGF challenge, an increase in fluorescein leakage indicated partial efficacy was observed. When ranibizumab was administered 18 days prior to hVEGF challenge, no significant efficacy was achieved. In a separate study, NVS4 did not demonstrate significant efficacy when administered 19 days prior to hVEGF challenge.

Together with the rabbit traditional ocular PK data, these results indicate that ranibizumab and the unmodified/untagged VEGF antigen binding fragment, NVS4, have similar ocular retention and efficacy duration in rabbits. In the following studies a peptide tagged antibody (e.g.: NVS4 linked to a peptide tag that binds HA) was compared to ranibizumab.

### Example 3

### Generation of Tagged Antibodies

Numerous peptide tags were generated that bind to a variety of ocular targets, for example, peptide tags that bind collagen II, hyaluronan, fibronectin, laminin, integrin, elastin, vitronectin. These peptide tags were tested for their ability to increase half-life of antibodies in the eye. The methods below describe the generation and characterization of single and double tagged antibodies.

### Single-Tagged Antibodies or Fabs

NVS4 fusion proteins were made containing a single peptide tag that binds one of the ocular targets listed above, the peptide tag sequences (e.g.: HA-binding tag sequences) were fused to the C-terminal of the heavy chain of NVS4 using a GSGGG (SEQ ID NO: 31) or GSGG (SEQ ID NO: 124, for example see NVS5 and NVS11) linker. Production of candidates entails synthesis of a nucleotide sequence encoding the amino acid of light chain and heavy chain Fab fused to the tag sequence. Nucleotides were synthesized to encode the amino acids of the heavy chain variable region up to the last cysteine of the CH1 constant domain, followed by the GSGGG (SEQ ID NO: 31) or GSGG (SEQ ID NO: 124) linker described above, and the tag sequence. However, fusion of the tag sequence is not limited to C-terminal end of the heavy chain fab. The tag may be engineered to fuse at the C-terminal end of the light chain as well as the N-terminal end of the heavy or light chain or combinations of the two chains.

### Double-Tagged Antibodies or Fabs

Multiple tagged versions of NVS4 were made by fusing two or more peptide tags to NVS4. The peptide tag sequences were linked to either:

1) the C-terminus of the heavy chain of NVS4 using a GSGGG linker (SEQ ID NO: 31) and the C-terminus of the light chain of NVS4 using a GSGGG linker (SEQ ID NO: 31) (e.g.: NVS1d),
2) the C-terminus of the heavy chain of NVS4 using a GSGGG linker (SEQ ID NO: 31) and the N-terminus of the light chain of NVS4 using a GSGGG linker (SEQ ID NO: 31) (e.g.: NVS1f),
3) the N-terminus of the heavy chain of NVS4 using a GSGGG linker (SEQ ID NO: 31) and the N-terminus of the light chain of NVS4 using a GSGGG linker (SEQ ID NO: 31) (e.g.: NVS1c), or
4) the N-terminus of the heavy chain of NVS4 using a GSGGG linker (SEQ ID NO: 31) and the C-terminus of the light chain of NVS4 using a GSGGG linker (SEQ ID NO: 31).
5) the C-terminus of the light chain of NVS4 using a GSGGG linker (SEQ ID NO: 31) in tandem (e.g. NVS1e)
6) the C-terminus of the heavy chain of NVS4 using a GSGGG linker (SEQ ID NO: 31) in tandem (e.g. NVS1h)
7) the C-terminus of the heavy chain of NVS4 using a GSGGG linker (SEQ ID NO: 31) in tandem (e.g. NVS1g)

Nucleotides encoding the amino acid sequence of light chain and heavy chain Fab fused to the peptide tag sequence were synthesized. Nucleotides were synthesized to encode the amino acids of the heavy chain variable region up to the last cysteine of the CH1 constant domain and the entire light chain, preceded or followed by the GSGGG (SEQ ID NO: 31) or GSGG (SEQ ID NO: 124) linker and the peptide tag sequence as described.

### Example 4

### Selection of Peptide Tags

The following example describes methods that may be used to measure the binding and/or affinity of the peptide tags to their ocular targets when fused to an anti-VEGF antibody (e.g.: NVS4). These and other methods of measuring binding affinity are known in the art.

Determination of Binding and/or Affinity of HA-Binding Peptide Tags by Octet®

Assessment of binding of peptide tags, and/or tagged VEGF antibodies or antigen binding fragments, to biotinylated-HA was performed using an Octet® (ForteBio®) as per the manufacturer's instructions. A biosensor, a tip of a fiber, is coated with a special optical layer and a capturing molecule is then attached to the tip. The tip is dipped into the sample containing target molecule which binds to the capture molecule, and the two form a molecular layer. A white light is directed into the fiber and two beams will be reflected to the back end. The first beam comes from the tip as a reference. The second light comes from the molecular layer. The difference of the two beams will cause a spectrum color pattern and the phase is a function of the molecular layer thickness and corresponding to the number of molecules on the tip surface. When the molecules bind to the sensor, the reflections on the internal reference will remain constant and the interface between the molecular layer on the fiber and the solution changes with the addition of bound molecules. The biolayer interferometry within the sensor monitors this change in wavelength shift over time. As molecules bind, the spectrum of signal will change as a function of the layer increasing on the sensor. This real-time binding measurement can be used to calculate kinetics of an interaction, the on and off rates and ultimately concentration by plotting rates against concentration.

In the following described method, the streptavidin biosensor (ForteBio®, Cat. No. 18-5019) was presoaked for 10 minutes in 1× Kinetic Buffer (FortBio®, Cat. No. 18-5032) to remove the protected sucrose layer on the tip of the biosensor. Then, it was dipped into wells containing 200 ul of biotinylated 17 kDa hyaluronic acid (HA) at 5 ug/ml diluted with 1× Kinetic Buffer and allowed biotinylated HA to be loaded onto the streptavidin biosensor for 900 seconds. The captured HA biosensor was then dipped into 200 ul of 1× Kinetic Buffer well for 300 seconds to remove residual biotinylated HA not captured by the streptavidin. Afterward, the bound HA biosensor was dipped into wells containing the engineered antibody at a concentration of 200 nM for single point binding screen or serial titration for determining kinetics. The modified antibody of interest was allowed to associate with the captured HA on the biosensor for 900 seconds, and after which was transferred and dipped in well containing 200 ul 1× Kinetic Buffer for 2100 seconds to allow dissociation of the engineered antibody from the antigen, HA. Binding kinetics was determined from the ForteBio's® Analysis Program.

Determination of Binding and/or Affinity of Peptide Tags to their Ocular Targets by ELISA Binding

The binding of various peptide tags fused to an anti-VEGF Fab (NVS4) to ocular target proteins including collagen II, laminin, integrin, fibronectin, and elastin were measured using Meso Scale Discovery® ELISAs as described below.

Twenty-five microliters of 2 ug/ml of protein is coated on 384-well MSD plate (Cat.# L21XA, Meso Scale Discovery®) overnight at 4° C. The plate is washed 3× in TBS/0.05% Tween-20, (Thermo Scientific® #28360) and blocked with buffer containing TBS/5% BSA Fraction V (Fisher® Cat#ICN16006980)/0.1% Tween-20/0.1% TritonX-100 for minimum of two hours at room temperature or overnight at 4° C. The plate is washed 1×. A titration of the fab is diluted in buffer containing TBS/2% BSA Fraction V/0.1% Tween-20/0.1% TritonX-100 and 25 ul per well is added to the washed plate for a 1 hour incubation at room temperature. Afterward, the plate is washed 3× and 25 ul per well is added of the 1:1000 diluted anti-human IgG-Sulfo tag labeled detection antibody (Cat. # R32AJ, Meso Scale Discovery®). After 1 hour incubation at room temperature, the plate is washed three times and 25 ul per well of 1×MSD® Read Buffer (Cat. # R92TC) is added. The plate is immediately read on the SECTOR Imager 6000® Meso Scale Discovery® instrument. The electrochemiluminescent signal data is analyzed using GraphPad Prism®.

### Results

In all, 90 peptide tags were linked to an anti-VEGF Fab (see Example 3) and assessed for in vitro binding to their respective putative ocular targets of Octet or ELISA.

Fifty putative HA-binding peptide tag sequences were linked to an anti-VEGF Fab and assessed for in vitro HA-binding. Only 27 of the 50 putative HA-binding peptide tags demonstrated measurable in vitro binding to HA.

Twenty three putative collagen-binding tags were linked to an anti-VEGF Fab and assessed for in vitro binding to collagen II. Only 3 of 23 putative collagen-binding peptide tags demonstrated in vitro binding to collagen II.

Seven putative integrin-binding peptide tags were linked to an anti-VEGF Fab and assessed for in vitro binding to integrin. Only 1 of the 7 putative integrin-binding peptide tags demonstrated in vitro binding to integrin.

None of the other fibronectin-binding, laminin-binding, elastin-binding, or vitronectin-binding binding tags demonstrated significant measurable binding to their respective targets.

Peptide tags with positive target binding were subsequently assessed in the rat PET/CT-based imaging PK model.

### Example 5

### PK Assessment of Peptide Tags with Positive Binding to Collagen II, Integrin or HA

PET/CT Imaging of Rats Injected IVT with I-124 Labeled Fab Proteins

The ocular PK of tagged antibodies that demonstrated measurable binding to HA, or collagen II, or integrin by Octet and/or ELISA were measured using a rat PET/CT imaging method as described herein.

Radiolabeling of the proteins that were injected in rat eyes was performed using the lodogen method (1), which employs the use of iodogen coated tubes (THERMO SCIENTIFIC®, Rockford, Ill.). Typically, a radiolabeling efficiency >85% and a specific activity of approximately 7 mCi/mg were achieved. To prepare rats for intravitreal (IVT) injections, the animals were anesthetized with 3% isoflurane gas. The eyes were then dilated with two drops of Cyclopentolate (1% preferred concentration) and 2.5-10% Phenylephrine. A drop of local anesthetic was also applied (0.5% Proparacaine). Under a dissecting microscope, an incision was made with a 30 gauge needle approximately 4 mm below the limbus of the cornea with the angle directed towards the middle of the eye. A blunt end Hamilton syringe (e.g. 33 gauge) containing the radioactively labeled protein was then inserted through this opening into the vitreous cavity and approximately 3.5 μL of radiolabeled protein was injected. The eye was examined for hemorrhage or cataract. The procedure was then repeated on the fellow eye. Immediately after injecting the radiolabeled protein into a rat eye, the anesthetized animal was placed on the preheated PET imaging bed, lying on its abdomen. The bed was supplied with a nose cone for gas anesthesia. The immobilized and secured animal was then moved in the scanner with vital functions (e.g. respiration) being monitored using a breathing sensor placed under the animal's chest. A static 10 min PET scan, followed by a 10 min CT scan were performed on a GE Triumph LabPET-8 trimodality small animal scanner (Gamma Medica, Northridge, Calif.). After completion of the CT scan, the animal was removed from the bed, placed in a warm cage and monitored until complete recovery of normal physiological functions. Typical time points of PET/CT imaging post IVT injection were 0, 3, 6, 21, 29, 46, 52, 72, 94, 166, 190, and 214 h. Shorter studies with fewer time points of imaging (e.g. 0, 6, 24, 48, 72, and 96 h) were also conducted. After the last imaging time point, anesthetized animals were euthanized by cardiac puncture, exsanguinations, and cervical dislocation. Eyes and other organs/tissues (blood, liver, spleen, kidneys, stomach, lungs, heart, muscle, and bone) were dissected out and counted for remaining radioactivity in a gamma counter. Counts were converted to % injected dose/gram (% ID/g) of the counted tissue/organ.

All PET images were then reconstructed using MLEM reconstruction algorithm and then co-registered with the CT anatomical scans. For analysis, the image of the head was separated into right and left hemisphere. AMIRA® (Visualization Sciences Groupe, Burlington, Mass.) and Amide (Sourceforge.net) analysis software packages were used to draw 3D regions of interest (ROI) on the PET image based on the CT defined eye location. The PET signal in the region of interest was represented as a standard uptake value (SUV), taking into consideration the decay corrected injected dose and weight of the animal eyes (measured post mortem), and normalizing for the volume of the ROI. The data was then plotted to calculate the clearance kinetics (e.g. half, life or mean residence time) of the injected protein in the rat eyes.

To assess the ocular clearance of unmodified (e.g.: un-tagged) antibodies, or antigen binding fragments, and tagged antibodies, ¹²⁴I-labeled antibodies were injected into rat eyes, and relative antibody levels were determined over time using PET/CT-based imaging. The signal intensity, as a measure of relative antibody levels, was determined immediately after intravitreal (IVT) injection, and also at 24, 48 and 96 hours post-injection. The signal intensity for a unmodified antibody (e.g., ranibizumab) declines to 1% of the initial value by 48 hrs post-injection. At 96 hours post-injection, the signal intensity of an unmodified antibody, (e.g., ranibizumab) was below the limit of detection. Thus, the rat model is a useful short term in vivo screening model for identifying molecules with an increased retention time in the eye.

Twenty seven peptide tagged antibodies were tested for longer retention time in the rat model. Longer retention time was defined by presence of >1% of injected dose remaining at 96 hours post-IVT. Nine peptide tagged antibodies had <1% of injected dose remaining at 96 hours. In contrast, 18/27 peptide tagged antibodies demonstrated longer retention in rat eyes as defined by presence of >1% of injected dose remaining at 96 hours post-IVT. These 18 tagged antibodies were subsequently assessed for efficacy in the rabbit leakage model. The rabbit is a longer term model that is more clinically relevant than the short term rat model.

### Example 6

### Rabbit Efficacy: Only One HA-Binding Peptide Tag is Efficacious

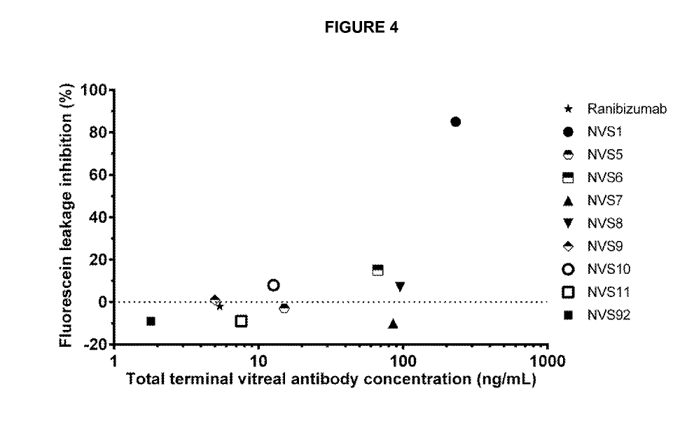

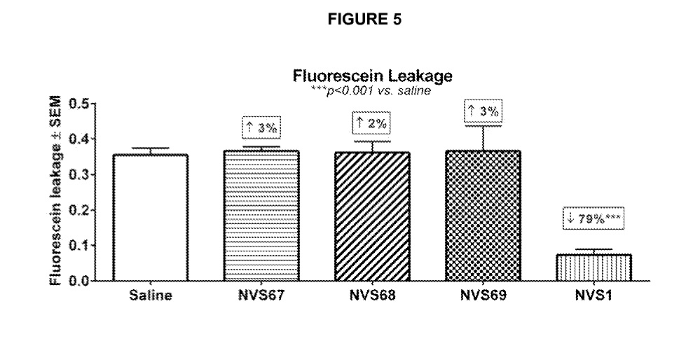

The rabbit leakage model (described in Example 2) was used to assess whether anti-VEGF antibodies, or Fabs, linked to a peptide tag that binds either collagen or HA could inhibit vessel leakage at 20 days post-injection (FIGS. 4 and 5). The rabbit provides a larger, more human scale eye, in which to test the long term efficacy of peptide tags and peptide tagged molecules. Twenty-two anti-VEGF Fabs linked to either a collagen-binding peptide tag or HA binding peptide tag were administered at an equimolar dose to 5 μg/eye ranibizumab. Forty-eight hours post hVEGF challenge, fluorescein leakage was assessed as described above. Addition of a collagen-binding peptide tag did not result in significant fluorescein leakage inhibition for any of the VEGF Fabs tested (FIG. 5: NVS67, NVS68 and NVS69). In contrast, addition of an HA binding peptide tag exhibited significant inhibition of fluorescein leakage under the same conditions (NVS1, FIG. 5). The results demonstrate that linking a peptide tag that binds collagen to an anti-VEGF Fab was not sufficient to suppress hVEGF and block vessel leakage longer than the untagged anti-VEGF Fab, NVS4. In contrast, addition of an HA binding peptide tag was able to demonstrate significant efficacy. Thus, the ability of a peptide tag to increase half-life and produce an efficacious effect in vivo is unique to the peptide fragments that binds HA of the invention, and as described herein.

### Rabbit Terminal PK Quantitation by ELISA

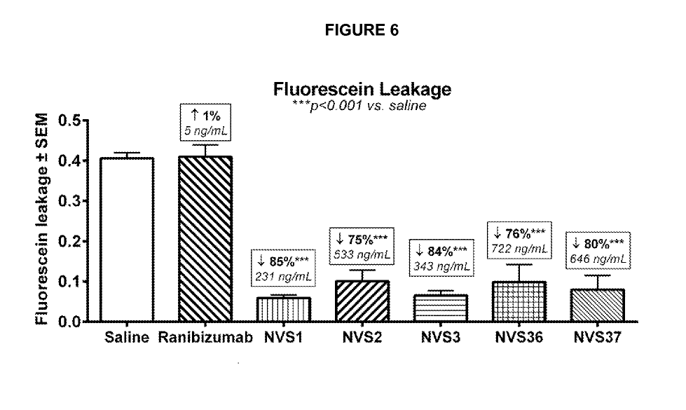

Upon completion of the imaging analysis to measure vessel leakage, the animals were sacrificed on either the day of or day after imaging, eyes were enucleated, and processed for quantitation of antibody concentration (FIG. 4 and FIG. 6) in the vitreous as described above in example 2. The terminal vitreal concentration of ranibizumab was approximately 5 ng/mL. In contrast, the terminal vitreal concentration of the efficacious tagged antibody NVS1 was 231 ng/mL. Higher terminal drug levels correlated with inhibition of leakage at day 20, lower terminal drug levels correlated with a lack of efficacy. The terminal drug levels of all molecules that did not exhibit efficacy at day 20 were less than 100 ng/mL (FIG. 4), while the terminal drug levels of the molecule that inhibited fluorescein leakage (NVS1) was greater than 100 ng/mL. Three of the tagged antibodies linked to different peptide tags that bind HA had drug levels 10-20 fold higher than ranibizumab at day 20 and yet did not exhibit efficacy (e.g.: NVS6, NVS7, NVS8). The drug levels at day 20 of the efficacious molecule NVS1 were more than 40-fold higher than the untagged antibody (e.g.: ranibizumab) drug levels, indicating a significantly slower rate of ocular clearance of the HA-binding peptide tagged antibody as compared to the untagged antibody.

Only NVS1 demonstrated measurable binding to HA by octet, longer retention in rat eyes as measured by PET/CT imaging, and longer duration of efficacy in the rabbit leakage model as defined by statistically significant inhibition of fluorescein leakage when administered 18 days prior to the VEGF challenge. None of the collagen II-binding peptide tags were efficacious. Thus, the peptide fragment that binds HA having a sequence of SEQ IS NO: 32 was selected for optimization.

TABLE 3
Summary of in vitro and in vivo data for 14 tagged antibodies.
The untagged antibody NVS4 was modified with the sequences
shown (linker + peptide tag) to produce the 14
tagged antibodies tested. (Linker sequence underlined)

| | | | | >1% | |
|---|---|---|---|---|---|
| | Sequence of GSGGG | | | injected | |
| | linker (SEQ ID NO: 31) + | | | dose at | Positive |
| | peptide tag linked to | Origin of | HA | 96 hrs in rat | Rabbit |
| NVS ID | NVS4 (SEQ ID NO:) | peptide tag | binding | PET/CT PK | Efficacy |
| NVS1 | GSGGGGVYHREARSGKYKLTY | Tumor necrosis | Yes | Yes | Yes |
| | AEAKAVCEFEGGHLATYKQLE | factor-inducible | | | |
| | AARKIGFHVCAAGWMAKGR | gene 6 protein | | | |
| | VGYPIVKPGPNCGFGKTGIIDY | (TNFAIP6/TSG6 | | | |
| | GIRLNRSERWDAYCYNPHAK | aa 36-129) | | | |
| | (SEQ ID NO: 127) | | | | |
| NVS16 | GSGGGKQKIKHVVKLKGSGG | Hyaluronan | Yes | Yes | No |
| | GKLKSQLVKRK | mediated | | | |
| | (SEQ ID NO: 128) | motility receptor | | | |
| | | (HMMR aa 401- | | | |
| | | 411, 423-432) | | | |
| NVS17 | GSGGGKNGRYSISRGSGGGR | CD44 antigen | Yes | Yes | No |
| | DGTRYVQKGEYRGSGGGRRR | (CD44 aa 38- | | | |
| | CGQKKK (SEQ ID NO: 129) | 46,150-162,292- | | | |
| | | 300) | | | |
| NVS18 | GSGGGVFPYHPRGGRYKLTFA | Hyaluronan and | Yes | Yes | No |
| | EAQRACAEQDGILASAEQLHA | proteoglycan link | | | |
| | AWRDGLDWCNAGWLRDGS | protein 4 | | | |
| | VQYPVNRPREPCGGLGGTGS | (HAPLN4 aa163- | | | |
| | AGGGGDANGGLRNYGYRHN | 267) | | | |
| | AEERYDAFCF (SEQ ID NO: | | | | |
| | 130) | | | | |
| NVS5 | GSGGEVFYVGPARRLTLAGAR | Neurocan core | Yes | Yes | No |
| | AQCRRQGAALASVGQLHLA | protein (NCAN | | | |
| | WHEGLDQCDPGWLADGSVR | Link 2 aa 259- | | | |
| | YPIQTPRRRCGGPAPGVRTVY | 357) | | | |
| | RFANRTGFPSPAERFDAYCFR | | | | |
| | (SEQ ID NO 131) | | | | |
| NVS11 | GSGGLKQKIKHVVKLKDENSQ | Hyaluronan | Yes | Yes | No |
| | LKSEVSKLRSQLVKRKQNGSG | mediated | | | |
| | GAHWQFNALTVRGGGSSTM | motility receptor | | | |
| | MSRSHKTRSHHV (SEQ ID | (HMMR and HA | | | |
| | NO: 132) | phage peptide) | | | |
| NVS8 | GSGGGVFHLRSPLGQYKLTFD | Stabilin-2 (Stab2 | Yes | Yes | No |
| | KAREACANEAATMATYNQLS | aa 2199-2296) | | | |
| | YAQKAKYHLCSAGWLETGRV | | | | |
| | AYPTAFASQNCGSGVVGIVDY | | | | |
| | GPRPNKREMWDVFCYRMKD | | | | |
| | VN (SEQ ID NO: 133) | | | | |
| NVS9 | GSGGGHQNLKQKIKHVVKLK | Hyaluronan | Yes | Yes | No |
| | DENSQLKSEVSKLRSQLAKKK | mediated | | | |
| | QSETKLQ (SEQ ID NO: 134) | motility receptor | | | |
| | | (HMMR aa 516- | | | |
| | | 559) | | | |
| NVS10 | GSGGGGVYHREARSGKYKLTY | Tumor necrosis | Yes | Yes | No |
| | AEAKAVCEFEGGHLATYKQLE | factor-inducible | | | |
| | AARKIGFHVCSAGWLETGRV | gene 6 protein | | | |
| | AYPTAFASQNCGSGVVGIVDY | (TNFAIP6/TSG6) | | | |
| | GIRLQRSERWDAYCYNPHAK | and Stabilin-2 | | | |
| | AHP (SEQ ID NO: 135) | (Stab2) Chimeric | | | |
| NVS7 | GSGGGKVGKSPPVRGSGGGH | HUMAN GHAP | Yes | Yes | No |
| | REARSGKYK (SEQ ID NO: | S4,TSG6 aa 39- | | | |
| | 136) | 48 | | | |
| NVS6 | GSKQKIKHVVKLKGGGSREAR | RHAMM/TSG6 | Yes | Yes | No |
| | SGKYK (SEQ ID NO: 137) | BX7B Link | | | |
| NVS92 | GSGGGKGGNGEPRGDTYRAY | Bone | Yes | Yes | No |
| | GSGGGKGGPQVTRGDVFTM | Sialoprotein and | | | |
| | P (SEQ ID NO: 138) | Vitronectin | | | |
| | | | | >1% | |
| | Sequence of GSGGG | | | injected | |
| | linker (SEQ ID NO: 31) + | | Collagen | dose at | Positive |
| | peptide tag linked to | Origin of | II | 96 hrs in rat | Rabbit |
| NVS ID | NVS4 (SEQ ID NO:) | peptide tag | binding | PET/CT PK | Efficacy |
| NVS67 | Not applicable* | Not applicable* | Yes | Yes | No |
| NVS68 | GSGGGRRANAALKAGELYKSI | Osteopontin/B | Yes | Yes | No |
| | LYG (SEQ ID NO: 139) | (X)7 B | | | |
| NVS69 | GSGGGRRANAALKAGELYKSI | SLRP | Yes | Yes | No |
| | LYG (SEQ ID NO: 140) | | | | |

*NVS67 is an anti-VEGF scFv fused with an anti-collagen II scFv in a tandem manner.

In attempt to improve the affinity of peptide tags that failed to demonstrate extension in duration of efficacy in the rabbit leakage model, 16 additional double-tagged Fabs were generated by linking eight putative HA-binding peptides onto the C-terminus of both the heavy and light chain of two different Fabs, NVS4 (an anti-VEGF Fab) and NVS00 (an anti-chicken lysozyme Fab negative control). In all, 8 double tagged Fabs were generated with the NVS4 and an additional 8 double tagged Fabs were generated with NVS00. No difference in binding was observed for any of these peptide tags when they were linked to NVS4 or NVS00 and there was no significant improvement in binding of these 16 peptide tagged Fabs for HA. Thus, multimerization of peptide tags that did not achieve positive rabbit efficacy as monomers did not improve the activity of these peptide tags when multiple tags were linked to the NVS4 anti-VEGF Fab.

The HA binding affinity of select peptide tagged molecules (e.g.: NVS1, NVS2, NVS36, NVS37, NVS1b and NVS7) were determined by isothermal calorimetry as per manufacturer's protocols (MicroCal®, GE Healthcare). The affinities of peptide tagged molecules with a single peptide tag, for example, NVS1, NVS2, NVS36, and NVS37 was 5.5±2 uM, 8.0±1 uM, 6.0±1.2 uM and 7.2±1.5 uM, respectively. Adding multiple peptide tags, for example in NVS1d, (NVS1d: described in Example 13) improved binding affinity. NVS1d had a KD of 0.48±0.04 uM. In contrast, the affinity for NVS7, which was not efficacious in the rabbit model, only binds HA with an affinity of 44±19 uM. Thus, the efficacious peptide tags of the invention exhibit a binding affinity of less than or equal to 9.0 uM.

### Example 7

### Optimization of the HA-Binding Peptide Tag in NVS1 to Remove Glycosylation and Protease Sensitivity

In silico analysis identified position N311 of NVS1 (SEQ ID NO: 21) as an N-linked glycosylation site. To prevent glycosylation at this site six single-site variants of NVS1 (NVS12, NVS19, NVS20, NVS21, NVS22, and NVS23) and twelve double-site variants (NVS2a, NVS3a, NVS28, NVS31, NVS49, NVS50, NVS51, NVS52, NVS53, NVS54, NVS55, and NVS56) were expressed and characterized for HA-binding.

In addition, protease sensitivity assays conducted using conditioned media identified positions R236, K241, and R268 in NVS1 (SEQ ID NO: 21) as protease sites. To prevent protease clipping at position R236, K241, and R268, several single, double, triple, quadruple, and quintuple variants of the peptide tag were expressed and characterized for HA binding (Table 4). In addition, an additional disulfide bond was engineered into the peptide tag to produce two tagged variants NVS36 and NVS37. The sequence of the peptide tag variant in NVS36 or NVS37 is SEQ ID NO: 35 or SEQ ID NO: 36, respectively.

### Biacore Affinity Determination

Affinity of optimized HA-binding peptide tags for HA and human VEGF were measured by Biacore. In order to determine HA kinetics, biotinylated HA was used in a BIOCAP Biacore format in which biotinylated HA is captured and the sample proteins flowed over at various concentrations. This method will be described in detail below. In order to determine target kinetics, two different formats were utilized. The first format is the BIOCAP method which utilizes biotinylated target ligands which are captured and the protein samples were flowed over at various concentrations. The second format is an anti-fab capture method in which the fab protein samples are captured and the target proteins flowed over at various concentrations.

### HA Binding Kinetics and Affinity:

For HA kinetics, 2 different methods were utilized where contact times and dissociation times were different depending on the affinities for HA-biotin and human VEGF. In both methods the sample compartment was kept at 15° C. but the analysis compartment was run at either 25° C. or 37° C. In this method, four flow cells were utilized for the run. Flow cell 1 (fc1) served as the reference cell, where no ligand was captured, to assess for non-specific binding of the tagged proteins to the modified streptavidin-BIOCAP® reagent on the coated chip surface. On the second, third and fourth flow cell, both the BIOCAP® reagent and either the biotinylated HA ligand or other biotinylated ligands were captured. Then the tagged proteins and the parental proteins were flowed over at different concentrations

### Step 1 BIOCAP Capture Step:

The BIOCAP® reagent was provided in the Biotin CAPture® kit (GE® 2892034) and was diluted 1:3 into the HBS-EP+ running buffer (teknova H8022). The flow rate was 2 ul/min and it flowed for 60 seconds. The capture level was approximately 1500RU. Step 2 Biotinylated ligand Capture step:

All of the ligands were flowed over at a rate of 10 μl/min for approximately 20 seconds or to achieve capture levels that would give an Rmax of 20. The biotinylated ligands tested in this method include biotinylated HA and biotinylated human VEGF that was generated internally. An example on how to calculate an Rmax is included below for HA but in this case we used higher capture levels and used an Rmax of approximately 60. The following equations represent the calculations to achieve a relative Rmax of 20:

HA-17 kDa: Rmax=RL*(MWanalyte/MWligand)*stoichiometry 20=RL*(50/17)*1=7RL

### Step 3 Protein Dilutions (Analyte):

For the HA kinetics of samples having higher affinities with faster off rates, the protein analytes were run at a flow rate of 60 ul/min for a contact time of 30 seconds. The analyte concentrations started at 25 nM and included 4 dilutions at 1:2 (1 part dilution to 1 part buffer). Dissociation times of 85 seconds were included for all the dilutions due to the fast off rates. However it should be noted that the protein samples reached baseline prior to 85 seconds.

For the HA kinetics of samples having lower affinities including slow off rates, the protein analytes were run at a flow rate of 30 ul/min for 240 seconds. The protein analyte concentrations started at 25 nM and included 6 dilutions at 1:2 (1 part dilution to 1 part buffer). Dissociation times of 1000 seconds were included for all the dilutions due to the slow off rates.

### Step 4 Regeneration:

Regeneration was performed at the end of each cycle on all flow cells. Regeneration condition for the Biotin CAPture® Kit was as follows. The regeneration buffer was prepared by mixing 3 parts of Regeneration Stock 1 (8M guanidine-HCL, GE® 28-9202-33) to 1 part Regeneration Stock 2 (1M NaOH, GE® 28-9202-33). This flowed over the flow cells at 20 ul/min for 120 seconds.

### Target Protein Kinetics and Affinity Using the BIOTIN CAPture Method:

In order to determine target/ligand kinetics, two flow cells were used for this method. Flow cell 1 served as the reference cell which only contained the BIOCAP® reagent and flow cell 2 served as the binding cell which contained both the BIOCAP® reagent and the biotinylated target (eg. human VEGF-biotin). The method consists of 4 steps.

### Step 1 Biotin CAPture Reagent:

This reagent was provided in the kit and was diluted 1:3 into the running buffer. The flow rate was 2 ul/min and it flowed for 60 sec. The capture level was approximately 1500RU.

### Step 2 Biotinylated Ligand Capture Step:

Biotinylated target/ligand was flowed over at a rate of 10 ul/min for a set contact time to reach the desired Resonce Unit for an Rmax of 20.

The following equations represent the calculations to achieve a relative Rmax of 20:

VEGF Example: Rmax=RL*(MWanalyte/MWligand)*stoichiometry 20=RL*(50/50)*1=20RL

### Step 3 Antibody Dilutions (Analyte):

Since the protein analytes have strong affinities for their targets, the starting concentrations would be 10 nM and would include 8 serial dilution points. For example, for VEGF kinetics of some protein analytes, the starting concentration was 1.25 nM and included 7 dilutions at 1:2. Short dissociations and longer dissociations depend on the protein analyte. Overall for target kinetics for these lower affinity protein analytes, the protein analytes were flowed over at 60 ul/min for 240 seconds and had longer dissociation times greater than 1000 seconds.

### Step 4 Regeneration:

Regeneration was performed at the end of each cycle on all flow cells. Regeneration condition for the Biotin CAPture Kit is as follows. The regeneration buffer is prepared by mixing 3 parts of Regeneration Stock 1 (8M guanidine-HCL) to 1 part Regeneration Stock 2 (1M NaOH). This flowed over the flow cells at 20 ul/min for 120 seconds.

The sample compartment which includes the analytes, ligands, and regeneration buffer, is kept at 15° C. All other running conditions were carried out at either 25° C. or 37° C. in 1× HBSE+P buffer. The final results reflect a double referencing, subtraction of both the refraction index values from the reference flow cell and the blank binding step with no analyte. Data was collected at 10 Hz and analyzed using the Biacore T200 Evaluation Software (GE Healthcare®). This program uses a global fitting analysis method for the determination of rate and affinity constants for each interaction.

### Protease Sensitivity Assay

To assess for proteolytic clipping of the HA-binding peptide tag, NVS4 fused with various variants of the HA-binding peptide tag listed in table 4 below were site-specifically labeled with Invitrogen AlexaFluor488 on N-terminus of the light chain using Sortase-A mediated reaction. The labeled protein (1 mg/ml or greater) is mixed with CHO K1 PD spent medium in ratio of 1:10 of labeled protein to spent medium containing 0.05% sodium azide. The reaction mix is incubated at 37° C. with shaking. Twenty microliters are removed on different days, starting on Day 0 and frozen away. After the sample are taken out on the last designated day of incubation, 16 ul (12 ul of sample+4 ul of SDS loading dye) is loaded on Invitrogen's 12-16% 17-well NuPAGE Tris-Bis gel. The gel is scanned using BioRad Gel Doc 2000 under the AlexaFluor488 setting. Proteolytic clipping of the protein is analyzed by mass shift of the band to a lower molecular weight.

Two variants, NVS2a and NVS3a that had similar binding (Table 4) to the parent NVS1 and were subsequently assessed in the rabbit leakage model. Neither NVS2a and NVS3a demonstrated any efficacy in the rabbit model indicating that the glycosylation at position N311 was important for in vivo activity. The four variants that had similar binding (Table 4: NVS2, NVS3, NVS36, and NVS37) to the parent NVS1 were assessed in the rabbit leakage model. All four molecules, NVS2, NVS3, NVS36, and NVS37 demonstrated efficacy in the rabbit model similar to the parent NVS1. However, compared to NVS1, the four variants NVS2, NVS3, NVS36, and NVS37 showed increased protein stabilization, reduced or eliminated proteolytic clipping, and an increased melting point, key factors that improve developability of the tagged proteins.

These results indicated that following sequence modification to alter proteolytic cleavage only NVS2, NVS3, and NVS36, and NVS37 retained unique in vivo properties of having slower ocular clearance and extended efficacy duration.

TABLE 4
Optimization variants of NVS1. Variations listed are found in the peptide tag
sequence linked to the heavy chain of NVS1 (SEQ ID NO: 21). The peptide tag
corresponds to amino acids 229 to 326 of SEQ ID NO: 21.

| | | | Kd | | Positive Rabbit |
|---|---|---|---|---|---|
| NVS ID | Variation | Ka (1/M * s) | (1/s) | Affinity (M) | Efficacy |
| NVS1 | none | 5.00E+06 | 3.73E−01 | 7.47E−08 | Yes |
| NVS19 | N311E | 1.66E+07 | 6.19E−01 | 3.73E−08 | No |
| NVS20 | N311R | 7.11E+05 | 3.72E−02 | 5.23E−08 | Not assessed |
| NVS21 | N311T | 2.37E+06 | 1.93E−01 | 8.14E−08 | Not assessed |
| NVS22 | N311Y | 4.14E+06 | 2.48E−01 | 5.99E−08 | Not assessed |
| NVS12 | S313A | 2.08E+06 | 1.12E−01 | 5.38E−08 | No |
| NVS23 | S313K | 1.60E+06 | 1.93E−01 | 1.21E−07 | Not assessed |
| NVS24 | A235T | Low signal/minimal binding | | | Not assessed |
| NVS2 | R236Q | 4.13E+06 | 9.53E−01 | 2.31E−07 | Yes |
| NVS2a | R236A + N311E | 1.22E+06 | 9.21E−01 | 7.55E−07 | No |
| NVS3 | R236A | 7.27E+05 | 0.1355 | 1.86E−07 | Yes |
| NVS3a | R236Q + N311E | 1.01E+06 | 2.62E−01 | 2.59E−07 | No |
| NVS25 | S237T | No measurable binding | | | Not assessed |
| NVS26 | S237V | 8.87E+05 | 1.96E−01 | 2.21E−07 | Not assessed |
| NVS27 | S237Y | 1.33E+06 | 1.72E−01 | 1.29E−07 | Not assessed |
| NVS28 | R236Q + S313A | 6.11E+04 | 3.67E−01 | 6.00E−06 | Not Assessed |
| NVS29 | R236I + K241Y + R268Q | 3.23E+06 | 5.58E−01 | 1.73E−07 | Not assessed |
| NVS30 | R236I + K241Y + R268Q + | 1.02E+06 | 0.5475 | 5.35E−07 | Not assessed |
| | K29Q | | | | |
| NVS31 | R236I + K241Y + R268Q + | No expression | | | Not assessed |
| | K29Q + N311Q | | | | |
| NVS33 | R236I + K241Y + E265L + | 3.58E+06 | 5.20E−01 | 1.45E−07 | Not assessed |
| | R268Q | | | | |
| NVS34 | R236I + K241Y + E265Q + | 1.13E+06 | 3.05E−01 | 2.70E−07 | Not assessed |
| | R268Q | | | | |
| NVS35 | R236I + K241Y + I270P | 1.53E+06 | 1.96E−01 | 1.28E−07 | Not assessed |
| NVS36 | R236Q + ins | 2.52E+06 | 3.69E−01 | 1.46E−07 | Yes |
| | Cys_A267C | | | | |
| NVS37 | R236Q + | 3.91E+06 | 3.63E−01 | 9.28E−08 | Yes |
| | A259C + Y321C | | | | |
| NVS38 | R236Q + insCA | 4.47E+06 | 3.20E−01 | 7.16E−08 | Not assessed |
| | A267C | | | | |
| NVS39 | R236Q + | Affinity ≧500 nM | | | Not assessed |
| | K269Q | | | | |
| NVS40 | R236Q + K269H | Affinity ≧500 nM | | | Not assessed |
| NVS41 | R236Q + R268Y | Affinity ≧500 nM | | | Not assessed |
| NVS42 | R236Q + Q263C_Y321C | 3.16E+06 | 2.29E−01 | 7.25E−08 | Not assessed |
| NVS43 | R236Q + ins | No measurable binding | | | Not assessed |
| | SerGly_A267S | | | | |
| NVS49 | R236Q + N311Q | 1.15E+05 | 2E−01 | 1.74E−06 | Not assessed |
| NVS50 | N311Q + R312L | 1.21E+05 | 2.51E−01 | 2.08E−06 | Not assessed |
| NVS51 | R268Y + K269L | 4.43E+05 | 2.86E−01 | 6.45E−07 | Not assessed |
| NVS52 | N311Q + R312T | No expression | | | Not assessed |
| NVS53 | N311Q + R312S | No expression | | | Not assessed |
| NVS54 | N311Q + R312Q | No measurable binding | | | Not assessed |
| NVS55 | N311Q + R312H | No expression | | | Not assessed |
| NVS56 | N311Q + R312Y | No expression | | | Not assessed |
| NVS57 | R236I + | No measurable binding | | | Not assessed |
| | K241Y + K269Y | | | | |
| NVS58 | E234K | 2.95E+06 | 1.32E−01 | 4.47E−08 | Not assessed |
| NVS59 | K241Y | 4.71E+06 | 5.66E−01 | 1.20E−07 | Not assessed |
| NVS60 | K269Y | 1.80E+07 | 5.11E−01 | 2.84E−08 | Not assessed |
| NVS61 | A235N | 1.04E+07 | 6.50E−01 | 6.25E−08 | Not assessed |
| NVS62 | R236A + | No measurable binding | | | Not assessed |
| | K241Y + K269Y | | | | |
| NVS63 | R236I + K241Y | 1.02E+07 | 4.16E−01 | 4.09E−08 | Not assessed |
| NVS64 | E234D + K282E | 1.26E+06 | 9.55E−01 | 7.55E−07 | Not assessed |
| NVS65 | A235S + K262R | 1.04E+06 | 2.03E−01 | 1.95E−07 | Not assessed |
| NVS66 | Amino acids 229 | 2.79E+06 | 2.38E−01 | 8.54E−08 | Not assessed |
| | to 326 of SEQ ID | | | | |
| | NO: 21 were | | | | |
| | replaced with SEQ | | | | |
| | ID NO: 141 | | | | |

Select representative protease resistant or non-glycosylation variants that overall had the most favorable attributes in terms of biophysical properties, amino acid sequence, and HA binding were assessed in the rabbit model. More specifically, variants that had a decrease in pI, poor solubility due to the removal of glycosylation sites, and/or those variants that exhibited proteolytic clipping were not assessed.

SEQ ID NO: 141
GSGGGTCRYAGVYHREAQSGKYKLTYAEAKAVCEFEGGHLATYKQLEAAR
KIGFHVCAAGWMAKGRVGYPIVKPGPNCGFGKTGIIDYGIRLNRSERWDA
YCYNASAPPEEDCT

### Example 8

### Further Characterization of Optimized Antibodies: NVS1, NVS2, NVS3, NVS36, and NVS37

### 8a: Biacore Determination of Optimized VEGF Antibodies

The affinities of optimized VEGF antibodies for HA and human VEGF were measured by Biacore as described in example 7 above. Table 5 lists the mean on-rates (ka), off-rates (kd), and overall affinities (KD) for each molecule along for several experiments along with the range and standard error of the mean for each measured or calculated value. The overall affinities of NVS1, NVS2, NVS3, NVS36, and NVS37 for HA ranged from 5.75 uM to 31 nM measured at 25° C. and from 3.07 uM to 29 nM measured at 37° C. The affinity of all five peptide tagged fusions molecules are higher for VEGF as compared to the untagged Fab, NVS4 (Table 6).

TABLE 5
Binding Affinity to 17 kDa HA-biotin

| | ka Mean | ka +/− SEM | ka range | kd Mean | kd +/− SEM | kd range | KD Mean | KD +/− SEM | KD range | |
|---|---|---|---|---|---|---|---|---|---|---|
| NVS ID | (1/M*s) | (1/M*s) | (1/M*s) | (1/s) | (1/s) | (1/s) | (M) | (M) | (M) | Temperature |
| NVS1 | 1.91E+06 | 2.62E+05 | 6.05e4 to | 3.71E−01 | 4.81E−02 | 0.939 to | 3.88E−07 | 1.345E−07 | 3.14e−8 to | 25° C. |
| | | | 5e6 | | | 0.12 | | | 3.48e−6 | |
| NVS2 | 1.37E+06 | 2.86E+05 | 1.65e5 to | 3.38E−01 | 5.38E−02 | 0.309 to | 4.01E−07 | 8.498E−08 | 7.62e−8 to | 25° C. |
| | | | 4.07e6 | | | 0.183 | | | 1.11e−6 | |
| NVS3 | 2.48E+06 | 7.54E+05 | 4.1e4 to | 4.58E−01 | 4.92E−02 | 0.23 to | 6.61E−07 | 2.300E−07 | 4.91e−8 to | 25° C. |
| | | | 1.9e7 | | | 0.93 | | | 5.75e−6 | |
| NVS36 | 2.04E+06 | 4.85E+05 | 1.55E+6 to | 3.25E−01 | 4.45E−02 | 3.69E−1 to | 1.64E−07 | 1.73E−08 | 1.46E−7 to | 25° C. |
| | | | 2.52E+6 | | | 2.8E−1 | | | 1.81E−7 | |
| NVS37 | 2.58E+06 | 1.34E+06 | 1.24E+6 to | 3.89E−01 | 2.60E−02 | 4.15E−1 to | 2.14E−07 | 1.22E−07 | 3.36E−7 to | 25° C. |
| | | | 3.91E+6 | | | 3.63E−1 | | | 9.28E−8 | |
| NVS1 | 2.39E+06 | 1.12E+06 | 1.15e5 to | 2.13E−01 | 1.98E−02 | 0.154 to | 5.92E−07 | 4.957E−07 | 2.93e−8 to | 37° C. |
| | | | 5.25e6 | | | 0.239 | | | 2.08e−6 | |
| NVS2 | 1.37E+06 | 8.18E+05 | 8.51e4 to | 2.30E−01 | 3.00E−02 | 0.61 to | 1.09E−06 | 6.028E−07 | 8.6e−8 to | 37° C. |
| | | | 3.55e6 | | | 0.305 | | | 2.52e−6 | |
| NVS3 | 5.60E+05 | 3.60E+05 | 8.35e4 to | 3.86E−01 | 1.01E−01 | 0.214 to | 1.91E−06 | 4.988E−07 | 1.12e−7 to | 37° C. |
| | | | 1.98e6 | | | 0.511 | | | 3.07e−6 | |
| NVS36 | 4.69E+06 | n/a | n/a | 3.30E−01 | n/a | n/a | 7.02E−08 | n/a | n/a | 37° C. |
| NVS37 | 7.53E+05 | n/a | n/a | 1.46E−01 | n/a | n/a | 1.94E−07 | n/a | n/a | 37° C. |

na—not applicable, not run

TABLE 6
Affinities of NVS1, NVS2, NVS3, NVS4, NVS36, and NVS37 for
binding to the ocular target protein: human VEGF.

| NVS ID | Ka (1/M * s) | Kd (1/s) | Affinity (M) |
|---|---|---|---|
| NVS4 | 2.71E+06 | 1.38E−05 | 5.10E−12 |
| NVS1 | 5.75E+07 | 1.01E−05 | 1.76E−13 |
| NVS2 | 5.36E+07 | 1.05E−05 | 1.95E−13 |
| NVS3 | 7.39E+07 | 1.19E−05 | 1.61E−13 |
| NVS36 | 3.81E+07 | 2.50E−05 | 6.56E−13 |
| NVS37 | 2.35E+07 | 7.21E−05 | 3.07E−12 |

### 8b: Rabbit Efficacy of Optimized VEGF Antibodies

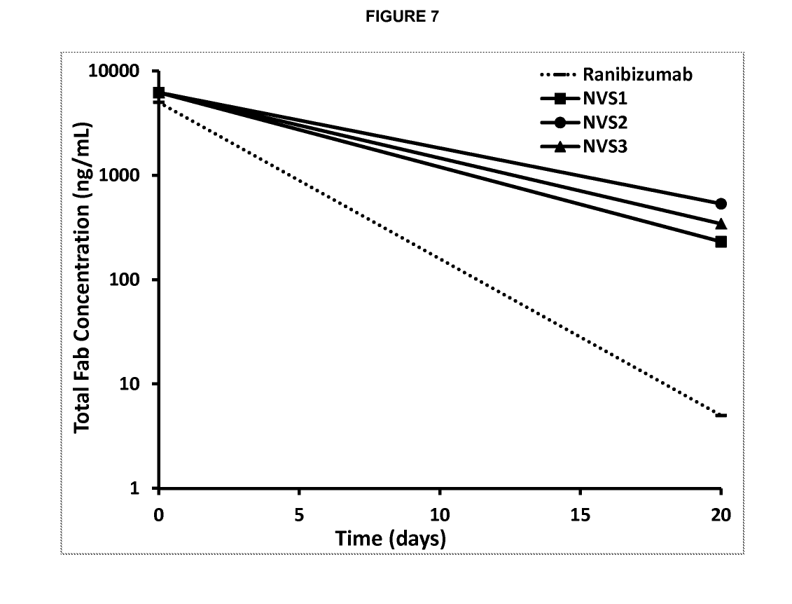

The rabbit leakage model (described in Example 2) was used to assess whether optimized anti-VEGF antibodies inhibit vessel leakage at 20 days post-injection (FIG. 6). The peptide tagged antibodies NVS1, NVS2, NVS3, NVS36 and NVS37 all significantly inhibited fluorescein leakage while equimolar ranibizumab did not (FIG. 6). The HA binding peptide tagged antibodies all had higher terminal drug concentrations at day 20 as compared to the untagged antibody, ranibizumab (FIG. 6). The terminal vitreal concentration of ranibizumab was 5 ng/ml, while the terminal vitreal concentrations of NVS1, NVS2, NVS3, NVS36, and NVS37 were 231, 533, 343, 722, and 646 ng/ml respectively. For ranibizumab this represented 0.2% of the injected dose, while the terminal vitreal concentrations of the optimized anti-VEGF antibodies represented 5.6-17.4% of the injected doses. Thus, the percent injected dose of the peptide tagged antibody was approximately 28-87-fold higher than the percent injected dose of ranibizumab at day 20. The terminal drug levels and starting doses were used to calculate 2-point PK curves (FIG. 7). The results indicated that the half-life values for ranibizumab, NVS1, NVS2, NVS3, NVS36 and NVS37 were 2, 4.2, 5.6, 4.8, 5.7, and 6.8 days respectively. Thus, an HA-binding peptide tag linked to an antibody improved half-life by ˜2-3.5-fold for NVS1, NVS2, NVS3, NVS36 and NVS37 compared to an untagged antibody (e.g.: ranibizumab).

This indicates that the clearance of the peptide tagged antibodies were slower than the untagged antibodies, and the slower clearance from the eye leads to higher drug levels at later times which are correlated with increased efficacy. The tagged antibodies were engineered to bind to hyaluronic acid, thereby slowing clearance of the antibody from the eye. The higher terminal drug levels, the higher percent injected dose at day 20 of the tagged antibody, and longer ocular half-life are consistent with this mechanism of action. Because there were higher levels of the antibodies tagged with peptide fragments that bind HA, dosing with a tagged antibody resulted in a greater suppression of VEGF levels. Lower VEGF levels correlate with reduction in the amount of vessel leakage and increased the duration of efficacy (FIGS. 6 and 7). In human wet AMD patients, suppression of VEGF levels is necessary to prevent recurrence of neovascularization activity, and increases in VEGF levels correlate with the return of disease activity (Muether et al., 2012). Thus, treatment of a patient with a retinal vascular disease (e.g., wet AMD) with an antibody tagged with a peptide fragment that binds HA is expected to have longer duration of action compared to an untagged anti-VEGF antibody, thereby benefiting patients by maintaining efficacy while providing a reduction in dosing frequency.

### Example 9

### Day 20 to Day 30 Longitudinal Efficacy and Terminal PK in Rabbits for NVS1 and NVS2

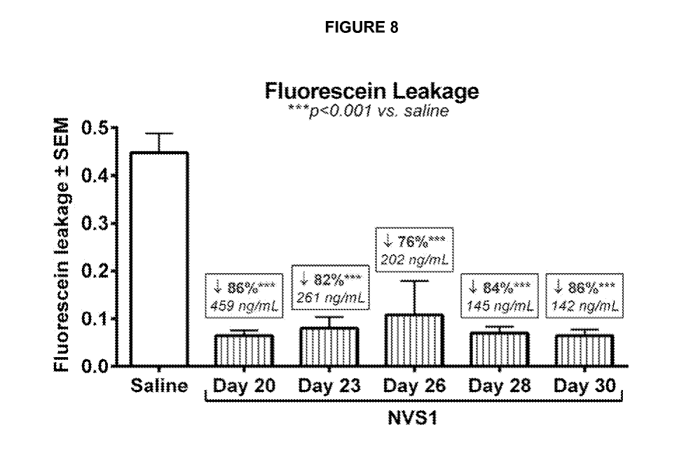

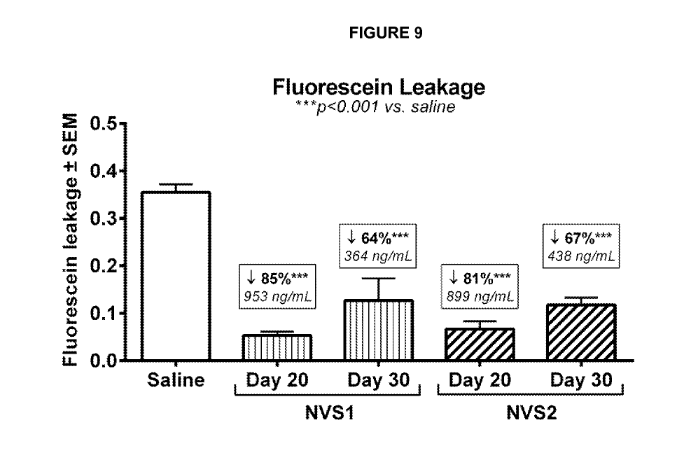

To determine the extent of increase in duration of efficacy of NVS1 and NVS2, the rabbit leakage model was modified to assess the efficacy of NVS1 and NVS2 as described below (FIG. 8 and FIG. 9). In these studies, 6.2 μg/eye of NVS1 and NVS2 (equimolar to 5 μg/eye ranibizumab) were intravitreally administered to different cohorts of rabbits at either 18, 21, 24, 26 or 28 days prior to the hVEGF challenge. Forty-eight hours post hVEGF challenge, fluorescein leakage was assessed as described above. NVS1 achieved similar efficacy (76-86%) at all time points in one study (FIG. 8). NVS1 and NVS2 both achieved similar efficacy at day 20 (81-85%) and day 30 (64-67%) (FIG. 9). In contrast, an equimolar dose of ranibizumab at day 20 days prior to hVEGF challenge did not inhibit vessel leakage (FIG. 3).

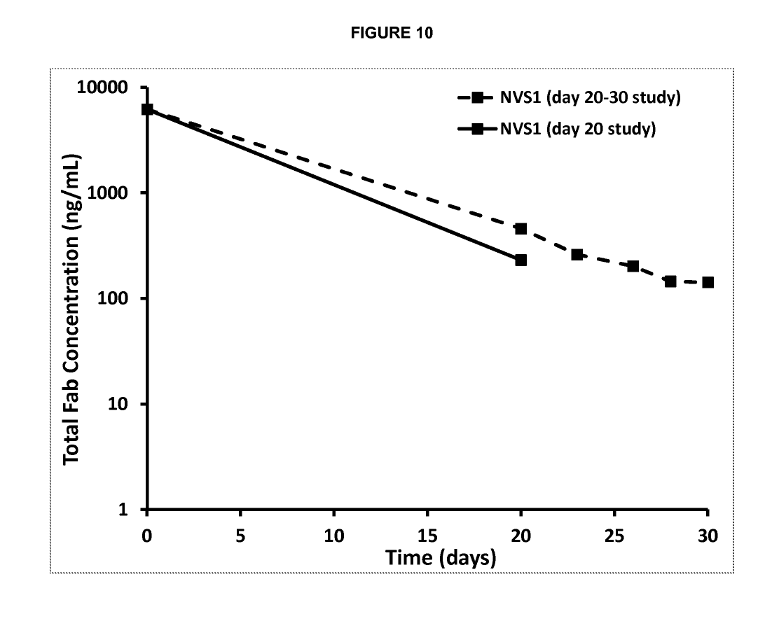

Upon completion of imaging to measure vessel leakage, the animals were sacrificed and the eyes were enucleated and processed for quantitation of total antibody concentration in the vitreous as described above. At day 20-21 post-IVT dosing, vitreal concentration of ranibizumab was approximately 5 ng/ml (FIGS. 6 and 7). In contrast, the terminal vitreal concentrations of NVS1 were 459, 261, 202, 145, and 142 at day 20, 23, 26, 28, and 30 respectively indicating a significant improvement in ocular retention. These terminal vitreal concentrations were used to calculate 2 and 6-point PK curves for NVS1 (FIG. 10). The results are that the ocular half-life values for NVS1 were 4.2 and 5.2 days respectively, indicating an improvement of half-life by about 2-2.5-fold of NVS1 compared to ranibizumab, which is similar to the results in FIG. 7.

The antibodies engineered with an HA binding peptide tag, showed a decrease in the clearance of the antibody from the eye as compared to the antibody without an HA-binding peptide tag. The higher terminal drug levels and longer ocular half-life are consistent with this mechanism of action. Because there were higher NVS1 levels at later time points, dosing with a peptide tagged antibody resulted in a greater suppression of VEGF levels for a longer period of time as compared to an untagged antibody. In human wet AMD patients, suppression of VEGF levels is necessary to prevent recurrence of neovascularization activity, and increases in VEGF levels correlate with the return of disease activity (Muether et al., 2012). Thus, treatment of a wet AMD patient with an anti-VEGF antibody linked to an HA-binding peptide tag is expected to have longer duration of action compared to an unmodified anti-VEGF antibody, thereby benefiting patients by maintaining efficacy while providing a reduction in dosing frequency.

### Example 10

### 28 Day Study of Tolerability, Efficacy, and Terminal PK of NVS1 and NVS4 in Cynomolgus Monkeys

Cynomolgus model of thermal laser-induced choroidal neovascularization

In the cynomolgus thermal-laser induced choroidal neovascularization model, a laser was used to disrupt the membrane barrier (Bruch's membrane) between the RPE and the choroid, which results in neovascularization at the site of the laser burn. Lesion size and leakage at the lesion can be measured using fluorescein angiography. To determine duration of action, an anti-VEGF molecule can be administered at various times prior to the thermal laser procedure. The interval between administration of the anti-VEGF molecule and laser treatment determines the duration of action of the anti-VEGF molecule.

Focal thermal laser ablation to the peri-macular retina is a common method for creating choroidal neovascular (CNV) lesions for evaluating therapeutics for age related macular degeneration (AMD). Based on previous benchmarking studies it was determined that use of a 657 nm krypton red laser was more effective than an argon green laser (532 nm) in creating clinically relevant grade IV CNV lesions (a scale of I-IV was used to grade lesion severity). With the 675 nm krypton laser, an extended duration of leakage beyond four weeks post laser could be achieved, and was therefore deemed suitable to evaluate the duration of action of anti-VEGF drugs over a period of several weeks to months.

### Intravitreal (IVT) Injections in Monkeys

Naïve non-human primates (Macaca fascicularis) (N=3, 2.4-5.8 kg) were sedated with an IM cocktail of Ketamine (5-20 mg/kg), Midazolam (0.05-0.5 mg/kg) and Glycopyrrolate (0.005 mg/kg). If necessary, depth of anesthesia was maintained with small supplemental IV doses (0.25-0.5 ml) of Propofol (2-5 mg/kg). The monkey was placed supine on a heated surgical table under a surgical microscope (Zeiss-Meditec®). The eyelids and adjacent tissues were cleaned with a betadine swab stick and sterile drape was positioned over the experimental eye. Each eye was instilled with 0.5% proparacaine ocular anesthetic to effect prior to receiving 1-2 drops of 0.5% ophthalmic betadine. Eyes were rinsed with sterile BSS and microsponges were used to wick away excess fluid. A pediatric eyelid speculum was positioned to retract the eyelids. GenTeal® Gel (Novartis®) was placed in the corneal aperture of a surgical magnifying contact lens (Ocular Instruments) to enhance visualization of the vitreous and retina through the surgical microscope. Fine forceps were used to grasp the conjunctiva and gently rotate the eye to expose the injection site at 3 mm behind the limbus. A 0.3 cc monoject syringe with 29G attached needle was inserted, bevel down, and angled toward the retina. Once the bevel was visualized and positioned for mid-vitreous delivery of the test article, the plunger was slowly depressed to deliver the 50 ul volume of material. The needle was slowly withdrawn and the injection site pinched with fine forceps to minimize or prevent any reflux of test article or vitreous. All eyes received 1-2 drops of topical ocular Vigamox (Alcon) to prevent infection. All injection observations were recorded. Animals were given anesthetic reversals and preventative analgesics prior to being returned to housing.

### Thermal Laser Procedure

The monkeys were sedated with an IM cocktail of Ketamine (5-20 mg/kg), Midazolam (0.05-0.5 mg/kg) and Glycopyrrolate (0.005 mg/kg). During procedures, depth of anesthesia was maintained with small supplemental IV doses (0.25-0.5 ml) of Propofol (2-5 mg/kg). A baseline color fundus photo is acquired prior to laser and used to pre-position the laser burns to ensure that they are equidistant from the fovea and from each other to minimize such effects as focal retinal vessel hemorrhages, CNV lesion coalescence and infringement on the function of the fovea. The sedated animals were placed on their ventral side on a custom designed inclined mobile imaging platform to position the head in alignment with the slit lamp mounted laser or imaging system camera lenses for each procedure. A single topical ocular drop of Alcaine (0.5% proparacaine, Alcon) was instilled in each eye prior to placement of a 1× Reichel Mainster contact lens (Ocular Instruments®) with GenTeal Gel (Novartis®) in the aperture. Using the krypton red laser settings at 600 mW, 75 um spot size; 0.01-0.1 sec single pulse duration (Novus Varia Three Mode Laser System, Lumenis®) four laser burns are made outside the fovea in both eyes. The monkeys were given reversals and preventative analgesic for 24 hours post procedure.

### Image Acquisition

The monkeys were given IM Zofran (0.1 mg/kg) and Benadryl (2.2 mg/kg), 30 minutes prior to anesthesia to minimize the occurrence of unpredictable sodium fluorescein-induced emesis. The monkeys were sedated with an IM cocktail of Ketamine (5-20 mg/kg), Midazolam (0.05-0.5 mg/kg) and Glycopyrrolate (0.005 mg/kg). During procedures, depth of anesthesia was maintained with small supplemental IV doses (0.25-0.5 ml) of Propofol (2-5 mg/kg). All imaging modalities were performed at baseline, post laser and two weeks post laser to document the appearance, thickness and leakage of the CNV lesions. Color funduscopy (Zeiss ff450+N camera, Carl Zeiss Meditec) was used to document the clinical appearance of the central 50 degrees of the retina. Infrared funduscopy, fluorescein angiography and SD-OCT (Spectralis, Heidelberg Engineering) were also implemented. CNV leakage was assessed using late phase fluorescein angiography at five minutes post IV bolus of 0.1-0.2 ml/kg of 10% AK-Fluor® (Akorn®). CNV lesion thickness was also measured using a single line within a 5°×15° 7-line SD-OCT grid to cover the approximate area occupied by each laser burn. The distance from the RPE to the ILM was measured with the Spectralis® HEYEX® software. The average thickness was calculated per group and an additional endpoint to evaluated efficacy of the drug treated groups and the control.

### CNV Grading Scheme

Late phase fluorescein angiography images acquired at five minutes post injection of IV fluorescein were used to subjectively grade the CNV lesions using a widely accepted four point grading scale (Covance and Krystolik M E, et al. Arch Ophthalmol 2002; 12:338). The masked, trained graders scored each lesion using the following subjective grading scale (Table 7). Grade I: No Hyperfluorscence; Grade II: Exhibited hyperfluorescence without leakage; Grade III: Hyperfluorescence in the early or midtransit images and late leakage; Grade IV: Show bright hyperfluroescence in the transit and late leakage beyond the treated areas

Grade IV lesions were defined as clinically significant. The average number of Grade IV lesions were counted per treatment group and used to calculate percent inhibition from the total number of laser burns created per treatment group.

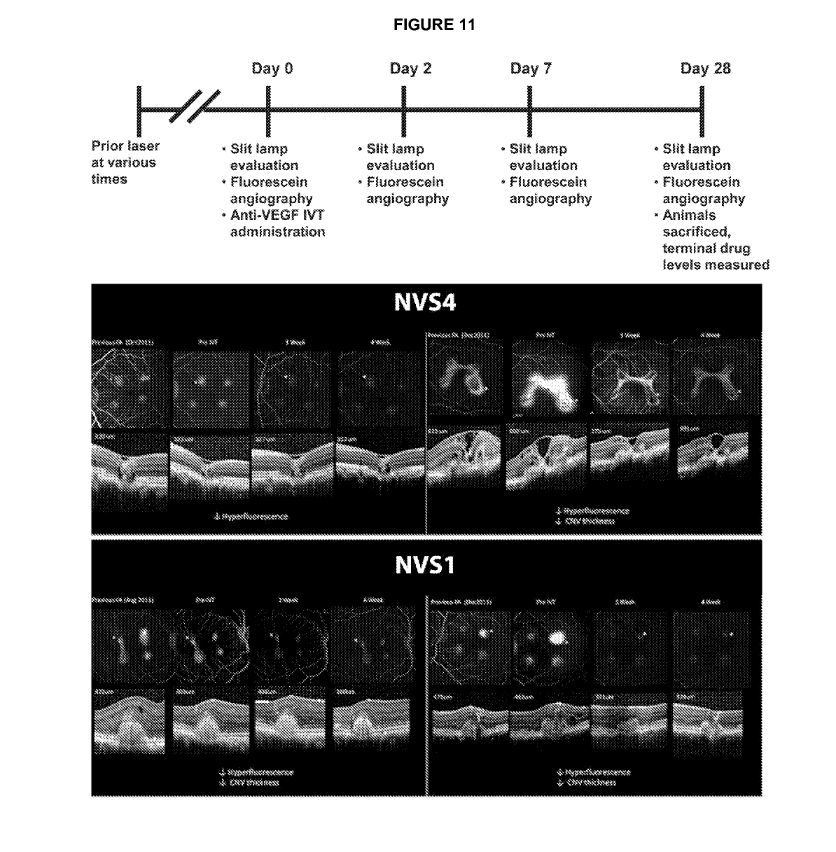

A pilot study was conducted in cynomolgus monkeys using non-naive cynomolgus monkeys that had been lasered previously and used as saline controls (FIG. 11). The primary readout was ocular tolerability of NVS1. Further, in these animals there was persistent vessel leakage as measured by fluorescein angiography, so a preliminary assessment of pharmacologic activity was also performed. A total of two animals per group (4 eyes total) received intravitreal anti-VEGF antibodies, either 200 μg/eye of NVS1 or 214 μg/eye of NVS2. Evaluations (slit lamp, fluorescein angiography) were done prior to drug administration on day, then on days 2, 7 and 28. On day 28, animals were sacrificed, eyes enucleated, and vitreous extracted for determination of terminal drug levels as described.

### Cyno Terminal Ocular PK Quantitation by ELISA

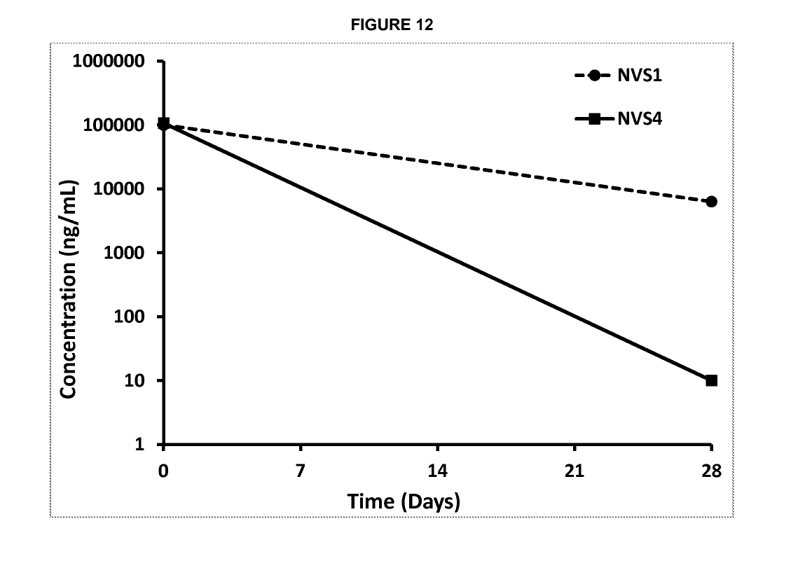

Ocular PK profiles of NVS1 and NVS4 in cyno vitreous were compared using standard methods as described below and shown in FIG. 12.

The enucleated eyes were dissected and the vitreous was separated from other tissues and further homogenized mechanically using a TissueLyzer (QIAGEN®). Antibody levels in the vitreous were measured by ELISA. The Maxisorp 384 well plates (Nunc 464718) were coated with VEGF (NOVARTIS® May 10, 2011) in carbonate buffer (PIERCE® 28382) overnight at 4 C. In between incubations, plates were washed 3 times with TBST (THERMO SCIENTIFIC® 28360) using a BioTek® plate washer. The next day, the plates were blocked for 2 hours at room temperature (or overnight at 4 C) with blocking buffer (5% BSA (SIGMA® A4503), 0.1% Tween-20 (SIGMA® P1379), 0.1% Triton X-100 (SIGMA®P234729)) in TBS. Samples were diluted in diluent (2% BSA (SIGMA® A4503), 0.1% Tween-20 (SIGMA® P1379), 0.1% Triton X-100 (SIGMA® P234729) in TBS) and incubated on the plate for 1 hour at room temperature with gentle shaking. Then, a goat anti-human antibody (bethyl A80-319A) was added to the plate for 1 hour at room temperature with gentle shaking. The detection antibody was a Rabbit Anti-Goat IgG (H+L), conjugated to HRP (THERMO FISHER® 31402). The detection antibody was added to the plate for 1 hour at room temperature with gentle shaking. Ultra TMB was added for 15 minutes (THERMO FISHER® 34028). The reaction was quenched with 2N sulfuric acid (Ricca 8310-32). The absorbance of the samples was read on the SpectraMax® (450-570 nm). To back-calculate Fab recovery levels from eye tissues, a purified standard was used. For the standard, the highest concentration used was 200 ng/mL with 2-fold dilutions.

Terminal drug levels were measured in vitreous extracts and used to generate 2-point PK curves (FIG. 12). The untagged antibody, NVS4, had an ocular half-life of 2.09 days, while NVS1 had an ocular half-life of 7.03 days. Thus, the HA-binding peptide tag improved the ocular PK by more than 3-fold. These results indicate that: 1) a protein linked to an HA-binding peptide tag can be administered safely in a non-human primate, 2) a protein linked to an HA-binding peptide tag can be efficacious in a model of wet AMD, and 3) high drug levels can be maintained for a longer period of time by linking a protein to an HA-binding peptide tag.

### Example 11

### 51 Day Terminal PK of NVS1 and Ranibizumab in Cynomolqus Monkeys

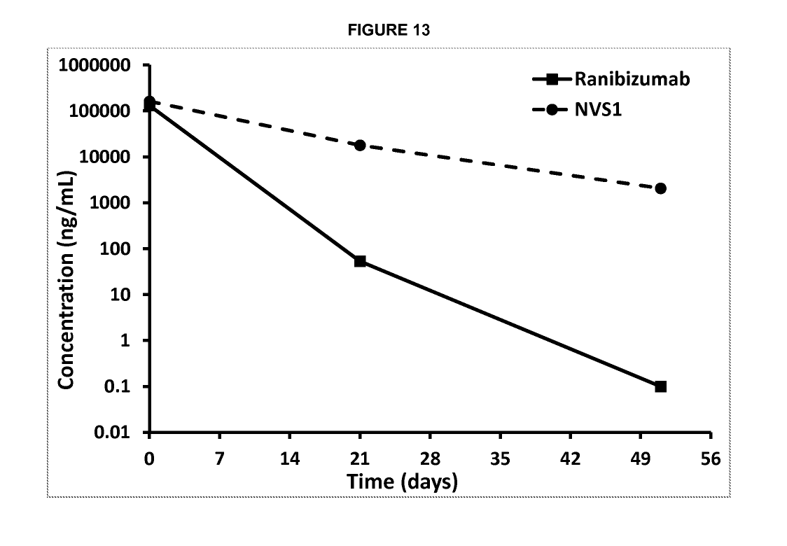

Either 263 μg/eye ranibizumab or 324 μg/eye NVS1 (NVS1: equimolar to ranibizumab dose) were administered intravitreally to groups (3 animals/group=6 eyes/group) of cynomolgus monkeys. At 21 or 51 days post-administration, animals were sacrificed, eyes enucleated, and terminal drug concentrations were measured by Gyrolab ELISA (FIG. 13).

### Cyno Terminal PK Quantitation by Gyrolab ELISA

Vitreous samples were thawed at room temperature for 10 minutes. NVS1 samples were diluted 1:10 in Rexxip AN buffer (Gyros®, Inc. Cat P0004994) in a 96-well PCR plate (THERMO SCIENTIFIC® AB-800, 0.2 mL Skirted 96-well PCR plate) while Ranibizumab™ samples were diluted 1:4 in Rexxip AN buffer. Samples were sealed (GYROS®, Inc. microplate foil Cat P0003313) and mixed thoroughly in a plate shaker for 1 minute. Ensuring that no bubbles are found in the bottom of the wells, the samples were placed in the Gyrolab™ xP workstation. A 3-step C-A-D method is executed on the Gyrolab™ xP workstation; capture antibody was flowed through the system first, followed by the analyte (samples), and then detector with washes of PBS 0.01% Tween20 (Calbiochem, Inc. Cat 655206) was performed in between each step. The standard curve for free (not bound to VEGF) NVS1 measurement is prepared in a diluent containing 10% rabbit vitreous (BioReclamation®, LLC. Cat Cyno-Vitreous) in Rexxip AN. The standard was serially diluted 1:6 from 6000 ng/mL to 0.129 ng/mL.

The standard curve for Ranibizumab™ measurement was prepared in a diluent containing 25% rabbit vitreous (BioReclamation®, LLC. Cat Cyno-Vitreous) in Rexxip AN. The standard was serially diluted 1:6 from 6000 ng/mL to 0.129 ng/mL.

On day 51, the mean concentrations of NVS1 and ranibizumab were 2070 ng/mL and <0.1 ng/mL, respectively. The data indicates that for NVS1, vitreous concentrations at day 51 are higher than those for ranibizumab at day 21. The starting doses and the day 21 and day 51 ocular drug levels were used to calculate 3-point PK curves (FIG. 13). These curves show that the ocular half-life values for ranibizumab and NVS1 were 2.6 and 8.2 days respectively, and demonstrate that linking a peptide tag that binds HA to an antibody can improve the ocular PK in a monkey about 3-fold. These results indicate a significant increase in the ocular half-life for the antibody tagged with the HA-binding peptide tag. The NVS1 antibody was engineered to bind to hyaluronic acid, thereby slowing clearance of the antibody from the eye. The higher drug levels over time and longer ocular half-life are consistent with this mechanism of action.

This extended duration of efficacy could be tested in an animal model such as the cynomolgus laser CNV, which is a model of wet AMD. Animals would be dosed at various times prior to thermal laser treatment (e.g., between 0 and 8 weeks). Dose groups would for example, include a vehicle control group (e.g., saline), a group treated with a control untagged antibody (e.g., ranibizumab or NVS4), and a group treated with the antibody tagged with HA-binding peptide (e.g., NVS2). Treatment of sufficient numbers of animals (e.g., 15-20 animals per treatment group) will allow a statistical differentiation in the duration of efficacy between an untagged antibody and an antibody tagged with an HA-binding peptide tag.

### Example 12

### Use of an Anti-VEGF Protein Linked to an HA-Binding Peptide Tag to Increase Half-Life, Terminal Concentrations and Duration of Efficacy of Anti-VEGF Proteins in Human Subjects

### 12a: Peptide Tag Increases in Higher Terminal Concentration and Duration of Action

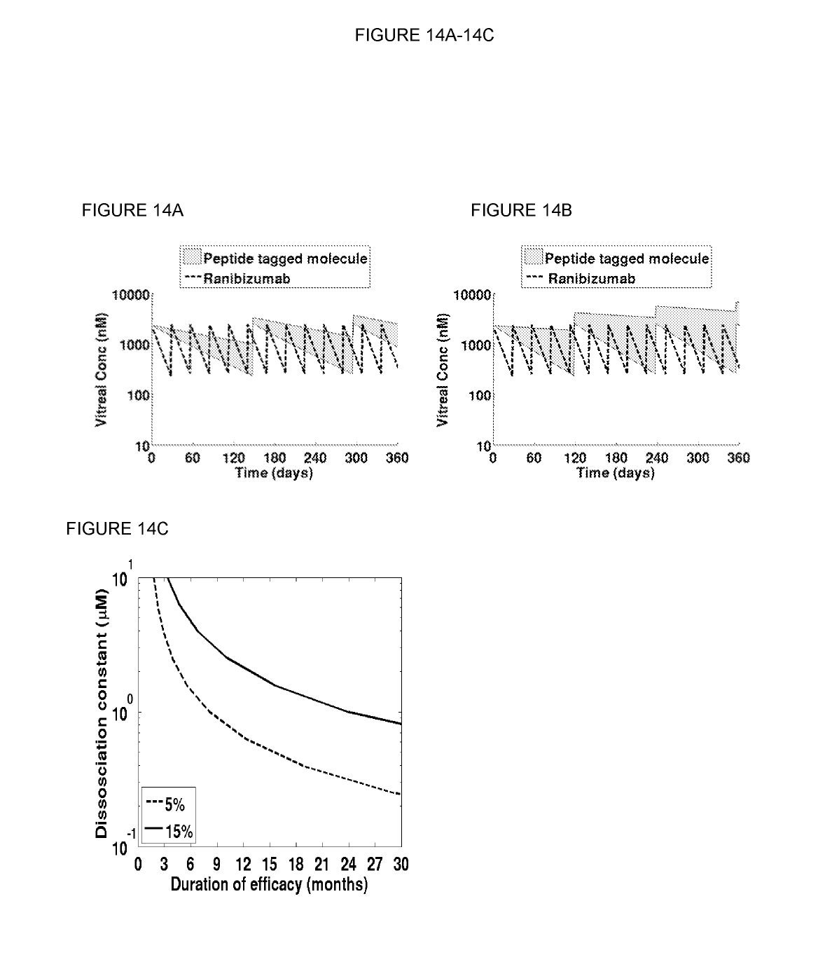

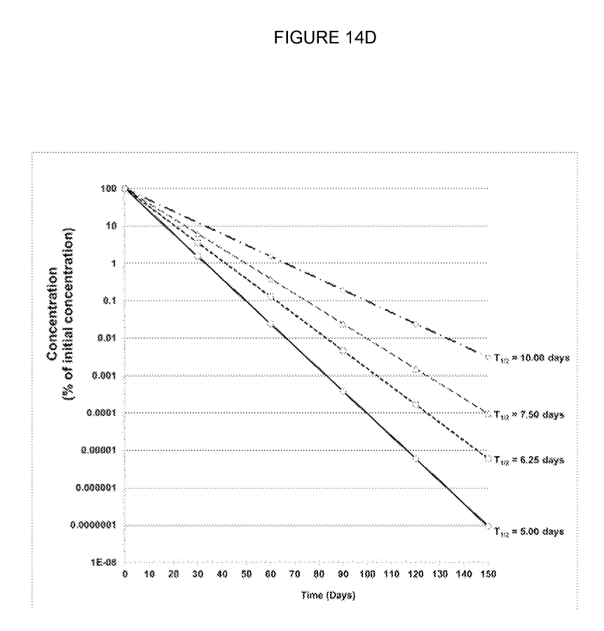

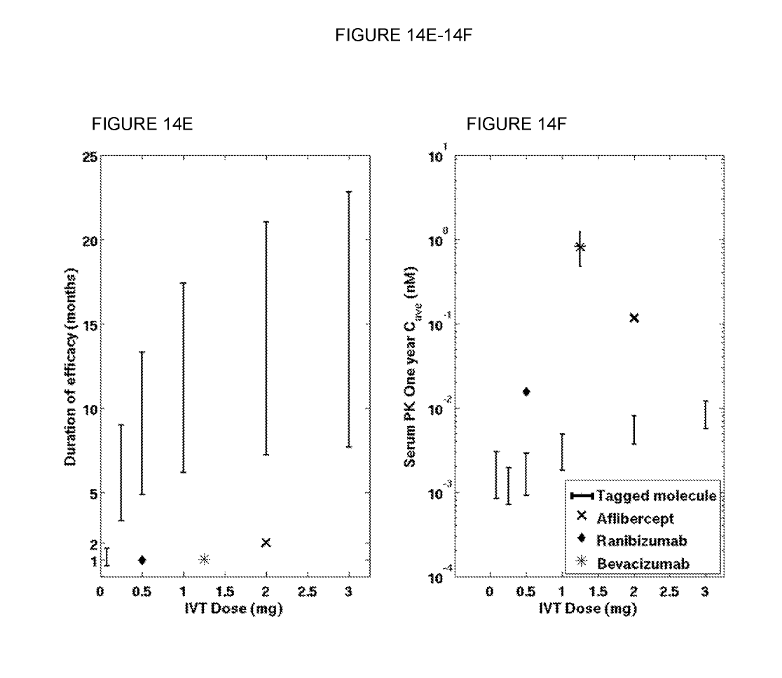

Treatment with an anti-VEGF protein (for example, antibodies or antigen binding fragments) linked to a peptide tag that binds HA (for example, peptide tags with the sequence of SEQ ID NO: 32, 33, 34, 35 or 36) results in higher drug levels at later times as compared to an untagged protein, thus there is a greater suppression of free VEGF levels for longer a period of time. Lower free VEGF levels correlate with reduction in the amount of disease pathology and increased duration of efficacy. In human wet AMD patients, suppression of VEGF levels is necessary to prevent recurrence of neovascularization activity, and increases in VEGF levels correlate with the return of disease activity (Muether et al., 2012). Thus, treatment of a wet AMD patient with an anti-VEGF protein linked to a HA-binding peptide tag as described herein (e.g.: NVS1, NVS2, NVS3, NVS36 or NVS37) will have longer duration of action compared to an untagged anti-VEGF protein, thereby benefiting patients by maintaining efficacy while providing a reduction in dosing frequency. An example of such a dosing scheme is shown in FIG. 14A-C. Currently, 500 ug/eye ranibizumab is dosed IVT every 28 days in human wet AMD patients to achieve maximum VEGF suppression and most visual improvement. An equimolar concentration of peptide tagged anti-VEGF protein (0.62 mg) was dosed IVT, such that vitreal concentration was greater than the ranibizumab concentration at day 28. In FIG. 14A the grey band for the simulation denotes range of predictions for an peptide tagged anti-VEGF protein that binds to 5-15% of the human vitreal HA (250 μg/mL) with a K_{D} of 1.7 μM. In FIG. 14B the grey band for the simulation denotes range of predictions for an peptide tagged anti-VEGF protein that binds to 15% of the vitreal HA with a K_{D} ranging from 0.48 to 7.2 μM. Duration of efficacy was plotted against K_{D} for HA (FIG. 14C), for an peptide tagged anti-VEGF protein that bind to 5% or 15% of the human vitreal HA. Duration of efficacy was defined as the time taken to reach the vitreal concentration of ranibizumab day 28. All simulations assume reversible binding of the peptide tagged anti-VEGF protein with vitreal HA. No clearance of the peptide tagged anti-VEGF protein was assumed, except for the dissociation of the peptide tagged anti-VEGF protein to form free HA and free peptide tagged anti-VEGF protein. Similar extension in duration of action and reduced dosing frequency is expected for other ocular therapeutics tagged with an HA-binding peptide tag including, for example, other anti-VEGF antibodies and antibodies binding other ocular targets.

A peptide tagged molecule, such as NVS1, NVS2, NVS3, NVS36 or NVS37 dosed at 500 ug/eye every 4 months is expected to achieve a similar amount of VEGF suppression and a concomitant improvement in vision as compared to dosing of ranizumab or other untagged anti-VEGF molecules monthly or bi-monthly. In human patients with other retinal vascular diseases, similar correlations of free VEGF levels and disease activity are likely, therefore similar extended duration of efficacy with a tagged anti-VEGF antibody is expected with similar doses of tagged anti-VEGF antibodies.

### 12b: Effects of Increase Half-Life on Ocular Drug Concentrations and Dosing Intervals.

Linking a peptide tag of the invention to a molecule for intraocular delivery can increase its ocular half-life relative to a molecule without a peptide tag. Increasing the ocular half-life of a molecule with an HA-binding peptide tag, can significantly increase the post-dosing drug levels compared to an untagged molecule, and an HA-binding peptide tagged molecule will take longer compared to an untagged molecule to reach a trough concentration level in the vitreous at which it is no longer therapeutically effective.

Clearance from the vitreous of an intravitreally administered biologic molecule has been shown to fit a first-order exponential decay function (equation 1) (Krohne et al., 2008; Krohne et al., 2012; Bakri et al., 2007b; Bakri et al., 2007a; Gaudreault et al., 2007; Gaudreault et al., 2005).

Ct=Cₜ₌₀*e^{-kt}
The rate constant k is:

$$k = \frac{ln 2}{{t}_{1 \text{/} 2} }$$

Cₜ is the concentration at time t after intravitreal administration.
Cₜ₌₀ is the concentration at time 0 after intravitreal administration.
T_{1/2} is the ocular half-life after intravitreal administration.

The effects of increasing the intravitreal half-life of a molecule with an HA-binding peptide tag can be modeled using the equations above. For the purposes of this example, an untagged molecule is presumed to have an ocular T_{1/2} of 5 days. In FIG. 14D the curves show the relative amounts of drug remaining at various times (100% of drug at time=0), and the effects of increasing the ocular T_{1/2} by 25%, 50%, 75% and 100%. FIG. 14D shows that increasing the ocular half-life results in higher concentrations of the intravitreally administered molecule at all times after the initial dose. Table 7a shows the amount of a molecule remaining (as a percentage of the initial dose) at 30-day intervals. Table 7b show the amounts of molecules remaining relative to the model untagged half-life of 5 days. For example, increasing the half-life by 25% (e.g.: from 5.0 to 6.25 days) results in a 2.3-fold increase in drug levels at day 30, a 5.28-fold increase in drug levels at day 60, a 12.13-fold increase in drug levels at day 90, a 27.86-fold increase in drug levels at day 120, and a 64-fold increase in drug levels at day 150. Increasing the half-life by 50% (e.g.: from 5.0 to 7.5 days) results in a 4-fold increase in drug levels at day 30, a 16-fold increase in drug levels at day 60, a 64-fold increase in drug levels at day 90, a >250-fold increase in drug levels at day 120, and a >1000-fold increase in drug levels at day 150. Increasing the half-life by 75% (e.g.: from 5.0 to 7.5 days) results in a 4-fold increase in drug levels at day 30, a 16-fold increase in drug levels at day 60, a 64-fold increase in drug levels at day 90, a >250-fold increase in drug levels at day 120, and a >1000-fold increase in drug levels at day 150. Increasing the half-life by 100% (e.g.: from 5.0 to 10.0 days) results in an 8-fold increase in drug levels at day 30, a 64-fold increase in drug levels at day 60, a >500-fold increase in drug levels at day 90, a >4000-fold increase in drug levels at day 120, and a >32,000-fold increase in drug levels at day 150.

### Table 7:

TABLE 7a

| | Drug Remaining in Vitreous (% of initial dose) | | | |
|---|---|---|---|---|
| | T_{1/2} (days) | | | |
| | 5.00 | 6.25 | 7.50 | 10.00 |
| Time Interval | days | days | days | days |
| day 30 | 1.56E+00 | 3.59E+00 | 6.25E+00 | 1.25E+01 |
| day 60 | 2.44E−02 | 1.29E−01 | 3.91E−01 | 1.56E+00 |
| day 90 | 3.81E−04 | 4.63E−03 | 2.44E−02 | 1.95E−01 |
| day 120 | 5.96E−06 | 1.66E−04 | 1.53E−03 | 2.44E−02 |
| day 150 | 9.31E−08 | 5.96E−06 | 9.54E−05 | 3.05E−03 |

TABLE 7b

Relative (vs. 5 day T_{1/2}) Concentration of
Drug Remaining in Vitreous
T_{1/2} (days)

| | 5.00 | 6.25 | 7.50 | 10.00 |
|---|---|---|---|---|
| Time Interval | days | days | days | days |
| day 30 | 1.00 | 2.30 | 4.00 | 8.00 |
| day 60 | 1.00 | 5.28 | 16.00 | 64.00 |
| day 90 | 1.00 | 12.13 | 64.00 | 512.00 |
| day 120 | 1.00 | 27.86 | 256.00 | 4096.00 |
| day 150 | 1.00 | 64.00 | 1024.00 | 32768.00 |

Thus, a peptide tag that increases the ocular half-life of a molecule (e.g.: and HA-binding peptide tag) can significantly improve the drug concentrations in the eye (i.e.: terminal drug concentration) and therefore lead to increase duration of efficacy and prolonged dosing intervals.

### 12c: Peptide Tags Increase, Half-Life, Duration of Efficacy and Decrease Plasma Exposure

The ocular clearance or pharmacokinetics of a molecule delivered to the eye (i.e.: a peptide tagged molecule or untagged molecule) can be measured directly in the eye using labeled molecules and non-invasive imaging techniques such as PET or fluorescence microscopy or by extracting intraocular fluids such as vitreous or aqueous humor and measuring concentrations using standard ELISAs, MSD assays, or mass spectrometry that are known in the art. For a molecule delivered to the eye, the appearance of the molecule in systemic circulation depends on the rate of clearance from the eye. The rate of appearance and concentration of such a molecule in systemic circulation can be used to determine the pharmacokinetics of the molecule in the eye (Xu L et al., Invest Ophthalmol Vis Sci., 54(3): 1616-24 (2013)).

The ocular pharmacokinetics of a peptide tagged molecule can similarly be assessed and predicted using a ocular PK binding model. In this model, the Fab binds to a fraction of the vitreal HA with a specific Kon and Koff rate. When not bound to HA, the Fab will leave the eye and enter serum, at the same rate as ranibizumab (8.6 day half-life). Based on fitting the HA-binding model to terminal vitreal concentration data from the Cynomolgus monkey IVT study, it was estimated that approximately 15% of monkey vitreal HA was binding to the Fab.

This model can be used to predict ocular and serum pharmacokinetics of peptide tagged molecules, such as NVS2 in a 4.5 mL human vitreous, assuming that the Fab binds to about 5-15% of the human vitreal HA (250 ug/mL), with a 4:1 HA to Fab stoichiometry, a Kon of 2×10⁶M⁻¹ sec⁻¹ and a KD of 1.7 μM. In the serum, the peptide tagged molecules will have the same systemic disposition as ranibizumab. Using this binding model, ocular and serum model predictions for the tagged peptide molecule were compared with other anti-VEGF molecules such as ranibizumab, aflibercept and bevacizumab.

The ranibizumab ocular-serum PK model was based on Xu L et al., Invest Ophthalmol Vis Sci., 2013. The bevacizumab ocular-serum PK model was based on a 9.82 day ocular half-life (Krohne T U et al., Am J Ophthalmol, 146(4): 508-12 (2008)), bioavailability F=0.65-0.95 and systemic disposition as described in bevacizumab Clinical Pharmacology review, STN-12085/0. The aflibercept model used a ocular half-life ˜4 days, and a systemic disposition as modeled in Thai H T et al., Br J Clin Pharmacol, 72(3): 402-14 (2011).

The duration of efficacy in the eye in this prediction was defined as the time taken for each molecule to reach an ocular concentration of ranibizumab 28 days after a 0.5 mg IVT administration. The error bar on the peptide tagged molecule simulation denotes range of predictions for the NVS2 peptide tagged molecule. A peptide tagged molecule (e.g.: NVS2) is predicted to achieve one-month efficacy with a low IVT dose of 0.08 mg. The peptide tagged molecule is also predicted to provide lower serum exposure than 0.5 mg ranibizumab. The 2-month duration for aflibercept was plotted based on the dosing interval used in the aflibercept label. The aflibercept serum prediction corresponds to free PK, after 3q4w followed by q8w administrations, as described in the aflibercept label.

Tagging a molecule (for example, and anti-VEGF protein) with an HA-binding peptide tag results in a slower clearance from the eye. Slower ocular clearance results in the delayed appearance of the peptide tagged molecule in systemic circulation and the maximum serum concentration reached is lower than that of the molecule without a peptide tag, illustrated in FIG. 14E. Systemic exposure of a peptide tagged molecule (e.g.: NVS2) is significantly less than an untagged molecule (e.g.: ranibizumab), when the tagged and untagged molecules are administered at equimolar doses. The serum concentrations of NVS2 are significant lower than that of ranibizumab at all equimolar doses. Similar results would be expected for tagged versions of aflibercept (e.g.: NVS80T) and bevacizumab (e.g.: NVS81T).

### Example 13

### Generation of Additional Proteins and Nucleic Acids Linked to an HA-Binding Peptide Tag

To test the ability of the HA-binding peptide tags to extend the half-life of proteins or nucleic acids in the eye, the peptide tags of the invention were linked to numerous antibodies, proteins and nucleic acids which bind a variety of ocular protein targets.

### Generation of Peptide Tagged Antibodies and Proteins

Tagged and untagged recombinant antibodies and proteins were expressed by transient transfections of mammalian expression vectors in HEK293 cells and purified using standard affinity resins for example, KappaSelect (Cat #17-5458-01, GE Healthcare Biosciences®) and HisTrap (Cat #17-5255-01, GE Healthcare Biosciences®). Various antibody and protein formats were tested, including: Fabs, IgGs, Fc Traps and proteins. These antibodies and proteins targets several ocular targets, for example, C5, Factor P, EPO, EPOR, TNFα, Factor D, IL-1β, IL-17A, FGFR2, or IL-10.

Fabs linked to single peptide tags were generated as described above by linking the HA-binding tag sequence to the C-terminal of the heavy chain of a Fab using a GSGGG linker (e.g.: SEQ ID NO: 31). To generate peptide tagged IgGs (e.g.: IgG fusions that contain HA-binding tag sequences) the HA-binding tag sequence was fused to the C-terminal of the heavy chain or light chain of an IgG using a GSGGG linker (e.g.: SEQ ID NO: 31). To generate peptide tagged proteins than contain an Fc portion, for example, Fc trap protein linked to an HA-binding tag, the HA-binding tag was linked to the C-terminal of the Fc portion of the protein using a GSGGG linker (e.g.: SEQ ID NO: 31). To generate additional peptide tagged proteins, the HA-binding tag was linked to the C-terminus of the protein of interest using a GSGGG linker (e.g.: SEQ ID NO: 31). In all cases described above, production of candidates entails nucleotide synthesis encoding the amino acid of desired proteins followed by expression and purification using mammalian expression systems described above.

The peptide tagged antibodies and peptide antigen binding fragments exemplified herein may also be converted and used in alternate antibody formats. For example, peptide tagged IgGs, can be converted to peptide tagged Fabs or peptide tagged scFvs, or vice versa.

### Generation of Peptide Tagged Nucleic Acids

Nucleic acids including RNA or DNA aptamers can be conjugated an HA-binding peptide as described below. In to a solution of B-3-(2-carboxyethyl)-1-(1-(2-hydrazinyl-4-methylpentanoyl)pyrrolidin-2-yl)-6-(1-hydroxyethyl)-1,4,7,10-tetraoxo-2, 5,8,11-tetraazatridecan-13-oic acid (198 mg, 0.280 mmol) in ACN (Volume: 1.75 mL) at room temperature is added DIPEA (0.098 mL, 0.559 mmol) and a solution of A-(3S,6S)-1-((S)-1-((S)-2-amino-4-methylpentanoyl)pyrrolidin-2-yl)-3-(2-carboxyethyl)-6-((R)-1-hydroxyethyl)-1,4,7,10-tetraoxo-2,5,8,11-tetraazatridecan-13-oic acid (32 mg, 0.056 mmol) in DMSO (Volume: 1.75 mL). The mixture is stirred at room temperature for 1 h and then purified using Sunfire Prep C18 eluting with 10 to 90% ACN-water+0.1% TFA to afford 27 mg pure desired product C-(3S,6S)-3-(2-carboxyethyl)-1-((S)-1-((S)-34-((2,5-dioxopyrrolidin-1-yl)oxy)-2-isobutyl-4,34-dioxo-7,10,13,16,19,22,25,28,31-nonaoxa-3-azatetratriacontan-1-oyl)pyrrolidin-2-yl)-6-((R)-1-hydroxyethyl)-1,4,7,10-tetraoxo-2,5,8,11-tetraazatridecan-13-oic acid. To a solution of D-ARC126-NH₂ (25 mg/ml in NaHCO3 pH-8.5 buffer) (18.63 mg, 230 μk, 1.807 μmol) is added C-(3S,6S)-3-(2-carboxyethyl)-1-((S)-1-((S)-34-((2,5-dioxopyrrolidin-1-yl)oxy)-2-isobutyl-4,34-dioxo-7,10,13, 16, 19,22,25,28,31-nonaoxa-3-azatetratriacontan-1-oyl)pyrrolidin-2-yl)-6-((R)-1-hydroxyethyl)-1,4,7,10-tetraoxo-2,5,8,11-tetraazatridecan-13-oic acid (100 mg/ml in DMSO) (5.26 mg, 52.6 μl, 4.52 μmol). The reaction is stirred at room temperature for 1.5 hr. The crude is passed through a 3K MW CO Amicon filter column (3K MW cut-off) and simultaneously buffer exchanged to sortase buffer 0.1M Tris pH8.0+CaCl2 0.01M+NaCl 0.15M. To a solution of F—the HA-peptide tag (287 μL, 0.047 μmol) in Tris 0.25M pH 7.4+CaCl2 5 mM and NaCl 150 mM (Volume: 313 μL) is added E (57.4 μL, 0.703 μmol) followed by immobilized Sortase A on beads (87 μL, 0.016 μmol). The mixture is agitated at 20° C. for 2 days. The resultant aptamer-HA binding peptide conjugate was NVS79T.

TABLE 8
Examples of proteins and nucleic acids linked to a peptide tag that binds HA.
The proteins and nucleic acids exemplified cover various examples of proteins and
nucleic acids that bind different targets in the eye.

| NVS ID | Ocular Target | HA Tag | Format | Location of HA tag |
|---|---|---|---|---|
| NVS70 | C5 | None | Fab | None |
| NVS70T | C5 | SEQ ID NO: 33 | Fab | C-terminus of NVS70 |
| | | | | heavy chain |
| NVS71 | Factor P | None | Fab | None |
| NVS71T | Factor P | SEQ ID NO: 33 | Fab | C-terminus of NVS71 |
| | | | | heavy chain |
| NVS72 | EPO | None | Fab | None |
| NVS72T | EPO | SEQ ID NO: 33 | Fab | C-terminus of NVS72 |
| | | | | heavy chain |
| NVS73 | TNFα | None | Fab | None |
| NVS73T | TNFα | SEQ ID NO: 33 | Fab | C-terminus of NVS73 |
| | | | | heavy chain |
| NVS74 | Factor D | None | Fab | None |
| NVS74T | Factor D | SEQ ID NO: 33 | Fab | C-terminus of NVS74 |
| | | | | heavy chain |
| NVS75 | IL-1β | None | Fab | None |
| NCS75T | IL-1β | SEQ ID NO: 33 | Fab | C-terminus of NVS75 |
| | | | | heavy chain |
| NVS76 | IL-17A | None | Fab | None |
| NVS76T | IL-17A | SEQ ID NO: 33 | Fab | C-terminus of NVS76 |
| | | | | heavy chain |
| NVS77 | FGFR2 | None | Fab | None |
| NVS77T | FGFR2 | SEQ ID NO: 33 | Fab | C-terminus of NVS77 |
| | | | | heavy chain |
| NVS78 | EPO | None | Fc Trap | None |
| NVS78T | EPO | SEQ ID NO: 33 | Fc Trap | C-terminus of Fc of NVS78 |
| NVS90 | EPOR | None | Protein | None |
| NVS90T | EPOR | SEQ ID NO: 33 | Protein | C-terminus of NVS90 |
| NVS79 | PDGF- | None | Aptamer | None |
| | BB | | | |
| NVS79T | PDGF- | SEQ ID NO: 33 | Aptamer | Chemically conjugated |
| | BB | | | to NVS79 |
| NVS91 | IL-10R | None | Protein | None |
| NVS91T | IL-10R | SEQ ID NO: 33 | Protein | C-terminus |

TABLE 8b
Sequences of peptide tagged molecules.

| | | Light Chain | |
|---|---|---|---|
| NVS ID | Ocular Target | (or single chain) | Heavy Chain |
| NVS70 | C5 | SEQ ID NO: 51 | SEQ ID NO: 42 |
| NVS7OT | C5 | SEQ ID NO: 51 | SEQ ID NO: 44 |
| NVS71 | Factor P | SEQ ID NO: 73 | SEQ ID NO: 61 |
| NVS71T | Factor P | SEQ ID NO: 73 | SEQ ID NO: 63 |
| NVS72 | EPO | SEQ ID NO: 95 | SEQ ID NO: 83 |
| NVS72T | EPO | SEQ ID NO: 95 | SEQ ID NO: 85 |
| NVS73 | TNFα | SEQ ID NO: 122 | SEQ ID NO: 113 |
| NVS73T | TNFα | SEQ ID NO: 122 | SEQ ID NO: 115 |
| NVS74 | Factor D | SEQ ID NO: 142 | SEQ ID NO: 143 |
| | | DIQVTQSPSSLSASVGDRVTIT | QLVQSGPELKKPGASVKVSC |
| | | CITSTDIDDDMNWYQQKPGK | KASGYTFTNYGMNWVRQAP |
| | | VPKLLISGGNTLRPGVPSRFS | GQGLEWMGWINTYTGETTYA |
| | | GSGSGTDFTLTISSLQPEDVA | DDFKGRFVFSLDTSVSTAYLQ |
| | | TYYCLQSDSLPYTFGQGTKVE | ISSLKAEDTAVYYCEREGGVN |
| | | IKRTVAAPSVFIFPPSDEQLKS | NWGQGTLVTVSSASTKGPSV |
| | | GTASWCLLNNFYPREAKVQ | FPLAPSSKSTSGGTAALGCLV |
| | | WKVDNALQSGNSQESVTEQD | KDYFPEPVTVSWNSGALTSG |
| | | SKDSTYSLSSTLTLSKADYEK | VHTFPAVLQSSGLYSLSSVVT |
| | | HKVYACEVTHQGLSSPVTKSF | VPSSSLGTQTYICNVNHKPSN |
| | | NRGEC | TKVDKRVEPKSC |
| NVS74T | Factor D | SEQ ID NO: 144 | SEQ ID NO: 145 |
| | | DIQVTQSPSSLSASVGDRVTIT | QLVQSGPELKKPGASVKVSC |
| | | CITSTDIDDDMNWYQQKPGK | KASGYTFTNYGMNWVRQAP |
| | | VPKLLISGGNTLRPGVPSRFS | GQGLEWMGWINTYTGETTYA |
| | | GSGSGTDFTLTISSLQPEDVA | DDFKGRFVFSLDTSVSTAYLQ |
| | | TYYCLQSDSLPYTFGQGTKVE | ISSLKAEDTAVYYCEREGGVN |
| | | IKRTVAAPSVFIFPPSDEQLKS | NWGQGTLVTVSSASTKGPSV |
| | | GTASWCLLNNFYPREAKVQ | FPLAPSSKSTSGGTAALGCLV |
| | | WKVDNALQSGNSQESVTEQD | KDYFPEPVTVSWNSGALTSG |
| | | SKDSTYSLSSTLTLSKADYEK | VHTFPAVLQSSGLYSLSSVVT |
| | | HKVYACEVTHQGLSSPVTKSF | VPSSSLGTQTYICNVNHKPSN |
| | | NRGEC | TKVDKRVEPKSCGSGGGGVY |
| | | | HREAQSGKYKLTYAEAKAVC |
| | | | EFEGGHLATYKQLEAARKIGF |
| | | | HVCAAGWMAKGRVGYPIVKP |
| | | | GPNCGFGKTGIIDYGIRLNRSE |
| | | | RWDAYCYNPHA |
| NVS75 | IL-1β | SEQ ID NO: 194 | SEQ ID NO: 202 |
| NCS75T | IL-1β | SEQ ID NO: 196 | SEQ ID NO: 202 |
| NVS78 | EPOR | SEQ ID NO: 146 | None |
| | | GGGGGPPPNLPDPKFESKAA | |
| | | LLAARGPEELLCFTERLEDLV | |
| | | CFWEEAASAGVGPGNYSFSY | |
| | | QLEDEPWKLCRLHQAPTARG | |
| | | AVRFWCSLPTADTSSFVPLEL | |
| | | RVTAASGAPRYHRVIHINEVVL | |
| | | LDAPVGLVARLADESGHVVLR | |
| | | WLPPPETPMTSHIRYEVDVSA | |
| | | GNGAGSVQRVEILEGRTECVL | |
| | | SNLRGRTRYTFAVRARMAEP | |
| | | SFGGFWSAWSEPVSLLTPSD | |
| | | LDPRIPKVDKKVEPKSCDKTH | |
| | | TCPPCPAPELLGGPSVFLFPP | |
| | | KPKDTLMISRTPEVTCVVVDV | |
| | | SHEDPEVKFNWYVDGVEVHN | |
| | | AKTKPREEQYNSTYRVVSVLT | |
| | | VLHQDWLNGKEYKCRVSNKA | |
| | | LPAPIEKTISKAKGQPREPQVY | |
| | | TLPPSRDELTKNQVSLTCLVK | |
| | | GFYPSDIAVEWESNGQPENN | |
| | | YKTTPPVLDSDGSFFLYSKLT | |
| | | VDKSRWQQGNVFSCSVMHE | |
| | | ALHNHYTQKSLSLSP | |
| NVS78T | EPOR | SEQ ID NO: 147 | None |
| | | GGGGGPPPNLPDPKFESKAA | |
| | | LLAARGPEELLCFTERLEDLV | |
| | | CFWEEAASAGVGPGNYSFSY | |
| | | QLEDEPWKLCRLHQAPTARG | |
| | | AVRFWCSLPTADTSSFVPLEL | |
| | | RVTAASGAPRYHRVIHINEVVL | |
| | | LDAPVGLVARLADESGHVVLR | |
| | | WLPPPETPMTSHIRYEVDVSA | |
| | | GNGAGSVQRVEILEGRTECVL | |
| | | SNLRGRTRYTFAVRARMAEP | |
| | | SFGGFWSAWSEPVSLLTPSD | |
| | | LDPRIPKVDKKVEPKSCDKTH | |
| | | TCPPCPAPELLGGPSVFLFPP | |
| | | KPKDTLMISRTPEVTCVVVDV | |
| | | SHEDPEVKFNWYVDGVEVHN | |
| | | AKTKPREEQYNSTYRVVSVLT | |
| | | VLHQDWLNGKEYKCRVSNKA | |
| | | LPAPIEKTISKAKGQPREPQVY | |
| | | TLPPSRDELTKNQVSLTCLVK | |
| | | GFYPSDIAVEWESNGQPENN | |
| | | YKTTPPVLDSDGSFFLYSKLT | |
| | | VDKSRWQQGNVFSCSVMHE | |
| | | ALHNHYTQKSLSLSPGSGGG | |
| | | GVYHREAQSGKYKLTYAEAK | |
| | | AVCEFEGGHLATYKQLEAARK | |
| | | IGFHVCAAGWMAKGRVGYPI | |
| | | VKPGPNCGFGKTGIIDYGIRLN | |
| | | RSERWDAYCYNPHA | |
| NVS90 | EPOR | SEQ ID NO: 148 | None |
| | | APPRLICDSRVLERYLLEAKEA | |
| | | ENITTGCAEHCSLNENITVPDT | |
| | | KVNFYAWKRMEVGQQAVEV | |
| | | WQGLALLSEAVLRGQALLVNS | |
| | | SQPWEPLQLHVDKAVSGLRS | |
| | | LTTLLRALGAQKEAISPPDAAS | |
| | | AAPLRTITADTFRKLFRVYSNF | |
| | | LRGKLKLYTGEACRTGDR | |
| NVS90T | EPOR | SEQ ID NO: 149 | None |
| | | APPRLICDSRVLERYLLEAKEA | |
| | | ENITTGCAEHCSLNENITVPDT | |
| | | KVNFYAWKRMEVGQQAVEV | |
| | | WQGLALLSEAVLRGQALLVNS | |
| | | SQPWEPLQLHVDKAVSGLRS | |
| | | LTTLLRALGAQKEAISPPDAAS | |
| | | AAPLRTITADTFRKLFRVYSNF | |
| | | LRGKLKLYTGEACRTGDRGS | |
| | | GGGGVYHREAQSGKYYLTYA | |
| | | EAKAVCEFEGGHLATYKQLEA | |
| | | ARKIGFHVCAAGWMAKGRVG | |
| | | YPIVKPGPNCGFGKTGIIDYGI | |
| | | RLNRSERWDAYCYNPHAGSH | |
| | | HHHHH | |
| NVS79 | PDGF-BB | SEQ ID NO: 150 | None |
| | | 5′-(C6-NH2)-dC-dA-dG-dG- | |
| | | dC-fU-dA-fC-mG-HEG-dC- | |
| | | dG-T-dA-mG-dA-mG-dC-dA- | |
| | | fU-fC-mA-HEG-T-dG-dA-T- | |
| | | fC-fC-fU-mG-3′-dT-3′ | |
| | | HEG = hexaethylene glycol | |
| | | phosphoamidite | |
| NVS79T | PDGF-BB | SEQ ID NOS: 150 and 151, | None |
| | | respectively | |
| | | 5′-(C6-NH2)-dC-dA-dG-dG- | |
| | | dC-fU-dA-fC-mG-HEG-dC-dG- | |
| | | T-dA-mG-dA-mG-dC-dA-fU- | |
| | | fC-mA-HEG-T-dG-dA-T-fC4C- | |
| | | fU-mG-3′-dT-3′- | |
| | | LPETGGGGGGSGGGGVYHR | |
| | | EAQSGKYYLTYAEAKAVCEFE | |
| | | GGHLATYKQLEAARKGFHVC | |
| | | AAGMAKGRVGYPN+1KPGPN | |
| | | CGFGKTGIIDYGRLNRSERW | |
| | | DAYCYNPHAGGSHHHHHH | |
| | | HEG = hexaethylene glycol | |
| | | phosphoamidite | |
| NVS91 | IL-10R | SEQ ID NO: 152 | None |
| | | SPGQGTQSENSCTHFPGNLP | |
| | | NMLRDLRDAFSRVKTFFQMK | |
| | | DQLDNLLLKESLLEDFKGYLG | |
| | | CQALSEMIQFYLEEVMPQAEN | |
| | | QDPDIKAHVNSLGENLKTLRL | |
| | | RLRRCHRFLPCENKSKAVEQ | |
| | | VKNAFNKLQEKGIYKAMSEFD | |
| | | IFINYIEAYMTMKIRN | |
| NVS91T | IL-10R | SEQ ID NO: 153 | None |
| | | SPGQGTQSENSCTHFPGNLP | |
| | | NMLRDLRDAFSRVKTFFQMK | |
| | | DQLDNLLLKESLLEDFKGYLG | |
| | | CQALSEMIQFYLEEVMPQAEN | |
| | | QDPDIKAHVNSLGENLKTLRL | |
| | | RLRRCHRFLPCENKSKAVEQ | |
| | | VKNAFNKLQEKGIYKAMSEFD | |
| | | IFINYIEAYMTMKIRNGSGGGG | |
| | | VYHREAQSGKYKLTYAEAKAV | |
| | | CEFEGGHLATYKQLEAARKIG | |
| | | FHVCAAGWMAKGRVGYPIVK | |
| | | PGPNCGFGKTGI | |
| | | IDYGIRLNRSERWDAYCYNPH | |
| | | AGSGGHHHHHH | |

Underlined sequences indicate additional optional sequence used for cloning (i.e.: GGGGG, SEQ ID NO: 187) or purification methods (e.g.: a hexa-histidine peptide, HHHHHH, SEQ ID NO: 188) described.

TABLE 9
Examples of VEGF binding proteins linked to a peptide tag that binds HA.
Examples include scFv, Fabs, full-length antibodies, DARPins, and Fc trap proteins
linked to a peptide fragment that binds HA.

| | | HA binding | | Position of HA binding |
|---|---|---|---|---|
| NVS ID | Target | peptide Tag | Protein Format | peptide tag |
| NVS80 | VEGF | None | Fc trap | None |
| NVS80T | VEGF | SEQ ID NO: 33 | Fc Trap | C-terminus of NVS80 heavy |
| | | | | chain |
| NVS81 | VEGF | None | IgG | None |
| NVS81T | VEGF | SEQ ID NO: 33 | IgG | C-terminus of NVS81 heavy |
| | | | | chain |
| NVS82 | VEGF | None | IgG | None IgG of NVS4 |
| NVS82T | VEGF | SEQ ID NO: 33 | IgG | C-terminus of NVS82 light |
| | | | | chain |
| NVS83 | VEGF | None | scFv | None. scFv of NVS4 |
| NVS83T | VEGF | SEQ ID NO: 33 | scFv | C-terminus of NVS83 |
| NVS84 | VEGF | None | DARPin | None |
| NVS84T | VEGF | SEQ ID NO: 33 | DARPin | C-terminus of NVS84 |
| NVS1b | VEGF | SEQ ID NO: 32 | Fab | C-terminus of heavy chain. |
| | | | | NVS1 Fab with 5 extra Gs |
| | | | | on N-terminus of light chain |
| NVS1c | VEGF | SEQ ID NO: 32 | Fab | N-terminus of both heavy |
| | | | | and light chains of NVS4 |
| NVS1d | VEGF | SEQ ID NO: 32 | Fab | C-terminus of both heavy |
| | | | | and light chains of NVS4 |
| NVS1e | VEGF | SEQ ID NO: 32 | Fab | Two tandem tags on the C- |
| | | | | terminus of the light chain of |
| | | | | NVS4 |
| NVS1f | VEGF | SEQ ID NO: 32 | Fab | One tag on the C-terminus |
| | | | | of the heavy chain and one |
| | | | | tag on the N-terminus of the |
| | | | | light chain of NVS4 |
| NVS1g | VEGF | SEQ ID NO: 32 | Fab | Four tandem tags on the C- |
| | | | | terminus of the heavy chain |
| | | | | of NVS4 |
| NVS1j | VEGF | SEQ ID NO: 32 | Fab | C-terminus of the light chain |
| | | | | of NVS4 |

TABLE 9b
Sequences of VEGF binding proteins linked to a peptide tag that binds HA.

| NVS ID | Single Chain or Heavy chain | Light Chain |
|---|---|---|
| NVS80 | SEQ ID NO: 154 | None |
| | SDTGRPFVEMYSEIPEIIHMTEGRELVIPC | |
| | RVTSPNITVTLKKFPLDTLIPDGKRIIWDSR | |
| | KGFIISNATYKEIGLLTCEATVNGHLYKTNY | |
| | LTHRQTNTIIDVVLSPSHGIELSVGEKLVLN | |
| | CTARTELNVGIDFNWEYPSSKHQHKKLVN | |
| | RDLKTQSGSEMKKFLSTLTIDGVTRSDQG | |
| | LYTCAASSGLMTKKNSTFVRVHEKDKTHT | |
| | CPPCPAPEAAGGPSVFLFPPKPKDTLMIS | |
| | RTPEVTCVVVDVSHEDPEVKFNWYVDGV | |
| | EVHNAKTKPREEQYNSTYRVVSVLTVLHQ | |
| | DWLNGKEYKCKVSNKALPAPIEKTISKAK | |
| | GQPREPQVYTLPPSRDELTKNQVSLTCLV | |
| | KGFYPSDIAVEWESNGQPENNYKTTPPVL | |
| | DSDGSFFLYSKLTVDKSRWQQGNVFSCS | |
| | VMHEALHNHYTQKSLSLSPGK | |
| NVS80T | SEQ ID NO: 156 | None |
| | SDTGRPFVEMYSEIPEIIHMTEGRELVIPC | |
| | RVTSPNITVTLKKFPLDTLIPDGKRIIWDSR | |
| | KGFIISNATYKEIGLLTCEATVNGHLYKTNY | |
| | LTHRQTNTIIDVVLSPSHGIELSVGEKLVLN | |
| | CTARTELNVGIDFNWEYPSSKHQHKKLVN | |
| | RDLKTQSGSEMKKFLSTLTIDGVTRSDQG | |
| | LYTCAASSGLMTKKNSTFVRVHEKDKTHT | |
| | CPPCPAPEAAGGPSVFLFPPKPKDTLMIS | |
| | RTPEVTCVVVDVSHEDPEVKFNWYVDGV | |
| | EVHNAKTKPREEQYNSTYRVVSVLTVLHQ | |
| | DWLNGKEYKCKVSNKALPAPIEKTISKAK | |
| | GQPREPQVYTLPPSRDELTKNQVSLTCLV | |
| | KGFYPSDIAVEWESNGQPENNYKTTPPVL | |
| | DSDGSFFLYSKLTVDKSRWQQGNVFSCS | |
| | VMHEALHNHYTQKSLSLSPGGSGGGGVY | |
| | HREAISGKYYLTYAEAKAVCEFEGGHLAT | |
| | YKQLEAAQQIGFHVCAAGWMAKGRVGYP | |
| | IVKPGPNCGFGKTGIIDYGIRLQRSERWD | |
| | AYCYNPHA | |
| NVS81 | SEQ ID NO: 157 | SEQ ID NO: 158 |
| | EVQLVESGGGLVQPGGSLRLSCAASGYT | DIQMTQSPSSLSASVGDRVTITCSASQ |
| | FTNYGMNWVRQAPGKGLEVVVGWINTYT | DISNYLNWYQQKPGKAPKVLIYFTSSL |
| | GEPTYAADFKRRFTFSLDTSKSTAYLQMN | HSGVPSRFSGSGSGTDFTLTISSLQP |
| | SLRAEDTAVYYCAKYPHYYGSSHWYFDV | EDFATYYCQQYSTVPVVTFGQGTKVEI |
| | WGQGTLVTVSSASTKGPSVFPLAPSSKS | KRTVAAPSVFIFPPSDEQLKSGTASVV |
| | TSGGTAALGCLVKDYFPEPVTVSWNSGA | CLLNNFYPREAKVQWKVDNALQSGN |
| | LTSGVHTFPAVLQSSGLYSLSSWTVPSS | SQESVTEQDSKDSTYSLSSTLTLSKA |
| | SLGTQTYICNVNHKPSNTKVDKKVEPKSC | DYEKHKVYACEVTHQGLSSPVTKSFN |
| | DKTHTCPPCPAPELLGGPSVFLFPPKPKD | RGEC |
| | TLMISRTPEVTCVVVDVSHEDPEVKFNWY | |
| | VDGVEVHNAKTKPREEQYNSTYRVVSVL | |
| | TVLHQDWLNGKEYKCKVSNKALPAPIEKT | |
| | ISKAKGQPREPQVYTLPPSREEMTKNQVS | |
| | LTCLVKGFYPSDIAVEWESNGQPENNYKT | |
| | TPPVLDSDGSFFLYSKLTVDKSRWQQGN | |
| | VFSCSVMHEALHNHYTQKSLSLSPGK | |
| NVS81T | SEQ ID NO: 159 | SEQ ID NO: 160 |
| | EVQLVESGGGLVQPGGSLRLSCAASGYT | DIQMTQSPSSLSASVGDRVTITCSASQ |
| | FTNYGMNWVRQAPGKGLEVVVGWINTYT | DISNYLNWYQQKPGKAPKVLIYFTSSL |
| | GEPTYAADFKRRFTFSLDTSKSTAYLQMN | HSGVPSRFSGSGSGTDFTLTISSLQP |
| | SLRAEDTAVYYCAKYPHYYGSSHWYFDV | EDFATYYCQQYSTVPVVTFGQGTKVEI |
| | WGQGTLVTVSSASTKGPSVFPLAPSSKS | KRTVAAPSVFIFPPSDEQLKSGTASVV |
| | TSGGTAALGCLVKDYFPEPVTVSWNSGA | CLLNNFYPREAKVQWKVDNALQSGN |
| | LTSGVHTFPAVLQSSGLYSLSSWTVPSS | SQESVTEQDSKDSTYSLSSTLTLSKA |
| | SLGTQTYICNVNHKPSNTKVDKKVEPKSC | DYEKHKVYACEVTHQGLSSPVTKSFN |
| | DKTHTCPPCPAPELLGGPSVFLFPPKPKD | RGEC |
| | TLMISRTPEVTCVVVDVSHEDPEVKFNWY | |
| | VDGVEVHNAKTKPREEQYNSTYRVVSVL | |
| | TVLHQDWLNGKEYKCKVSNKALPAPIEKT | |
| | ISKAKGQPREPQVYTLPPSREEMTKNQVS | |
| | LTCLVKGFYPSDIAVEWESNGQPENNYKT | |
| | TPPVLDSDGSFFLYSKLTVDKSRWQQGN | |
| | VFSCSVMHEALHNHYTQKSLSLSPGKGS | |
| | GGGGVYHREAQSGKYKLTYAEAKAVCEF | |
| | EGGHLATYKQLEAARKIGFHVCAAGWMA | |
| | KGRVGYPIVKPGPNCGFGKTGIIDYGIRLN | |
| | RSERWDAYCYNPHA | |
| NVS82 | SEQ ID NO: 161 | SEQ ID NO: 162 |
| | EVQLVESGGGLVQPGGSLRLSCTASGFS | EIVMTQSPSTLSASVGDRVIITCQASEII |
| | LTDYYYMTWVRQAPGKGLEVVVGFIDPDD | HSWLAWYQQKPGKAPKLLIYLASTLA |
| | DPYYATWAKGRFTISRDNSKNTLYLQMN | SGVPSRFSGSGSGAEFTLTISSLQPD |
| | SLRAEDTAVYYCAGGDHNSGWGLDIWG | DFATYYCQNVYLASTNGANFGQGTKL |
| | QGTLVTVSSASTKGPSVFPLAPSSKSTSG | TVLKRTVAAPSVFIFPPSDEQLKSGTA |
| | GTAALGCLVKDYFPEPVTVSWNSGALTS | SVVCLLNNFYPREAKVQWKVDNALQS |
| | GVHTFPAVLQSSGLYSLSSVVTVPSSSLG | GNSQESVTEQDSKDSTYSLSSTLTLS |
| | TQTYICNVNHKPSNTKVDKRVEPKSCDKT | KADYEKHKVYACEVTHQGLSSPVTKS |
| | HTCPPCPAPEAAGGPSVFLFPPKPKDTLM | FNRGEC |
| | ISRTPEVTCVVVDVSHEDPEVKFNWYVD | |
| | GVEVHNAKTKPREEQYNSTYRVVSVLTVL | |
| | HQDWLNGKEYKCKVSNKALPAPIEKTISK | |
| | AKGQPREPQVYTLPPSREEMTKNQVSLT | |
| | CLVKGFYPSDIAVEWESNGQPENNYKTT | |
| | PPVLDSDGSFFLYSKLTVDKSRWQQGNV | |
| | FSCSVMHEALHNHYTQKSLSLSPGK | |
| NVS82T | SEQ ID NO: 163 | SEQ ID NO: 164 |
| | EVQLVESGGGLVQPGGSLRLSCTASGFS | EIVMTQSPSTLSASVGDRVIITCQASEII |
| | LTDYYYMTWVRQAPGKGLEVVVGFIDPDD | HSWLAWYQQKPGKAPKLLIYLASTLA |
| | DPYYATWAKGRFTISRDNSKNTLYLQMN | SGVPSRFSGSGSGAEFTLTISSLQPD |
| | SLRAEDTAVYYCAGGDHNSGWGLDIWG | DFATYYCQNVYLASTNGANFGQGTKL |
| | QGTLVTVSSASTKGPSVFPLAPSSKSTSG | TVLKRTVAAPSVFIFPPSDEQLKSGTA |
| | GTAALGCLVKDYFPEPVTVSWNSGALTS | SVVCLLNNFYPREAKVQWKVDNALQS |
| | GVHTFPAVLQSSGLYSLSSVVTVPSSSLG | GNSQESVTEQDSKDSTYSLSSTLTLS |
| | TQTYICNVNHKPSNTKVDKRVEPKSCDKT | KADYEKHKVYACEVTHQGLSSPVTKS |
| | HTCPPCPAPEAAGGPSVFLFPPKPKDTLM | FNRGECGSGGGGVYHREAQSGKYKL |
| | ISRTPEVTCVVVDVSHEDPEVKFNWYVD | TYAEAKAVCEFEGGHLATYKQLEAAR |
| | GVEVHNAKTKPREEQYNSTYRVVSVLTVL | KIGFHVCAAGWMAKGRVGYPIVKPGP |
| | HQDWLNGKEYKCKVSNKALPAPIEKTISK | NCGFGKTGIIDYGIRLNRSERWDAYC |
| | AKGQPREPQVYTLPPSREEMTKNQVSLT | YNPHA |
| | CLVKGFYPSDIAVEWESNGQPENNYKTT | |
| | PPVLDSDGSFFLYSKLTVDKSRWQQGNV | |
| | FSCSVMHEALHNHYTQKSLSLSPGK | |
| NVS83 | SEQ ID NO: 165 | None |
| | MEIVMTQSPSTLSASVGDRVIITCQASEIIH | |
| | SWLAWYQQKPGKAPKLLIYLASTLASGVP | |
| | SRFSGSGSGAEFTLTISSLQPDDFATYYC | |
| | QNVYLASTNGANFGQGTKLTVLGGGGGG | |
| | SGGGGSGGGGSSGGGSEVQLVESGGGL | |
| | VQPGGSLRLSCTASGFSLTDYYYMTVVVR | |
| | QAPGKGLEWVGFIDPDDDPYYATWAKGR | |
| | FTISRDNSKNTLYLQMNSLRAEDTAVYYC | |
| | AGGDHNSGWGLDIWGQGTLVTVSSHHH | |
| | HHH | |
| NVS83T | SEQ ID NO: 166 | None |
| | MEIVMTQSPSTLSASVGDRVIITCQASEIIH | |
| | SWLAWYQQKPGKAPKLLIYLASTLASGVP | |
| | SRFSGSGSGAEFTLTISSLQPDDFATYYC | |
| | QNVYLASTNGANFGQGTKLTVLGGGGGG | |
| | SGGGGSGGGGSSGGGSEVQLVESGGGL | |
| | VQPGGSLRLSCTASGFSLTDYYYMTVVVR | |
| | QAPGKGLEWVGFIDPDDDPYYATWAKGR | |
| | FTISRDNSKNTLYLQMNSLRAEDTAVYYC | |
| | AGGDHNSGWGLDIWGQGTLVTVSSGSG | |
| | GGGVYHREAQSGKYKLTYAEAKAVCEFE | |
| | GGHLATYKQLEAARKIGFHVCAAGWMAK | |
| | GRVGYPIVKPGPNCGFGKTGIIDYGIRLNR | |
| | SERWDAYCYNPHAHHHHHH | |
| NVS84 | SEQ ID NO: 167 | None |
| | SDLGKKLLEAARAGQDDEVRILMANGADV | |
| | NTADSTGWTPLHLAVPWGHLEIVEVLLKY | |
| | GADVNAKDFQGWTPLHLAAAIGHQEIVEV | |
| | LLKNGADVNAQDKFGKTAFDISIDNGNED | |
| | LAEILQKAAGSLPETGGGSGHHHHHH | |
| NVS84T | SEQ ID NO: 168 | None |
| | SDLGKKLLEAARAGQDDEVRILMANGADV | |
| | NTADSTGWTPLHLAVPWGHLEIVEVLLKY | |
| | GADVNAKDFQGWTPLHLAAAIGHQEIVEV | |
| | LLKNGADVNAQDKFGKTAFDISIDNGNED | |
| | LAEILQKAAGSGGGGVYHREAQSGKYKL | |
| | TYAEAKAVCEFEGGHLATYKQLEAARKIG | |
| | FHVCAAGWMAKGRVGYPIVKPGPNCGF | |
| | GKTGIIDYGIRLNRSERWDAYCYNPHAGS | |
| | GGHHHHHH | |
| NVS85 | SEQ ID NO: 169 | None |
| | SDLGKKLLEAARAGQDDEVRILMANGADV | |
| | NAFDWMGVVTPLHLAAHEGHLEIVEVLLK | |
| | NGADVNATDVSGYTPLHLAAADGHLEIVE | |
| | VLLKYGADVNTKDNTGWTPLHLSADLGRL | |
| | EIVEVLLKYGADVNAQDKFGKTAFDISIDN | |
| | GNEDLAEILQKAAHHHHHH | |
| NVS85T | SEQ ID NO: 170 | None |
| | SDLGKKLLEAARAGQDDEVRILMANGADV | |
| | NAFDWMGVVTPLHLAAHEGHLEIVEVLLK | |
| | NGADVNATDVSGYTPLHLAAADGHLEIVE | |
| | VLLKYGADVNTKDNTGWTPLHLSADLGRL | |
| | EIVEVLLKYGADVNAQDKFGKTAFDISIDN | |
| | GNEDLAEILQKAAGSGGGGVYHREAQSG | |
| | KYKLTYAEAKAVCEFEGGHLATYKQLEAA | |
| | RKIGFHVCAAGWMAKGRVGYPIVKPGPN | |
| | CGFGKTGIIDYGIRLNRSERWDAYCYNPH | |
| | AGSGGHHHHHH | |
| NVS1b | SEQ ID NO: 171 | SEQ ID NO: 172 |
| | EVQLVESGGGLVQPGGSLRLSCTASGFS | GGGGGEIVMTQSPSTLSASVGDRVIIT |
| | LTDYYYMTWVRQAPGKGLEVVVGFIDPDD | CQASEIIHSWLAWYQQKPGKAPKLLIY |
| | DPYYATWAKGRFTISRDNSKNTLYLQMN | LASTLASGVPSRFSGSGSGAEFTLTIS |
| | SLRAEDTAVYYCAGGDHNSGWGLDIWG | SLQPDDFATYYCQNVYLASTNGANFG |
| | QGTLVTVSSASTKGPSVFPLAPSSKSTSG | QGTKLTVLKRTVAAPSVFIFPPSDEQL |
| | GTAALGCLVKDYFPEPVTVSWNSGALTS | KSGTASVVCLLNNFYPREAKVQWKVD |
| | GVHTFPAVLQSSGLYSLSSVVTVPSSSLG | NALQSGNSQESVTEQDSKDSTYSLSS |
| | TQTYICNVNHKPSNTKVDKRVEPKSCGS | TLTLSKADYEKHKVYACEVTHQGLSS |
| | GGGGVYHREAQSGKYKLTYAEAKAVCEF | PVTKSFNRGEC |
| | EGGHLATYKQLEAARKIGFHVCAAGWMA | |
| | KGRVGYPIVKPGPNCGFGKTGIIDYGIRLN | |
| | RSERWDAYCYNPHA | |
| NVS1c | SEQ ID NO: 173 | SEQ ID NO: 174 |
| | VYHREARSGKYKLTYAEAKAVCEFEGGH | VYHREARSGKYKLTYAEAKAVCEFEG |
| | LATYKQLEAARKIGFHVCAAGWMAKGRV | GHLATYKQLEAARKIGFHVCAAGWMA |
| | GYPIVKPGPNCGFGKTGIIDYGIRLNRSER | KGRVGYPIVKPGPNCGFGKTGIIDYGI |
| | WDAYCYNPHAKGGGSEVQLVESGGGLV | RLNRSERWDAYCYNPHAKGGGSEIV |
| | QPGGSLRLSCTASGFSLTDYYYMTVVVRQ | MTQSPSTLSASVGDRVIITCQASEIIHS |
| | APGKGLEVVVGFIDPDDDPYYATWAKGRF | WLAWYQQKPGKAPKLLIYLASTLASG |
| | TISRDNSKNTLYLQMNSLRAEDTAVYYCA | VPSRFSGSGSGAEFTLTISSLQPDDFA |
| | GGDHNSGWGLDIWGQGTLVTVSSASTK | TYYCQNVYLASTNGANFGQGTKLTVL |
| | GPSVFPLAPSSKSTSGGTAALGCLVKDYF | KRTVAAPSVFIFPPSDEQLKSGTASVV |
| | PEPVTVSWNSGALTSGVHTFPAVLQSSG | CLLNNFYPREAKVQWKVDNALQSGN |
| | LYSLSSVVTVPSSSLGTQTYICNVNHKPS | SQESVTEQDSKDSTYSLSSTLTLSKA |
| | NTKVDKRVEPKSCGS | DYEKHKVYACEVTHQGLSSPVTKSFN |
| | | RGEC |
| NVS1d | SEQ ID NO: 175 | SEQ ID NO: 176 |
| | EVQLVESGGGLVQPGGSLRLSCTASGFS | EIVMTQSPSTLSASVGDRVIITCQASEII |
| | LTDYYYMTWRQAPGKGLEVVVGFIDPDD | HSWLAWYQQKPGKAPKLLIYLASTLA |
| | DPYYATWAKGRFTISRDNSKNTLYLQMN | SGVPSRFSGSGSGAEFTLTISSLQPD |
| | SLRAEDTAVYYCAGGDHNSGWGLDIWG | DFATYYCQNVYLASTNGANFGQGTKL |
| | QGTLVTVSSASTKGPSVFPLAPSSKSTSG | TVLKRTVAAPSVFIFPPSDEQLKSGTA |
| | GTAALGCLVKDYFPEPVTVSWNSGALTS | SVVCLLNNFYPREAKVQWKVDNALQS |
| | GVHTFPAVLQSSGLYSLSSVVTVPSSSLG | GNSQESVTEQDSKDSTYSLSSTLTLS |
| | TQTYICNVNHKPSNTKVDKRVEPKSCGS | KADYEKHKVYACEVTHQGLSSPVTKS |
| | GGGGVYHREAQSGKYKLTYAEAKAVCEF | FNRGECGSGGGGVYHREAQSGKYKL |
| | EGGHLATYKQLEAARKIGFHVCAAGWMA | TYAEAKAVCEFEGGHLATYKQLEAAR |
| | KGRVGYPIVKPGPNCGFGKTGIIDYGIRLN | KIGFHVCAAGWMAKGRVGYPIVKPGP |
| | RSERWDAYCYNPHA | NCGFGKTGIIDYGIRLNRSERWDAYC |
| | | YNPHA |
| NVS1e | SEQ ID NO: 177 | SEQ ID NO: 178 |
| | EVQLVESGGGLVQPGGSLRLSCTASGFS | EIVMTQSPSTLSASVGDRVIITCQASEII |
| | LTDYYYMTWVRQAPGKGLEVVVGFIDPDD | HSWLAWYQQKPGKAPKLLIYLASTLA |
| | DPYYATWAKGRFTISRDNSKNTLYLQMN | SGVPSRFSGSGSGAEFTLTISSLQPD |
| | SLRAEDTAVYYCAGGDHNSGWGLDIWG | DFATYYCQNVYLASTNGANFGQGTKL |
| | QGTLVTVSSASTKGPSVFPLAPSSKSTSG | TVLKRTVAAPSVFIFPPSDEQLKSGTA |
| | GTAALGCLVKDYFPEPVTVSWNSGALTS | SVVCLLNNFYPREAKVQWKVDNALQS |
| | GVHTFPAVLQSSGLYSLSSVVTVPSSSLG | GNSQESVTEQDSKDSTYSLSSTLTLS |
| | TQTYICNVNHKPSNTKVDKRVEPKSCGS | KADYEKHKVYACEVTHQGLSSPVTKS |
| | | FNRGECGSGGGGVYHREAQSGKYKL |
| | | TYAEAKAVCEFEGGHLATYKQLEAAR |
| | | KIGFHVCAAGWMAKGRVGYPIVKPGP |
| | | NCGFGKTGIIDYGIRLNRSERWDAYC |
| | | YNPHAGSGGGGVYHREAQSGKYKLT |
| | | YAEAKAVCEFEGGHLATYKQLEAARKI |
| | | GFHVCAAGWMAKGRVGYPIVKPGPN |
| | | CGFGKTGIIDYGIRLNRSERWDAYCY |
| | | NPHA |
| NVS1f | SEQ ID NO: 179 | SEQ ID NO: 180 |
| | EVQLVESGGGLVQPGGSLRLSCTASGFS | VYHREARSGKYKLTYAEAKAVCEFEG |
| | LTDYYYMTWVRQAPGKGLEVVVGFIDPDD | GHLATYKQLEAARKIGFHVCAAGWMA |
| | DPYYATWAKGRFTISRDNSKNTLYLQMN | KGRVGYPIVKPGPNCGFGKTGIIDYGI |
| | SLRAEDTAVYYCAGGDHNSGWGLDIWG | RLNRSERWDAYCYNPHAKGGGSEIV |
| | QGTLVTVSSASTKGPSVFPLAPSSKSTSG | MTQSPSTLSASVGDRVIITCQASEIIHS |
| | GTAALGCLVKDYFPEPVTVSWNSGALTS | WLAWYQQKPGKAPKLLIYLASTLASG |
| | GVHTFPAVLQSSGLYSLSSVVTVPSSSLG | VPSRFSGSGSGAEFTLTISSLQPDDFA |
| | TQTYICNVNHKPSNTKVDKRVEPKSCGS | TYYCQNVYLASTNGANFGQGTKLTVL |
| | GGGGVYHREARSGKYKLTYAEAKAVCEF | KRTVAAPSVFIFPPSDEQLKSGTASVV |
| | EGGHLATYKQLEAARKIGFHVCAAGWMA | CLLNNFYPREAKVQWKVDNALQSGN |
| | KGRVGYPIVKPGPNCGFGKTGIIDYGIRLN | SQESVTEQDSKDSTYSLSSTLTLSKA |
| | RSERWDAYCYNPHA | DYEKHKVYACEVTHQGLSSPVTKSFN |
| | | RGEC |
| NVS1g | SEQ ID NO: 181 | SEQ ID NO: 182 |
| | EVQLVESGGGLVQPGGSLRLSCTASGFS | EIVMTQSPSTLSASVGDRVIITCQASEII |
| | LTDYYYMTWVRQAPGKGLEVVVGFIDPDD | HSWLAWYQQKPGKAPKLLIYLASTLA |
| | DPYYATWAKGRFTISRDNSKNTLYLQMN | SGVPSRFSGSGSGAEFTLTISSLQPD |
| | SLRAEDTAVYYCAGGDHNSGWGLDIWG | DFATYYCQNVYLASTNGANFGQGTKL |
| | QGTLVTVSSASTKGPSVFPLAPSSKSTSG | TVLKRTVAAPSVFIFPPSDEQLKSGTA |
| | GTAALGCLVKDYFPEPVTVSWNSGALTS | SVVCLLNNFYPREAKVQWKVDNALQS |
| | GVHTFPAVLQSSGLYSLSSVVTVPSSSLG | GNSQESVTEQDSKDSTYSLSSTLTLS |
| | TQTYICNVNHKPSNTKVDKRVEPKSCGS | KADYEKHKVYACEVTHQGLSSPVTKS |
| | GGGGVYHREARSGKYKLTYAEAKAVCEF | FNRGEC |
| | EGGHLATYKQLEAARKIGFHVCAAGWMA | |
| | KGRVGYPIVKPGPNCGFGKTGIIDYGIRLN | |
| | RSERWDAYCYNPHAGGGGGGSGVYHRE | |
| | ARSGKYKLTYAEAKAVCEFEGGHLATYK | |
| | QLEAARKIGFHVCAAGWMAKGRVGYPIV | |
| | KPGPNCGFGKTGIIDYGIRLNRSERWDAY | |
| | CYNPHAGSGGGGVYHREARSGKYKLTYA | |
| | EAKAVCEFEGGHLATYKQLEAARKIGFHV | |
| | CAAGWMAKGRVGYPIVKPGPNCGFGKT | |
| | GIIDYGIRLNRSERWDAYCYNPHAGSGGG | |
| | GVYHREARSGKYKLTYAEAKAVCEFEGG | |
| | HLATYKQLEAARKIGFHVCAAGWMAKGR | |
| | VGYPIVKPGPNCGFGKTGIIDYGIRLNRSE | |
| | RWDAYCYNPHA | |
| NVS1h | SEQ ID NO: 183 | SEQ ID NO: 184 |
| | EVQLVESGGGLVQPGGSLRLSCTASGFS | EIVMTQSPSTLSASVGDRVIITCQASEII |
| | LTDYYYMTWVRQAPGKGLEVVVGFIDPDD | HSWLAWYQQKPGKAPKLLIYLASTLA |
| | DPYYATWAKGRFTISRDNSKNTLYLQMN | SGVPSRFSGSGSGAEFTLTISSLQPD |
| | SLRAEDTAVYYCAGGDHNSGWGLDIWG | DFATYYCQNVYLASTNGANFGQGTKL |
| | QGTLVTVSSASTKGPSVFPLAPSSKSTSG | TVLKRTVAAPSVFIFPPSDEQLKSGTA |
| | GTAALGCLVKDYFPEPVTVSWNSGALTS | SVVCLLNNFYPREAKVQWKVDNALQS |
| | GVHTFPAVLQSSGLYSLSSVVTVPSSSLG | GNSQESVTEQDSKDSTYSLSSTLTLS |
| | TQTYICNVNHKPSNTKVDKRVEPKSCGS | KADYEKHKVYACEVTHQGLSSPVTKS |
| | GGGGVYHREARSGKYKLTYAEAKAVCEF | FNRGEC |
| | EGGHLATYKQLEAARKIGFHVCAAGWMA | |
| | KGRVGYPIVKPGPNCGFGKTGIIDYGIRLN | |
| | RSERWDAYCYNPHAGSGGGGVYHREAR | |
| | SGKYKLTYAEAKAVCEFEGGHLATYKQLE | |
| | AARKIGFHVCAAGWMAKGRVGYPIVKPG | |
| | PNCGFGKTGIIDYGIRLNRSERWDAYCYN | |
| | PHA | |
| NVS1j | SEQ ID NO: [[9]]215 | SEQ ID NO: 185 |
| | EVQLVESGGGLVQPGGSLRLSCTASGFS | EIVMTQSPSTLSASVGDRVIITCQASEII |
| | LTDYYYMTWVRQAPGKGLEVVVGFIDPDD | HSWLAWYQQKPGKAPKLLIYLASTLA |
| | DPYYATWAKGRFTISRDNSKNTLYLQMN | SGVPSRFSGSGSGAEFTLTISSLQP |
| | SLRAEDTAVYYCAGGDHNSGWGLDIWG | DDFATYYCQNVYLASTNGANFGQGT |
| | QGTLVTVSSASTKGPSVFPLAPSSKSTSG | KLTVLKRTVAAPSVFIFPPSDEQLKSG |
| | GTAALGCLVKDYFPEPVTVSWNSGALTS | TASVVCLLNNFYPREAKVQWKVDNAL |
| | GVHTFPAVLQSSGLYSLSSVVTVPSSSLG | QSGNSQESVTEQDSKDSTYSLSSTLT |
| | TQTYICNVNHKPSNTKVDKRVEPKSCGS | LSKADYEKHKVYACEVTHQGLSSPVT |
| | | KSFNRGECGSGGGGVYHREAQSGKY |
| | | KLTYAEAKAVCEFEGGHLATYKQLEA |
| | | ARKIGFHVCAAGWMAKGRVGYPIVKP |
| | | GPNCGFGKTGIIDYGIRLNRSERWDA |
| | | YCYNPHA |

### Example 14

### Further Characterization of the Peptide Tagged Proteins and Nucleic Acids of Example 13

Binding affinity of proteins, Fc Traps, full-length antibodies, DARPins, and scFvs fused with HA binding peptide tags were measured by Biacore as described in example 7.

### 14a: Biacore Affinity Determination

Affinity of peptide tagged proteins and the parental untagged protein were analyzed on Biacore to determine kinetics for their primary targets as described above in example 7(ex: Factor P, C5, TNFα, FGFR2, VEGF, Factor D, EPO, IL-17, IL-10R) as well as for HA binding. In order to determine HA kinetics, biotinylated HA was used in a BIOCAP Biacore format in which biotinylated HA is captured and the sample proteins flowed over at various concentrations. Biotinylated target ligands and biotinylated-HA were used in affinity measurements as described in Example 7: Biacore Affinity Determination.

### Target Kinetics and Affinity Using the Anti-Fab Method:

For the anti-Fab capture method, the Human Fab Capture® kit from GE® was used (GE 28958325). Refer to the catalog number more detailed information. For this method, HBS-EP+ running buffer (teknova H8022) was used. A CM5 chip (GE®, BR-1005-30) was used and to this the anti-Fab polyclonal was immobilized to achieve approximately 5,000 RU according to the GE® protocol. Refer to the catalog number on the GE® website to get more detailed information. Two flow cells were used for this method. Flow cell 1 served as the reference cell which only contained the immobilized anti-fab reagent and flow cell 2 served as the binding cell which contained both the anti-fab reagent and the protein samples. The protein samples tested in this method were against C5, Factor P and EPO specific. The protein samples were captured at a flow rate of 10 ul/min for a specific contact time in order to achieve an RU signal for an Rmax of 20. Since the protein analytes have strong affinities for their targets, the starting concentrations of the target analytes started at approximately 10 nM and would include 8 serial dilution points. The target analytes were flowed over at 60 ul/min for 240 seconds with short and longer dissociations times greater than 1000 seconds depending on the sample.

TABLE 10
Binding affinity of various peptide tagged molecules. Both HA and protein
target binding was measured.

| NVS ID | Ligand | ka (1/M * s) | kd (1/s) | Affinity (M) | Temperature |
|---|---|---|---|---|---|
| NVS72T | 17 kDa HA-biotin | 7.88E+05 | 2.82E−01 | 3.58E−07 | 25° C. |
| | EPO-biotin | 1.06E+07 | 2.44E−04 | 2.29E−11 | 25° C. |
| NVS70T | 17 kDa HA-biotin | 4.21E+06 | 1.91E−01 | 4.54E−08 | 25° C. |
| | C5-biotin | 1.52E+07 | 2.22E−05 | 1.46E−12 | 25° C. |
| NVS71T | 17 kDa HA-biotin | 2.42E+06 | 1.15E−01 | 4.76E−08 | 25° C. |
| | Factor P-biotin | 4.31E+06 | 4.12E−04 | 9.55E−11 | 25° C. |
| NVS73T | 17 kDa HA-biotin | 1.03E+07 | 8.75E−01 | 8.51E−08 | 25° C. |
| | TNFα-biotin | 3.33E+07 | 2.47E−05 | 7.42E−13 | 37° C. |
| NVS77T | 17 kDa HA-biotin | 1.45E+06 | 9.42E−02 | 6.49E−08 | 25° C. |
| | FGFR2-biotin | 5.44E+07 | 1.21E−02 | 2.22E−10 | 25° C. |
| NVS76T | 17 kDa HA-biotin | 4.82E+06 | 2.19E−01 | 4.54E−08 | 25° C. |
| | IL17A-biotin | 4.51E+06 | 3.46E−05 | 7.68E−12 | 25° C. |
| NVS75T | 17 kDa HA-biotin | 5.43E+04 | 6.76E−02 | 1.25E−06 | 25° C. |
| | IL1β-biotin | 4.95E+06 | 6.90E−05 | 1.40E−11 | 25° C. |
| NVS78T | 17 kDa HA-biotin | 3.42E+06 | 9.35E−04 | 2.73E−10 | 25° C. |
| | EPO-biotin | 1.52E+07 | 2.73E−04 | 1.80E−11 | 25° C. |
| NVS74T | 17 kDa HA-biotin | 3.93E+05 | 3.05E−01 | 7.76E−07 | 25° C. |
| | Factor D-biotin | 1.01E+07 | 9.54E−05 | 9.42E−12 | 25° C. |
| NVS90T | 17 kDa HA-biotin | 1.51E+07 | 3.45E−01 | 2.29E−08 | 25° C. |
| | NVS78 | 1.34E+07 | 1.02E−03 | 7.63E−11 | 25° C. |
| NVS91T | 17 kDa HA-biotin | 1.87E+07 | 6.36E−01 | 3.40E−08 | 25° C. |
| NVS79T | 17 kDa HA-biotin | 8.35E+02 | 2.54E−03 | 3.04E−06 | 25° C. |

All Fabs and proteins linked with the HA-binding peptide tag exhibited similar HA binding affinity and retained binding to their primary target (Table 10). In fact, the presence of the peptide tag improved the molecule's primary target binding affinity compared to the untagged molecule (see Example 15b).

TABLE 11
HA and VEGF Binding Affinity of peptide tagged molecules.

| NVS ID | Ligand | ka (1/M * s) | kd (1/s) | Affinity | Temperature |
|---|---|---|---|---|---|
| NVS80T | 17 kDa HA-biotin | 1.94E+06 | 1.17E−03 | 6.03E−10 | 37° C. |
| | hVEGF-biotin | 2.71E+08 | 9.06E−05 | 3.35E−13 | 37° C. |
| NVS81T | 17 kDa HA-biotin | 6.49E+05 | 2.29E−04 | 3.52E−10 | 37° C. |
| | hVEGF-biotin | 2.07E+07 | 1.75E−04 | 8.46E−12 | 37° C. |
| NVS82T | 17 kDa HA-biotin | 2.53E+06 | 5.00E−03 | 1.97E−09 | 37° C. |
| | hVEGF-biotin | 8.52E+07 | 3.19E−05 | 3.75E−13 | 37° C. |
| NVS83T | 17 kDa HA-biotin | 2.11E+05 | 3.75E−01 | 1.78E−06 | 25° C. |
| | hVEGF-biotin | 8.12E+07 | 8.49E−05 | 1.05E−12 | 25° C. |
| NVS84T | 17 kDa HA-biotin | 1.93E+07 | 1.93E−01 | 9.96E−09 | 25° C. |
| | hVEGF-biotin | 1.59E+07 | 1.26E−03 | 7.95E−11 | 25° C. |
| NVS85T | 17 kDa HA-biotin | 6.25E+06 | 6.96E−02 | 1.11E−08 | 25° C. |
| | hVEGF-biotin | 1.18E+07 | 1.10E−04 | 9.37E−12 | 25° C. |
| NVS1c | 17 kDa HA-biotin | 4.38E+06 | 1.94E−03 | 4.42E−10 | 25° C. |
| | hVEGF-biotin | 2.13E+06 | 2.83E−05 | 1.33E−11 | 25° C. |
| NVS1c | 17 kDa HA-biotin | 1.06E+06 | 1.11E−02 | 1.05E−08 | 37° C. |
| | hVEGF-biotin | 2.03E+06 | 2.81E−05 | 1.39E−11 | 37° C. |
| NVS1d | 17 kDa HA-biotin | 2.18E+06 | 7.41E−04 | 3.40E−10 | 25° C. |
| | hVEGF-biotin | 1.70E+07 | 2.36E−05 | 1.39E−12 | 25° C. |
| NVS1d | 17 kDa HA-biotin | 6.41E+06 | 2.82E−03 | 4.40E−10 | 37° C. |
| | hVEGF-biotin | 9.78E+06 | 2.58E−05 | 2.63E−12 | 37° C. |
| NVS1e | 17 kDa HA-biotin | 2.44E+06 | 1.24E−01 | 5.08E−08 | 37° C. |
| | hVEGF-biotin | 1.40E+07 | 1.26E−05 | 9.00E−13 | 37° C. |
| NVS1f | 17 kDa HA-biotin | 3.68E+06 | 4.31E−03 | 1.17E−09 | 25° C. |
| | hVEGF-biotin | 5.63E+06 | 2.79E−05 | 4.95E−12 | 25° C. |
| NVS1f | 17 kDa HA-biotin | 5.91E+05 | 2.19E−02 | 3.71E−08 | 37° C. |
| | hVEGF-biotin | 5.23E+06 | 2.43E−05 | 4.64E−12 | 37° C. |
| NVS1g | 17 kDa HA-biotin | Binding, but cannot be analyzed | | | 25° C. |
| | hVEGF-biotin | 6.14E+05 | 25° C. | 5.46E−12 | 25° C. |
| NVS1h | 17 kDa HA-biotin | 1.95E+05 | 4.08E−04 | 2.09E−09 | 25° C. |
| | hVEGF-biotin | 1.57E+07 | 8.76E−06 | 5.58E−13 | 25° C. |
| NVS1j | 17 kDa HA-biotin | 2.65E+05 | 3.72E−01 | 1.40E−06 | 25° C. |
| | hVEGF-biotin | 3.05E+07 | 1.72E−04 | 5.64E−12 | 25° C. |

All Fabs and proteins linked with the HA-binding peptide tag exhibited similar HA binding affinity and retained binding to their primary target (Table 10). In fact, the presence of the peptide tag improved the molecule's primary target binding affinity compared to the untagged molecule (see Example 15b).

### 14b: Rabbit Traditional Ocular PK Determination

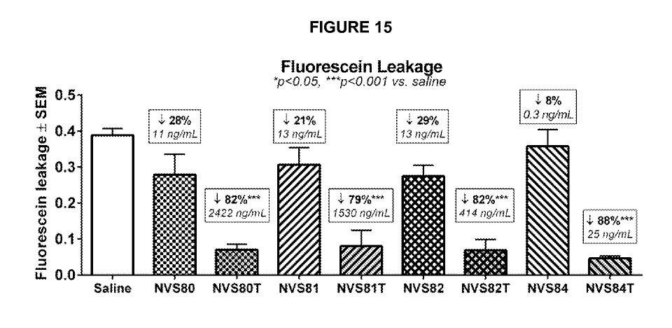

Ocular terminal copncentrations of antibodies, Fc traps, and proteins linked to an HA-binding peptide tag in rabbit vitreous were compared to their untagged versions using standard methods as described belowand shown in FIG. 15 and Table 12.

5 μg/eye (˜105 pmoles) un-tagged antibodies and 6.2 ug/eye (˜105 pmoles) of tagged antibodies were injected intravitreally into rabbit eyes (N=6 eyes per antibody). Rabbits were sacrificed 21 days after injection and eyes were enucleated. The enucleated eyes were dissected and the vitreous was separated from other tissues and further homogenized mechanically using a TissueLyzer (QIAGEN®). Antibody levels in the vitreous were measured by ELISA or mass spectrometry.

### ELISA Method

The Maxisorp 384 well plates (Nunc 464718) were coated with a Goat Anti-Human IgG (H+L) (Thermo Fisher 31119) in carbonate buffer (Pierce 28382) overnight at 4 C. In between incubations, plates were washed 3 times with TBST (THERMO SCIENTIFIC® 28360) using a BioTek plate washer. The next day, the plates were blocked for 2 hours at room temperature (or overnight at 4 C) with blocking buffer (5% BSA (SIGMA® A4503), 0.1% Tween-20 (SIGMA® P1379), 0.1% Triton X-100 (SIGMA® P234729) in TBS. Samples were diluted in diluent (2% BSA (SIGMA® A4503), 0.1% Tween-20 (SIGMA® P1379), 0.1% Triton X-100 (SIGMA® P234729) in TBS). Samples were incubated on the plate for 1 hour at room temperature with gentle shaking. The detection antibody was a Goat Anti-Human IgG [F(ab′)2]) conjugated to HRP (Thermo Fisher 31414). The detection antibody was added to the plates for 30 minutes at room temperature with gentle shaking. Ultra TMB is added for 15 minutes (Thermo Fisher 34028). The reaction was quenched with 2N sulfuric acid (Ricca 8310-32). The absorbance of the samples was read on the SpectraMax (450-570 nm). To back-calculate Fab recovery levels from eye tissues, a purified standard was used. For the standard, the top concentration used was 200 ng/mL with 2-fold dilutions. Different pairs of antibodies can be used for Fab recovery from rabbit tissues.

### ELISA Method for NVS90 and NVS90T

Assays were performed using standard binding MSD plates (Meso-Scale Discovery®, 384-well: MSD cat#L21XA), using coating buffer (PBS) and incubation buffer (PBS with 2% BSA (Sigma cat#A4503) and 0.1% Tween-20 and 0.1% Triton-X). Capture antibody, EP026 (Cell Sceinces, Cat #26G9C10) was coated at 1 μg/ml in PBS (25 μl), and incubated overnight at 4° C. Plates were washed 3× in wash buffer (PBS with 0.05% Tween-20), and blocked with 25 μl incubation buffer at RT for 2 hrs. Plates were washed 3× in wash buffer. Vitreous dilutions in incubation buffer were added to the plate (25 μl), and incubated for 60 min at room temperature. Human recombinant Darbepoietin was used as a standard (11096-26-7, A000123, starting at 5 μg/ml) for Darbepoietin samples. NVS90T was used as a standard (starting at 5 ug/ml) for NVS90T samples. Plates were washed 3× in wash buffer. 25 μl primary antibody was added (1 μg/ml in incubation buffer), and incubated at room temperature for 60 min. Plates were washed 3× in wash buffer. 25 μl of anti-species secondary Sulfo-TAG antibody (MSD Cat # R32AJ-1) was added (1:1000 in incubation buffer), and incubated at RT for 60 min. Plates were washed 3× in wash buffer, and 25 μl of 1×MSD Read buffer T was added (with surfactant, MSD cat#R92TC-1). Plates were read on a MSD Spector Imager 6000®.

### ELISA Method for NVS78 and NVS78T

Assays were performed using 384 well MaxiSorp ELISA plate (Thermo Scientific, 464718), using Carbonate-Bicarbonate coating buffer (made by using BuPH Carbonate-Bicarbonate buffer Packs, Thermo Scientific®, 28382), blocking buffer (TBST with 5% BSA (Sigma, A4503) and diluent buffer (TBST with 2% BSA). Streptavidin (Rockland®, S000-01) was coated at 1 μg/ml in coating buffer (20 ul/well), and incubated overnight at 4° C. Plates were washed 3× in wash buffer (PBS with 0.05% Tween-20), and blocked with blocking buffer (50 ul/well) at RT for 2 hrs. Plates were washed 3× in wash buffer. 1 ug/ml huEpo-biotin (Novartis) in diluent were added to plate (20 ul/well), and incubated for 1 hr at RT. Plates were washed 3× in wash buffer. Vitreous dilutions in diluent were added to the plate (20 μl/well). EpoR or EpoR-HA (Novartis) was used as a standard starting at concentration of 1 ug/ml. Incubate plates at RT for 1 hr. Plates were washed 3× in wash buffer. 20 μl detection antibody (goat anti-human Fc-HRP, Thermo Scientific®, Cat #31413) was added (1:5000 in diluent) to the plate, and incubated at RT for >30 min. Plates were washed 3× in wash buffer. 20 ul of 1-step Ultra TMB substrate solution (Pierce®, 34028) was added. When solution color in positive wells turn into dark blue, add 10 ul of 2N sulfuric acid stop solution (RICCA, 8310-32) into each well to stop the reaction. Plates were read immediately on spectrometer plate reader (Molecular Device®, SpectroMax PLUS 384) at OD 450-570 nm.

### Mass Spectrometry Method

### Reduction, Alkylation and Digestion:

60 uL of vitreous sample in each well was thawed at room temperature for 10 minutes. 150 uL of 8M Urea (FisherScientific®, Cat No. U15-500) in 50 mM Tris-HCl (Fisher Scientific®, BP153-500) was added to each sample well, followed by addition of 4 uL of 2M DTT (SigmaAldrich®, Cat. No. D9779) to a final concentration of 40 mM DTT. The plate was heated at 58 deg C. for 45 minutes to denature the proteins. Subsequently, cool the plate to room temperature, then add 8 uL of 1M Iodoacetamide (SigmaAldrich®, Cat. No. 11149) for a final concentration of 40 mM and incubate at room temperature for 45 minutes in the dark. Dilute final concentration of urea to below 2M by adding 1.3 mL of 50 mM ammonium bicarbonate (Fisher Scientific®, Cat. No. BP2413-500). Add 10 uL of 0.1 ug/uL trypsin (Promega®, Cat. No. V5111) and incubate at 37° C. overnight.

### SPE Cleanup and Filtration:

After digestion, add formic acid (Fluka, Cat. No. 56302-50ML-F) to each sample to a final concentration of 1% (v/v) to quench trypsin digestion. Oasis® MCX plate (Waters, Cat. No. 186000259) is used to clean up the digested sample. The collected sample solution from cleanup was dried down completely using SpeedVac (ThermoFisher Savant). Once the sample is dried, 60 uL of buffer (0.1% formic acid, 1% ACN (Sigma Aldrich, Cat. No. 34998-4L) and 20 pg/uL heavy labeled internal standard (custom made by ThermoFisher) solution is added to each well, and the plate was shaked for 20 minutes. The reconstituted peptide solution was filtered using AcroPrep™ advanced 96-well filter plates for ultrafiltration (Pall Life Sciences, Cat. No. 8164) filter with 10 KDa MWCO.

### LC-MS/MS Analysis:

5 uL of each filtered samples was loaded to a 300 um×150 mm Symmetry® C18 column (Waters®, Cat. No. 186003498). Separation was achieved by applying a 5 min gradient from 5% B (acetonitrile in 0.1% formic acid) to 20% B with a flow rate of 5 uL/min. Two peptides (HC_T3: GPSVFPLAPSSK (SEQ ID NO: 210) and DDA2: TGIIDYGIR (SEQ ID NO: 211)), and two transitions for each peptide (HC_T3: 594.19/699.82 and 594.19/847; DDA2: 504.58/623.68 and 504.58/736.84) were monitored for each sample using Waters Xevo TQS mass spectrometer (Waters). For Eylea and Eylea containing constructs, two transitions (560.28/697.76 and 560.28/709.28) from FNWYVDGVEVHNAK (SEQ ID NO: 212) were monitored on the same mass spectrometer using the same LC columns and conditions. Drug molecules containing these peptides were quantified using MS signals resulted from these transitions.

### Gyrolab Method

### Sample Preparation

Vitreous samples were thawed at room temperature for 10 minutes. 5 uL of vitreous sample is then diluted 1:2 in Rexxip AN Buffer (Gyros AB®, Inc. Cat P0004994) in a 96-well PCR plate (Thermo Scientific® AB-800, 0.2 mL Skirted 96-well PCR plate). Samples were sealed (Gyros AB®, Inc. microplate foil Cat P0003313) and mixed thoroughly in a plate shaker for 1 minute. Ensuring that no bubbles are found in the bottom of the wells, the samples were placed in the Gyrolab™ xP workstation. A 3-step C-A-D method is executed on the Gyrolab™ xP workstation; capture antibody is flowed through the system first, followed by the analyte (samples), and then the detector antibody. The Gyrolab™ xP workstation performs washes of PBS 0.01% Tween20 (Calbiochem®, Inc. Cat 655206) in between each step. The standard curve for free Fc drug measurement was prepared in a diluent containing 50% rabbit vitreous (BioReclamation®, LLC. Cat Rabb-Vitreous) in Rexxip AN. The standard was serially diluted 1:6 from 6000 ng/mL to 0.129 ng/mL. The standard curve for Fab drug measurement was prepared in a diluent containing 10% rabbit vitreous (BioReclamation®, LLC. Cat Rabb-Vitreous) in Rexxip AN. The standard was serially diluted 1:6 from 6000 ng/mL to 0.129 ng/mL.

### Detection of Fabs

Total and free purified drug constructs were analyzed in the Gyrolab™ xP workstation using a Bioaffy1000 CD (Gyros AB, Inc. Cat P0004253). Gyros AB

Free drug is measured by applying 100 ug/mL biotin-labeled VEGF (Novartis) to a column containing streptavidin coated particles. Vitreous samples are applied to the activated columns and detected by capillary action with 25 nM alexafluor-647 labeled goat anti-Human IgG-heavy and light chain antibody (Bethyl Laboratories®, Cat A80-319A). Note that alexafluor labeling was performed using Life Technologies labeling kit (Cat A-20186). The capture reagent was prepared in PBS 0.01% Tween20 and the detector reagent in Rexxip F (Gyros AB®, Inc. P0004825).

Total drug is measured by applying 100 ug/mL biotin-labeled goat anti-Human IgG-heavy and light chain antibody (Bethyl Laboratories®, Cat A80-319B). Vitreous samples are applied to the activated columns and detected by capillary action with 10 nM alexafluor-647 labeled goat anti-Human IgG-heavy and light chain antibody (Bethyl Laboratories®, Cat A80-319A).

### Detection of Fc Proteins

Total and free purified drug constructs were analyzed in the Gyrolab™ xP workstation using a Bioaffy1000 CD (Gyros AB, Inc. Cat P0004253). Free drug is measured by applying 100 ug/mL biotin-labeled VEGF (Novartis) to a column containing streptavidin coated particles. Vitreous samples are applied to the activated columns and detected by capillary action with 25 nM alexafluor-647 labeled anti-Human Fc-specific antibody (R10, Novartis). Total drug is measured by applying 25 ug/mL biotin-labeled goat anti-Human IgG-heavy and light chain antibody (Bethyl Laboratories®, Cat A80-319B). Vitreous samples are applied to the activated columns and detected by capillary action with 12.5 nM alexafluor-647 labeled goat anti-Human IgG-heavy and light chain antibody (Bethyl Laboratories, Cat A80-319A).

### Detection of DARPins

Free purified drug constructs were analyzed in the Gyrolab™ xP workstation using a Bioaffy1000 CD (Gyros AB, Inc. Cat P0004253). Free drug is measured by applying 25 ug/mL biotin-labeled VEGF (Novartis) to a column containing streptavidin coated particles.

Vitreous samples are applied to the activated columns and detected by capillary action with 6.25 nM alexafluor-647 labeled Penta HIS (SEQ ID NO: 213) antibody (Qiagen®, Cat 35370).

TABLE 12
Terminal vitreal concentrations and calculated 2-point ocular
half-life (t½) values.

| | Terminal | | | |
|---|---|---|---|---|
| | vitreal conc. | 2-point | Terminal | 2-point |
| | by ELISA | ocular t_{1/2} | vitreal conc. | ocular t_{1/2} |
| NVS ID | (ng/ml) | (days) | by MS (ng/ml) | (days) |
| NVS70T | 710 | 6.73 | 792 | 7.1 |
| NVS71T | 432 | 5.46 | 607 | 6.2 |
| NVS73T | 212 | 4.3 | 223 | 4.4 |
| NVS77T | 357 | 5.1 | 2553 | 16.5 |
| NVS76T | 92.5 | 3.47 | 466 | 5.6 |

| | | | |
|---|---|---|---|
| NVS72T | n/a | 108 | 3.59 |
| NVS74T | n/a | 969 | 7.88 |

| | | | |
|---|---|---|---|
| NVS90T | 145 | 1.15 | Not done |
| NVS78T | 267 | 3.71 | Not done |

| | | | |
|---|---|---|---|
| NVS77T | n/a | 2553 | 16.5 |

Molecules without HA-binding peptide tag

| | | | | |
|---|---|---|---|---|
| NVS77 | 0.1 | 1.35 | 20 | 2.6 |
| NVS73 | 12 | 2.41 | 20 | 2.6 |

| NVS79 | Not done | | Not done |
|---|---|---|---|
| NVS90 | 17.2 | 0.54 | Not done |

Fusing an HA-binding peptide tag (SEQ ID #33) to antigen binding fragments including NVS70, NVS71, NVS72, NVS73, NVS74, NVS75, NVS76, and NVS77, etc., Fc trap proteins NVS78 and NVS80, etc. and proteins NVS84 and NVS90, etc., resulted in higher ocular terminal concentrations of these molecules as compared to untagged Fabs and proteins. These data indicate that the fusion of the HA-binding peptide tag confers improvement in and ocular half-life (t½) independent of the molecule it is fused to. Consequently, fusion of an HA-binding peptide tag appears to universally increase the ocular retention and ocular half-life of molecules administered intravitreally.

### 14c: Rabbit Duration of Efficacy

The rabbit leakage model was used to assess whether engineering VEGF binding biologics to bind HA could inhibit vessel leakage at 20 days post-injection (FIG. 15). In this study, various anti-VEGF molecules tagged with HA-binding peptide tags were administered at an equimolar dose to their respective parental molecules 18 days prior to the hVEGF. Forty-eight hours post hVEGF challenge, fluorescein leakage was assessed as described above. NVS80T, NVS81T, NVS82T, and NVS84T, which are fusions of untagged proteins NVS80, NVS81, NVS82, and NVS84 with an HA-binding peptide tag (e.g.: SEQ ID NO: 33) had significant efficacy at day 20 as compared to their untagged parent molecules. Animals were sacrificed the day after imaging, eyes enucleated, processed, and free drug (not bound to VEGF) levels measured by Gyrolab as described above. Free terminal vitreal concentrations of NVS80T, NVS81T, NVS82T, and NVS84T ranged from 25 ng/ml to 2422 ng/ml and were 31-220 fold higher than their untagged parental molecules NVS80, NVS81, NVS82, and NVS84 (FIG. 15).

These data indicate that fusion of the HA-binding tag confers improvement in ocular retention and efficacy duration independent of the molecule it is fused to. Addition of an HA-binding moiety increased fluorescein inhibition versus all respective parental molecules. Thus, the amount of HA-tagged construct in the vitreous was sufficient to suppress hVEGF and block vessel leakage while the amount of untagged parental molecule was not.

TABLE 13
Doses utilized in Example 15c

| NVS ID | Dose (pmoles/eye) |
|---|---|
| NVS81 | 105 |
| NVS81T | 105 |
| NVS82 | 105 |
| NVS82T | 105 |
| NVS80 | 105 |
| NVS80T | 105 |
| NVS84 | 315 |
| NVS84T | 315 |

The increase in duration of efficacy indicates that binding to hyaluronic-acid in the eye, reduces clearance from the eye leading to higher protein levels at later time points and suppression of VEGF for longer duration. In human wet AMD patients, suppression of VEGF levels is necessary to prevent recurrence of neovascularization activity, and increases in VEGF levels correlate with the return of disease activity (Muether et al., 2012). Thus, treatment of a wet AMD patient with an HA-binding anti-VEGF antibody or protein is expected to have longer duration of action compared to an unmodified anti-VEGF antibody or protein, thereby benefiting patients by maintaining efficacy while providing a reduction in dosing frequency. These experiments demonstrate that the HA-binding peptide tags of the invention can be used to extend the half-life, increase the terminal concentration, decrease the clearance, and increase the mean residence time of an anti-VEGF protein drug in the vitreous.

### 14d: Day 20 Duration of Efficacy and Terminal PK in Rabbits for NVS1 and NVS1d

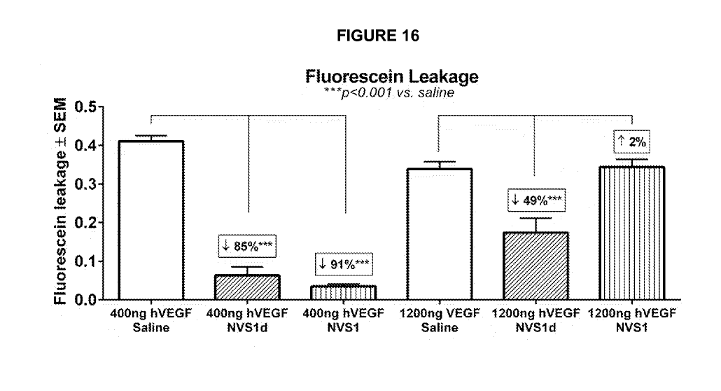

The rabbit leakage model was used to assess whether a molecule with two HA-binding moieties (NVS1d) would increase efficacy versus a singly-tagged construct (NVS1) (FIG. 16). The amount of VEGF was increased from 400 ng/eye to 1200 ng/eye in half of the study groups in order to test molecules with an increased half life without requiring an increase in study duration. Equimolar amounts of NVS1 and NVS1d (both equimolar to 5 μg/eye ranibizumab) were intravitreally administered 18 days prior to the hVEGF challenge. Half of the groups received 1200 ng/eye hVEGF while the remainder received 400 ng/eye hVEGF as in previously described examples. Forty-eight hours post hVEGF challenge, fluorescein leakage was assessed as described above. NVS1 and NVS1d both achieved similar efficacy at day 20 with a 400 ng/eye hVEGF injection (85-91% leakage inhibition). In NVS1d groups challenged with 1200 ng/eye hVEGF, significant inhibition of fluorescein leakage was achieved (49%). In contrast, NVS1 groups challenged with 1200 ng/eye hVEGF were not efficacious (−2%).

In general, terminal vitreal concentrations of rabbits injected with NVS1 challenged with 400 ng of hVEGF 18 days post-dosing ranged between 598 and 953 ng/ml, while terminal vitreal concentrations of rabbits injected with NVS1d challenged with 400 ng of hVEGF 18 days post-dosing ranged between 1048 and 3054 ng/mL. Thus the amount of NVS1d in the vitreous was sufficient to suppress an increased amount of hVEGF in the vitreous compared to that achieved with NVS1. These data indicate that an antibody with two HA-binding peptide tags (NVS1d) that has higher affinity for HA has a significantly longer duration of efficacy compared to an antibody that only has one HA-binding peptide tag (NVS1).

### Example 15

### Biophysical Properties of HA Binding Peptide Tagged Molecules

### 15a: Improvement of Isoelectric Point and Solubility

Linking the HA-binding tag to proteins of various types (e.g.: scFvs, Fabs, IgGs, and Fc traps) increased the overall isolectric point and solubility of the parent proteins to which the HA-binding tag was linked. Table 14 shows the isolectric points of the naked untagged proteins along with the isoelectric points of the same protein linked with the HA-binding peptide tag.

The HA-binding peptide tag also increased the affinity of the protein to its primary target/ligand. Table 15 shows the affinity of various proteins for their primary target/ligand compared with the affinity of these same proteins linked to the HA-binding tag. Surprisingly, proteins linked to the HA-binding tag had 1.2-75-fold increase in affinity for the primary ocular protein target/ligand compared to the parent protein without the HA-binding tag.

TABLE 14
Isoelectric points of proteins compared to proteins linked to the
HA-binding peptide tag

| NVS ID | Calculated isoelectric point | Increase |
|---|---|---|
| NVS4 | 6.67 | 1.72 |
| NVS3 | 8.39 | |
| NVS4 | 6.67 | 1.83 |
| NVS1 | 8.5 | |
| NVS4 | 6.67 | 1.72 |
| NVS2 | 8.39 | |
| NVS71 | 7.93 | 0.74 |
| NVS71T | 8.67 | |
| NVS73 | 8.6 | 0.27 |
| NVS73T | 8.87 | |
| NVS81 | 8.03 | 0.5 |
| NVS81T | 8.53 | |
| NVS82 | 6.96 | 1.27 |
| NVS82T | 8.23 | |
| NVS84 | 5.16 | 1.22 |
| NVS84T | 6.38 | |
| NVS80 | 8.2 | 0.27 |
| NVS80T | 8.47 | |
| NVS83 | 4.93 | 2.8 |
| NVS83T | 7.73 | |
| NVS72 | 8.19 | 0.53 |
| NVS72T | 8.72 | |
| NVS70 | 6.44 | 1.93 |
| NVS70T | 8.37 | |
| NVS74 | 6.51 | 1.87 |
| NVS74T | 8.38 | |

### 15b: Ocular Target Binding

TABLE 15
Target ligand binding affinity of a protein compared to
the same protein linked to a peptide tag that binds HA.

| | | Temp | ka | kd | Affinity | Fold |
|---|---|---|---|---|---|---|
| Ligand | NVS ID | (° C.) | (1/M*s) | (1/s) | (M) | improvement |
| Hu factor | NVS71 | 37 | 2.96E+06 | 1.61E−03 | 5.43E−10 | 5.7 |
| P-biotin | NVS71T | 37 | 4.31E+06 | 4.12E−04 | 9.55E−11 | |
| Hu TNFα- | NVS73 | 37 | 3.08E+06 | 8.01E−05 | 2.60E−11 | 35 |
| biotin | NVS73T | 37 | 3.33E+07 | 2.47E−05 | 7.42E−13 | |
| Hu VEGF- | NVS81 | 37 | 2.79E+05 | 1.78E−04 | 6.39E−10 | 75.5 |
| biotin | NVS81T | 37 | 2.07E+07 | 1.75E−04 | 8.46E−12 | |
| | NVS82 | 37 | 3.06E+06 | 2.34E−05 | 7.64E−12 | 20.3 |
| | NVS82T | 37 | 8.52E+07 | 3.19E−05 | 3.75E−13 | |
| | NVS84 | 25 | 4.01E+06 | 1.68E−03 | 4.19E−10 | 5.2 |
| | NVS84T | 25 | 1.59E+07 | 1.26E−03 | 7.95E−11 | |
| | NVS80 | 37 | 2.62E+06 | 1.15E−04 | 4.40E−11 | 131 |
| | NVS80T | 37 | 2.71E+08 | 9.06E−05 | 3.35E−13 | |
| | NVS83 | 25 | 1.36E+06 | 9.18E−06 | 6.75E−12 | 6.4 |
| | NVS83T | 25 | 8.12E+07 | 8.49E−05 | 1.05E−12 | |
| Hu EPO- | NVS72 | 25 | 3.34E+06 | 1.25E−04 | 3.75E−11 | 3.2 |
| biotin | NVS72T | 25 | 1.15E+07 | 1.31E−04 | 1.14E−11 | |
| Hu EPOR- | NVS78 | 25 | 9.31E+05 | 5.17E−04 | 5.56E−10 | 41 |
| Fc-biotin | NVS78T | 25 | 3.24E+07 | 4.36E−04 | 1.35E−11 | |
| | NVS90 | 25 | 1.05E+07 | 1.41E−03 | 1.35E−10 | 1.76 |
| | NVS90T | 25 | 1.34E+07 | 1.02E−03 | 7.63E−11 | |
| Hu C5- | NVS70 | 25 | 2.05E+06 | 3.96E−05 | 1.93E−11 | 13.2 |
| biotin | NVS70T | 25 | 1.52E+07 | 2.22E−05 | 1.46E−12 | |

These results clearly demonstrate that linking a peptide tag that binds HA to an antigen binding fragment, full-length antibody, Fc trap, DARPin, scFvs, and proteins increases the affinity of that protein molecule for its main target, for example, VEGF. This is an unexpected property as the HA-binding peptide tag which is spatially quite far from the target binding regions of these anti-VEGF proteins.

### Example 16

### Biodistribution of I-124 Labeled Ranibizumab and HA-Tagged Antibody in Rats

The biodistribution of ranibizumab and an antibody tagged with an HA-binding peptide of the invention (NVS1) were measured using I-124 labeled proteins as described below. The results demonstrate that the HA-binding peptide tags are useful for extending the duration of action of ocular therapies, without any significant effect on clearance in extra-ocular environments.

Radiolabeling of the proteins that were injected in rat eyes was performed using the lodogen method (1), which employs the use of iodogen coated tubes (Thermo Scientific, Rockford, Ill.). Typically, a radiolabeling efficiency >85% and a specific activity of approximately 7 mCi/mg were achieved. To prepare rats for intravitreal (IVT) injections, the animals were anesthetized with 3% isoflurane gas. The eyes were then dilated with two drops of Cyclopentolate (1% preferred concentration) and 2.5-10% Phenylephrine. A drop of local anesthetic was also applied (0.5% Proparacaine). Under a dissecting microscope, an incision was made with a 30 gauge needle approximately 4 mm below the limbus of the cornea with the angle directed towards the middle of the eye. A blunt end Hamilton syringe (e.g. 33 gauge) containing the radioactively labeled protein was then inserted through this opening into the vitreous cavity and approximately 3.5 μL of radiolabeled protein was injected. The eye was examined for hemorrhage or cataract. The procedure was then repeated on the fellow eye. Immediately after injecting the radiolabeled protein into a rat eye, the anesthetized animal was placed on the preheated PET imaging bed, lying on its abdomen. The bed was supplied with a nose cone for gas anesthesia. The immobilized and secured animal was then moved in the scanner with vital functions (e.g. respiration) being monitored using a breathing sensor placed under the animal's chest. For the animals injected with I-124 labeled ranibizumab, animals were euthanized 72 hours post-IVT injection by cardiac puncture, exsanguinations, and cervical dislocation. Eyes and other organs/tissues (blood, liver, spleen, kidneys, stomach, lungs, heart, muscle, and bone) were dissected out and counted for remaining radioactivity in a gamma counter. Counts were converted to % injected dose/gram (% ID/g) of the counted tissue/organ. For the animals injected with I-124 labeled HA-tagged antibody (NVS1), animals were euthanized 72 hours post-IVT injection by cardiac puncture, exsanguinations, and cervical dislocation. Eyes and other organs/tissues (blood, liver, spleen, kidneys, stomach, lungs, heart, muscle, and bone) were dissected out and counted for remaining radioactivity in a gamma counter. Counts were converted to % injected dose/gram (% ID/g) of the counted tissue/organs.

Immediately after the last PET/CT imaging time point, rats were euthanized and blood was collected via cardiac puncture. Blood was withdrawn from the animals in order to reduce the amount of blood associated radioactivity trapped in organs and tissues. Individual organs and tissues including left eye, right eye, blood, liver, spleen, kidneys, lungs, heart, muscle, stomach, bone and brain were dissected, weighed and counted for remaining radioactivity in a gamma counter set to the appropriate energy widow for I-124 (350-750 keV). Two standards for gamma counting were prepared by making a 1/100 dilution of the injected dose in each eye, and in both eyes. Standards were used to calculate the total activity injected in the animal in terms of counts per minute (cpm) as well as the cpm injected in each eye. Two saline filled tubes were counted in the gamma counter to obtain background activity. Background cpm were subtracted from the tissue cpm. Background subtracted cpm were then decay corrected to the time of injection, divided by the total injected cpm and multiplied by 100 to calculate % Injected Dose (% ID). The decay corrected cpm in each eye were divided by the cpm injected in that eye, and multiplied by 100 to calculate the % ID in that eye. To calculate % ID/gram, each calculated % ID was divided by the corresponding tissue/organ weight. The reference below describes the % ID/g calculation in more detail: Yazaki P J, et al. 2001.

### RESULTS AND CONCLUSION

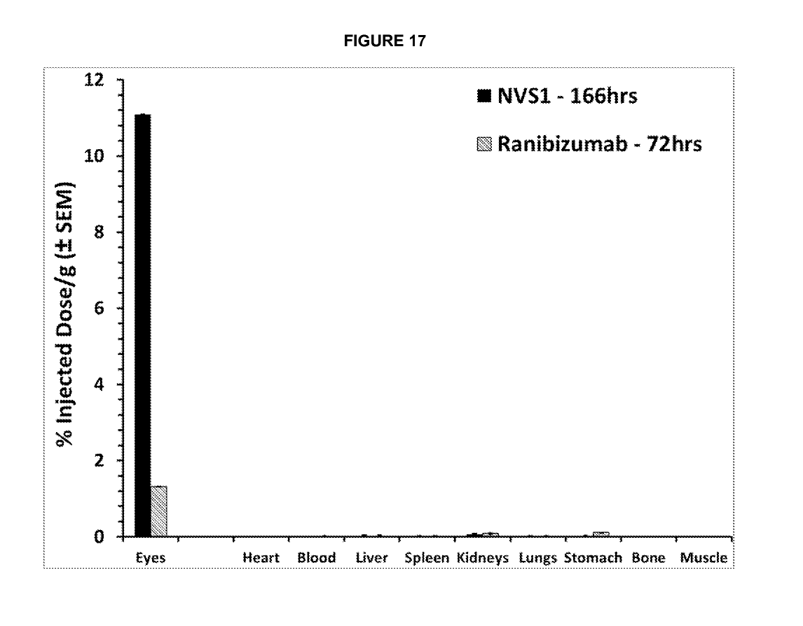

The biodistribution of radiolabeled IVT administered ranibizumab and NVS1 was assessed using a gamma counter (FIG. 17). 72 hrs post-IVT of I-124 labeled ranibizumab, ˜1.4% of the injected dose was measured in the eyes. In contrast, 166 hrs post-IVT of I-124 labeled HA-tagged antibody, ˜11% of the injected dose was measured in the eyes indicating a 10-fold higher ocular retention of the HA binding peptide tagged antibody compared to ranibizumab. In the remaining non-ocular tissues that were analyzed, similar amounts of both ranibizumab and the HA binding peptide tagged antibody were measured outside of the eye indicating no significant differences in extra-ocular retention of the HA binding peptide tagged antibody compared to ranibizumab. These data demonstrate the surprising finding that I-124 labeled peptide tagged molecules have significantly higher ocular retention, lower ocular clearance, and increased terminal ocular concentration as compared to untagged molecules. However, the half-life extension effect of the HA binding peptide tag was not seen outside of the eye.

### REFERENCE LIST

Campochiaro, P. A., Channa, R., Berger, B. B., Heier, J. S., Brown, D. M., Fiedler, U., Hepp, J., and Stumpp, M. T. (2012). Treatment of Diabetic Macular Edema Wth a Designed Ankyrin Repeat Protein That Binds Vascular Endothelial Growth Factor: A Phase 1/2 Study. Am J Ophthalmol.
Day, S., Acquah, K., Mruthyunjaya, P., Grossman, D. S., Lee, P. P., and Sloan, F. A. (2011). Ocular Complications After Anti-Vascular Endothelial Growth Factor Therapy in Medicare Patients With Age-Related Macular Degeneration. Am J Ophthalmol.
Ferrara, N., Damico, L., Shams, N., Lowman, H., and Kim, R. (2006). Development of ranibizumab, an anti-vascular endothelial growth factor antigen binding fragment, as therapy for neovascular age-related macular degeneration. Retina 26, 859-870.
Ferrara, N., Hillan, K. J., Gerber, H. P., and Novotny, W. (2004). Discovery and development of bevacizumab, an anti-VEGF antibody for treating cancer. Nat Rev Drug Discov 3, 391-400.
Levin, A. M. and Weiss, G. A. (2006). Optimizing the affinity and specificity of proteins with molecular display. Mol Biosyst. 2, 49-57.
Lipovsek, D. (2011). Adnectins: engineered target-binding protein therapeutics. Protein Eng Des Sel 24, 3-9.
Muether, P. S., Hermann, M. M., Viebahn, U., Kirchhof, B., and Fauser, S. (2012). Vascular Endothelial Growth Factor in Patients with Exudative Age-related Macular Degeneration Treated with Ranibizumab. Ophthalmology 119, 2082-2086.
Ng, E. W., Shima, D. T., Calias, P., Cunningham, E. T., Jr., Guyer, D. R., and Adamis, A. P. (2006). Pegaptanib, a targeted anti-VEGF aptamer for ocular vascular disease. Nat Rev Drug Discov 5, 123-132.
Patel, R. D., Momi, R. S., and Hariprasad, S. M. (2011). Review of ranibizumab trials for neovascular age-related macular degeneration. Semin. Ophthalmol 26, 372-379.
Pluckthun, A. (2012). Ribosome display: a perspective. Methods Mol Biol 805, 3-28.
Rosenfeld, P. J., Brown, D. M., Heier, J. S., Boyer, D. S., Kaiser, P. K., Chung, C. Y., and Kim, R. Y. (2006). Ranibizumab for neovascular age-related macular degeneration. N. Engl. J. Med. 355, 1419-1431.
Stewart, M. W., Grippon, S., and Kirkpatrick, P. (2012). Aflibercept. Nat Rev Drug Discov 11, 269-270.
Wittrup, K. D. (2001). Protein engineering by cell-surface display. Curr. Opin. Biotechnol. 12, 395-399.
Zhang, M., Zhang, J., Yan, M., Li, H., Yang, C., and Yu, D. (2008). Recombinant anti-vascular endothelial growth factor fusion protein efficiently suppresses choridal neovasularization in monkeys. Mol. Vis. 14, 37-49.
Laurent and Fraser, Functions of the proteoglycans, Ciba Foundation Symposium 124, 1986, p 9-29
J. Necas, L. Bartosikova, P. Brauner, and J. Kolar. Veterinarni Medicina, 53, 2008 (8): 397-411
Mao H, Hart S A, Schink A, Pollok B A. J Am Chem Soc. 2004 Mar. 10; 126(9):2670-1
Yazaki P J, Wu A M, Tsai S W, et al. Tumor targeting of radiometal labeled anti-CEA recombinant T84.66 diabody and t84.66 minibody: comparison to radioiodinated fragments. Bioconjug Chem 2001; 12:220-8.)
Aiello, L. P. (2005). Angiogenic pathways in diabetic retinopathy. N Engl J Med 353, 839-841.
Anand-Apte, B., Ebrahem, Q., Cutler, A., Farage, E., Sugimoto, M., Hollyfield, J., and Folkman, J. (2010). Betacellulin induces increased retinal vascular permeability in mice. PLoS. ONE. 5, e13444.
Boyer, David S. A Phase 2b Study of Fovista™, a Platelet Derived Growth Factor (PDGF) inhibitor in combination with a Vascular Endothelial Growth Factor (VEGF) inhibitor for Neovascular Age-Related Macular Degeneration (AMD). ARVO 2013. 2013.
Ref Type: Abstract
Hara, C., Kasai, A., Gomi, F., Satooka, T., Sakimoto, S., Nakai, K., Yoshioka, Y., Yamamuro, A., Maeda, S., and Nishida, K. (2013). Laser-induced choroidal neovascularization in mice attenuated by deficiency in the apelin-APJ system. Invest Ophthalmol. Vis. Sci. 54, 4321-4329.
Kaiser, P. K. (2013). Emerging therapies for neovascular age-related macular degeneration: drugs in the pipeline. Ophthalmology 120, S11-S15.
Kaiser, Peter K., Boyer, David S., and Campochiaro, Peter A. Integrin Peptide Therapy: The First Wet AMD Experience. ARVO 2013. 2013.
Ref Type: Abstract
Nussenblatt, R. B., Liu, B., Wei, L., and Sen, H. N. (2013). The immunological basis of degenerative diseases of the eye. Int. Rev. Immunol. 32, 97-112.
Oliner, J. D., Bready, J., Nguyen, L., Estrada, J., Hurh, E., Ma, H., Pretorius, J., Fanslow, W., Nork, T. M., Leedle, R. A., Kaufman, S., and Coxon, A. (2012). AMG 386, a selective angiopoietin 1/2-neutralizing peptibody, inhibits angiogenesis in models of ocular neovascular diseases. Invest Ophthalmol. Vis. Sci. 53, 2170-2180.
Patel, R. D., Momi, R. S., and Hariprasad, S. M. (2011). Review of ranibizumab trials for neovascular age-related macular degeneration. Semin. Ophthalmol 26, 372-379.
Patel, S. (2009a). Combination therapy for age-related macular degeneration. Retina 29, S45-S48.
Patel, S. (2009b). Combination therapy for age-related macular degeneration. Retina 29, S45-S48.
Rosenfeld, P. J., Brown, D. M., Heier, J. S., Boyer, D. S., Kaiser, P. K., Chung, C. Y., and Kim, R. Y. (2006). Ranibizumab for neovascular age-related macular degeneration. N. Engl. J. Med. 355, 1419-1431.
Watanabe, D., Suzuma, K., Matsui, S., Kurimoto, M., Kiryu, J., Kita, M., Suzuma, I., Ohashi, H., Ojima, T., Murakami, T., Kobayashi, T., Masuda, S., Nagao, M., Yoshimura, N., and Takagi, H. (2005). Erythropoietin as a Retinal Angiogenic Factor in Proliferative Diabetic Retinopathy. N Engl J Med 353, 782-792.

1) A peptide tag that binds hyaluronan (HA), wherein said peptide tag comprises the sequence of:
a) SEQ ID NO: 33, SEQ ID NO: 34, SEQ ID NO: 35 or SEQ ID NO: 36; or
b) 95 consecutive amino acids of the sequence of SEQ ID NO: 33, SEQ ID NO: 34, SEQ ID NO: 35 or SEQ ID NO: 36.

2) A peptide tagged molecule comprising a peptide tag as claimed in claim 1, linked to a protein or nucleic acid.

3) The peptide tagged molecule as claimed in claim 2, wherein said peptide tag is linked at the N terminus and/or the C terminus to said protein or at the 5′ and/or 3′ terminus of said nucleic acid.

4) The peptide tagged molecule as claimed in claim 2, wherein said peptide tag is linked directly to said protein or nucleic acid.

5) The peptide tagged molecule as claimed in claim 2, wherein said peptide tag is linked indirectly to said protein or nucleic acid via a linker.

6) The peptide tagged molecule as claimed in claim 2, wherein the protein is:
a) an isolated antibody, or antigen binding fragment thereof;
b) a therapeutic protein,
c) a protein receptor, or
d) a darpin.

7) The peptide tagged molecule as claimed in claim 2, wherein the nucleic acid is an aptamer.

8) The peptide tagged molecule as claimed in claim 2, wherein the molecule is a protein that binds VEGF, C5, Factor P, Factor D, EPO, EPOR, IL-1β, IL-17A, II-10, TNFα, or FGFR2.

9) The peptide tagged molecule as claimed in claim 2, wherein the molecule is a nucleic acid that binds PDGF-BB.

10) The peptide tagged molecule as claimed in claim 2, wherein the protein is an isolated antibody or antigen binding fragment:
a) that binds VEGF and comprises heavy chain CDR1, 2, and 3 sequences of SEQ ID NOs: 1, 2 and 3, respectively and light chain CDR1, 2, and 3 sequences of SEQ ID NOs: 11, 12 and 13, respectively; or
b) that binds C5 and comprises heavy chain CDR1, 2, and sequences of SEQ ID NOs: 37, 38, and 39 respectively and light chain CDR1, 2, and 3 sequences of SEQ ID NOs: 46, 47, and 48, respectively; or
c) that binds Factor P and comprises heavy chain CDR1, 2, and 3 sequences of SEQ ID NOs: 53, 54, and 55 respectively and light chain CDR1, 2, and 3 sequences of SEQ ID NOs: 65, 66, and 67, respectively; or
d) that binds EPO and comprises heavy chain CDR1, 2, and 3 sequences of SEQ ID NOs: 75, 76, and 77 respectively and light chain CDR1, 2, and 3 sequences of SEQ ID NOs: 86, 87, and 88, respectively; or
e) that binds TNFα and comprises heavy chain CDR1, 2, and 3 sequences of SEQ ID NOs: 108, 109, and 110 respectively and light chain CDR1, 2, and 3 sequences of SEQ ID NOs: 117, 118, and 119, respectively; or
f) that binds IL-1β and comprises heavy chain CDR1, 2, and 3 sequences of SEQ ID NOs: 189, 190, and 191 respectively and light chain CDR1, 2, and 3 sequences of SEQ ID NOs: 198, 199 and 200, respectively.

11) The peptide tagged molecule as claimed in claim 10, comprising an antibody variable heavy chain domain and a variable light chain domain having the sequences of:
a) SEQ ID NO: 7 and SEQ ID NO: 17, respectively; or
b) SEQ ID NO: 40 and SEQ ID NO: 49, respectively; or
c) SEQ ID NO: 59 and SEQ ID NO: 71, respectively; or
d) SEQ ID NO: 81 and SEQ ID NO: 92, respectively; or
e) SEQ ID NO: 111 and SEQ ID NO: 120, respectively; or
f) SEQ ID NO: 193 and SEQ ID NO: 201, respectively.

12) The peptide tagged molecule as claimed in claim 10, comprising an antibody heavy chain and a light chain sequence of:
a) SEQ ID NO: 9 and SEQ ID NO: 19, respectively; or
b) SEQ ID NO: 42 and SEQ ID NO: 51, respectively; or
c) SEQ ID NO: 61 and SEQ ID NO: 73, respectively; or
d) SEQ ID NO: 83 and SEQ ID NO: 95, respectively; or
e) SEQ ID NO: 113 and SEQ ID NO: 122, respectively; or
f) SEQ ID NO: 194 and SEQ ID NO: 202, respectively.

13) The peptide tagged molecule as claimed in claim 10, comprising the sequences of:
a) SEQ ID NOs: 21 and 19; or
b) SEQ ID NOs: 23 and 19; or
c) SEQ ID NOs: 25 and 19; or
d) SEQ ID NOs: 27 and 19; or
e) SEQ ID NOs: 29 and 19; or
f) SEQ ID NOs: 44 and 51; or
g) SEQ ID NOs: 63 and 73; or
h) SEQ ID NOs: 85 and 95; or
i) SEQ ID NOs: 115 and 122; or
j) SEQ ID NOs: 196 and 202.

14) A composition comprising a peptide tagged molecule as claimed in claim 2 and a pharmaceutically acceptable excipient, diluent or carrier.

15) The composition as claimed in claim 14 formulated for intraocular delivery.

16) The composition as claimed in claim 14 comprising 12 mg/eye of the peptide tagged molecule.

17) A nucleic acid comprising a sequence encoding a peptide tag of claim 1.

18) A nucleic acid comprising a sequence encoding a peptide tagged molecule as claimed in claim 2.

19) An expression vector comprising the nucleic acid as claimed in claim 17.

20) A host cell comprising the expression vector as claimed in claim 19.

21) The host cell as claimed in claim 20 wherein said host cell is a mammalian cell line.

22) A process for the production of a peptide tagged molecule, comprising culturing the host cell of claim 20 under appropriate conditions for the production of said peptide tagged molecule, and isolating said peptide tagged molecule.

23) A method of treating a condition or disorder of the eye in a subject, the method comprising administering to the subject a peptide tag that binds HA linked to a protein or nucleic acid.

24) A method of treating a condition or disorder associated with retinal vascular disease in a subject, the method comprising administering to the subject a peptide tag that binds HA linked to a protein or nucleic acid.

25) A method of treating a condition or disorder associated with retinal vascular disease in a subject, the method comprising administering to the subject the composition of claim 14.

26) The method of claim 24, wherein the condition or disorder associated with retinal vascular disease is neovascular age-related macular degeneration (wet AMD), diabetic retinopathy, diabetic macular edema, proliferative diabetic retinopathy, non-proliferative diabetic retinopathy, macular edema, retinal vein occlusion, multifocal choroiditis, myopic choroidal neovascularization or retinopathy of prematurity.

27) A method of treating a condition or disorder associated with macular edema in a subject, the method comprising administering a peptide tag that binds HA linked to a protein or nucleic acid.

28) A method of treating a condition or disorder associated with macular edema in a subject, the method comprising administering to the subject a composition as claimed in claim 14.

29) The method of claim 27 wherein the condition or disorder associated with macular edema is diabetic retinopathy, diabetic macular edema, proliferative diabetic retinopathy, non-proliferative diabetic retinopathy, neovascular age-related macular degeneration, retinal vein occlusion, multifocal choroiditis, myopic choroidal neovascularization, or retinopathy of prematurity.

30) A method of treating a VEGF-mediated disorder in a subject, the method comprising the step of administering to the subject a composition comprising a peptide tag as claimed in claim 1 linked to an anti-VEGF antibody or antigen binding fragment thereof.

31) The method of claim 30, wherein said anti-VEGF antibody or antigen binding fragment thereof comprises heavy chain CDR1, 2, and 3 sequences of SEQ ID NOs: 1, 2 and 3, respectively and light chain CDR1, 2, and 3 sequences of SEQ ID NOs: 11, 12 and 13, respectively.

32) The method as claimed in claim 31, wherein the VEGF-mediated disorder in a subject is age-related macular degeneration, neovascular glaucoma, diabetic retinopathy, macular edema, diabetic macular edema, pathologic myopia, retinal vein occlusions, retinopathy of prematurity, retrolental fibroplasia, abnormal vascular proliferation associated with phakomatoses, edema (such as that associated with brain tumors), Meigs' syndrome, rheumatoid arthritis, psoriasis and atherosclerosis.

33) A method of making a peptide tagged molecule, said method comprising linking a peptide tag as claimed in claim 1 to a protein or nucleic acid.

34) A method of increasing ocular half-life of a protein or nucleic acid comprising the step of linking said protein or nucleic acid to a peptide tag that binds HA.

35) The method of claim 34, wherein said peptide tag that binds HA comprises the sequence of:
a) SEQ ID NO: 33, SEQ ID NO: 34, SEQ ID NO: 35 or SEQ ID NO: 36; or
b) 95 consecutive amino acids of the sequence of SEQ ID NO: 33, SEQ ID NO: 34, SEQ ID NO: 35 or SEQ ID NO: 36


SEQUENCE LISTING (215 sequences)

SEQ ID NO: 1 - protein, 6 aa, Artificial Sequence
  Description of Artificial Sequence Synthetic peptide
DYYYMT

SEQ ID NO: 2 - protein, 16 aa, Artificial Sequence
  Description of Artificial Sequence Synthetic peptide
FIDPDDDPYYATWAKG

SEQ ID NO: 3 - protein, 11 aa, Artificial Sequence
  Description of Artificial Sequence Synthetic peptide
GDHNSGWGLDI

SEQ ID NO: 4 - protein, 8 aa, Artificial Sequence
  Description of Artificial Sequence Synthetic peptide
GFSLTDYY

SEQ ID NO: 5 - protein, 5 aa, Artificial Sequence
  Description of Artificial Sequence Synthetic peptide
DPDDD

SEQ ID NO: 6 - protein, 11 aa, Artificial Sequence
  Description of Artificial Sequence Synthetic peptide
GDHNSGWGLDI

SEQ ID NO: 7 - protein, 120 aa, Artificial Sequence
  Description of Artificial Sequence Synthetic polypeptide
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 8 - DNA, 360 bp, Artificial Sequence
  Description of Artificial Sequence Synthetic polynucleotide
gaggtgcagctggtggaatcaggcggcggactggtgcagcctggcggtagcctgagactgagctgcaccgctagtggctttagcctgaccgactactactatatgacctgggtcagacaggcccctggtaaaggcctggagtgggtcggctttatcgaccccgacgacgacccctactacgctacctgggctaagggccggttcactatctctagggataactctaagaacaccctgtacctgcagatgaatagcctgagagccgaggacaccgccgtctactactgcgccggcggcgatcacaatagcggctggggcctggatatctggggtcagggcaccctggtcaccgtgtctagc

SEQ ID NO: 9 - protein, 223 aa, Artificial Sequence
  Description of Artificial Sequence Synthetic polypeptide
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 10 - DNA, 669 bp, Artificial Sequence
  Description of Artificial Sequence Synthetic polynucleotide
gaggtgcaattggttgaatctgggggcggactggtgcagcccggtggatctttgcgcctgtcctgtacagcttctggcttctccttgaccgactactattacatgacttgggttcgccaagccccaggcaaagggcttgaatgggtggggttcattgaccccgacgatgatccttactacgccacatgggcaaagggccggtttactatcagccgggataattccaaaaacacattgtatttgcaaatgaactcactgagagcagaagatacggctgtgtactattgcgcaggcggcgatcataactccggctggggcctggacatctgggggcaggggaccctggtgacagtcagctcagcctcaacgaaggggcccagcgtgtttcctttggccccaagcagcaagtccacgtccggtgggactgcagctcttggttgtctggtcaaggattatttcccagaacccgtgaccgtgtcttggaacagtggtgcattgacatcaggagtgcatacattcccagctgtgctgcagagctctggcctgtatagcctttcctctgttgtcacggtgcccagctccagcctggggacgcagacctatatttgtaacgtgaaccataaaccctccaacaccaaggttgataaaagagtggagcccaagtcttgt

SEQ ID NO: 11 - protein, 11 aa, Artificial Sequence
  Description of Artificial Sequence Synthetic peptide
QASEIIHSWLA

SEQ ID NO: 12 - protein, 7 aa, Artificial Sequence
  Description of Artificial Sequence Synthetic peptide
LASTLAS

SEQ ID NO: 13 - protein, 12 aa, Artificial Sequence
  Description of Artificial Sequence Synthetic peptide
QNVYLASTNGAN

SEQ ID NO: 14 - protein, 7 aa, Artificial Sequence
  Description of Artificial Sequence Synthetic peptide
SEIIHSW

SEQ ID NO: 15 - protein, 3 aa, Artificial Sequence
  Description of Artificial Sequence Synthetic peptide
LAS

SEQ ID NO: 16 - protein, 9 aa, Artificial Sequence
  Description of Artificial Sequence Synthetic peptide
VYLASTNGA

SEQ ID NO: 17 - protein, 111 aa, Artificial Sequence
  Description of Artificial Sequence Synthetic polypeptide
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLK

SEQ ID NO: 18 - DNA, 333 bp, Artificial Sequence
  Description of Artificial Sequence Synthetic polynucleotide
gagatcgtgatgactcagtcacctagcaccctgagcgctagtgtgggcgatagagtgattatcacctgtcaggctagtgaaattattcactcctggctggcctggtatcagcagaagcccggtaaagcccctaagctgctgatctacctggcctctaccctggctagtggcgtgccctctaggtttagcggtagcggtagtggcgccgagttcaccctgactatctctagcctgcagcccgacgacttcgctacctactactgtcagaacgtctacctggctagtactaacggcgctaacttcggtcagggcactaagctgaccgtgctgaag

SEQ ID NO: 19 - protein, 218 aa, Artificial Sequence
  Description of Artificial Sequence Synthetic polypeptide
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - DNA, 654 bp, Artificial Sequence
  Description of Artificial Sequence Synthetic polynucleotide
gagatcgtgatgactcagtcacctagcaccctgagcgctagtgtgggcgatagagtgattatcacctgtcaggctagtgaaattattcactcctggctggcctggtatcagcagaagcccggtaaagcccctaagctgctgatctacctggcctctaccctggctagtggcgtgccctctaggtttagcggtagcggtagtggcgccgagttcaccctgactatctctagcctgcagcccgacgacttcgctacctactactgtcagaacgtctacctggctagtactaacggcgctaacttcggtcagggcactaagctgaccgtgctgaagcggaccgtggccgctcctagtgtgtttatcttcccacctagcgacgagcagctgaagtcaggcaccgctagtgtcgtgtgcctgctgaacaacttctaccctagagaagctaaggtgcagtggaaagtggataacgccctgcagtcaggtaatagtcaggaatcagtcaccgagcaggactctaaggatagcacctatagcctgtctagcacactgaccctgtctaaggccgactacgagaagcacaaggtctacgcctgcgaagtgactcaccagggactgtctagccccgtgactaagtcctttaatagaggcgagtgc

SEQ ID NO: 21 - protein, 325 aa, Artificial Sequence
  Description of Artificial Sequence Synthetic polypeptide
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 22 - DNA, 975 bp, Artificial Sequence
  Description of Artificial Sequence Synthetic polynucleotide
gaggtgcagctggtggaatcaggcggcggactggtgcagcctggcggtagcctgagactgagctgcaccgctagtggctttagcctgaccgactactactatatgacctgggtcagacaggcccctggtaaaggcctggagtgggtcggctttatcgaccccgacgacgacccctactacgctacctgggctaagggccggttcactatctctagggataactctaagaacaccctgtacctgcagatgaatagcctgagagccgaggacaccgccgtctactactgcgccggcggcgatcacaatagcggctggggcctggatatctggggtcagggcaccctggtcaccgtgtctagcgcctctactaagggacctagcgtgttccccctggcccctagctctaagtctactagcggcggcaccgccgctctgggctgcctggtcaaggactacttccccgagcccgtgaccgtcagctggaatagcggcgctctgactagcggagtgcacaccttccccgccgtgctgcagtctagcggcctgtatagcctgtctagcgtcgtgaccgtgcctagctctagcctgggcactcagacctatatctgtaacgtgaaccacaagccctctaacactaaggtggacaagcgggtggaacctaagtcctgcggtagcggcggaggcggagtctatcacagagaggctagatcaggcaagtataagctgacctacgccgaggctaaggccgtgtgcgagttcgagggcggtcacctggctacctataagcagctggaagccgctagaaagatcggctttcacgtgtgcgccgctggctggatggctaagggtagagtgggctaccctatcgtgaagcctggccctaactgcggcttcggtaaaaccggaattatcgactacgggattaggctgaatagatcagagcgctgggacgcctactgctataaccctcacgct

SEQ ID NO: 23 - protein, 325 aa, Artificial Sequence
  Description of Artificial Sequence Synthetic polypeptide
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 24 - DNA, 975 bp, Artificial Sequence
  Description of Artificial Sequence Synthetic polynucleotide
gaggtgcagctggtggaatcaggcggcggactggtgcagcctggcggtagcctgagactgagctgcaccgctagtggctttagcctgaccgactactactatatgacctgggtcagacaggcccctggtaaaggcctggagtgggtcggctttatcgaccccgacgacgacccctactacgctacctgggctaagggccggttcactatctctagggataactctaagaacaccctgtacctgcagatgaatagcctgagagccgaggacaccgccgtctactactgcgccggcggtgatcacaatagcggctggggcctggatatctggggtcaaggcaccctggtcaccgtgtctagcgcctctactaagggcccctcagtgttccccctggcccctagctctaagtctactagcggcggcaccgccgctctgggctgcctggtcaaggactacttccccgagcccgtgaccgtcagctggaatagcggcgctctgactagcggagtgcacaccttccccgccgtgctgcagtctagcggcctgtatagcctgtctagcgtcgtgaccgtgcctagctctagcctgggcactcagacctatatctgtaacgtgaaccacaagccctctaacactaaggtggacaagcgggtggaacctaagtcctgcggtagcggcggaggcggagtctatcacagagaggctcagtcaggcaagtataagctgacctacgccgaggctaaggccgtgtgcgagttcgagggcggtcacctggctacctataagcagctggaagccgctagaaagatcggctttcacgtgtgcgccgctggctggatggctaagggtagagtgggctaccctatcgtgaagcctggccctaactgcggcttcggtaaaaccggaattatcgactacgggattaggctgaatagatcagagcgctgggacgcctactgctataaccctcacgcc

SEQ ID NO: 25 - protein, 325 aa, Artificial Sequence
  Description of Artificial Sequence Synthetic polypeptide
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAASGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 26 - DNA, 975 bp, Artificial Sequence
  Description of Artificial Sequence Synthetic polynucleotide
gaggtgcagctggtggaatcaggcggcggactggtgcagcctggcggtagcctgagactgagctgcaccgctagtggctttagcctgaccgactactactatatgacctgggtcagacaggcccctggtaaaggcctggagtgggtcggctttatcgaccccgacgacgacccctactacgctacctgggctaagggccggttcactatctctagggataactctaagaacaccctgtacctgcagatgaatagcctgagagccgaggacaccgccgtctactactgcgccggcggtgatcacaatagcggctggggcctggatatctggggtcaaggcaccctggtcaccgtgtctagcgcctctactaagggcccctcagtgttccccctggcccctagctctaagtctactagcggcggcaccgccgctctgggctgcctggtcaaggactacttccccgagcccgtgaccgtcagctggaatagcggcgctctgactagcggagtgcacaccttccccgccgtgctgcagtctagcggcctgtatagcctgtctagcgtcgtgaccgtgcctagctctagcctgggcactcagacctatatctgtaacgtgaaccacaagccctctaacactaaggtggacaagcgggtggaacctaagtcctgcggtagcggcggaggcggagtctatcacagagaggctgctagcggtaaatacaagctgacctacgccgaggctaaggccgtgtgcgagttcgagggcggtcacctggctacctataagcagctggaagccgctagaaagatcggctttcacgtgtgcgccgctggctggatggctaagggtagagtgggctaccctatcgtgaagcctggccctaactgcggcttcggtaaaaccggaattatcgactacgggattaggctgaatagatcagagcgctgggacgcctactgctataaccctcacgcc

SEQ ID NO: 27 - protein, 327 aa, Artificial Sequence
  Description of Artificial Sequence Synthetic polypeptide
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGACGVYHREAQSGKYKLTYAEAKAVCEFEGGHLATYKQLECARKIGFHVCAAGWMAKGRVGYPIVKPGPNCGFGKTGIIDYGIRLNRSERWDAYCYNPHA

SEQ ID NO: 28 - DNA, 981 bp, Artificial Sequence
  Description of Artificial Sequence Synthetic polynucleotide
gaagtgcagctggtggaaagcggcggaggcctggtgcagcctggcggatctctgagactgagctgtaccgccagcggcttcagcctgaccgactactactacatgacctgggtccgacaggcccctggcaagggactggaatgggtcggattcatcgaccccgacgacgacccctactacgccacatgggccaagggccggttcaccatcagccgggacaacagcaagaacaccctgtacctgcagatgaacagcctgcgggccgaggacaccgccgtgtactattgtgccggcggagatcacaacagcggctggggcctggatatctggggacagggaacactggtcaccgtgtctagcgccagcaccaagggccctagcgtgttccctctggcccctagcagcaagagcacatctggcggaacagccgccctgggctgcctggtcaaggactactttcccgagcccgtgaccgtgtcctggaactctggcgctctgacaagcggcgtgcacacctttccagccgtgctgcagagcagcggcctgtactctctgagcagcgtggtcacagtgcccagctctagcctgggaacccagacctacatctgcaacgtgaaccacaagcccagcaacaccaaggtggacaagcgggtggaacccaagagctgcggatccggcggaggcgcctgtggcgtgtatcacagggaggcccagagcggcaagtacaagctcacctacgccgaggccaaggccgtgtgcgaattcgagggcggccacctggccacctacaagcagctggagtgcgccaggaagatcggcttccacgtgtgtgccgccggctggatggccaaaggcagagtgggctaccccatcgtgaaacccggccccaactgcggcttcggcaagacaggcatcatcgactacggcatcaggctgaacaggagcgagaggtgggacgcctactgctacaacccccacgcc

SEQ ID NO: 29 - protein, 325 aa, Artificial Sequence
  Description of Artificial Sequence Synthetic polypeptide
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAQSGKYKLTYAEAKAVCEFEGGHLCTYKQLEAARKIGFHVCAAGWMAKGRVGYPIVKPGPNCGFGKTGIIDYGIRLNRSERWDAYCCNPHA

SEQ ID NO: 30 - DNA, 975 bp, Artificial Sequence
  Description of Artificial Sequence Synthetic polynucleotide
gaagtgcagctggtggaaagcggcggaggcctggtgcagcctggcggatctctgagactgagctgtaccgccagcggcttcagcctgaccgactactactacatgacctgggtccgacaggcccctggcaagggactggaatgggtcggattcatcgaccccgacgacgacccctactacgccacatgggccaagggccggttcaccatcagccgggacaacagcaagaacaccctgtacctgcagatgaacagcctgcgggccgaggacaccgccgtgtactattgtgccggcggagatcacaacagcggctggggcctggatatctggggacagggaacactggtcaccgtgtctagcgccagcaccaagggccctagcgtgttccctctggcccctagcagcaagagcacatctggcggaacagccgccctgggctgcctggtcaaggactactttcccgagcccgtgaccgtgtcctggaactctggcgctctgacaagcggcgtgcacacctttccagccgtgctgcagagcagcggcctgtactctctgagcagcgtggtcacagtgcccagctctagcctgggaacccagacctacatctgcaacgtgaaccacaagcccagcaacaccaaggtggacaagcgggtggaacccaagagctgcggatccggcggcggcggagtgtatcacagagaggcccagagcggcaagtacaagctgacctacgccgaggccaaggccgtgtgtgagttcgagggcggccacctgtgcacctacaagcagctggaggccgccaggaagatcggcttccacgtgtgtgccgccggctggatggctaaaggcagggtgggctaccccattgtgaagcccggccccaattgcggcttcggcaagaccggcatcatcgactacggcatcaggctgaacaggagcgagaggtgggacgcctactgctgcaacccccacgcc

SEQ ID NO: 31 - protein, 5 aa, Artificial Sequence
  Description of Artificial Sequence Synthetic peptide
GSGGG

SEQ ID NO: 32 - protein, 98 aa, Artificial Sequence
  Description of Artificial Sequence Synthetic polypeptide
GVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAK

SEQ ID NO: 33 - protein, 97 aa, Artificial Sequence
  Description of Artificial Sequence Synthetic polypeptide
GVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 34 - protein, 97 aa, Artificial Sequence
  Description of Artificial Sequence Synthetic polypeptide
GVYHREAASGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 35 - protein, 99 aa, Artificial Sequence
  Description of Artificial Sequence Synthetic polypeptide
ACGVYHREAQSGKYKLTYAEAKAVCEFEGGHLATYKQLECARKIGFHVCAAGWMAKGRVGYPIVKPGPNCGFGKTGIIDYGIRLNRSERWDAYCYNPHA

SEQ ID NO: 36 - protein, 97 aa, Artificial Sequence
  Description of Artificial Sequence Synthetic polypeptide
GVYHREAQSGKYKLTYAEAKAVCEFEGGHLCTYKQLEAARKIGFHVCAAGWMAKGRVGYPIVKPGPNCGFGKTGIIDYGIRLNRSERWDAYCCNPHA

SEQ ID NO: 37 - protein, 5 aa, Artificial Sequence
  Description of Artificial Sequence Synthetic peptide
SYAIS

SEQ ID NO: 38 - protein, 17 aa, Artificial Sequence
  Description of Artificial Sequence Synthetic peptide
GIGPFFGTANYAQKFQG

SEQ ID NO: 39 - protein, 7 aa, Artificial Sequence
  Description of Artificial Sequence Synthetic peptide
DTPYFDY

SEQ ID NO: 40 - protein, 116 aa, Artificial Sequence
  Description of Artificial Sequence Synthetic polypeptide
EVQLVQSGAEVKKPGSSVKVSCKASGGTFSSYAISWVRQAPGQGLEWMGGIGPFFGTANYAQKFQGRVTITADESTSTAYMELSSLRSEDTAVYYCARDTPYFDYWGQGTLVTVSS

SEQ ID NO: 41 - DNA, 348 bp, Artificial Sequence
  Description of Artificial Sequence Synthetic polynucleotide
gaggtgcaattggttcagtctggcgcggaagtgaaaaaaccgggcagcagcgtgaaagtgagctgcaaagcctccggaggcactttttcttcttatgccatttcttgggtgcgccaagcccctgggcagggtctcgagtggatgggcggtatcggtccgttttttggcactgcgaattacgcgcagaagtttcagggccgggtgaccattaccgcggatgaaagcaccagcaccgcgtatatggaactgagcagcctgcgtagcgaagatacggccgtgtattattgcgcgcgtgatactccttattttgattattggggccaaggcaccctggtgacggttagctca

SEQ ID NO: 42 - protein, 219 aa, Artificial Sequence
  Description of Artificial Sequence Synthetic polypeptide
EVQLVQSGAEVKKPGSSVKVSCKASGGTFSSYAISWVRQAPGQGLEWMGGIGPFFGTANYAQKFQGRVTITADESTSTAYMELSSLRSEDTAVYYCARDTPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 43 - DNA, 657 bp, Artificial Sequence
  Description of Artificial Sequence Synthetic polynucleotide
gaggtgcaattggtccaaagcggcgctgaggtcaagaagcctggcagcagcgtgaaggtctcctgcaaggccagcggcggcacattctccagctatgctatcagctgggtcagacaagcccccggccaaggactggaatggatgggaggaatcggccctttcttcggaaccgccaactacgcccagaagtttcagggaagggtgaccatcaccgccgatgagagcacatccacagcctatatggagctctccagcctgagatccgaagacaccgccgtctactactgcgctagggacaccccctacttcgactattggggccagggcacactcgtgaccgtgagctcagccagcaccaaaggccctagcgtcttccccctggctccttccagcaagagcacaagcggaggaacagctgctctcggctgcctggtcaaggactacttccccgagcctgtcacagtgtcctggaatagcggagccctgaccagcggcgtgcatacattccccgctgtgctccagagctccggcctctacagcctcagctccgtggtcaccgtccctagctcctccctgggcacacagacctacatctgcaacgtcaaccacaagccctccaacaccaaggtggacaagagggtggagcccaaaagctgt

SEQ ID NO: 44 - protein, 321 aa, Artificial Sequence
  Description of Artificial Sequence Synthetic polypeptide
EVQLVQSGAEVKKPGSSVKVSCKASGGTFSSYAISWVRQAPGQGLEWMGGIGPFFGTANYAQKFQGRVTITADESTSTAYMELSSLRSEDTAVYYCARDTPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 45 - DNA, 963 bp, Artificial Sequence
  Description of Artificial Sequence Synthetic polynucleotide
gaggtgcaattggtccaaagcggcgctgaggtcaagaagcctggcagcagcgtgaaggtctcctgcaaggccagcggcggcacattctccagctatgctatcagctgggtcagacaagcccccggccaaggactggaatggatgggaggaatcggccctttcttcggaaccgccaactacgcccagaagtttcagggaagggtgaccatcaccgccgatgagagcacatccacagcctatatggagctctccagcctgagatccgaagacaccgccgtctactactgcgctagggacaccccctacttcgactattggggccagggcacactcgtgaccgtgagctcagccagcaccaaaggccctagcgtcttccccctggctccttccagcaagagcacaagcggaggaacagctgctctcggctgcctggtcaaggactacttccccgagcctgtcacagtgtcctggaatagcggagccctgaccagcggcgtgcatacattccccgctgtgctccagagctccggcctctacagcctcagctccgtggtcaccgtccctagctcctccctgggcacacagacctacatctgcaacgtcaaccacaagccctccaacaccaaggtggacaagagggtggagcccaaaagctgtggatccggaggaggcggcgtgtatcatagagaggcccagtccggcaagtacaagctgacctacgccgaagccaaggccgtgtgtgagttcgagggcggacacctggctacctacaaacagctcgaagccgctaggaagatcggattccacgtgtgcgccgccggatggatggccaaaggcagagtgggctaccccattgtcaagcccggacccaactgcggattcggcaagaccggcatcatcgactacggcatcaggctcaacaggtccgagagatgggacgcttactgctacaatccccacgcc

SEQ ID NO: 46 - protein, 11 aa, Artificial Sequence
  Description of Artificial Sequence Synthetic peptide
SGDSIPNYYVY

SEQ ID NO: 47 - protein, 7 aa, Artificial Sequence
  Description of Artificial Sequence Synthetic peptide
DDSNRPS

SEQ ID NO: 48 - protein, 11 aa, Artificial Sequence
  Description of Artificial Sequence Synthetic peptide
QSFDSSLNAEV

SEQ ID NO: 49 - protein, 108 aa, Artificial Sequence
  Description of Artificial Sequence Synthetic polypeptide
SYELTQPLSVSVALGQTARITCSGDSIPNYYVYWYQQKPGQAPVLVIYDDSNRPSGIPERFSGSNSGNTATLTISRAQAGDEADYYCQSFDSSLNAEVFGGGTKLTVL

SEQ ID NO: 50 - DNA, 324 bp, Artificial Sequence
  Description of Artificial Sequence Synthetic polynucleotide
tcctatgaactcacacagcccctgagcgtgagcgtggccctgggccagaccgcccggatcacctgctccggcgacagcatccccaactactacgtgtactggtaccagcagaagcccggccaggcccccgtgctggtgatctacgacgacagcaaccggcccagcggcatccccgagcggttcagcggcagcaacagcggcaacaccgccaccctgaccatttccagagcacaggcaggcgacgaggccgactactactgccagagcttcgacagcagcctgaacgccgaggtgttcggcggagggaccaagttaaccgtccta

SEQ ID NO: 51 - protein, 214 aa, Artificial Sequence
  Description of Artificial Sequence Synthetic polypeptide
SYELTQPLSVSVALGQTARITCSGDSIPNYYVYWYQQKPGQAPVLVIYDDSNRPSGIPERFSGSNSGNTATLTISRAQAGDEADYYCQSFDSSLNAEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 52 - DNA, 642 bp, Artificial Sequence
  Description of Artificial Sequence Synthetic polynucleotide
agctacgagctgacccagcccctgagcgtgagcgtggccctgggccagaccgccaggatcacctgcagcggcgacagcatccccaactactacgtgtactggtatcagcagaagcccggccaggcccccgtgctggtgatctacgacgacagcaacaggcccagcggcatccccgagaggttcagcggcagcaacagcggcaacaccgccaccctgaccatcagcagagcccaggccggcgacgaggccgactactactgccagagcttcgacagctcactgaacgccgaggtgttcggcggagggaccaagctgaccgtgctgggccagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 53 - protein, 5 aa, Artificial Sequence
  Description of Artificial Sequence Synthetic peptide
SYAIS

SEQ ID NO: 54 - protein, 17 aa, Artificial Sequence
  Description of Artificial Sequence Synthetic peptide
RIIPIFGTANYAQKFQG

SEQ ID NO: 55 - protein, 8 aa, Artificial Sequence
  Description of Artificial Sequence Synthetic peptide
HGGYSFDS

SEQ ID NO: 56 - protein, 7 aa, Artificial Sequence
  Description of Artificial Sequence Synthetic peptide
GGTFNSY

SEQ ID NO: 57 - protein, 6 aa, Artificial Sequence
  Description of Artificial Sequence Synthetic peptide
IPIFGT

SEQ ID NO: 58 - protein, 8 aa, Artificial Sequence
  Description of Artificial Sequence Synthetic peptide
HGGYSFDS

SEQ ID NO: 59 - protein, 117 aa, Artificial Sequence
  Description of Artificial Sequence Synthetic polypeptide
EVQLVQSGAEVKKPGSSVKVSCKASGGTFNSYAISWVRQAPGQGLEWMGRIIPIFGTANYAQKFQGRVTITADESTSTAYMELSSLRSEDTAVYYCARHGGYSFDSWGQGTLVTVSS

SEQ ID NO: 60 - DNA, 351 bp, Artificial Sequence
  Description of Artificial Sequence Synthetic polynucleotide
gaggtgcagctggtgcagagcggagccgaagtgaagaaacccggcagcagcgtgaaggtgtcctgcaaggccagcggcggcaccttcaacagctacgccatcagctgggtgcgccaggctcctggacagggcctggaatggatgggccggatcatccccatcttcggcaccgccaactacgcccagaaattccagggcagagtgaccatcaccgccgacgagagcaccagcaccgcctacatggaactgagcagcctgagaagcgaggacaccgccgtgtactactgtgcccggcacggcggctacagcttcgatagctggggccagggcaccctggtgaccgtgagctca

SEQ ID NO: 61 - protein, 220 aa, Artificial Sequence
  Description of Artificial Sequence Synthetic polypeptide
EVQLVQSGAEVKKPGSSVKVSCKASGGTFSSYAISWVRQAPGQGLEWMGRIIPIFGTANYAQKFQGRVTITADESTSTAYMELSSLRSEDTAVYYCARHGGYSFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 62 - DNA, 660 bp, Artificial Sequence
  Description of Artificial Sequence Synthetic polynucleotide
gaggtgcagctggtgcagagcggagccgaagtgaagaaacccggcagcagcgtgaaggtgtcctgcaaggccagcggcggcaccttcaacagctacgccatcagctgggtgcgccaggctcctggacagggcctggaatggatgggccggatcatccccatcttcggcaccgccaactacgcccagaaattccagggcagagtgaccatcaccgccgacgagagcaccagcaccgcctacatggaactgagcagcctgagaagcgaggacaccgccgtgtactactgtgcccggcacggcggctacagcttcgatagctggggccagggcaccctggtgaccgtgagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgt

SEQ ID NO: 63 - protein, 322 aa, Artificial Sequence
  Description of Artificial Sequence Synthetic polypeptide
EVQLVQSGAEVKKPGSSVKVSCKASGGTFSSYAISWVRQAPGQGLEWMGRIIPIFGTANYAQKFQGRVTITADESTSTAYMELSSLRSEDTAVYYCARHGGYSFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 64 - DNA, 966 bp, Artificial Sequence
  Description of Artificial Sequence Synthetic polynucleotide
gaggtgcaattggtgcagagcggagctgaggtgaagaagcccggcagctccgtcaaggtgagctgcaaagcctccggaggcaccttttcctcctacgctatctcctgggtgaggcaagcccccggacaaggactggagtggatgggcaggatcatccccatcttcggaaccgccaactacgcccagaaattccagggcagggtgaccatcaccgccgacgaaagcaccagcaccgcctacatggagctctccagcctgaggagcgaggacaccgctgtgtactactgcgccagacacggcggctactatttcgacagctggggccagggcacactggtgaccgtgagctcagcaagcaccaaaggaccctccgtctttcctctggcccccagcagcaagtccacaagcggaggaaccgctgccctgggatgtctcgtgaaggactacttccctgagcccgtgacagtgtcctggaatagcggcgccctgacaagcggcgtgcacacatttcccgccgtcctgcaaagctccggcctctatagcctgagctccgtcgtgacagtcccctccagctccctgggaacccagacctacatctgcaacgtcaaccacaagcccagcaacacaaaggtggacaagagggtcgagcctaagagctgtggatccggcggcggaggagtgtaccatagggaggcccagagcggaaagtacaagctgacctatgccgaggctaaggccgtctgcgaattcgagggcggccatctggccacctacaagcaactggaggccgctaggaagatcggcttccacgtctgcgccgctggatggatggccaagggcagagtgggctatcccatcgtgaagcccggccccaactgcggcttcggaaagacaggcatcatcgactacggcatcaggctcaacaggagcgagaggtgggacgcttactgctacaacccccatgcc

SEQ ID NO: 65 - protein, 11 aa, Artificial Sequence
  Description of Artificial Sequence Synthetic peptide
SGDNLGSKYVD

SEQ ID NO: 66 - protein, 7 aa, Artificial Sequence
  Description of Artificial Sequence Synthetic peptide
SDNNRPS

SEQ ID NO: 67 - protein, 10 aa, Artificial Sequence
  Description of Artificial Sequence Synthetic peptide
QTYTSGNNYL

SEQ ID NO: 68 - protein, 7 aa, Artificial Sequence
  Description of Artificial Sequence Synthetic peptide
DNLGSKY

SEQ ID NO: 69 - protein, 3 aa, Artificial Sequence
  Description of Artificial Sequence Synthetic peptide
SDN

SEQ ID NO: 70 - protein, 8 aa, Artificial Sequence
  Description of Artificial Sequence Synthetic peptide
YTSGNNYL

SEQ ID NO: 71 - protein, 108 aa, Artificial Sequence
  Description of Artificial Sequence Synthetic polypeptide
SYELTQPPSVSVAPGQTARISCSGDNLGSKYVDWYQQKPGQAPVLVIYSDNNRPSGIPERFSGSNSGNTATLTISGTQAEDEADYYCQTYTSGNNYLVFGGGTKLTVL

SEQ ID NO: 72 - DNA, 324 bp, Artificial Sequence
  Description of Artificial Sequence Synthetic polynucleotide
agctacgagctgactcagcccccttctgtgtctgtggcccctggccagaccgccagaatcagctgcagcggcgacaacctgggcagcaaatacgtggactggtatcagcagaagcccggccaggctcccgtgctggtgatctacagcgacaacaaccggcccagcggcatccctgagcggttcagcggcagcaacagcggcaataccgccaccctgaccatcagcggcacccaggccgaggacgaggccgactactactgccagacctacaccagcggcaacaactacctggtgttcggaggcggaacaaagttaaccgtccta

SEQ ID NO: 73 - protein, 214 aa, Artificial Sequence
  Description of Artificial Sequence Synthetic polypeptide
SYELTQPPSVSVAPGQTARISCSGDNLGSKYVDWYQQKPGQAPVLVIYSDNNRPSGIPERFSGSNSGNTATLTISGTQAEDEADYYCQTYTSGNNYLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 74 - DNA, 642 bp, Artificial Sequence
  Description of Artificial Sequence Synthetic polynucleotide
agctacgagctgactcagcccccttctgtgtctgtggcccctggccagaccgccagaatcagctgcagcggcgacaacctgggcagcaaatacgtggactggtatcagcagaagcccggccaggctcccgtgctggtgatctacagcgacaacaaccggcccagcggcatccctgagcggttcagcggcagcaacagcggcaataccgccaccctgaccatcagcggcacccaggccgaggacgaggccgactactactgccagacctacaccagcggcaacaactacctggtgttcggaggcggaacaaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 75 - protein, 5 aa, Artificial Sequence
  Description of Artificial Sequence Synthetic peptide
SYWIG

SEQ ID NO: 76 - protein, 17 aa, Artificial Sequence
  Description of Artificial Sequence Synthetic peptide
WIDPYRSEIRYSPSFQG

SEQ ID NO: 77 - protein, 8 aa, Artificial Sequence
  Description of Artificial Sequence Synthetic peptide
VSSEPFDS

SEQ ID NO: 78 - protein, 7 aa, Artificial Sequence
  Description of Artificial Sequence Synthetic peptide
GYSFTSY

SEQ ID NO: 79 - protein, 6 aa, Artificial Sequence
  Description of Artificial Sequence Synthetic peptide
DPYRSE

SEQ ID NO: 80 - protein, 8 aa, Artificial Sequence
  Description of Artificial Sequence Synthetic peptide
VSSEPFDS

SEQ ID NO: 81 - protein, 117 aa, Artificial Sequence
  Description of Artificial Sequence Synthetic polypeptide
EVQLVQSGAEVKKPGESLKISCKGSGYSFTSYWIGWVRQMPGKGLEWMGWIDPYRSEIRYSPSFQGQVTISADKSISTAYLQWSSLKASDTAMYYCARVSSEPFDSWGQGTLVTVSS

SEQ ID NO: 82 - DNA, 351 bp, Artificial Sequence
  Description of Artificial Sequence Synthetic polynucleotide
gaggtccaattggtccaatccggagccgaagtcaagaaacccggcgagtccctcaaaatcagctgcaagggctccggctactccttcaccagctactggatcggatgggtgaggcagatgcccggcaaaggcctcgagtggatgggctggatcgacccctataggtccgagattaggtacagcccctccttccagggccaggtcaccatctccgccgacaagagcatcagcaccgcctacctccaatggtcctccctcaaggcctccgataccgccatgtattactgcgccagggtcagcagcgagccctttgacagctggggccagggaaccctcgtgaccgtcagctca

SEQ ID NO: 83 - protein, 220 aa, Artificial Sequence
  Description of Artificial Sequence Synthetic polypeptide
EVQLVQSGAEVKKPGESLKISCKGSGYSFTSYWIGWVRQMPGKGLEWMGWIDPYRSEIRYSPSFQGQVTISADKSISTAYLQWSSLKASDTAMYYCARVSSEPFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 84 - DNA, 660 bp, Artificial Sequence
  Description of Artificial Sequence Synthetic polynucleotide
gaggtccaattggtccaatccggagccgaagtcaagaaacccggcgagtccctcaaaatcagctgcaagggctccggctactccttcaccagctactggatcggatgggtgaggcagatgcccggcaaaggcctcgagtggatgggctggatcgacccctataggtccgagattaggtacagcccctccttccagggccaggtcaccatctccgccgacaagagcatcagcaccgcctacctccaatggtcctccctcaaggcctccgataccgccatgtattactgcgccagggtcagcagcgagccctttgacagctggggccagggaaccctcgtgaccgtcagctcagccagcaccaaaggacctagcgtgttccccctcgctccctcctccaagagcacatccggcggaaccgctgctctgggatgtctcgtcaaggactacttccccgagcccgtgaccgtgagctggaatagcggcgccctgacctccggagtccacacattccccgctgtcctgcagagcagcggcctgtatagcctgtcctccgtcgtgaccgtccctagcagctccctgggaacccagacctacatctgcaacgtcaaccacaagcctagcaacaccaaggtggacaagagggtggagcccaaatcctgc

SEQ ID NO: 85 - protein, 322 aa, Artificial Sequence
  Description of Artificial Sequence Synthetic polypeptide
EVQLVQSGAEVKKPGESLKISCKGSGYSFTSYWIGWVRQMPGKGLEWMGWIDPYRSEIRYSPSFQGQVTISADKSISTAYLQWSSLKASDTAMYYCARVSSEPFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 86 - DNA, 966 bp, Artificial Sequence
  Description of Artificial Sequence Synthetic polynucleotide
gaggtccaattggtccaatccggagccgaagtcaagaaacccggcgagtccctcaaaatcagctgcaagggctccggctactccttcaccagctactggatcggatgggtgaggcagatgcccggcaaaggcctcgagtggatgggctggatcgacccctataggtccgagattaggtacagcccctccttccagggccaggtcaccatctccgccgacaagagcatcagcaccgcctacctccaatggtcctccctcaaggcctccgataccgccatgtattactgcgccagggtcagcagcgagccctttgacagctggggccagggaaccctcgtgaccgtcagctcagccagcaccaaaggacctagcgtgttccccctcgctccctcctccaagagcacatccggcggaaccgctgctctgggatgtctcgtcaaggactacttccccgagcccgtgaccgtgagctggaatagcggcgccctgacctccggagtccacacattccccgctgtcctgcagagcagcggcctgtatagcctgtcctccgtcgtgaccgtccctagcagctccctgggaacccagacctacatctgcaacgtcaaccacaagcctagcaacaccaaggtggacaagagggtggagcccaaatcctgcggatccggaggaggcggcgtgtatcacagagaggcccagagcggcaagtacaagctcacatacgctgaggccaaagccgtgtgcgaattcgagggcggacatctggccacatataagcagctggaggccgccaggaagatcggcttccacgtgtgcgctgccggctggatggccaaaggcagagtgggctaccctatcgtcaagcccggccccaactgcggctttggcaagaccggcatcatcgactacggcatcaggctcaacaggtccgaaaggtgggatgcctactgctacaatccccacgcc

SEQ ID NO: 87 - protein, 11 aa, Artificial Sequence
  Description of Artificial Sequence Synthetic peptide
SGDKLGDHYAY

SEQ ID NO: 88 - protein, 7 aa, Artificial Sequence
  Description of Artificial Sequence Synthetic peptide
DDSKRPS

SEQ ID NO: 89 - protein, 10 aa, Artificial Sequence
  Description of Artificial Sequence Synthetic peptide
ATWTFEGDYV

SEQ ID NO: 90 - protein, 7 aa, Artificial Sequence
  Description of Artificial Sequence Synthetic peptide
DKLGDHY

SEQ ID NO: 91 - protein, 3 aa, Artificial Sequence
  Description of Artificial Sequence Synthetic peptide
DDS

SEQ ID NO: 92 - protein, 7 aa, Artificial Sequence
  Description of Artificial Sequence Synthetic peptide
WTFEGDY

SEQ ID NO: 93 - protein, 107 aa, Artificial Sequence
  Description of Artificial Sequence Synthetic polypeptide
SYVLTQPPSVSVAPGKTARITCSGDKLGDHYAYWYQQKPGQAPVLVIYDDSKRPSGIPERFSGSNSGNTATLTISRVEAGDEADYYCATWTFEGDYVFGGGTKLTVL

SEQ ID NO: 94 - DNA, 321 bp, Artificial Sequence
  Description of Artificial Sequence Synthetic polynucleotide
tcctacgtcctgacacaacctcccagcgtgagcgtcgctcctggcaagacagccagaatcacctgcagcggcgacaagctgggcgaccactacgcctactggtatcagcagaaacccggccaagctcccgtgctggtgatctatgacgacagcaagagaccctccggcatccctgagagattcagcggaagcaactccggcaacaccgccaccctgaccatcagcagggtcgaagccggcgatgaggccgactactactgcgccacctggacctttgagggcgactacgtgttcggaggcggcaccaagttaaccgtccta

SEQ ID NO: 95 - protein, 213 aa, Artificial Sequence
  Description of Artificial Sequence Synthetic polypeptide
SYVLTQPPSVSVAPGKTARITCSGDKLGDHYAYWYQQKPGQAPVLVIYDDSKRPSGIPERFSGSNSGNTATLTISRVEAGDEADYYCATWTFEGDYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 96 - DNA, 639 bp, Artificial Sequence
  Description of Artificial Sequence Synthetic polynucleotide
tcctacgtcctgacacaacctcccagcgtgagcgtcgctcctggcaagacagccagaatcacctgcagcggcgacaagctgggcgaccactacgcctactggtatcagcagaaacccggccaagctcccgtgctggtgatctatgacgacagcaagagaccctccggcatccctgagagattcagcggaagcaactccggcaacaccgccaccctgaccatcagcagggtcgaagccggcgatgaggccgactactactgcgccacctggacctttgagggcgactacgtgttcggaggcggcaccaagttaaccgtcctaggacagcctaaggccgctccctccgtgacactgtttccccctagcagcgaggagctgcaggccaacaaggccaccctcgtgtgcctcatctccgacttctaccctggcgccgtcacagtcgcctggaaagccgacagctcccccgtcaaagctggcgtggagaccaccacccccagcaagcagagcaacaacaagtacgccgcctcctcctatctgagcctgacccccgagcagtggaagagccacaggagctactcctgccaggtgacacacgagggcagcaccgtcgagaagaccgtcgctcccaccgagtgcagc

SEQ ID NO: 97 - protein, 206 aa, Artificial Sequence
  Description of Artificial Sequence Synthetic polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 98 - protein, 166 aa, Artificial Sequence
  Description of Artificial Sequence Synthetic polypeptide
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 99 - protein, 1658 aa, Artificial Sequence
  Description of Artificial Sequence Synthetic polypeptide
QEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 100 - protein, 442 aa, Artificial Sequence
  Description of Artificial Sequence Synthetic polypeptide
DPVLCFTQYEESSGKCKGLLGGGVSVEDCCLNTAFAYQKRSGGLCQPCRSPRWSLWSTWAPCSVTCSEGSQLRYRRCVGWNGQCSGKVAPGTLEWQLQACEDQQCCPEMGGWSGWGPWEPCSVTCSKGTRTRRRACNHPAPKCGGHCPGQAQESEACDTQQVCPTHGAWATWGPWTPCSASCHGGPHEPKETRSRKCSAPEPSQKPPGKPCPGLAYEQRRCTGLPPCPVAGGWGPWGPVSPCPVTCGLGQTMEQRTCNHPVPQHGGPFCAGDATRTHICNTAVPCPVDGEWDSWGEWSPCIRRNMKSISCQEIPGQQSRGRTCRGRKFDGHRCAGQQQDIRHCYSIQHCPLKGSWSEWSTWGLCMPPCGPNPTRARQRLCTPLLPKYPPTVSMVEGQGEKNVTFWGRPLPRCEELQGQKLVVEEKRPCLHVPACKDPEEEEL

SEQ ID NO: 101 - protein, 157 aa, Artificial Sequence [sequence omitted]

SEQ ID NO: 102 - protein, 269 aa, Artificial Sequence
  Description of Artificial Sequence Synthetic polypeptide
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 103 - DNA, 294 bp, Artificial Sequence
  Description of Artificial Sequence Synthetic polynucleotide
ggagtctatcacagagaggctagatcaggcaagtataagctgacctacgccgaggctaaggccgtgtgcgagttcgagggcggtcacctggctacctataagcagctggaagccgctagaaagatcggctttcacgtgtgcgccgctggctggatggctaagggtagagtgggctaccctatcgtgaagcctggccctaactgcggcttcggtaaaaccggaattatcgactacgggattaggctgaatagatcagagcgctgggacgcctactgctataaccctcacgctaag

SEQ ID NO: 104 - DNA, 291 bp, Artificial Sequence
  Description of Artificial Sequence Synthetic polynucleotide
ggagtctatcacagagaggctcagtcaggcaagtataagctgacctacgccgaggctaaggccgtgtgcgagttcgagggcggtcacctggctacctataagcagctggaagccgctagaaagatcggctttcacgtgtgcgccgctggctggatggctaagggtagagtgggctaccctatcgtgaagcctggccctaactgcggcttcggtaaaaccggaattatcgactacgggattaggctgaatagatcagagcgctgggacgcctactgctataaccctcacgcc

SEQ ID NO: 105 - DNA, 291 bp, Artificial Sequence
  Description of Artificial Sequence Synthetic polynucleotide
ggagtctatcacagagaggctgctagcggtaaatacaagctgacctacgccgaggctaaggccgtgtgcgagttcgagggcggtcacctggctacctataagcagctggaagccgctagaaagatcggctttcacgtgtgcgccgctggctggatggctaagggtagagtgggctaccctatcgtgaagcctggccctaactgcggcttcggtaaaaccggaattatcgactacgggattaggctgaatagatcagagcgctgggacgcctactgctataaccctcacgcc

SEQ ID NO: 106 - DNA, 300 bp, Artificial Sequence
  Description of Artificial Sequence Synthetic polynucleotide
ggcgcctgtggcgtgtatcacagggaggcccagagcggcaagtacaagctcacctacgccgaggccaaggccgtgtgcgaattcgagggcggccacctggccacctacaagcagctggagtgcgccaggaagatcggcttccacgtgtgtgccgccggctggatggccaaaggcagagtgggctaccccatcgtgaaacccggccccaactgcggcttcggcaagacaggcatcatcgactacggcatcaggctgaacaggagcgagaggtgggacgcctactgctacaacccccacgcc

SEQ ID NO: 107 - DNA, 291 bp, Artificial Sequence
  Description of Artificial Sequence Synthetic polynucleotide
ggagtgtatcacagagaggcccagagcggcaagtacaagctgacctacgccgaggccaaggccgtgtgtgagttcgagggcggccacctgtgcacctacaagcagctggaggccgccaggaagatcggcttccacgtgtgtgccgccggctggatggctaaaggcagggtgggctaccccattgtgaagcccggccccaattgcggcttcggcaagaccggcatcatcgactacggcatcaggctgaacaggagcgagaggtgggacgcctactgctgcaacccccacgcc

SEQ ID NO: 108 - protein, 11 aa, Artificial Sequence
  Description of Artificial Sequence Synthetic peptide
GFTISRSYWIC

SEQ ID NO: 109 - protein, 18 aa, Artificial Sequence
  Description of Artificial Sequence Synthetic peptide
CIYGDNDITPLYANWAKG

SEQ ID NO: 110 - protein, 10 aa, Artificial Sequence
  Description of Artificial Sequence Synthetic peptide
LGYADYAYDL

SEQ ID NO: 111 - protein, 121 aa, Artificial Sequence
  Description of Artificial Sequence Synthetic polypeptide
EVQLVESGGGSVQPGGSLRLSCTASGFTISRSYWICWVRQAPGKGLEWVGCIYGDNDITPLYANWAKGRFTISRDTSKNTVYLQMNSLRAEDTATYYCARLGYADYAYDLWGQGTTVTVSS

SEQ ID NO: 112 - DNA, 363 bp, Artificial Sequence
  Description of Artificial Sequence Synthetic polynucleotide
gaggtccagctggtggagagcggaggaggaagcgtccagcctggaggcagcctgagactgagctgcaccgccagcggcttcaccatcagcaggagctactggatctgctgggtgaggcaggctcctggcaagggactcgagtgggtgggctgcatctacggcgacaacgacatcacccccctctacgccaactgggctaagggcaggttcaccattagcagggacaccagcaagaacaccgtgtacctccagatgaacagcctgagggccgaggataccgccacctactattgcgccaggctgggctacgccgattacgcctatgacctctggggccagggcaccacagtgaccgtcagctca

SEQ ID NO: 113 - protein, 224 aa, Artificial Sequence
  Description of Artificial Sequence Synthetic polypeptide
EVQLVESGGGSVQPGGSLRLSCTASGFTISRSYWICWVRQAPGKGLEWVGCIYGDNDITPLYANWAKGRFTISRDTSKNTVYLQMNSLRAEDTATYYCARLGYADYAYDLWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 114 - DNA, 672 bp, Artificial Sequence
  Description of Artificial Sequence Synthetic polynucleotide
gaggtccagctggtggagagcggaggaggaagcgtccagcctggaggcagcctgagactgagctgcaccgccagcggcttcaccatcagcaggagctactggatctgctgggtgaggcaggctcctggcaagggactcgagtgggtgggctgcatctacggcgacaacgacatcacccccctctacgccaactgggctaagggcaggttcaccattagcagggacaccagcaagaacaccgtgtacctccagatgaacagcctgagggccgaggataccgccacctactattgcgccaggctgggctacgccgattacgcctatgacctctggggccagggcaccacagtgaccgtcagctcagcctccaccaagggaccttccgtgttccccctggcccctagctccaagtccaccagcggaggaacagccgctctgggctgtctggtgaaggactacttccccgagcctgtgaccgtgtcctggaattccggcgccctcacaagcggagtgcataccttccccgccgtgctgcaaagctccggactgtactccctctccagcgtggtgacagtgccttccagcagcctcggcacccagacctacatctgcaacgtgaaccacaagccctccaataccaaggtggacaagagggtcgagcctaaaagctgt

SEQ ID NO: 115 - protein, 326 aa, Artificial Sequence
  Description of Artificial Sequence Synthetic polypeptide
EVQLVESGGGSVQPGGSLRLSCTASGFTISRSYWICWVRQAPGKGLEWVGCIYGDNDITPLYANWAKGRFTISRDTSKNTVYLQMNSLRAEDTATYYCARLGYADYAYDLWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 116 - DNA, 978 bp, Artificial Sequence
  Description of Artificial Sequence Synthetic polynucleotide
gaggtccagctggtggagagcggaggaggaagcgtccagcctggaggcagcctgagactgagctgcaccgccagcggcttcaccatcagcaggagctactggatctgctgggtgaggcaggctcctggcaagggactcgagtgggtgggctgcatctacggcgacaacgacatcacccccctctacgccaactgggctaagggcaggttcaccattagcagggacaccagcaagaacaccgtgtacctccagatgaacagcctgagggccgaggataccgccacctactattgcgccaggctgggctacgccgattacgcctatgacctctggggccagggcaccacagtgaccgtcagctcagcctccaccaagggaccttccgtgttccccctggcccctagctccaagtccaccagcggaggaacagccgctctgggctgtctggtgaaggactacttccccgagcctgtgaccgtgtcctggaattccggcgccctcacaagcggagtgcataccttccccgccgtgctgcaaagctccggactgtactccctctccagcgtggtgacagtgccttccagcagcctcggcacccagacctacatctgcaacgtgaaccacaagccctccaataccaaggtggacaagagggtcgagcctaaaagctgtggatccggaggaggcggcgtgtatcatagagaggcccagtccggcaagtacaagctgacctacgccgaagccaaggccgtgtgtgagttcgagggcggacacctggctacctacaaacagctcgaagccgctaggaagatcggattccacgtgtgcgccgccggatggatggccaaaggcagagtgggctaccccattgtcaagcccggacccaactgcggattcggcaagaccggcatcatcgactacggcatcaggctcaacaggtccgagagatgggacgcttactgctacaatccccacgcc

SEQ ID NO: 117 - protein, 13 aa, Artificial Sequence
  Description of Artificial Sequence Synthetic peptide
QSSQSVYGNIWMA

SEQ ID NO: 118 - protein, 7 aa, Artificial Sequence
  Description of Artificial Sequence Synthetic peptide
QASKLAS

SEQ ID NO: 119 - protein, 11 aa, Artificial Sequence
  Description of Artificial Sequence Synthetic peptide
QGNFNTGDRYA

SEQ ID NO: 120 - protein, 113 aa, Artificial Sequence
  Description of Artificial Sequence Synthetic polypeptide
EIVMTQSPSTLSASVGDRVIITCQSSQSVYGNIWMAWYQQKPGRAPKLLIYQASKLASGVPSRFSGSGSGAEFTLTISSLQPDDFATYYCQGNFNTGDRYAFGQGTKLTVLKR

SEQ ID NO: 121 - DNA, 339 bp, Artificial Sequence
  Description of Artificial Sequence Synthetic polynucleotide
gagatcgtcatgacccagagccccagcacactcagcgcctccgtgggagacagggtgatcatcacctgccagtcctcccagtccgtgtacggcaacatctggatggcctggtaccagcagaagcccggcagagcccccaagctgctgatctaccaggccagcaagctcgcctccggagtgcccagcagattttccggctccggatccggagccgagttcacactgaccatcagcagcctgcagcccgatgacttcgccacctactattgccagggcaacttcaacaccggcgacaggtacgcctttggccagggcaccaagctgaccgtcctcaagcgt

SEQ ID NO: 122 - protein, 219 aa, Artificial Sequence
  Description of Artificial Sequence Synthetic polypeptide
EIVMTQSPSTLSASVGDRVIITCQSSQSVYGNIWMAWYQQKPGRAPKLLIYQASKLASGVPSRFSGSGSGAEFTLTISSLQPDDFATYYCQGNFNTGDRYAFGQGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - DNA, 657 bp, Artificial Sequence
  Description of Artificial Sequence Synthetic polynucleotide
gagatcgtcatgacccagagccccagcacactcagcgcctccgtgggagacagggtgatcatcacctgccagtcctcccagtccgtgtacggcaacatctggatggcctggtaccagcagaagcccggcagagcccccaagctgctgatctaccaggccagcaagctcgcctccggagtgcccagcagattttccggctccggatccggagccgagttcacactgaccatcagcagcctgcagcccgatgacttcgccacctactattgccagggcaacttcaacaccggcgacaggtacgcctttggccagggcaccaagctgaccgtcctcaagcgtacggtggctgctcccagcgtcttcatcttcccccccagcgatgagcagctcaagagcggcacagcctccgtggtgtgcctcctgaacaacttctaccctagggaggccaaggtgcaatggaaggtggacaacgccctgcagagcggcaacagccaggagtccgtgaccgagcaggactccaaggacagcacctacagcctgagcagcacactcaccctgagcaaagccgactacgagaagcacaaggtctacgcctgcgaggtgacccatcagggcctgtccagccccgtgaccaagagcttcaacagaggcgagtgc

SEQ ID NO: 124 - protein, 4 aa, Artificial Sequence
  Description of Artificial Sequence Synthetic peptide
GSGG

SEQ ID NO: 125 - protein, 108 aa, Homo sapiens
KRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 103 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 127 - protein, 103 aa, Artificial Sequence
  Description of Artificial Sequence Synthetic polypeptide
GSGGGGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAK

SEQ ID NO: 128 - protein, 31 aa, Artificial Sequence
  Description of Artificial Sequence Synthetic polypeptide
GSGGGKQKIKHVVKLKGSGGGKLKSQLVKRK

SEQ ID NO: 129 - protein, 46 aa, Artificial Sequence
  Description of Artificial Sequence Synthetic polypeptide
GSGGGKNGRYSISRGSGGGRDGTRYVQKGEYRGSGGGRRRCGQKKK

SEQ ID NO: 130 - protein, 109 aa, Artificial Sequence
  Description of Artificial Sequence Synthetic polypeptide
GSGGGVFPYHPRGGRYKLTFAEAQRACAEQDGILASAEQLHAAWRDGLDWCNAGWLRDGSVQYPVNRPREPCGGLGGTGSAGGGGDANGGLRNYGYRHNAEERYDAFCF

SEQ ID NO: 131 - protein, 101 aa, Artificial Sequence
  Description of Artificial Sequence Synthetic polypeptide
GSGGEVFYVGPARRLTLAGARAQCRRQGAALASVGQLHLAWHEGLDQCDPGWLADGSVRYPIQTPRRRCGGPAPGVRTVYRFANRTGFPSPAERFDAYCFR

SEQ ID NO: 132 - protein, 73 aa, Artificial Sequence
  Description of Artificial Sequence Synthetic polypeptide
GSGGLKQKIKHVVKLKDENSQLKSEVSKLRSQLVKRKQNGSGGAHWQFNALTVRGGGSSTMMSRSHKTRSHHV

SEQ ID NO: 133 - protein, 103 aa, Artificial Sequence
  Description of Artificial Sequence Synthetic polypeptide
GSGGGVFHLRSPLGQYKLTFDKAREACANEAATMATYNQLSYAQKAKYHLCSAGWLETGRVAYPTAFASQNCGSGVVGIVDYGPRPNKREMWDVFCYRMKDVN

SEQ ID NO: 134 - protein, 48 aa, Artificial Sequence
  Description of Artificial Sequence Synthetic polypeptide
GSGGGHQNLKQKIKHVVKLKDENSQLKSEVSKLRSQLAKKKQSETKLQ

SEQ ID NO: 135 - protein, 106 aa, Artificial Sequence
  Description of Artificial Sequence Synthetic polypeptide
GSGGGGVYHREARSGKYKLTYAEAKAVCEFEGGHLATYKQLEAARKIGFHVCSAGWLETGRVAYPTAFASQNCGSGVVGIVDYGIRLQRSERWDAYCYNPHAKAHP

SEQ ID NO: 136 - protein, 29 aa, Artificial Sequence
  Description of Artificial Sequence Synthetic peptide
GSGGGKVGKSPPVRGSGGGHREARSGKYK

SEQ ID NO: 137 - protein, 26 aa, Artificial Sequence
  Description of Artificial Sequence Synthetic peptide
GSKQKIKHVVKLKGGGSREARSGKYK

SEQ ID NO: 138 - protein, 40 aa, Artificial Sequence
  Description of Artificial Sequence Synthetic polypeptide
GSGGGKGGNGEPRGDTYRAYGSGGGKGGPQVTRGDVFTMP

SEQ ID NO: 139 - protein, 24 aa, Artificial Sequence
  Description of Artificial Sequence Synthetic peptide
GSGGGRRANAALKAGELYKSILYG

SEQ ID NO: 140 - protein, 24 aa, Artificial Sequence
  Description of Artificial Sequence Synthetic peptide
GSGGGRRANAALKAGELYKSILYG

SEQ ID NO: 141 - protein, 114 aa, Artificial Sequence
  Description of Artificial Sequence Synthetic polypeptide
GSGGGTCRYAGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNASAPPEEDCT

SEQ ID NO: 142 - protein, 214 aa, Artificial Sequence
  Description of Artificial Sequence Synthetic polypeptide
DIQVTQSPSSLSASVGDRVTITCITSTDIDDDMNWYQQKPGKVPKLLISGGNTLRPGVPSRFSGSGSGTDFTLTISSLQPEDVATYYCLQSDSLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 216 aa, Artificial Sequence
  Description of Artificial Sequence Synthetic polypeptide
QLVQSGPELKKPGASVKVSCKASGYTFTNYGMNWVRQAPGQGLEWMGWINTYTGETTYADDFKGRFVFSLDTSVSTAYLQISSLKAEDTAVYYCEREGGVNNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 144 - protein, 214 aa, Artificial Sequence; identical to SEQ ID NO: 142
  Description of Artificial Sequence Synthetic polypeptide

SEQ ID NO: 145 - protein, 318 aa, Artificial Sequence
  Description of Artificial Sequence Synthetic polypeptide
QLVQSGPELKKPGASVKVSCKASGYTFTNYGMNWVRQAPGQGLEWMGWINTYTGETTYADDFKGRFVFSLDTSVSTAYLQISSLKAEDTAVYYCEREGGVNNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 146 - protein, 469 aa, Artificial Sequence
  Description of Artificial Sequence Synthetic polypeptide
GGGGGPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPRIPKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCRVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 147 - protein, 571 aa, Artificial Sequence
  Description of Artificial Sequence Synthetic polypeptide
GGGGGPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPRIPKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCRVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 148 - protein, 166 aa, Artificial Sequence; identical to SEQ ID NO: 98
  Description of Artificial Sequence Synthetic polypeptide

SEQ ID NO: 149 - protein, 276 aa, Artificial Sequence
  Description of Artificial Sequence Synthetic polypeptide
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDRGSGGGGVYHREAQSGKYYLTYAEAKAVCEFEGGHLATYKQLEAARKIGFHVCAAGWMAKGRVGYPIVKPGPNCGFGKTGIIDYGIRLNRSERWDAYCYNPHAGSHHHHHH

SEQ ID NO: 150 - DNA, 30 bp, Artificial Sequence
  Description of Artificial Sequence Synthetic oligonucleotide
caggcuacgcgtagagcaucatgatccugt

SEQ ID NO: 151 - protein, 120 aa, Artificial Sequence
  Description of Artificial Sequence Synthetic polypeptide
LPETGGGGGGSGGGGVYHREAQSGKYYLTYAEAKAVCEFEGGHLATYKQLEAARKIGFHVCAAGWMAKGRVGYPIVKPGPNCGFGKTGIIDYGIRLNRSERWDAYCYNPHAGGSHHHHHH

SEQ ID NO: 152 - protein, 160 aa, Artificial Sequence
  Description of Artificial Sequence Synthetic polypeptide
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 153 - protein, 272 aa, Artificial Sequence
  Description of Artificial Sequence Synthetic polypeptide
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHAGSGGHHHHHH

SEQ ID NO: 154 - protein, 432 aa, Artificial Sequence
  Description of Artificial Sequence Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 4 aa, Artificial Sequence
  Description of Artificial Sequence Synthetic peptide
SGGG

SEQ ID NO: 156 - protein, 533 aa, Artificial Sequence
  Description of Artificial Sequence Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGSGGGGVYHREAISGKYYLTYAEAKAVCEFEGGHLATYKQLEAAQQIGFHVCAAGWMAKGRVGYPIVKPGPNCGFGKTGIIDYGIRLQRSERWDAYCYNPHA

SEQ ID NO: 157 - protein, 453 aa, Artificial Sequence
  Description of Artificial Sequence Synthetic polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 214 aa, Artificial Sequence
  Description of Artificial Sequence Synthetic polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - protein, 555 aa, Artificial Sequence
  Description of Artificial Sequence Synthetic polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 160 - protein, 214 aa, Artificial Sequence; identical to SEQ ID NO: 158
  Description of Artificial Sequence Synthetic polypeptide

SEQ ID NO: 161 - protein, 450 aa, Artificial Sequence
  Description of Artificial Sequence Synthetic polypeptide
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 218 aa, Artificial Sequence; identical to SEQ ID NO: 19
  Description of Artificial Sequence Synthetic polypeptide

SEQ ID NO: 163 - protein, 450 aa, Artificial Sequence; identical to SEQ ID NO: 161
  Description of Artificial Sequence Synthetic polypeptide

SEQ ID NO: 164 - protein, 320 aa, Artificial Sequence
  Description of Artificial Sequence Synthetic polypeptide
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 165 - protein, 259 aa, Artificial Sequence
  Description of Artificial Sequence Synthetic polypeptide
MEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGGSGGGGSGGGGSSGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSHHHHHH

SEQ ID NO: 166 - protein, 361 aa, Artificial Sequence
  Description of Artificial Sequence Synthetic polypeptide
MEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGGSGGGGSGGGGSSGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHAHHHHHH

SEQ ID NO: 167 - protein, 142 aa, Artificial Sequence
  Description of Artificial Sequence Synthetic polypeptide
SDLGKKLLEAARAGQDDEVRILMANGADVNTADSTGWTPLHLAVPWGHLEIVEVLLKYGADVNAKDFQGWTPLHLAAAIGHQEIVEVLLKNGADVNAQDKFGKTAFDISIDNGNEDLAEILQKAAGSLPETGGGSGHHHHHH

SEQ ID NO: 168 - protein, 237 aa, Artificial Sequence
  Description of Artificial Sequence Synthetic polypeptide
SDLGKKLLEAARAGQDDEVRILMANGADVNTADSTGWTPLHLAVPWGHLEIVEVLLKYGADVNAKDFQGWTPLHLAAAIGHQEIVEVLLKNGADVNAQDKFGKTAFDISIDNGNEDLAEILQKAAGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHAGSGGHHHHHH

SEQ ID NO: 169 - protein, 164 aa, Artificial Sequence
  Description of Artificial Sequence Synthetic polypeptide
SDLGKKLLEAARAGQDDEVRILMANGADVNAFDWMGWTPLHLAAHEGHLEIVEVLLKNGADVNATDVSGYTPLHLAAADGHLEIVEVLLKYGADVNTKDNTGWTPLHLSADLGRLEIVEVLLKYGADVNAQDKFGKTAFDISIDNGNEDLAEILQKAAHHHHHH

SEQ ID NO: 170 - protein, 270 aa, Artificial Sequence
  Description of Artificial Sequence Synthetic polypeptide
SDLGKKLLEAARAGQDDEVRILMANGADVNAFDWMGWTPLHLAAHEGHLEIVEVLLKNGADVNATDVSGYTPLHLAAADGHLEIVEVLLKYGADVNTKDNTGWTPLHLSADLGRLEIVEVLLKYGADVNAQDKFGKTAFDISIDNGNEDLAEILQKAAGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHAGSGGHHHHHH

SEQ ID NO: 171 - protein, 325 aa, Artificial Sequence; identical to SEQ ID NO: 23
  Description of Artificial Sequence Synthetic polypeptide

SEQ ID NO: 172 - protein, 223 aa, Artificial Sequence
  Description of Artificial Sequence Synthetic polypeptide
GGGGGEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 326 aa, Artificial Sequence
  Description of Artificial Sequence Synthetic polypeptide
VYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGS

SEQ ID NO: 174 - protein, 319 aa, Artificial Sequence
  Description of Artificial Sequence Synthetic polypeptide
VYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKGGGSEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 325 aa, Artificial Sequence; identical to SEQ ID NO: 23
  Description of Artificial Sequence Synthetic polypeptide

SEQ ID NO: 176 - protein, 320 aa, Artificial Sequence; identical to SEQ ID NO: 164
  Description of Artificial Sequence Synthetic polypeptide

SEQ ID NO: 177 - protein, 225 aa, Artificial Sequence
  Description of Artificial Sequence Synthetic polypeptide
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGS

SEQ ID NO: 178 - protein, 422 aa, Artificial Sequence
  Description of Artificial Sequence Synthetic polypeptide
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHAGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 179 - protein, 325 aa, Artificial Sequence; identical to SEQ ID NO: 21
  Description of Artificial Sequence Synthetic polypeptide

SEQ ID NO: 180 - protein, 319 aa, Artificial Sequence; identical to SEQ ID NO: 174
  Description of Artificial Sequence Synthetic polypeptide

SEQ ID NO: 181 - protein, 633 aa, Artificial Sequence
  Description of Artificial Sequence Synthetic polypeptide
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAGGGGGGSGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAGSGGGGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAGSGGGGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 182 - protein, 218 aa, Artificial Sequence; identical to SEQ ID NO: 19
  Description of Artificial Sequence Synthetic polypeptide

SEQ ID NO: 183 - protein, 427 aa, Artificial Sequence
  Description of Artificial Sequence Synthetic polypeptide
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAGSGGGGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 184 - protein, 218 aa, Artificial Sequence; identical to SEQ ID NO: 19
  Description of Artificial Sequence Synthetic polypeptide

SEQ ID NO: 185 - protein, 320 aa, Artificial Sequence; identical to SEQ ID NO: 164
  Description of Artificial Sequence Synthetic polypeptide

SEQ ID NO: 186 - protein, 20 aa, Artificial Sequence
  Description of Artificial Sequence Synthetic peptide
GGGGSGGGGSGGGGSGGGGS

SEQ ID NO: 187 - protein, 5 aa, Artificial Sequence
  Description of Artificial Sequence Synthetic peptide
GGGGG

SEQ ID NO: 188 - protein, 6 aa, Artificial Sequence
  Description of Artificial Sequence Synthetic 6xHis tag
HHHHHH

SEQ ID NO: 189 - protein, 10 aa, Artificial Sequence
  Description of Artificial Sequence Synthetic peptide
GFTFSVYGMN

SEQ ID NO: 190 - protein, 17 aa, Artificial Sequence
  Description of Artificial Sequence Synthetic peptide
IIWYDGDNQYYADSVKG

SEQ ID NO: 191 - protein, 9 aa, Artificial Sequence
  Description of Artificial Sequence Synthetic peptide
DLRTGPFDY

SEQ ID NO: 192 - protein, 118 aa, Artificial Sequence [sequence omitted]

SEQ ID NO: 193 - DNA, 354 bp, Artificial Sequence
  Description of Artificial Sequence Synthetic polynucleotide
caggtgcagctggtggaatctggcggcggagtggtgcagcctggcagaagcctgagactgagctgtgccgccagcggcttcaccttcagcgtgtacggcatgaactgggtgcgccaggcccctggcaaaggcctggaatgggtggccatcatttggtacgacggcgacaaccagtactacgccgacagcgtgaagggccggttcaccatcagccgggacaacagcaagaacaccctgtacctgcagatgaacggcctgcgggccgaggataccgccgtgtactactgcgccagggacctgagaacaggccccttcgattattggggccagggcaccctcgtgaccgtgtctagc

SEQ ID NO: 194 - protein, 221 aa, Artificial Sequence
  Description of Artificial Sequence Synthetic polypeptide
QVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 195 - DNA, 663 bp, Artificial Sequence
  Description of Artificial Sequence Synthetic polynucleotide
caggtgcagctggtggaatctggcggcggagtggtgcagcctggcagaagcctgagactgagctgtgccgccagcggcttcaccttcagcgtgtacggcatgaactgggtgcgccaggcccctggcaaaggcctggaatgggtggccatcatttggtacgacggcgacaaccagtactacgccgacagcgtgaagggccggttcaccatcagccgggacaacagcaagaacaccctgtacctgcagatgaacggcctgcgggccgaggataccgccgtgtactactgcgccagggacctgagaacaggccccttcgattattggggccagggcaccctcgtgaccgtgtctagcgcctctacaaagggccccagcgtgttccctctggcccctagcagcaagtctaccagcggaggaacagccgccctgggctgcctcgtgaaggactactttcccgagcccgtgacagtgtcctggaactctggcgccctgacaagcggcgtgcacacctttccagccgtgctgcagagcagcggcctgtactctctgagcagcgtcgtgactgtgcccagcagctctctgggcacccagacctacatctgcaacgtgaaccacaagcccagcaacaccaaggtggacaagcgggtggaacccaagagctgt

SEQ ID NO: 196 - protein, 323 aa, Artificial Sequence
  Description of Artificial Sequence Synthetic polypeptide
QVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 197 - DNA, 663 bp, Artificial Sequence; identical to SEQ ID NO: 195
  Description of Artificial Sequence Synthetic polynucleotide

SEQ ID NO: 198 - protein, 11 aa, Artificial Sequence
  Description of Artificial Sequence Synthetic peptide
RASQSIGSSLH

SEQ ID NO: 199 - protein, 7 aa, Artificial Sequence
  Description of Artificial Sequence Synthetic peptide
YASQSFS

SEQ ID NO: 200 - protein, 9 aa, Artificial Sequence
  Description of Artificial Sequence Synthetic peptide
HQSSSLPFT

SEQ ID NO: 201 - protein, 108 aa, Artificial Sequence
  Description of Artificial Sequence Synthetic polypeptide
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIKR

SEQ ID NO: 202 - protein, 214 aa, Artificial Sequence
  Description of Artificial Sequence Synthetic polypeptide
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - DNA, 642 bp, Artificial Sequence
  Description of Artificial Sequence Synthetic polynucleotide
gagatcgtgctgacccagagccccgactttcagagcgtgacccccaaagaaaaagtgaccatcacctgtcgggccagccagagcatcggctctagcctgcactggtatcagcagaagcccgaccagtcccccaagctgctgattaagtacgccagccagtccttcagcggcgtgcccagcagattttctggcagcggctccggcaccgacttcaccctgaccatcaacagcctggaagccgaggacgccgctgcctactactgtcaccagagcagcagcctgcccttcacctttggccctggcaccaaggtggacatcaagcggacagtggccgctccctccgtgttcatcttcccacctagcgacgagcagctgaagtctggcacagccagcgtcgtgtgcctgctgaacaacttctacccccgcgaggccaaggtgcagtggaaagtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgc

SEQ ID NO: 204 - protein, 15 aa, Artificial Sequence
  Description of Artificial Sequence Synthetic peptide
GGGGSGGGGSGGGGS

SEQ ID NO: 205 - protein, 4 aa, Artificial Sequence
  Description of Artificial Sequence Synthetic peptide
GGGS

SEQ ID NO: 206 - protein, 5 aa, Artificial Sequence
  Description of Artificial Sequence Synthetic peptide
GGGGS

SEQ ID NO: 207 - protein, 5 aa, Artificial Sequence
  Description of Artificial Sequence Synthetic peptide
GGGGA

SEQ ID NO: 208 - protein, 4 aa, Artificial Sequence
  Description of Artificial Sequence Synthetic peptide
GGGA

SEQ ID NO: 209 - protein, 5 aa, Artificial Sequence
  Description of Artificial Sequence Synthetic peptide
EAAAK

SEQ ID NO: 210 - protein, 12 aa, Artificial Sequence
  Description of Artificial Sequence Synthetic peptide
GPSVFPLAPSSK

SEQ ID NO: 211 - protein, 9 aa, Artificial Sequence
  Description of Artificial Sequence Synthetic peptide
TGIIDYGIR

SEQ ID NO: 212 - protein, 14 aa, Artificial Sequence
  Description of Artificial Sequence Synthetic peptide
FNWYVDGVEVHNAK

SEQ ID NO: 213 - protein, 5 aa, Artificial Sequence
  Description of Artificial Sequence Synthetic peptide
HHHHH

SEQ ID NO: 214 - DNA, 324 bp, Artificial Sequence
  Description of Artificial Sequence Synthetic polynucleotide
gagatcgtgctgacccagagccccgactttcagagcgtgacccccaaagaaaaagtgaccatcacctgtcgggccagccagagcatcggctctagcctgcactggtatcagcagaagcccgaccagtcccccaagctgctgattaagtacgccagccagtccttcagcggcgtgcccagcagattttctggcagcggctccggcaccgacttcaccctgaccatcaacagcctggaagccgaggacgccgctgcctactactgtcaccagagcagcagcctgcccttcacctttggccctggcaccaaggtggacatcaagcgg

SEQ ID NO: 215 - protein, 225 aa, Artificial Sequence; identical to SEQ ID NO: 177
  Description of Artificial Sequence Synthetic polypeptide